(19) United States
(12) Patent Application Publication
(10) Pub. No.: US 2005/0214757 A1
(43) Pub. Date: Sep. 29, 2005

(54) Diagnosis and therapy of conditions by detection or modulation of the alms1 gene or protein

(76) Inventors: David Ian Wilson, Southampton (GB); Thomas Hearn, Southampton (GB); Mark Walker, Newcastle Upon Tyne (GB)

Correspondence Address: NIXON & VANDERHYE, PC, 1100 N GLEBE ROAD, 8TH FLOOR, ARLINGTON, VA, 22201-4714, US

(21) Appl. No.: 10/492,634

(22) PCT Filed: Oct. 15, 2002
(86) PCT No.: PCT/GB02/04658

§ 371 Date: Oct. 5, 2004

(30) Foreign Application Priority Data
Oct. 15, 2001 (GB) 01246214
Oct. 22, 2001 (GB) 01253186
Jan. 7, 2002 (GB) 02002483
Feb. 8, 2002 (GB) 2030393
Feb. 8, 2002 (GB) 02030401

(51) Int. Cl. C12Q 1/68

(52) U.S. Cl. 435/6

(57) ABSTRACT

A method of diagnosing the presence of, or susceptibility to, retinal dystrophy, cardiomyopathy endocrinopathy, diabetes or Alstrom disease in an individual, which method comprises typing in a sample from the individual the ALMS1 protein or ALMS1 gene region of the individual, or detecting aberrant ALMS1 activity, and thereby determining whether the individual has, or is susceptible to, retinal dystrophy, cardiomyopathy, endocrinopathy, diabetes or Alstrom disease. Use of an agent that modulates (i) the ALMS1 protein, or (ii) a component that affects or is affected by ALMS1, in the manufacture of a medicament for prevention or treating retinal dystrophy, cardiomyopathy, endocrinopathy, diabetes or Alstrom disease.

### FIELD OF THE INVENTION

The present invention relates to diagnosis and treatment of retinal dystrophy, cardiomyopathy, endocrinopathy and diabetes, and to novel polynucleotides and polypeptides.

### BACKGROUND OF THE INVENTION

Retinal dystrophy, cardiomyopathy, endocrinopathy and diabetes are complex disorders which may be caused by many factors, including environmental and genetic factors. However, it can be difficult to identify such factors as their individual contributions may be small.

### SUMMARY OF THE INVENTION

The inventors have found that mutations in a novel gene (named ALMS1) cause retinal dystrophy, cardiomyopathy, endocrinopathy and diabetes. In individuals with the mutations these conditions are present in the form of Alstrom syndrome. SEQ ID NO's 1 and 2 show the sequence of the ALMS1 coding sequence and protein.

Thus detection of mutations in the ALMS1 gene or protein, or detection of aberrant ALMS1 activity may be used to diagnose retinal dystrophy, cardiomyopathy, endocrinopathy or diabetes. In addition the present finding allows prevention or treatment of these conditions by modulation of ALMS1 activity.

Accordingly, the invention provides a method of diagnosing the presence of, or susceptibility to, retinal dystrophy, cardiomyopathy, endocrinopathy, diabetes or Alstrom syndrome in an individual, which method comprises
(i) typing in a sample from the individual the ALMS1 protein or ALMS1 gene region of the individual, or
(ii) detecting aberrant ALMS1 activity in a sample from the individual, and thereby determining whether the individual has, or is susceptible to retinal dystrophy, cardiomyopathy, diabetes, endocrinopathy or Alstrom syndrome.

The invention also provides use of an agent that modulates (i) the ALMS1 protein, or (ii) a component affected by the ALMS1 protein or which affects the ALMS1 protein in the manufacture of a medicament for preventing or treating retinal dystrophy, cardiomyopathy, diabetes, endocrinopathy or Alstrom syndrome.

In addition the invention provides an isolated polypeptide which is
(i) a polypeptide comprising the amino acid sequence of SEQ ID NO: 2,
(ii) a variant of (i) which is able to complement ALMSI activity or
(iii) a fragment of (i) or (ii) which has a length of at least 15 amino acids.

The invention also provides an isolated polynucleotide that comprises a sequence:
(a) which is the same as SEQ ID NO: 1 or complementary thereto;
(b) which hybridises to (a);
(c) that is degenerate as a result of the genetic code to a sequence as defined in (a) or (b); or
(d) having at least 80% identity to a sequence as defined in (a), (b) or (c).

The invention additionally provides a method of identifying a therapeutic agent comprising contacting a candidate substance with
(i) the ALMS1 protein, a component that regulates ALMS1 or a component that is affected by ALMS 1,
(ii) any part of the expression pathway for (i), or
(iii) a functional analogue of (i) or (ii),
   and determining whether the candidate substance binds or modulates (i), (ii) or (iii).

### DESCRIPTION OF THE DRAWINGS

FIG. 1 shows the gene structure of ALMS1 and protein. FIG. 1a shows the intron and exon organisation (not to scale). ALMS1 exons are dark and the copies of exons 16-20 in the duplicated region are in a lighter colour. CML1 exons are in the opposite orientation to ALMS1 (see arrows). The position of the 2p13 translocation breakpoint is shown as a dotted line. Genomic BAC contig is beneath: the sequence of RP11-434P11 is in 2 pieces each ˜100 kb (not to scale). FIG. 1b show the primary structure of ALMS1 and the position of premature STOP codons causing protein truncation.

FIG. 2 shows the tandem amino acid repeat. The alignment amino acids 498-2159 shows 34 copies of a 47 amino acid tandem repeat. The repeat unit is not perfect (length 45-50 and identity 40-90%). It is interrupted by an insertion of 60 amino acids (between 1958 and 2019) which for purposes of alignment, are not shown. The tandem repeats are coded entirely within exon 7 and contain no cysteine residues.

FIG. 3 shows the genomic sequence at the 5′ end of ALMS 1. The exon-intron boundary is shown and the GT splice donor site is underlined. RT-PCR between exon 1 and exon 2 confirms this splice site. The open reading frame is indicated and is shown continuing to the first upstream STOP codon. We propose the ATG under RT3 is the start codon. This appears to be the case because the context of the proposed ATG start codon [ACCAACATGG] is in good agreement with the optimal Kozak translation initiation sequence (GCCACCAUGG). This exon is also within a CpG island.

In addition CCCAGG (between Pr3F and RT1) corresponds with transcript initiator sequence (Inr) YYCARR for RNA Polymerase II. Also forward primers (RT1-7) that successfully generate PCR product of appropriate size using cDNA as template if reverse primer is within exon 2, 3, 4, 5, and 6, or 5′ end of exon 7. No PCR product is obtained with these primer combinations if genomic DNA is used as template. Forward primers (Pr1F, Pr2F and Ex1F) fail to generate a PCR product using cDNA as template and reverse primer within exon 1 or 2.

Reverse primer (Ex1R) in genomic sequence (intron 1). PCR is successful if this reverse primer is used with any red or black forward primer using genomic DNA as template. None are successful if cDNA is used as template. Pr3f shows sequence that is upstream of the proposed transcript initiator region. Due to high GC content we have been unable generate suitable primer sequence in the potential ORF (5′ of our proposed transcript initiator) to determine whether it is within an exon or not.

Thus the results from RT-PCR primer combinations are consistent with exon 1 extending from the Inr (CCCAGG) to intron/exon boundary (as shown). We have been unable to extend 5′ sequence using primer extension or 5′ RACE although the latter method is difficult in very GC rich regions.

EGR4 is the contiguous gene 5′ of ALMS1 (90 kb). Analysis of the sequence between these genes by NIX (www.hgmp.mrc.ac.uk) predicts putative exons although no prediction is “excellent”. We have designed forward primers to these and used them in combination with reverse primers in ALMS1 exons but no combination have been successful suggesting that the ORF predictions are not exons of ALMS 1. Total cDNA length approximates to ALMS1 Northern blot transcript size although size resolution of large (12.6 kb) fragments is difficult.

FIG. 4 shows the translocation breakpoint junction fragment. Southern blot of DNA from F1 Ch [child with 46, XY,t(2;11)(p13;q21)] and 2 controls digested with EcoR1 and HindIII. The filter was hybdridised with a labelled 346 bp PCR product (containing 90 bp of exon 3 and 256 bp of intron 3) that is within the 1.7 kb EcoR1 fragment. An altered sized fragment is detected in F1 Ch and shows that the breakpoint is within the 1.7 kb fragment

FIG. 5 shows ALMS1 expression. Expression was detected by RT-PCR performed on fetal tissues (listed above), embryonic heart, peripheral blood lymphocyte, WERI-retinoblastoma cell line, and placenta The negative controls (−RT, genomic DNA and water) did not amplify. The primers amplify a 464bp product from exon 19-22 of ALMS.

FIGS. 6 and 7 show dual immunofluorescence results with human fibroblasts. FIGS. 6a and 6b show fixed human fibroblasts from a normal individual showing expression of γ-tubulin (6a) and ALMS1 (6b). γ-tubulin localises to centrosomes and can be seen as white dots (indicated by arrows in 6a). ALMS1 is also detected (white dots indicated by arrows in 6b) and colocalises to the same cellular region/structure as γ-tubulin (white arrows). FIGS. 6c and 6d show results with fibroblasts from an individual with Alström syndrome (F1Ch). FIG. 6c shows γ-tubulin expression (indicated by white arrow) in a similar pattern to a normal individual (6a) but there is no expression of ALMS1 (6d).

FIG. 8 shows that cells from a patient with Alstrom syndrome (F1Ch in Table 1) do not react with an anti-ALMS1 antibody.

### DESCRIPTION OF THE SEQUENCES MENTIONED HEREIN

SEQ ID NO: 1 shows ALMS1 cDNA sequence (see Table 4).

SEQ ID NO: 2 shows the sequence of the ALMS1 protein (see Table 5).

SEQ ID NO: 3 shows sequence from the ALMS1 gene region (see Table 3).

### DETAILED DESCRIPTION OF THE INVENTION

The invention provides diagnosis or therapy of retinal dystrophy, cardiomyopathy, endocrinopathy and/or diabetes. The diabetes condition is preferably diabetes mellitus type II. The endocrinopathy typically includes liver disease, renal impairment and/or one or more other endocrine problems. These conditions may be present, or be suspected of being present in the individual to be diagnosed or treated. In one embodiment one or more of the conditions are present as symptoms of a syndrome, such as Alstrom syndrome.

Diagnosis

The present invention provides a method of diagnosis of particular conditions, typically in a mammal, such as a human individual. The ALMS1 protein, or a related component, or the gene region that expresses ALMS1 is typed, or aberrant ALMS1 activity is detected. Whether.the individual has retinal dystrophy, cardiomyopathy, endocrinopathy or diabetes or is susceptible to any of these conditions can thus be determined.

The novel gene identified by the inventors has been named ALMS 1. In the event that the International Naming Committee does not accept this name it would still be clear to the skilled person (given the disclosure of SEQ ID NO's 1 to 3 herein) which region of the genome corresponds to the ALMS1 gene region.

The typing of the gene region or protein (preferably ALMS1 protein or gene region) may comprise the measurement of any suitable characteristic of the gene region or protein to determine whether the individual has or is susceptible to retinal dystrophy, cardiomyopathy, endocrinopathy or diabetes. As discussed below such a characteristic includes a phenotype which is affected by the protein.

The term “ALMS1 protein” includes any naturally occurring isoform of this protein. Such an isoform will generally comprise all or part of the sequence of SEQ ID NO:2, or comprise sequence which has homology with (all or part of) SEQ ID NO:2.

In a preferred embodiment the typing comprises identifying whether the individual has a polymorphism which causes susceptibility to a retinal dystrophy, cardiomyopathy, endocrinopathy and diabetes, or a polymorphism which is in linkage disequilibrium with such a polymorphism, in (i) the ALMS1 gene region or (ii) the ALMS1 protein.

In one embodiment the method is carried out in vivo, however typically it is carried out in vitro on a sample from the individual. The sample typically comprises a body fluid of the individual and may for example be obtained using a swab, such as a mouth swab. The sample may be a blood, urine, saliva, cheek cell or hair root sample. The sample is typically processed before the method is carried out, for example DNA extraction may be carried out. The polynucleotide or protein in the sample may be cleaved either physically or chemically (e.g. using a suitable enzyme). In one embodiment the part of polynucleotide in the sample is copied (or amplified), e.g. by cloning or using a PCR based method. Polynucleotide produced in such a procedure is understood to be covered by the term “polynucleotide of the individual” herein.

Polymorphisms

Polymorphisms which are in linkage disequilibrium with each other in a population tend to be found together on the same chromosome. Typically one is found at least 30% of the times, for example at least 40 %, 50%, 70% or 90%, of the time the other is found on a particular chromosome in individuals in the population. Thus polymorphisms which are not functional susceptibility polymorphisms, but are in linkage disequilibrium with the functional polymorphisms, may act as a marker indicating the presence of the functional polymorphism. Polymorphisms which are in lincage disequilibrium with any of the polymorphisms mentioned herein are typically within 500kb, preferably within 400kb, 200kb, 100 kb, 50kb, 10kb, 5kb or 1 kb of the polymorphism. Similarly the term “gene region” generally encompasses any of these distances from 5′ to the transcription start site and 3′ to the transcription termination site of the gene.

The polymorphism is typically an insertion, deletion or substitution with a length of at least 1, 2, 5 or more base pairs or amino acids. In the case of a gene region polymorphism, the polymorphism is typically a substitution of 1 base pair, i.e. a single polynucleotide polymorphism (SNP). The polymorphism may be 5′ to the coding region, in the coding region, in an intron or 3′ to the coding region. The polymorphism which is detected is typically the functional mutation which contributes to a retinal dystrophy, cardiomyopathy, endocrinopathy or diabetes, but may be a polymorphism which is in linkage disequilibrium with the functional mutation.

Thus generally the polymorphism will be associated, with a retinal dystrophy, cardiomyopathy, endocrinopathy or diabetes, for example as can be determined in a case/control study (e.g. as discussed below). The polymorphism will generally cause a change in any of the characteristics of the ALMS1 protein discussed herein, such as expression, activity, expression variant, cellular localisation or the pattern of expression in different tissues.

The polymorphism may lead to an increase or decrease in the expression or activity of ALMS 1, although generally it will lead to a decrease in expression or activity.

Typically the polymorphism is selected from polymorphisms shown in Table 1 or is in linkage disequilibrium therewith.

The polymorphism may be a polymorphism at the same location as any of these particular polymorphisms (in the case of a SNP, it will be an A, T, C or G at any of the locations). The polymorphism may have a sequence which is different from or the same as the corresponding region in SEQ ID NO 1 or 3. Thus the sequence which is typed may correspond to a potion of SEQ ID NO: 1, 2 or 3.

A polymorphism which can be typed to determine susceptibility to a retinal dystrophy, cardiomyopathy, endocrinopathy or diabetes may be identified by detecting polymorphisms in ALMS1 or any of the components mentioned herein which affect or are affected by ALMS 1, or in the gene region expressing ALMS1 or he component. Such a polymorphism may be detected using any of suitable polymorphism detection method mentioned herein, such as sequencing. The detection method may be based on the detection of a difference in a characteristic of a polynucleotide or protein that carries the polymorphism and one which does not, typically mobility, such as mobility on a gel. The polymorphism may be identified in an individual who has or is suspected as having retinal dystrophy, cardiomyopathy, endocrinopathy or diabetes.

A polymorphism which can be typed to determine susceptibility to a retinal dystrophy, cardiomyopathy, endocrinopathy or diabetes may be identified by a method comprising determining whether a candidate polymorphism in the ALMS1 gene region or ALMS1 protein is (i) associated with the presence of, or susceptibility to a retinal dystrophy, cardiomyopathy, endocrinopathy or diabetes, or (ii) is in linkage disequilibrium with a polymorphism which is associated with retinal dystrophy, cardiomyopathy, endocrinopathy or diabetes, thereby determining whether the polymorphism can be used in said diagnosis.

Detection of Polymorphisms

The polymorphism is typically detected by directly determining the presence of the polymorphism sequence in a polynucleotide or protein of the individual. Such a polynucleotide is typically genomic DNA or mRNA, or a polynucleotide derived from these polynucleotides, such as in a library made using polynucleotide from the individual (e.g. a cDNA library). The processing of the polynucleotide or protein before the carrying out of the method is discussed further below.

Typically the presence of the polymorphism is determined in a method that comprises contacting a polynucleotide or protein of the individual with a specific binding agent for the polymorphism and determining whether the agent binds to a polymorphism in the polynucleotide or protein, the binding of the agent to the polymorphism indicating that the individual has or is susceptible to a retinal dystrophy, cardiomyopathy, endocrinopathy or diabetes.

Generally the agent will also bind to flanking nucleotides and amino acids on one or both sides of the polymorphism, for example at least 2, 5, 10, 15 or more flanking nucleotide or amino acids in total or on each side. Generally in the method determination of the binding of the agent to the polymorphism can be done by determining the binding of the agent to the polynucleotide or protein. However in one embodiment the agent is able to bind the corresponding wild-type sequence by binding the nucleotides or amino acids which flank the polymorphism position, although the manner of binding will be different to the binding of a polynucleotide or protein containing the polymorphism, and this difference will generally be detectable in the method (for example this may occur in sequence specific PCR as discussed below).

In the case where the presence of the polymorphism is being determined in a polynucleotide it may be detected in the double stranded form, but is typically detected in the single stranded form.

The agent may be a polynucleotide (single or double stranded) typically with a length of at least 10 nucleotides, for example at least 15, 20, 30 or more polynucleotides. The agent may be molecule which is structurally related to polynucleotides that comprises units (such as purines or pyrimidines) able to participate in Watson-Crick base pairing. The agent may be a polypeptide, typically with a length of at least 10 amino acids, such as at least 20, 30, 50, 100 or more amino acids. The agent may be an antibody (including a fragment of such an antibody which is capable of binding the polymorphism).

A polynucleotide agent which is used in the method will generally bind to the polymorphism, and flanking sequence, of the polynucleotide of the individual in a sequence specific manner (e.g. hybridise in accordance with Watson-Crick base pairing) and thus typically has a sequence which is fully or partially complementary to the sequence of the polymorphism and flanking region. The partially complementary sequence is homologous to the fully complementary sequence.

In one embodiment of the method the agent is as a probe. This may be labelled or may be capable of being labelled indirectly. The detection of the label may be used to detect the presence of the probe on (and hence bound to) the polynucleotide or protein of the individual. The binding of the probe to the polynucleotide or protein may be used to immobilise either the probe or the polynucleotide or protein (and thus to separate it from a composition or solution).

In one embodiment the polynucleotide or protein of the individual is immobilised on a solid support and then contacted with the probe. The presence of the probe immobilised to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The method may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In one embodiment the probe is used in a heteroduplex analysis based system to detect polynucleotide polymorphisms. In such a system when the probe is bound to polynucleotide sequence containing the polymorphism it forms a heteroduplex at the site where the polymorphism occurs (i.e. it does not form a double strand structure). Such a heteroduplex structure can be detected by the use of an enzyme which single or double strand specific. Typically the probe is an RNA probe and the enzyme used is RNAse H which cleaves the heteroduplex region, thus allowing the polymorphism to be detected by means of the detection of the cleavage products.

The method may be based on fluorescent-chemical cleavage mismatch analysis which is described for example in PCR Methods and Applications 3, 268-71 (1994) and Proc. Natl. Acad. Sci. 85, 4397-4401 (1998).

In one embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end of the primer. In one embodiment of this system the polynucleotide agent will bind to the wild-type sequence but will not act as a primer for a PCR reaction.

The method may be an RFLP based system. This can be used if the presence of the polymorphism in the polynucleotide creates or destroys a restriction site which is recognised by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may be determined based on the change which the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DDGE) is a similar system where the polynucleotide is subject to electrophoresis through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. Generally this assay uses an allele specific primer comprising the sequence around, and including, the polymorphism. The specific primer is labelled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridising to either allele 5′ of the polymorphism.

In the assay, when the allele comprising the polymorphism is present Taq DNA polymerase adds nucleotides to the non-specific primer until it reaches the specific primer. It then releases nucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye is not release from the quenching agent. Therefore by measuring the fluorescence emitted the presence or absence of the polymorphism can be determined.

In another method of detecting the polymorphism a polynucleotide comprising the polymorphic region is sequenced across the region which contains the polymorphism to determine the presence of the polymorphism.

Alternatively the diagnosis may be performed by measuring the expression or activity of an expression product of the ALMS1 gene, such as mRNA or the ALMS1 protein, or by measuring a phenotype which is affected by the ALMS1 protein.

Diagnostic Kit

The invention also provides a diagnostic kit that comprises an agent, probe, primer or antibody (including an antibody fragment) as defined herein. The kit may additionally comprise one or more other reagents or instruments which enable any of the embodiments of the method mentioned above to be carried out.

Such reagents or instruments include one or more of the following: a means to detect the binding of the agent to the polymorphism, a detectable label (such as a fluorescent label), an enzyme able to act on a polynucleotide (typically a polymerase, restriction enzyme, ligase, RNAse H or an enzyme which can attach a label to a polynucleotide), suitable buffer(s) (aqueous solutions) for enzyme reagents, PCR primers which bind to regions flanking the polymorphism (e.g. the primers discussed herein), a positive and/or negative control, a gel electrophoresis apparatus, a means to isolate DNA from sample, a means to obtain a sample from the individual (such as swab or an instrument comprising a needle) or a support comprising wells on which detection reactions can be done.

Therapy

The present invention provides use of an agent that modulates ALMS1 or a component which affects or is affected by ALMS 1, in the manufacture of a medicament for the prevention or treatment of retinal dystrophy, cardiomyopathy, endocrinopathy or diabetes. The individual to whom the agent is administered is typically a mammal, such as a human being. The individual may have, or be susceptible to, retinal dystrophy, cardiomyopathy, endocrinopathy or diabetes. The modulation enacted by the agent typically complements the effect of a polymorphism in the ALMS1 gene region/protein, and thus the modulation may result in an increase the expression or activity of ALMS1 or the component.

The invention also provides use of a polynucleotide, polypeptide or vector of the invention in the manufacture of a medicament for the prevention or treatment of a retinal dystrophy, cardiomyopathy, endocrinopathy or diabetes. The polynucleotide, polypeptide or vector may be in cellular form, such as in the form of any of the cells mentioned herein.

The agent typically modulates ALMS1, or has an effect on a component downstream of ALMS1 which is substantially similar to the effect that ALMS1 has on the component. Thus if ALMS1 activates the component then the agent will generally activate/agonise the component, and if ALMS1 inhibits the component then the agent will generally inhibit/antagonise the component.

The agent may modulate ALMS1 or the component directly. Such an agent generally binds ALMS1 or the component The agent may modulate expression of ALMS1 or the component, typically increasing expression of ALMS1 or of a component activated by ALMS1, and typically decreasing expression of a component inhibited by ALMS1. Thus the agent may increase the expression or activity of a component that complements the activity of ALMS1. The agent may modulate the recycling, sorting or maturation of the component.

The component is typically a carbohydrate, lipid, protein or polynucleotide (such as genomic DNA or unspliced or spliced mRNA). The component may be intracellular or extracellular.

In the case of an agent which modulates ALMS1 directly, the agent may modulate ALMS1 binding to other proteins or interaction between ALMS1 and a regulatory protein.

The agent may modulate a product which regulates or is part of the expression pathway of ALMS1 or the component. The product is preferably specific to that expression pathway and does not play a role in the expression of other proteins. The agent may act upon the product in any of the ways described herein in which the agent acts upon the component. The product may be the gene from which ALMS1 or the component is expressed, an RNA polymerase that can express mRNA from the gene, the unspliced mRNA which is transcribed from the gene, factors that aid splicing of the mRNA, the spliced mRNA, nuclear factors that bind to the mRNA and/or transport the mRNA from the nucleus to the cytoplasm, translation factors that contribute to translating the mRNA to protein.

Thus the agent may modulate transcription and/or translation of the ALMS1 or component gene. Preferably the agent is a specific activator of transcription, and does not activate transcription from other genes. The agent may bind to the gene 5′ to the coding sequence and/or to the coding sequence and/or 3′ to the coding sequence. Thus the agent may bind to the promoter, and activate the initiation of transcription. The agent may bind promoter or enhancer sequence present in SEQ ED NO: 3. The promoter typically comprises sequence up to 3 kb upstream from the initiation codon. As discussed above the agent may bind and activate the action of a protein that is required for transcription from the ALMS1 or component gene.

The agent may bind to the untranslated or translated regions of the mRNA. This could modulate the initiation of translation. The agent may modulate, in particular agonise, expression by modulating the rate at which ALMS1 or the component is broken down. The agent may modulate the expression of different variants of ALMS1 (e.g. the isoforms produced by different splicing of the mRNA).

The agent typically has an activity which directly or indirectly (e.g. mediated through any of the components discussed above) results in an effect on ALMS1, or components affected by AILMS 1, which is counter (opposite) to the effect of a polymorphism in the ALMS1 gene region or protein which causes susceptibility to retinal dystrophy, cardiomyopathy, endocrinopathy or diabetes.

Typically the activity of the agent will cause at least a 2, 5, 10, 20 or 50 fold increase in the expression or activity of (i) the component which it acts on and/or (ii) ALMS1 (directly or indirectly), for example as measured in any suitable in vitro or in vivo assay mentioned herein and typically at any of the administration doses mentioned herein. Agents may cause an increase of at least 10%, at least 25%, at least 50%, at least 100%, at least, 200%, at least 500% or at least 1000% in such expression or activity at a concentration of the agent of 1ng ml⁻¹, 1 μg ml⁻¹, 10 μg ml⁻¹, 100 μg ml⁻¹, 500 μg ml⁻¹, 1 mg ml^{-1,} 10mg ml⁻¹ or 100 mg ml⁻¹.

Typically the percentage increase represents the percentage increase in expression or activity in a comparison of assays in the presence and absence of the agent. Any combination of the above-mentioned degrees of percentage increase and concentration of agent may be used to define the agent, with a greater percentage increase at a lower concentration being preferred.

Typically the agent binds to ALMS1 or the component under physiological/cellular (in vivo) conditions. Generally the binding is specific. The binding is reversible or irreversible. An agent which binds irreversibly dissociates very slowly from the component because it would be very tightly bound; either covalently or non-covalently. Reversible binding, in contrast with irreversible binding, is characterised by a rapid dissociation of the complex.

Typically the agent will affect the binding of another substance to ALMS1 or the component (such as a substance which naturally binds them). The agent may bind at the same site as the substance binds. The agent is typically able to compete for, or inhibit, the binding of the substance to ALMS1 or the component.

The agent may or may not cause a change in the structure of ALMS1 or the component. In one embodiment the agent causes ALMS1 or the component to change to a less active or non-functional form. This change may be reversible or irreversible. Typically ALMS1 or the component only adopts such a changed form when bound to the agent. However the change may be irreversible, for example, if ALMS1 or the component is chemically modified or is broken down by the agent, for example by the breaking of peptide bonds.

The agent typically is or comprises a polypeptide or polynucleotide (such as DNA or RNA). In one embodiment the agent is an antisense polynucleotide, such as to any of the polynucleotides mentioned herein or to a polynucleotide from which any of the proteins mentioned herein is expressed. The agent may comprise the whole of or a fragment of any of the substances mentioned herein. The agent may be a small organic molecule (typically containing carbon, hydrogen and generally also oxygen), typically having a relative molecular weight of at least 100, such as at least 1000 or 10,000.

The invention may be carried out by administering a substance which provides an agent with any of the above properties in vivo. Such a substance is also included in the term ‘agent’. Typically the substance is an inactive or precursor form of the agent which can be processed in vivo to provide the agent. Thus the substance may comprise the agent associated, covalently or non-covalently, with a carrier. The substance can typically be modified or broken down to provide the agent.

Polypeptides, Polynucleotides, Cells and Antibodies

The invention also provides (i) the human ALMS1 protein (such as any naturally occurring isoform of ALMS1, e.g. due to different mRNA splicing) (ii) a polypeptide which comprises the amino acid sequence of SEQ ID NO: 2, (iii) a variant of (i) or (ii), or (iv) a fragment of (i), (ii) or (iii).

SEQ ID NO: 2 shows the sequence of an ALMS1 protein. Thus the polypeptide of the invention consists essentially of such a protein, a variant polypeptide of that protein (typically one which can complement ALMS1 activity), or a fragment of either. The variant may be a species homologue, such as a mammalian homologue (typically primate or rodent homologue).

The variant polypeptide may or may not have the same essential character or basic biological functionality as the ALMS1 protein. Thus the variant may be capable of complementing one or more activities of the ALMS1 protein, for example when expressed in a cell that does not express ALMS1. In one embodiment the variant is able to complement (prevent or treat) the cellular or physiological changes caused by mutations/polymorphisms in the ALMS1 gene (such as those shown in Table 1. Thus the variant may be able to prevent or treat retinal dystrophy, cardiomyopathy, endocrinopathy or diabetes (for example in the form of Alstrom syndrome) in a human individual or in an animal model of any of these diseases.

Preferably the variant polypeptide is capable of binding a product that can bind to ALMS1, such as an antibody specific to ALMS1. Variants which have particular activities or binding characteristics (of ALMS1) may be identified based on such activities or characteristics, for example from a library of polypeptides or variants. Generally the variant polypeptide is a homologue of ALMS1 or comprises sequence which is homologous to all or part of the ALMS1 protein sequence. Thus the variant polypeptide may be a fusion protein.

In one aspect of the invention, the variant polypeptide does not show the same activity as ALMS1, but inhibits an activity of ALMS1 (for example when expressed in a cell which expresses ALMS1). Such a variant typically binds to a cellular protein/component which binds ALMS1, and may inhibit the binding of ALMS1 to that protein/component.

The polypeptide is typically at least 10 amino acids long, such as at least 20, 50, 100, 300 or 500 amino acids long, up to, for example, 2000 amino acid in length. The polypeptide of the invention may be encoded by the polynucleotide of the invention.

The polypeptide of the invention may be chemically modified, e.g. post-translationally modified. For example, it may be glycosylated or comprise modified amino acid residues. It may also be modified by the addition of histidine residues for example to assist purification) or by the addition of a signal sequence to promote transport to a particular cellular location. Such a modified polypeptide falls within the scope of the term “polypeptide” of the invention.

The invention also provides a polynucleotide which is (a) all or part of the ALMS1 gene region or a polynucleotide expressed from the ALMS1 gene, (b) a polynucleotide whose sequence comprises the sequence of SEQ ID NO: 1 or 3, (c) a polynucleotide which selectively hybridises to (a) or (b), (d) a polynucleotide that encodes a polypeptide encoded by (a), (b) or (c), or (d) a polynucleotide which comprises sequence that is homologous to all or part of (a), (b), (c) or (d); or a polynucleotide which is complementary to any of (a), (b), (c) or (d).

A polynucleotide whose sequence comprises part of the ALMS1 gene region may comprise sequence 5′ to the coding sequence and/or coding sequence and/or intron sequence and/or sequence 3′ to the coding sequence.

The polynucleotide of the invention may comprise the ALMS1 promoter or promoter sequence which has the activity of the ALMS1 promoter. Typically such promoter sequence can bind one or more transcription factors and/or can drive transcription in a eukaryotic cell, e.g. a human cell.

The polynucleotide is typically at least 10, 15, 20, 30, 50, 100, 200, 500, bases long, such as at least (or up to) 1kb, 10kb, 100kb, 1000 kb or more in length. The polynucleotide may be RNA or DNA, including genomic DNA, synthetic DNA or cDNA. The polynucleotide may be single or double stranded.

The polynucleotide may comprise synthetic or modified nucleotides, such as methylphosphonate and phosphorothioate backbones or the addition of acridine or polylysine chains at the 3′ and/or 5′ ends of the molecule.

Selective hybridisation means that generally the polynucleotide can hybridize to the relevant polynucleotide, or portion thereof, at a level significantly above background. The signal level generated by the interaction between the polynucleotides is typically at least 10 fold, preferably at least 100 fold, as intense as interactions between other polynucleotides. The intensity of interaction may be measured, for example, by radiolabelling the polynucleotide, e.g. with ³²P. Selective hybridisation is typically achieved using conditions of medium to high stringency (for example 0.03 M sodium chloride and 0.003 M sodium citrate at from about 50° C. to about 60° C.).

A polynucleotide of the invention may be used as a primer (e.g. for PCR) or a probe. A polynucleotide or polypeptide of the invention may carry a revealing label. Suitable labels include radioisotopes such as ³²P or ³⁵S, fluorescent labels, enzyme labels or other protein labels such as biotin.

The polynucleotide or polypeptide of the invention may comprise (i) a polymorphism that causes susceptibility to a retinal dystrophy, cardiomyopathy, endocrinopathy and diabetes or (ii) a naturally occurring polymorphism that is in linkage disequilibrium with (i). Such a polymorphism may be any of the polymorphisms mentioned herein. The polymorphism that causes susceptibility may be one which is or which is not found in nature. Typically the polynucleotide or polypeptide will also include at least 2, 5, 10 or more of the bases or amino acids which flank the polymorphism from the naturally occurring polynucleotide or polypeptide in which the polymorphism is found.

The invention also provides expression vectors that comprise polynucleotides of the invention and are capable of expressing a polypeptide of the invention. Such vectors may also comprise appropriate initiators, promoters, enhancers and other elements, such as for example polyadenylation signals which may be necessary, and which are positioned in the correct orientation, in order to allow for protein expression.

Thus the coding sequence in the vector is operably linked to such elements so that they provide for expression of the coding sequence (typically in a cell). The term “operably linked” refers to a juxtaposition wherein the components described are in a relationship permitting them to function in their intended manner.

The vector may be for example, plasmid, virus or phage vector. Typically the vector has an origin of replication. The vector may comprise one or more selectable marker genes, for example an ampicillin resistance gene in the case of a bacterial plasmid or a resistance gene for a fin gal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or BPV promoters, particularly the HPV upstream regulatory region (URR).

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in-the art. Retrovirus vectors for example may be used to stably integrate the polynucleotide giving rise to the polynucleotide into the host genome. Replication-defective adenovirus vectors by contrast remain episomal and therefore allow transient expression.

The promoters and vectors (including viral vectors) which are mentioned herein may also be used with any of the therapeutic polynucleotides mentioned herein (including polynucleotides which express any of the therapeutic products mentioned herein). Such promoters may thus be operably linked to sequences which express the therapeutic product. As discussed above the polynucleotide and vectors of the invention may be used to treat or prevent a retinal dystrophy, cardiomyopathy, endocrinopathy or diabetes.

The invention also includes cells that have been modified to express the polypeptide of the invention. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention include mammalian HEK293T, CHO, HeLa and COS cells. Preferably the cell line selected will be one which is not only stable, but also allows for mature glycosylation of a polypeptide. Expression may be achieved in transformed oocytes.

The invention also provides antibodies specific for a polypeptide of the invention. The antibodies may be specific for wild-type ALMS1 protein (such as shown by SEQ ID NO: 2) or ALMS1 proteins which have a polymorphism, such as a polymorphism that causes a retinal dystrophy, cardiomyopathy, endocrinopathy or diabetes (e.g. a polymorphism shown in Table 1). Thus particular antibodies of the invention do not bind the sequence shown in SEQ ID NO:2 or fragments of that sequence, but do bind to ALMS1 proteins that have polymorphisms which cause a disease condition (such as retinal dystrophy, cardiomyopathy, endocrinopathy or diabetes.

The antibodies of the invention are for example useful in purification, isolation or screening methods involving immunoprecipitation techniques or, indeed, as therapeutic agents in their own right.

Antibodies may be raised against specific epitopes of the polypeptides of the invention. An antibody, or other compound, “specifically binds” to a polypeptide when it binds with preferential or high affinity to the protein for which it is specific but does substantially bind not bind or binds with only low affinity to other polypeptides. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

For the purposes of this invention, the term “antibody”, unless specified to the contrary, includes fragments which bind a polypeptide of the invention Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample (such as any such sample mentioned herein), which method comprises:
I providing an antibody of the invention;
II incubating a biological sample with said antibody under conditions which allow for the formation of an antibody-antigen complex; and
III determining whether antibody-antigen complex comprising said antibody is formed.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”. The fragment may be any of the fragments mentioned herein (typically at least 10 or at least 15 amino acids long) and may or may not comprise a polymorphism (such as any of the polymorphisms mentioned herein).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired,purified.

The polypeptides, polynucleotides, vectors, cells or antibodies of the invention may be present in a substantially isolated form. They may be mixed with carriers or diluents which will not interfere with their intended use and still be regarded as substantially isolated. They may also be in a substantially purified form, in which case they will generally comprise at least 90%, e.g. at least 95%, 98% or 99%, of the proteins, polynucleotides, cells or dry mass of the preparation.

Screening for Therapeutic Agents

The invention provides a method of screening which may be used to identify agents which can be used to prevent or treat retinal dystrophy, cardiomyopathy, endocrinopathy or diabetes. As discussed above in the section on therapy different agents may affect ALMS1 activity in different ways.

In one embodiment of the method of identifying an agent of the invention a candidate substance is contacted with a product which is (i) ALMS1 or (ii) a component which affects/regulates or is affected by ALMS1 or (iii) a functional analogue of (i) or (ii), and determining whether the substance modulates or binds (i), (ii) or (iii).

The method may comprise contacting a candidate substance with a product which is part of the expression pathway of ALMS1 or of the component; or a functional analogue thereof, and determining whether the candidate substance binds or modulates the product. The product may be a polypeptide or polynucleotide of the invention.

The modulation which is detected in the method is typically one which would (i) lead to an increase (directly or indirectly) in ALMS1 activity or expression, or in an activity which is agonised by ALMS1, or (ii) correct aberrant AMS1 activity.

The method may be carried out in vitro (inside or outside a cell) or in vivo. In one embodiment the method is carried out on a cell, cell culture or cell extract which comprises the product. The cell may be any of the cells mentioned herein, and is preferably the cell is one in which the product is naturally expressed, such as a brain, kidney, liver, pancreas, heart, skeletal muscle or placental cell, or a lymphoblast or fibroblast.

The method may be carried out in an animal (such as any animal mentioned herein) whose ALMS1 gene comprises a polymorphism which causes susceptibility to retinal dystrophy, cardiomyopathy, endocrinopathy or diabetes, such as any such polymorphism mentioned herein. Typically such a ALMS1 gene is a polynucleotide provided by the invention (as described below) or comprises sequence from such a polynucleotide.

In the case where a functional analogue is used the analogue will have some or all of the relevant activity of the original substance, or will have a surface that mimics the surface of the original substance. Typically the analogue is or comprises a fragment of the substance. In the case where the original substance is a polynucleotide or polypeptide the analogue typically has homology with the original substance.

Any suitable binding assay format can be used to determine whether the product binds the candidate substance, such as the formats discussed below.

The term ‘modulate’ includes any of the ways mentioned herein in which the agent of the invention is able to modulate ALMS1 or a component. Whether or not a candidate substance modulates the activity of the product may be determined by providing the candidate substance to the product under conditions that permit activity of the product, and determining whether the candidate substance is able to modulate the activity of the product.

The activity which is measured may be any of the activities which is mentioned herein, and may the measurement of a change in the product or in protein sorting, or an effect on a cell or animal in which the method is being carried out. The effect may be one which is associated with retinal dystrophy, cardiomyopathy, endocrinopathy or diabetes.

Typically the assay measures the effect of the candidate substance on the binding between ALMS1 or the component and another substance (such as a ligand). Suitable assays in order to measure the changes in such interactions include fluorescence imaging plate reader assays, and radioligand binding assays.

In the case where the activity is transcription from a gene the method may comprise measuring the ability of the candidate substance to modulate transcription, for example in a reporter gene assay. Typically such an assay comprises:
(a) providing a test construct comprising a first polynucleotide sequence with the promoter activity of the gene operably linked to a second polynucleotide sequence to be expressed in the form of mRNA;
(b) contacting the candidate substance with the test construct under conditions that would permit, the second polynucleotide sequence to be expressed in the form of mRNA in the absence of the substance; and
(c) determining whether the substance modulates expression from the construct.

Suitable candidate substances which tested in the above screening methods include antibody agents (for example, monoclonal and polyclonal antibodies, single chain antibodies, chimeric antibodies and CDR-grafted antibodies). Furthermore, combinatorial libraries, defined chemical identities, peptide and peptide mimetics, oligonucleotides and natural agent libraries, such as display libraries (e.g. phage display libraries) may also be tested. The candidate substances may be chemical compounds, which are typically derived from synthesis around small molecules which may have any of the properties of the agent mentioned herein (such as the organic compounds mentioned herein). Batches of the candidate substances may be used in an initial screen of, for example, ten substances per reaction, and the substances of batches which show modulation tested individually.

Homologues

Homologues of polynucleotide or protein sequences are referred to herein. Such homologues typically have at least 70% homology, preferably at least 80, 90%, 95%, 97% or 99% homology, for example over a region of at least 15, 20, 30, 100 more contiguous nucleotides or amino acids. The homology may be calculated on the basis of nucleotide or amino acid identity (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differ by at least 1, 2, 5, 10, 20 or more mutations (which may be substitutions, deletions or insertions of nucleotide or amino acids). These mutation may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Transgenic Animals

The invention also provides a non-human animal transgenic for a polynucleotide of the invention. Preferably the animal is transgenic for a polymorphism as mentioned above. The animal may be any suitable mammal such as a rodent (e.g. a mouse, rat or hamster) or primate. Typically the genome of all or some of the cells of the animal comprises a polynucleotide of the invention. Generally the animal expresses a protein of the invention. Typically the animal suffers from retinal dystrophy, cardiomyopathy, endocrinopathy or diabetes (such as in the form of Alstrom syndrome) and can be therefore used in a method to assess the efficacy of agents in relieving retinal dystrophy, cardiomyopathy, endocrinopathy or diabetes.

Treatment of Patients

The invention provides a method for treating a human or animal patient who has been diagnosed as having or being susceptible to retinal dystrophy, cardiomyopathy, endocrinopathy or diabetes by a method of the invention, comprising administering an effective amount of a substance which prevents or treats retinal dystrophy, cardiomyopathy, endocrinopathy or diabetes to the patient. The substance may be administered to a patient to prevent the onset of retinal dystrophy, cardiomyopathy, endocrinopathy or diabetes. The invention also provides:
use of the substance in the manufacture of a medicament for use in treating a patient who has been diagnosed as having or being susceptible to retinal dystrophy, cardiomyopathy, endocrinopathy or diabetes by a method of the invention; and
a pharmaceutical pack comprising the substance and instructions for administering of the substance to humans diagnosed by the method of the invention.

Typically the following substances may be used to prevent or treat cardiomyopathy: captopril, propranolol hydrochloride or verapamil hydrochloride.

Typically the following substances may be used to prevent or treat diabetes: a sulphonylurea (preferably chlorpropamide, glibenclamide, gliclazide or tolbutamide), a biguanide (preferably metformin), an inhibitor of intestinal alpha glucosidases (preferably acarbose), guar gum, nateglinide, repaglinide, a thiazolidinedione (preferably pioglitazone or rosiglitazone).

Administration

The formulation of any of the therapeutic substances mentioned herein will depend upon factors such as the nature of the substance and the condition to be treated. Any such substance may be administered in a variety of dosage forms. It may be administered orally (e.g. as tablets, troches, lozenges, aqueous or oily suspensions, dispersible powders or granules), parenterally, subcutaneously, intravenously, intramuscularly, intrasternally, transdermally or by infusion techniques. The substance may also be administered as suppositories. A physician will be able to determine the required route of administration for each particular patient.

Typically the substance is formulated for use with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of nixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective amount of substance is administered. The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the disease and the frequency and route of administration. Preferably, daily dosage levels are from 5 mg to 2 g.

The effectiveness of particular substances which prevent or treat retinal dystrophy, cardiomyopathy, endocrinopathy or diabetes may be affected by or dependent on whether the individual as particular polymorphisms in the ALMS1 gene region or protein. Thus the invention can allow the determination of whether an individual will respond to a particular agent by determining whether the individual has a polymorphism which affects the effectiveness of that agent. The, invention includes a method of treating a patient who has been identified as being able to respond to the agent comprising administering the agent to the patient.

Similarly certain agents may produce side effects in individuals with particular polymorphisms in the ALMS1 gene region or protein. Thus the invention can also allow the identification of a patient who is at increased risk of suffering side effects due to such an agent by identifying whether an individual has such a polymorphism.

In a further aspect, the invention may further be used in the development of new drug therapies which selectively target one or more allelic variants of the ALMS1 protein (i.e. which have different polymorphisms). Identification of a link between a particular allelic variant and predisposition to condition development or response to drug therapy may have a significant impact on the design of new drugs. Drugs may be designed to regulate the biological activity of the variants implicated in the condition process while minimising effects on other variants.

The following Examples illustrates the invention:

### EXAMPLE 1

### Detection of Mutations in the ALMS1 Gene of an Individual with Alstrom Syndrome

We have investigated an individual with Alstrom syndrome carrying a balanced reciprocal translocation within the critical region [46, XY,t(2;11)(p13;q21) mat]; although unaffected family members also have the translocation we postulated that this individual was a compound heterozygote. We have mapped the 2p13 breakpoint on the maternal allele to a 1.7 kb genomic fragment containing exon 3 and the start of exon 4 of a novel gene (ALMS1) and detected a frameshift mutation in the paternal copy of the gene. The 12.6 kb ALMS1 transcript encodes a 4,127 amino acid protein of unknown function, which contains 34 copies of a novel 47 amino acid tandem repeat.

We have detected five different mutations (2 nonsense and 3 frameshift mutations causing premature STOP codons) in 7 families, confirming ALMS1 as the gene responsible for Alström syndrome. We believe ALMS1 is the first human disease gene characterized by autosomal recessive inheritance to be identified as a result of a balanced reciprocal translocation.

Alström syndrome was mapped to a 6.1 cM interval between loci D2S286 and D2S327. Although several candidates genes have been investigated, no mutations have been identified. We have demonstrated that the 2p13 breakpoint in the individual with 46,XY,t(2;11)(p13;q21)mat, is between these loci by metaphase FISH analysis using probes BAC 355F16 (containing D2S286) and BAC 480F1 (located 150 kb proximal to D2S327). BAC 582H1 crossed the translocation (hybridises to chromosome 2, derivative 2 (der2) and derivative 11 (der11)) and was overlapped by BAC 79N18, which contains CCT7, a member of a chaperonin gene family. Mutations in MKKS (a putative chaperonin) have been detected in Bardet-Biedl syndrome, which has phenotypic overlap with Alström syndrome (e.g. obesity, insulin resistance and retinopathy). We therefore investigated Alström syndrome patients for mutations in CCT7 but no coding sequence changes were identified.

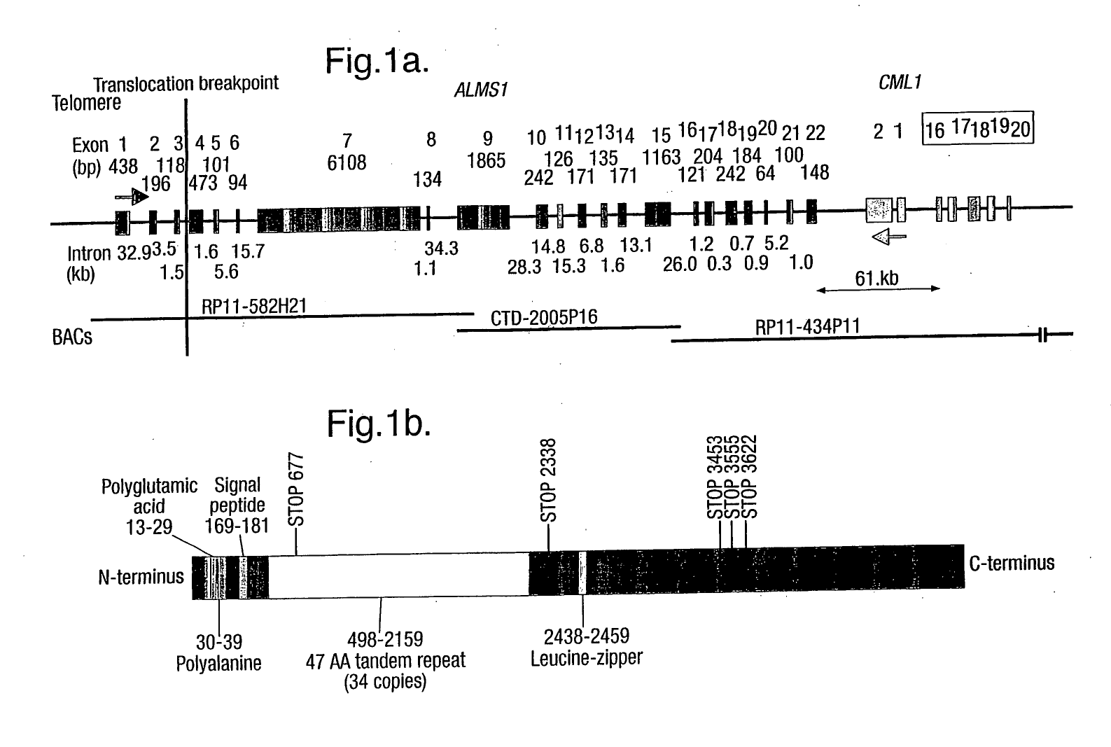

Sequence analysis of BAC 582H21, which crossed the translocation breakpoint, suggests the presence of a single gene represented by the cDNA KIAA0328 (Nagase T et al, DNA Res 4, 141-50 (1997)). Northern blot analysis using a 3.9 kb RT-PCR product derived from this cDNA detected a ˜12-13 kb transcript suggesting the 6.3 kb KIAA0328 database entry to be incomplete or partial sequence. Exon prediction and primer design permitted RT-PCR to confim a longer transcript. The extended sequence (ALMS1) is 12.6 kb in length and contains an open reading frame of 12.4 kb comprising 22 exons (FIG. 1a) over 224 kb of genomic DNA. Analysis of the ALMS1 transcript reveals the predicted start codon in an efficient Kozak site with suitable transcription initiation <100 base pairs upstream of the 5′ limit of our cDNA sequence by RT-PCR.

By analysis of genomic sequence, exons 16-20 lie within a 6.5 kb region which is duplicated 61 kb 3′ of ALMS1 (FIG. 1a). These regions share 94.5% sequence identity and are in the same orientation, although the derived copies of exons 16-20 contain nonsense mutations indicating that they are not expressed. PCR primers used for mutation screening of exons 16-20 were designed to amplify the coding copies only. In between ALMS1 and the duplicated region is CML1, a gene on the opposite strand with similarity to bacterial acetyltransferase.

Within the translocation patient, the maternally inherited 2p13 breakpoint was localised to a 9.0 kb long-range PCR product, derived from BAC 582H21, containing exons 2-4 and refined by Southern blotting to a 1.7 kb EcoR1 fragment containing exon 3 and the start of exon 4. In the same patient we also detected a two base pair deletion in exon 7 of the paternal ALMS1 allele (2015-2016delCT) predicted to cause premature termination 5 codons downstream. In keeping with these findings, sequencing of an RT-PCR product derived from lymphocyte mRNA demonstrated expression only of a mRNA transcript containing the 2015-2016delCT mutation consistent with monoallelic expression of the paternal allele and disruption of the maternal ALMS1 transcript between exons 3 and 4.

In the hybridisation experiments chromosome 2 PAC1011017 and chromosome 11 specific alphoid DNA were used as control probes.

Six other families with Alström syndrome (either affected individuals or obligate gene carriers) were investigated and 4 additional mutations were identified, each causing a premature STOP codon. In families 8 and 9 we have been unable to identify pathogenic coding sequence mutations. The results are summarised in table 1. In family 2, two mutations were identified in exon 15. The affected siblings are compound heterozygotes containing a G→A nonsense mutation (W3622stop) and a single base pair deletion, 10649delC with the predicted frameshift causing premature termination 5 codons downstream. DNA from the father (F2F) was heterozygous for 10649delC but lacked W3622stop consistent with carrier status. Maternal DNA was unavailable. Sequencing of RT-PCR products from lymphocyte mRNA demonstrated expression of the respective mutations in the father and children. 10649delC also was detected in three additional families who were not known to be related

In family 3, the affected individual inherited 10649delC from his mother, however sequencing the entire coding and splice site regions failed to detect a paternally inherited mutation. A paternally inherited heterozygous A→G transition in exon 7 causing an asparagine to aspartic acid substitution (N1746D) was identified but this change was also found in three out of seventy-two normal chromosomes suggesting it is not pathogenic.

Mutations were identified in two further affected individuals. One was homozygous for a C→T nonsense mutation in exon 15 (Q3453stop) and the other homozygous for a single base pair insertion in exon 7 (7006insA) causing a frameshift and premature termination 2 codons downstream. None of the truncating mutations identified in this study were found in 100 normal chromosomes.

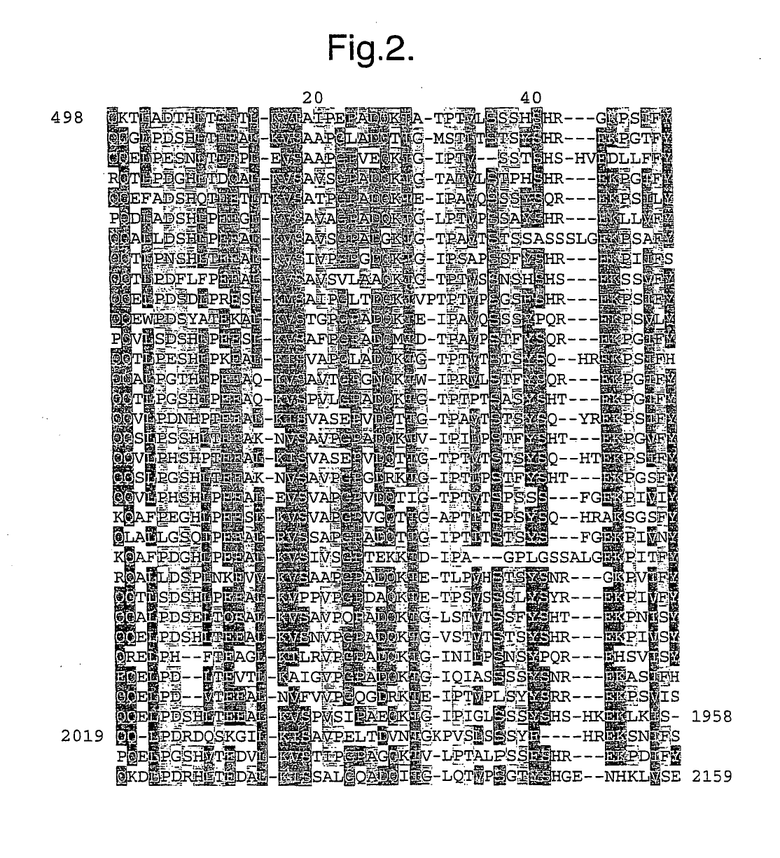

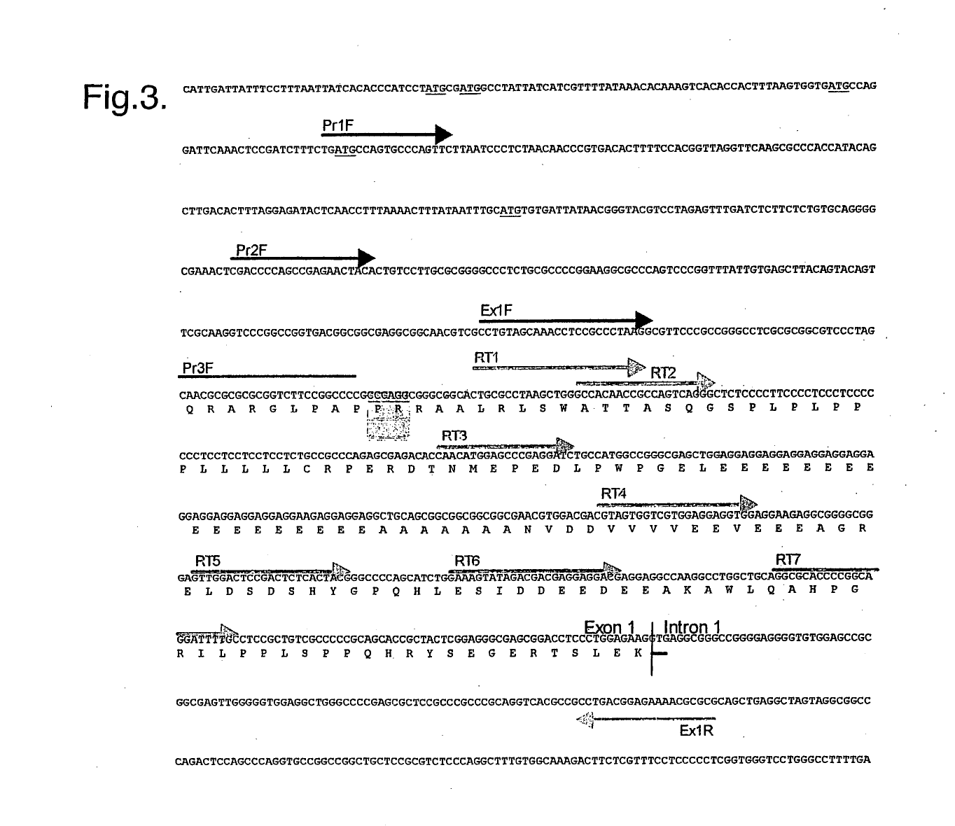

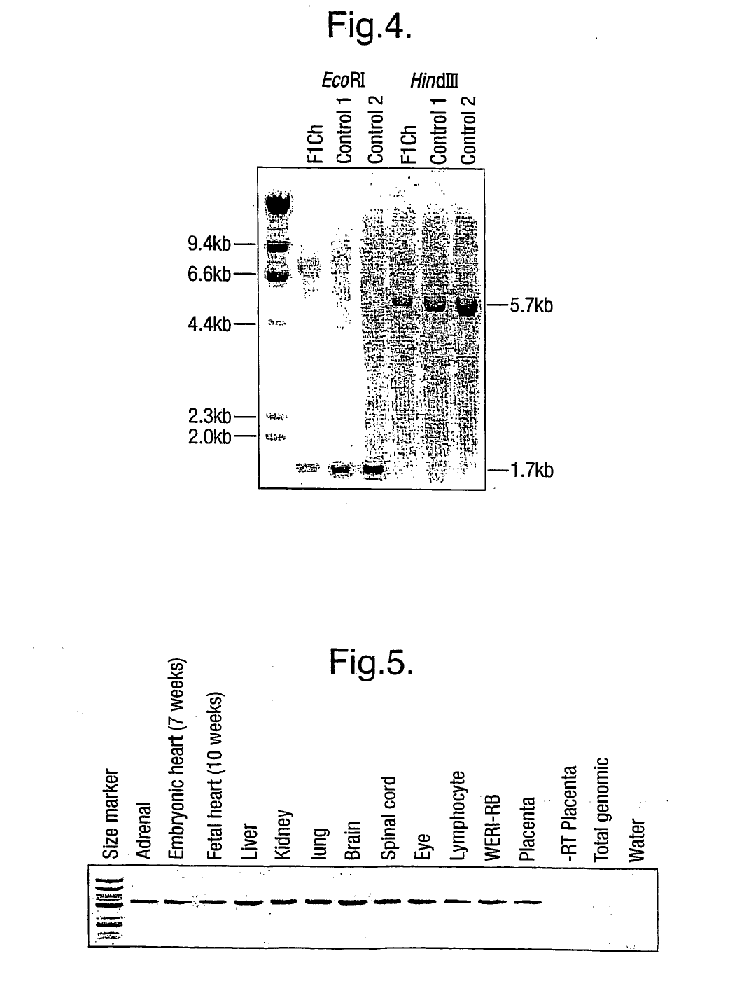

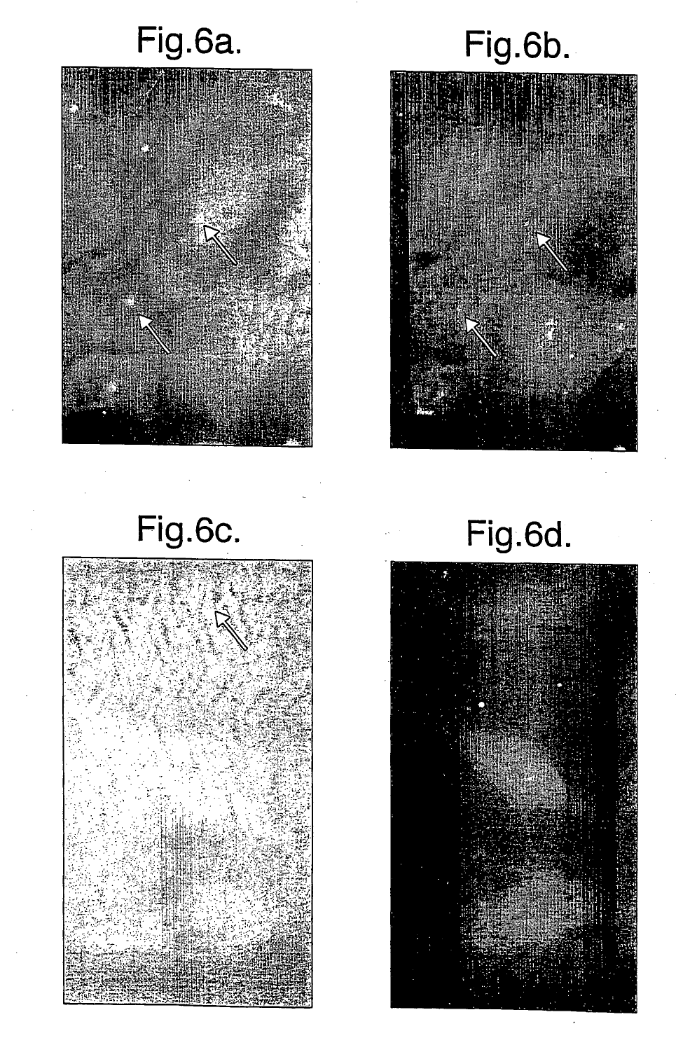

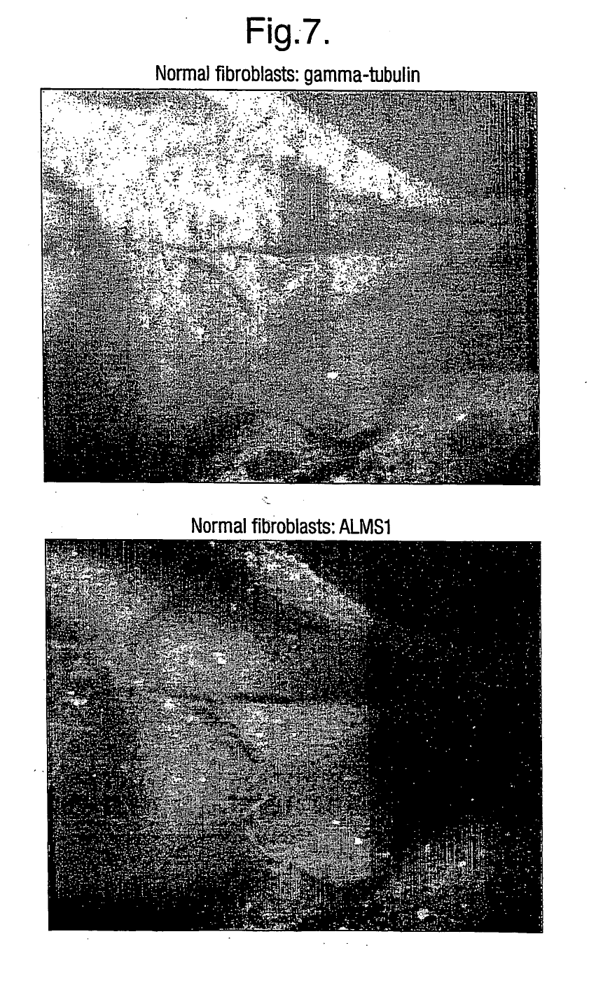

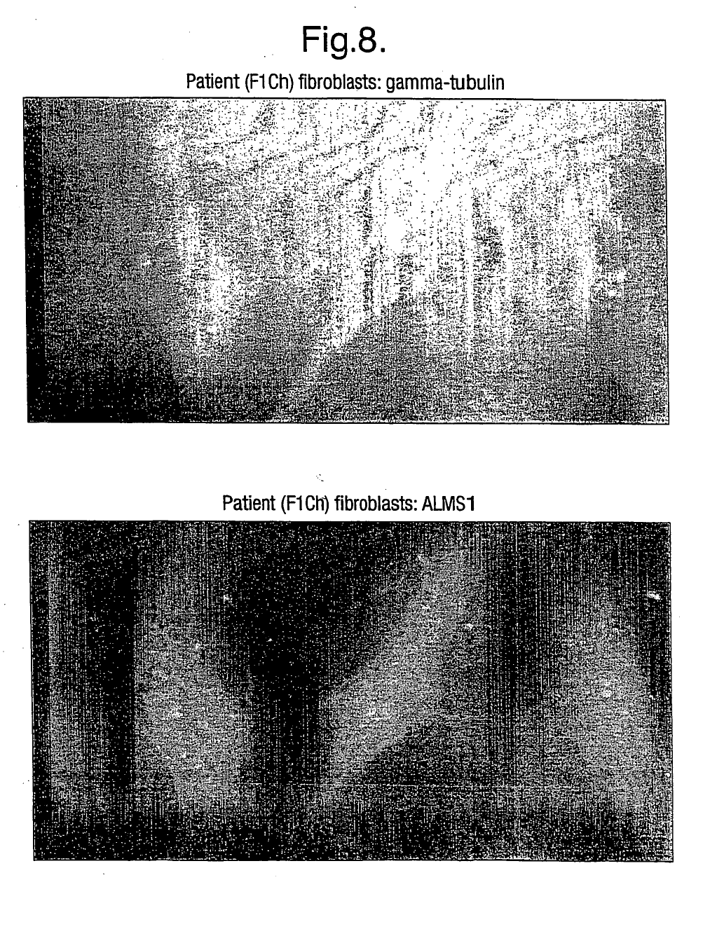

These data indicate that the ALMS1 gene is responsible for Alström syndrome, and thus also for causing retinal dystrophy, cardiomyopathy, endocrinopathy and diabetes. ALMS1 is a novel protein of 4127 amino acids with a predicted molecular weight of 456.5 Kd. In silico analysis predicts a leucine zipper at position 2438-2459 and a potential signal peptide at position 169-181 but no other known evolutionarily conserved sequence domains are apparent (FIG. 1b). A striking feature of the sequence is the presence of 34 tandemly arrayed copies of a novel 47 amino acid repeat (aa 498-2159) (FIG. 2) that contains no cysteine residues. This array constitutes ˜40% of the protein and is encoded entirely by exon 7. Repeat length varies from 45 to 50 amino acids and repeat identity from 40-90%. ALMS1 also contains a run of 17 glutamic acid residues (aa 13-29) encoded by (GAG),₁₃GAA(GAG)₃, followed by a run of seven alanine residues (aa 30-36).

To analyse other potential pathogenetic mechanisms we investigated the GAG trinucleotide repeat and polyalanine tract in exon 1 for size variation. Sequencing of seventy-two normal chromosomes revealed a size variation of 13-20 glutamic acids, however the GAG allele sizes of the obligate carriers (F3F, F8M, F8F) and affected individuals (F9Ch) in whom no other mutations have been detected lay within the normal variation range. No variation in the size of the polyalanine tract was detected in either normal individuals or obligate gene carriers.

Using RT-PCR we have demonstrated ALMS1 expression in all the tissues so far investigated (fetal heart, aorta, liver, kidney, lung, neural tube, eye, adrenal glands, placenta, lymphocytes, and WERI-retinoblastoma cell line).

Cloning breakpoints of balanced translocations has proved a successful strategy for dominant and X-linked disease gene identification but not autosomal recessive. We have demonstrated disruption of ALMS1 by a translocation and believe this is the first human autosomal recessive gene to be identified using this strategy. The 5 independent mutations predicted to cause protein truncation confirms that dysfunction of ALMS1 causes Alström syndrome. We have failed to identify mutations in 2 families which raises the possibility of genetic heterogeneity although our methods of mutation detection have not excluded larger DNA deletions.

The function of ALMS1 is not clear. There are similarities in the structural organisation of AMLS 1 with mucin (MUC) genes. Mucins are secreted proteins, which are heavily glycosylated and have a large tandem repeat domain encoded by a single exon. In common with ALMS1, mucin tandem repeats have a low cysteine residue content but in contrast have characteristic high threonine and serine content suggesting that ALMS1 is not a mucin. ALMS1 is therefore a novel protein for an insulin resistance syndrome.

### EXAMPLE 2

### Methods

Patients Samples.

Samples from nine families with Alström syndrome were available which included lymphoblastoid cell lines from members of 3 families from the European Collection of Cell Cultures (Salisbury UK) (BV0752, BV0754, BV0757, BV0791, BV0792, BV0764). In total, DNA was available from eight affected children; in two families DNA available was from only one parent. DNA from 50 unrelated normal individuals was used as control.

Sequence analysis.

BACs within the 6.1 cM critical region were identified using Basic Local Alignment Search Tool (BLAST) to query the nr and htgs databases with marker sequences. BAC contigs were then built electronically using the TIGR BAC end sequence database (www.tigr.org) and NIX analysis (www.hgmp.mrc.ac.uk). Genes were identified using NIX. Protein sequences were analysed using PIX (www.hgmp.mrc.ac.uk) and InterProScan (www.ebi.ac.uk).

Mutation detection.

PCR products were sequenced using BigDye Terminator Cycle Sequencing Kit (Applied Biosystems) according to manufacturers instructions and analysed on an ABI 377 sequencer. Primers were designed to amplify exons and splice site sequence from genomic DNA.

Fluoresence in situ Hybridisation (FISH).

Probes for FISH were labelled directly following manufacturers instructions (Vysis) and hybridised to metaphase chromosomes. RPCI-11 BACs were ordered from BAC/PAC Resources (Oakland Cailf.). Long-range PCR products were generated using an Expand Long Template PCR kit (Roche).

RT-PCR and RACE.

Total RNA was extracted from lymphoblastoid cells using Tri reagent (Sigma). First strand cDNA was generated using Superscript II (Invitrogen). 5′ RACE was carried out using the Step-Out PCR method (Matz et al, Nucleic Acids Research 27, 1558-60 (1999)).

Southern Blot Analysis.

Restriction digests of total genomic DNA were separated on a 1% agarose gel and transferred to membrane by alkali blotting. A 346 bp PCR product spanning part of exon 3 and part of the following intron was purified by gel-extraction and labelled with α-32P dCTP using a Rediprime II kit (Amersham-Pharmacia Biotech). Hybridisation and washing of filters followed a standard protocol.

Northern Blot Analysis.

A human multiple tissue Northern blot (Clontech) was probed with a 3.9 kb RT-PCR product spanning exons 3 to 13 (basepairs 8118 to 12035 of the ALMS1 coding sequence). The probe was purified and labelled as described above for Southern blot analysis. Hybridisation and washing conditions followed manufacturer's instructions (Clontech).

### EXAMPLE 3

### Localisation of ALMS1 Protein

A polyclonal antibody was raised by immunising a rabbit with 2 short peptide sequences CNKPISKKEMIQRSKR-COOH and CHREKPGTFYQQELK-CONH₂. A suitable immune response was raised CHREKPGTFYQQELK-CONH₂ and the immune sera was purified over an affinity purification column (loaded with CHREKPGTFYQQELK-CONH₂) and then used as primary antibody for immnunohistochernistry experiments.

Using a standard technique (Antibodies. A Laboratory Manual. E Harlow, D Lane. Cold Spring Harbor Laboratory. ISBN 0-87969-314-2. 1988) the antibody detected ALMS1 protein in all tissues studied so far; these include cultured fibroblast cells, cultured lymphoblastoid cells and the following fetal tissues: retina, eye lens, skeletal muscle, cardiac muscle, liver, kidney, and neuronal cells. The antibody shows that ALMS1 predominantly localises to the centrosomes of cells. This was confirmed by dual fluorescence immunohistochemistry demonstrating colocalisation of γ-tubulin using an antibody against this protein (Sigma product number T6557). γ-tubulin is known to localise to centrosomes. It was also shown that in fibroblast cells from a patient with Alstroms syndrome (F1 Ch in Table 1), the ALMS1 antibody did not show any staining but the γ-tubulin antibody did (FIG. 8). This confirms that the cells from F1 Ch did not have any ALMS1 that was immunoreactive to the antibody.

| | Status | DNA mutation | Protein change | Comments |
|---|---|---|---|---|
| Family 1 | | | | |
| F1M | Carrier (mother) | Translocation | | 46, XX, t (2; 11) (p13; q21) |
| F1F | Carrier (father) | c.2015-2016delCT | p.S672fs | |
| F1Ch | Affected | Translocation/c.2015-2016delCT | p.S672fs | 46, XY, t (2; 11) (p13; q21) |
| Family 2 | | | | |
| F2F (BV0757) | Carrier (father) | c.10649delC | p.T3550fs | No DNA from mother |
| F2Ch1 (BV0791) | Affected | [c.10649delC] + [c.10866G>A] | p.T3550fs + p.W3580X | |
| F2Ch2 (BV0792) | Affected | [c.10649delC] + [c.10866G>A] | p.T3550fs + p.W3580X | |
| Family 3 | | | | |
| F3M | Carrier (mother) | c.10649delC | p.T3550fs | |
| F3F | Carrier (father) | No coding changes | | |
| F3Ch | Affected | [c.10649delC] + [?] | p.T3550fs + ? | Paternal mutation unidentified |
| Family 4 | | | | |
| F4Ch | Affected | [c.10649delC + [c.10649delC] | p.T3550fs + p.T3550fs | No parental DNA |
| Family 5 | | | | |
| F5F (BV0764) | Carrier (father) | c.10649delC | p.T3550fs | No DNA from mother |
| F5Ch | Affected | [c.10649delC] + [c.6445-6448delTCAC] | p.T3550fs + p.S2149fs | |
| Family 6 | | | | |
| F6Ch | Affected | [c.10357C>T] + [c.10357C>T] | p.Q3411X + p.Q3411X | No parental DNA |
| Family 7 | | | | |
| F7Ch | Affected | [c.7006insA] + [c.7006insA] | p.T2336fs | No parental DNA |
| Family 8 | | | | |
| F8M (BV0752) | Carrier (mother) | No coding changes | | No DNA from affected child |
| F8F (BV0754) | Carrier (father) | No coding changes | | |
| Family 9 | | | | |
| F9Ch | Affected | No coding changes | | No parental DNA |

TABLE 2
Primers for PCR amplification of ALMS1 coding and
splice sites from genomic DNA.

| Primer | | Annealing | Product size |
|---|---|---|---|
| name | Primer sequence (5′ to 3′) | temperature | (bp) |
| Ex1F | cctgtagcaaacctccgccctaag | 63 | 630 |
| Ex1R | gcgcgttttctccgtcagg | | |
| Ex2F | gatgtgtagtaatgggaagaggtc | 57 | 612 |
| Ex2R | aacgggaagtgataaaatgagagg | | |
| Ex3F | gctgctactgctgttgctttta | 60 | 403 |
| Ex3R | actttgttaccttcagggcgtgtc | | |
| Ex4F | aggagagctgtgtttttcaataca | 61 | 742 |
| Bx4R | gtttctgggtggtgcaaagttc | | |
| Ex5F | ctgatataggccaaggtgaaagtc | 57 | 299 |
| Ex5R | acccaggaaacagtaaaaagtaaaat | | |
| Ex6F | tcatttcttcgtaggtgggtcatt | 57 | 230 |
| Ex6R | ttttcacaaggtatccgtaagtag | | |
| Ex7aF | tctcctttgatggctgtttcctta | 57 | 683 |
| Ex7aR | gtattcccgtcttctgctccact | | |
| Ex7bF | caactggcatgtcaactc | 57 | 666 |
| ExTbR | cttcgggtagatggctgtc | | |
| Ex7cF | gtacccacaggacttagca | 57 | 731 |
| Ex7cR | actcctgttgatagaaaatactgg | | |
| Ex7dF | ctgaccagaagactgtcccaacac | 60 | 515 |
| Ex7dR | caaggcctgctggtggaaaat | | |
| Ex7eF | cacaccagcagtaccgtctac | 65 | 527 |
| Ex7eR | tcagagcctcttcagttggatgatta | | |
| Ex7fF | gaaagtttcacctgttcttg | 60 | 1021 |
| Ex7fR | tggtccaggagcagaagaa | | |
| Ex7gF | tggcgcaccaactataacctctc | 60 | 491 |
| Ex7gR | gctggtagaaaatgacaggcttcc | | |
| Ex7hF | ctaaataaagaggttgtgaaag | 50 | 374 |
| Ex7hR | atgtgaatagaaagaggaagtta | | |
| Ex7iF | caggccctgcagaacagtgag | 65 | 564 |
| Ex7iR | ctggcaactcctgctgatgaaa | | |
| Ex7jF | ggttcctgggcctgctgac | 61 | 533 |
| Ex7jR | ttgggctttactgtttgagaatag | | |
| Ex7kF | tcacaaatagagaagcccaagat | 60 | 374 |
| Ex7kR | atgtgaaaaggaactaggaagagc | | |
| Ex7lF | atgtaactgaagatgtgctgaag | 57 | 473 |
| Ex7lR | ttctgcctccatcaaaagtgtc | | |
| Ex7mF | aaggatttgccagatagacat | 52 | 515 |
| Ex7mR | tcctctgtgaatggctgtctgt | | |
| Ex7nF | actcttaaggaaattcggacac | 57 | 391 |
| Ex7nR | Ttacagatttggctgcttgata | | |
| Ex7oF | cctcttccacgggtgtatctaa | 60 | 476 |
| Bx7oR | accctcttcctccctgctatct | | |
| Ex7pF | gcaatatcaagcagccaaatct | 55 | 511 |
| Ex7pR | gatcttgcttgtatttttcattg | | |
| Ex8F | ctaagcattgcagtgggtatt | 55 | 293 |
| Ex8R | ccattctttcatatttactttcttca | | |
| Ex9aF | ttttgcttgctgtgctgtttgtc | 60 | 455 |
| Ex9aR | agttctgaaatggggaatgactt | | |
| Ex9bF | Ttggctgtcagaattagtagaacc | 60 | 386 |
| Ex9bR | Gaaaagaacgctcaaaatcaacag | | |
| Ex9cF | ttttaaatgatgaaggggaagagg | 57 | 494 |
| Ex9cR | aatgaccagaggacggcagaac | | |
| Ex9dF | aggaaggtctggagctacataatc | 60 | 517 |
| Ex9dR | gggggtatttaattctcatttcac | | |
| Ex9eF | cactattcatcctcgctttagaag | 60 | 566 |
| Ex9eR | cccacgtgcagatgaccat | | |
| Ex9fF | ttagccagacacaacctccactca | 60 | 575 |
| Ex9fR | gcatctgcatctactcctccttca | | |
| Ex10F | acagctatgcaaataaaagacagata | 55 | 484 |
| Ex10R | atattccttgaaaccacttttgtaag | | |
| Ex11F | gaacactgcctgaaacatagaag | | |
| Ex11R | tcactgaaaaattaaagagcactg | 55 | 326 |
| Ex12F | atcgtgcctggcctgtagtgct | | |
| Ex12R | attggatagtaatctcatttaggatg | 57 | 363 |
| Ex13F | aattgtgggggaggattact | | |
| Ex13R | ctgtttctgttcttaagggaatag | 55 | 290 |
| Ex14F | accgctacctctttttctgactgt | | |
| Ex14R | cagaatgaaatggaaacactaaca | 55 | 469 |
| Ex15AF | gttctttcctgtcacccaactc | 55 | 625 |
| Ex15AR | gcaggcagtgaattttctgata | | |
| Ex15BF | Tgggaaacaaagaagtgatggata | 60 | 389 |
| Ex15BR | ctctaggcttttaaaccgcttctt | | |
| Ex15CF | agaagtggtgccggaaaggatg | 60 | 525 |
| Ex15CR | aaggtcaccccagagcaaacaac | | |
| Ex15DF | tctcactcttttccagttggtaag | 60 | 587 |
| Ex15DR | atcgagctgggaggtctaatcaaa | | |
| Ex16F | aacccgcttccctacactct | 60 | 437 |
| Ex16R | cctccaaatgcatctcaacag | | |
| Ex17F | cttggaaacacagagtcagcatc | 57 | 574 |
| Ex17R | acttgggatcagagttgaaatc | | |
| Ex18F | acccaaccctcgtgcgtgaaa | 60 | 509 |
| Ex18R | agctgagacccctgagaacctg | | |
| Ex19F | tggcaagcaaaaggtcact | 57 | 489 |
| Ex19R | gctgcagctctcccccatag | | |
| Ex20F | gccagagctcccgaccac | 61 | 505 |
| Ex20R | ctaagatttgaacccaggtgtgct | | |
| Ex21F | gggaggtatagggagggatgat | 60 | 416 |
| Ex21R | tggaaagggccttacaccat | | |
| Ex22F | gatttgaataggaccacactgatt | 60 | 350 |
| Ex22R | agaaagtattgctcccaaaccatt | | |
| Ex4SEQ1 | gaccatcggaagttagtgaa | | |
| Ex4SEQ2 | aatatgtttctgcctgcttag | | |
| Ex7aSEQF | ggaggcatagctaaagttactcaatc | | |
| Ex7bSEQF | ggcccagtggagcagaagacg | | |
| Ex7bSEQR | caggagtggctgaaactttagtaa | | |
| Ex7cSEQF | tcctctgcgtcctcttcacttg | | |
| Ex7cSEQR | agtcttctgggcagccaatacag | | |
| Ex7fSEQF1 | gtcatctaactgaagaggctaagaat | | |
| Ex7fSEQF2 | cacaacatacagagaagccgagta | | |
| Ex7fSEQR | tactcggcttctctgtatgtt | | |
| Ex7jSBQF | agaagactgggataccaataggac | | |
| Ex7jSBQR | agctgtctgggatggagtctt | | |
| Ex7oSEQR | ccacgagcaggaaccattt | | |
| Ex9dSEQR | acctgttgattttgagcgttct | | |
| Ex10SEQR | aaccacttttgtaagagatttcag | | |
| Ex15aSEQF | gcaggcagtgaattttctgatacc | | |
| Ex15aSEQR | tgttttggataatctctaacttga | | |
| Ex15cSEQF | ccttggtggaccgacttgat | | |
| Ex15cSEQR | ctctgagggccgacaatc | | |
| Ex15dSEQR | gaagagggcagtcacattg | | |
| Ex17SEQF | tccctcctactctcccctgtcctt | | |
| Ex18SEQR | actgcggaggtgctggaaaag | | |
| Ex20SEQF | ggctgctgtccctgttaccc | | |

Additional sequencing primers.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1-36. (canceled)

37. A method of diagnosing the presence of, or susceptibility to, retinal dystrophy, cardiomyopathy, endocrinopathy, diabetes or Alstrom syndrome in an individual, which method comprises:
(i) typing the ALMS1 protein or ALMS1 gene region of the individual or
(ii) detecting whether the individual has aberrant ALMS1 activity, and thereby determining whether the individual has, or is susceptible to, retinal dystrophy, cardiomyopathy, endocrinopathy, diabetes or Alstrom syndrome.

38. A method according to claim 37 wherein the typing comprises identifying whether the individual has a polymorphism that causes retinal dystrophy, cardiomyopathy, endocrinopathy, diabetes or Alstrom syndrome, or a polymorphism which is in linkage disequilibrium with such a polymorphism in (i) the ALMS1 gene region or (ii) the ALMS1 protein.

39. A method according to claim 38 wherein the said polymorphism is selected from a polymorphism as defined in Table 1, or is in linkage disequilibrium therewith.

40. A method according to claim 38 which comprises contacting a polynucleotide or protein of the individual with a specific binding agent for a said polymorphism and determining whether the agent binds to a said polymorphism in the polynucleotide or protein, the binding of the agent to the polymorphism indicating that the individual has or is susceptible to a retinal dystrophy, cardiomyopathy, endocrinopathy, diabetes or Alstrom syndrome.

41. A method according to claim 40 wherein the agent is a polynucleotide which is able to bind a polynucleotide containing the said polymorphism but which does not bind a polynucleotide with the corresponding wild-type sequence.

42. A method according to claim 38 wherein the polymorphism is detected by measuring the change caused by the polymorphism in the mobility of a polynucleotide or protein of the individual during gel electrophoresis.

43. A method according to claim 37 wherein the typing comprises measuring the expression or activity of (i) the ALMS1 protein or (ii) RNA expressed from the ALMS1 gene.

44. A method for treating a patient who has been diagnosed as having or being susceptible to retinal dystrophy, cardiomyopathy, endocrinopathy, diabetes or Alstrom syndrome by a method as defined in claim 37, comprising administering an agent which prevents or treats retinal dystrophy, cardiomyopathy, endocrinopathy diabetes or Alstrom syndrome.

45. A method for preventing or treating retinal dystrophy, cardiomyopathy, endocrinopathy, diabetes or Alstrom syndrome comprising administering an agent that modulates (i) the ALMS1 protein or (ii) a component that affects or is affected by ALMS1.

46. A method according to claim 45 wherein the agent counters the effect of a ALMS1 gene polymorphism which causes retinal dystrophy, cardiomyopathy, endocrinopathy, diabetes or Alstrom syndrome.

47. A method according to claim 45 wherein the agent prevents or treats Alstrom syndrome or type II diabetes.

48. An isolated polypeptide which is:
(a) a polypeptide comprising the amino acid sequence of SEQ ID NO: 2,
(b) a variant of (a) which is able to complement ALMS1 activity,
(c) a polypeptide which has at least 80% identity to the amino acid sequence of SEQ ID NO: 2,
(d) a fragment of (a), (b) or (c) which has a length of at least 15 amino acids, or
(e) a fusion protein which (i) comprises sequence which has at least 80% identity to the amino acid sequence of SEQ ID NO: 2, or (ii) comprises the said fragment (d).

49. An isolated polynucleotide encoding a polypeptide according to claim 48 or an isolated polynucleotide that comprises a sequence
(a) which the same SEQ ID NO: 1, or complementary thereto;
(b) which hybridises to (a);
(c) that is degenerate as a result of the genetic code to a sequence as defined in (a) or (b); or
(d) having at least 80% identity to a sequence as defined in (a), (b) or (c).

50. An isolated polynucleotide or polypeptide according to claim 48 or 49 that comprises (i) a polymorphism that causes susceptibility to retinal dystrophy, cardiomyopathy, endocrinopathy, diabetes or Alstrom syndrome or (ii) a naturally occurring polymorphism that is in linkage disequilibrium with (i).

51. An isolated polynucleotide or polypeptide according to claim 50 wherein the polymorphism (i) is selected from polymorphisms as defined in Table 1.

52. An expression vector comprising a polynucleotide according to claim 49.

53. A host cell comprising an expression vector according to claim 52.

54. An antibody specific for a polypeptide according to claim 48.

55. A method of identifying an agent as defined in claim 45 comprising: contacting a candidate substance with
(i) the ALMS1 protein, a component that regulates ALMS1 or a component that is affected by ALMS1,
(ii) a component of the ALMS1 pathway,
(iii) any part of the expression pathway for (i) or (ii), or
(iv) a functional analogue of (i), (ii) or (iii); and determining whether the candidate substance binds or modulates (i), (ii), (iii) or (iv).

56. A method according to claim 50 wherein the candidate substance is contacted with:
(i) a polypeptide which is
(a) a polypeptide comprising the amino acid sequence of SEQ ID NO: 2,
(b) a variant of (a) which is able to complement ALMS1 activity,
(c) a polypeptide which has at least 80% identity to the amino acid sequence of SEQ ID NO: 2,
(d) a fragment of (a), (b) or (c) which has a length of at least 15 amino acids, or
(e) a fusion protein which (1) comprises sequence which has at least 80% identity to the amino acid sequence of SEQ ID NO: 2, or (2) comprises the said fragment (d) or,
(ii) a polynucleotide which encodes said polypeptide (i).

57. A method according to claim 55 comprising administering the candidate substance to a non-human mammal whose ALMS1 gene comprises a polymorphism which causes retinal dystrophy, cardiomyopathy, endocrinopathy, diabetes or Alstrom syndrome and determining whether the substance counteracts the effect of the polymorphism.

58. A method according to claim 55 further comprising administering the identified agent to an individual to prevent or treat retinal dystrophy, cardiomyopathy, endocrinopathy, diabetes or Alstrom syndrome.

59. A method of producing a pharmaceutical composition suitable for preventing or treating retinal dystrophy, cardiomyopathy, endocrinopathy, diabetes or Alstrom syndrome comprising performing the method of claim 55 and formulating the agent identified by the method with a pharmaceutically acceptable carrier or diluent.

60. A method for preventing or treating retinal dystrophy, cardiomyopathy, endocrinopathy, diabetes or Alstrom syndrome comprising administering a polypeptide as defined in claim 48 or a polynucleotide encoding said polypeptide.

61. A non-human animal which is transgenic for a polynucleotide as defined in claim 49.

62. A probe, primer or antibody which is capable of detecting a polymorphism as defined in claim 38.

63. A probe or primer according to claim 62 which is a polynucleotide that has a length of at least 15 nucleotides.

64. A kit for diagnosing the presence of, or susceptibility to, retinal dystrophy, cardiomyopathy, endocrinopathy, diabetes or Alstrom syndrome comprising an agent, probe, primer or antibody capable of deleting a polymorphism as defined in claim 38.

65. A method of identifying a polymorphism which can be used to determine whether an individual has or is susceptible to retinal dystrophy, cardiomyopathy, endocrinopathy, diabetes or Alstrom syndrome comprising screening the ALMS1 protein, or the gene region expressing the ALMS1 protein of one or more individuals.

66. A method of determining whether a candidate polymorphism in the ALMS1 protein, or in the gene region expressing the ALMS1 protein can be typed to determine whether an individual has or is susceptible to retinal dystrophy, cardiomyopathy, endocrinopathy, diabetes or Alstrom syndrome, comprising determining whether the candidate polymorphism is (i) associated with retinal dystrophy, cardiomyopathy, endocrinopathy, diabetes or Alstrom syndrome or (ii) is in linkage disequilibrium with a polymorphism which is associated with retinal dystrophy, cardiomyopathy, endocrinopathy, diabetes, or Alstrom syndrome and thereby determining whether the polymorphism can be typed to determine whether an individual has or is susceptible to retinal dystrophy, cardiomyopathy, endocrinopathy, diabetes or Alstrom syndrome.


SEQUENCE LISTING (100 sequences)

SEQ ID NO: 1 - DNA, 354396 bp, Homo sapiens
gaattctaaaatccagaaaacatcaaagacaaggagcttccagagcagattacctgcaaaaggcacaagaaccatactgacattgcttttcccacagaaaaagtggatccaagaagccagttgagtgataattttagaacatgaaaagaaaataatttcaagtccagaaattttttatgcaaaaggtatagcctaaatgtaagaccttaataaagatgttctcaggcacaaaggccttggaaaattaccacataaaaaaacactttggaaatgctctaggggaagtattcaaataggaagggaaaccaagaggatgctgcaaggtattaggaaatgtgagggcatttatgtaacttagcaaatattattgtttaaaaacttaataaccaaaaataaaaaagagagaaagtatacctataacaatctggaattgattttctgcacaatatcaaaatgctcatggtcatgatagacataaaaatatgttaaaattattgttttgcttgaagagtaatatgttgataaataaattcatagtagtaaatgaacataaatataggacttaagggcaattatataacgaaaagcaaaagcattcaattttgtctatccagtgaaaacagaaaaggaaaaaagataaagtacaatacatagataatataaaataagtcccagaaatgttaataactttaaatgagtaaaattgatcaatgaattctagacacccttgaataaaatggaaaaaaacccaaacacaagatccaacaatgtgccatctacatgaaatgtactgaaagcaaagtaaaatgacaagttgaaaattaggccaggtgcagtggctcatgcctgtaatctcagcattttgggaggctaaggtgggagttgaggctgcagtgagccaccgcactccagcctgggtgacagagtgagactctttctctaaaaaaagaaaagtaaagaatagaaaaagaggccggtcgcggtggctcaagcctgtaatcccagcactttgggaggccaaggtgggtggatcacctgaggtaggagtccgagaccagcctgaccaacatggagaaaccccgtctctgctaaaaatacaaaattagccagatgtggtggtgcatgcctgtaatcccagctacttgggaggctgaggcaggagaatcgcttgaacccgggaggcagaggttgtggtgagccaagatcatgccattgcactccagcttggacaacaagagcaaaactccgtctaaaaaaaaaaaagaaaaagatatggacaaaaagaaaaaaagaaagttgggtaccagtctttctatctgatgaaacagatccttgacaaaaatcataacaagagacaaaagcattgttatatgctaagaaaaggaaccacagaacaagaacatatagtcattaagaatatatatacagtaaaaacatagcctcaagatatattaaatcacaactgactgcactcagaggggaatcaattatcaataatagagattttaactacaagctgatatatcagcaaataaaaactaagcagagaaatataaaatctgaataatattattaataatatccaggctgggtacagtggctcacacctgtaatccagcattttagaaggccaaggtgggaggttgaggctgcagtgagccactgcactccagcatgggtgacagagcaagaccatgtctcaagaaaataaaaaatacctagctgctaaatatatattgaattctacactaaacagagaatacattctttttttagttcacatgtaatatttacaaacatttaccatatactaaataacaaaggaaacctcaattaattctacagaatcaatattatacaggattttttttgtttttttgtaaaggaccagatagtaaatatttttggttttgtgagtgtctaccttaattactgaactgtgcagttgtagcacaaaatcagttgtagacaatacataagtgaatatgtgtggctgtgttttgtgagatatatataaattaaaggatacacttaaaactcattagaattattgcctgtgtctacacaggcagggtggtggtgtgggtgggaaaacaggctttggggaatagggctaaaaaaaataacaaactcaagaaatgccttgcccagaacaagtatgataatggccaggaaccgatgggaatgattaactctacatttgaggtccagaatgttacagaccagctgtactgtgttttcaactacttgtactcatttgtctctgagtgcactgatgtttaattggctgcccatttagcagttatagtctgataattacagtttaaacattattataatgttgtatttcatcttttggtaatttttttggtatagctatttttgcaaaagcatttgttgtctatgatttgtatctcattttaactacataatcaattgatgtaattgatttggctcctcctattacattgcagtcaaggaaatattgcccaaatatgcatagccctgtgaacaataagaaacaaaaatgttcagattttaaaatttcataaaggcttaacaccaatcttaaactgtacgtttatttattatacttatttacttttgttgtacccaatgactaaaataagcccctattaaagaccttgaaattgttcatcaatagcaaataaaattttcctgaggattcttttaaaaacagatttggcttgagtattgaaatctatttgctatcctaggatagggaaagatatctcacatgtatagagcaaataccacaagccaggtgctatacatgtgttattccaattaattttcaaaacagtttctagagcaggcagtataatcccacttaacagatggagaaactgagattcagataaatcaaataatctgcccacgggctcatatccagtaagtggtgatgttagaatacaaactgaataaacatgagttttagatcagtattcaatgtttaaaacattataacagaacttgagttccagcgtggtggtgtaagcctctacaggccatctctcttgccaattaaaactaaaattgcaagacaaataaaaaaaaaaaacaacactaccaaaggactctgaaaagttggcaggggggcaggtagatcatggaggggagtcaaaaattgaacaaacaaccaatgtggaggtgaacttccttttgtttctcctcttctcttgtggccttgcctcaaggctgagctgtggttgtagagcagtgcaaagacatctgtggctaaagttcaaacagaaacctattgtttatggctaagtaaaccaggaaaatgagctcctgcaggcctaagagtaggggatagaggagagaaccatagaaggggatccactaatttggcatatgagcccataccagtctcagcataagccctgagctatggatgcttggacaaatacaaatatttgaactaaatgattaaatattagagtgtctaactgctgaagaggacatgtgcaggatacatgcaaacaaaggtgacaaaggcttagaggactgaagcaggatttgaaccacctgcacaactttcagaataacctgtaagtgtaaatgaacaagatagacatgaagcagcataacaaagccttagagaactaaactgaactaccacagaaggcaaggtagaaattacattctgagcttaataatgttgattgcctgctaaaacaaaaacataaacatcctccagagaatgatgacgtgatccagtctatccaatacaaaattcacaattttcaagatacaatacaaaattactaaacataaagaaaaactaggagatgtgaataattatctagggaaaacgtaatcaacaaatgccaacctgaagataacctagatggtggaattatcagatagaaattttaaaacagcttttataactatgctccatgaggcaaggaaaaacaaacttgaatgaaaagagaggtattgttagcaaagaaatagaaactataaaaagaatcaaataaaaatttcagagctgaaaaatacaatctctgaaacaaacaataaaaccactggatgggctcaatagtaggatagacaagacagaggaaagaataagtgaaattgaagatagattaatagaattatctaataagaaccaatcaaaactagattaaaaaaataaacagagccttggggatctgtgaaaaaatataaaaagatatatgtgtagttggagttccaaaaggagaagaaaaagagattgttgcagtaaagctatttgaagaaataatggccaaaaatgtcccaagtttgatgaaagacacaaatttacaaattcaagaagcctagcaaaccctagacaagataaacttagagaaaaccacacttagaaacttcataataaaagtactcaaaaccaaaaactaaagaaaatatttgaaaccaaccagaaaaacaacactttacccacagaggaacaatgatttgaatgtttacaggtttttcactagaaattatggaggcgtgaacataatggaatcatcaacccaatttttgtttgtttgtttgttttgtttgtttgtttgtttgtttttatacagggtttcttactctgttgcccaggctggaatgcagtggcatgatcatagctcattgcagccttgacctccttggctcaagcagtcctctagccccagcctcccgaatagctgggactatagccacatgccaccatgcccagctaatttttattttcattttttgtagagacaaggtcttgctatgttgaccaggctggtcttaaacttctggcctcaagtgatcctcttgccttggcttcctaaaatgctggattacaggcatgaaccaccatgcttggcaacccagaattttatattcagcaaaaatattcttcaggaatgaaggtgaaagacagttaaagggaaacagaggaacaaaaatcagaaggaacaacatagaacaaataataaaataataagctaaatcaaaccatatcaataattacattgggcctggtacagtgactcacacctgtaatcccagcactctggtggatcactggagctcaggagatctagaccagcctaggcaatgtagcaaaaccctgtctctacaaaaaatacaaaaattagctgggcatggtggtgcatacctgtagtcccagctactccggggtgttgaggcaagaggaccccagctactccagggggctgaggtgggaggatcacttgatcccaggaagtcaaggctgcagtgagccgtgattgcatcactgcactccagcctgggtgacaaagtgagaccttgtctgaaaaaaacaaaaacaaaaacaaaaaaaaacttgaaaaaaaattacattaaatattagccaatttgtgctgggatcatagcaggaggaatacttaaggccaggaatttgagatgagcctgggcaacatagcaagacatagcctctaaaagaatttttaaaaattagccaggcctggtggcacacacttgtagtcccagctacttggaaggctgaggcaggaaggattgcttgagccctggtgtttgaggttgcagtgagcatgattgccctccagcctgggcaacagagcaagactctgtctgaaaaaaaaaaaaggaattagccaattaacatttatagattagtggaattgaattgagagtctggaaatgaaatcttacatatttatggccagttgctttcaacaaggctaccaagataattcaatgggagaaagaatggtctttccaaaaaatggtgctgggaaaactgtatgtccacatgcaaagaatgatgttggacccctacctcacaatatatacaaaaatcaacttgaaatggatcatagacctaaatgtaagagctacattaaatttccatataaattttagtaagtttgtcaattcctataaggaagccagctgagattttgatagggatttcatcgaatagatagataaatttggggaatattcccatcttaatcatattaagtcttcccatccatgaacgtggatgtctttctatttatttagatcttcttcaatttctttaaacaatgttttgtagttttcaggatataagtttgtacttcttttgttaaatctattcctaaggatcttaccctttttgaagttattgtaaatgaaattgttttcttaaattcttttttattattcattgcaaatgtacagaaatgcaattgatttgtatgtattcatgttgtattctgcaactttgctgaacttattagttttagtgattttttttttagtggattacctagtattttctgtatacaaaattatgtcatgtgtgaatagataaccttacttgttcctgtccaatttggatgcctcttatttcattgtgtacctaactactgtttgtaattacctaattacctgacagaacctccagtacaatgttgaagataagtggtaagagtggacatccttgtcttgttccaaatcttagggggaaagcatccagtctttcaccattatccatggatttttcatagatatcctttatgaggttaaataagtttccttctattcatagtttgttgagtgtttctatcatgaaattacattggattttatcaaatggtttttctgcatctattgagatgatcatggggttttgccctttattctcatgatatactgtataatgttgatttttggatgttaagccaaccttgttttcctgggacaaatccttcttggtcataatatataatctcttttaaggaaatggtttattagatgtgacaccaaaactacaaccaaccaaagaaaagcagacaaattgaacttcacaaaagttaaaaacttgtgtgcttcaaaggacaccatcaagaaagtaaaaagacaacccacagaatggaaaaaaaaatttgcaaatcatatatctgagaagagaattgtacttagaatatataaataacggttacaacttaataatgaaaatacaaataacccaatttacttgtatttaaataaaggatttgagtggacatttcactaaagaagatgtacaaatggccaataaacacatacaaagatgctcaatatcagctggacactgtggctcacacttgtaatcccaacactgtggagggccatggtgggaggattgtttgagccaggagtttgagatcagcctggacaacatagtgagaccctatcttaaaaaaatatgctcaatatcattagccaccagagaaatgcaaatgaaagccacaattagacaccacttcacacccactaggagagctaaaattaaaaagacagacaatgaaaagcattgatgaagatgtggaaaaagtaaaaccctcatacattgctggtgggattgcaaaatgatgcagccactttgggggacagtttggaagtatcttaaaaagttaaacatagttaaattaacagagacagaaagtagaatggtcgttgccaggggttggtggggcagataaaacaggaagttaccgtttaatgagtagagagtttcagttttgcaagtagaaaaaagttctggagatggatgctggtgatgtttccacgacagtgtgaatgtacttgatgccactgaactgtacacttaaaatttgttagagtggctaattttatgttatgtatgttttaccacaattaaataaaaagttaaacatagaattccataggacccaccaatggcaattgtggatatctactcaagaaaaatgaaaacgtatgtccacacaaaaacaagtacatgaatattcacagcagctctattcatgataactaaaaagtggaaacagcccaaatgttcatcaactaagcaaaatgtggaatatccatacattggaatattgcttggcaatacaaaggaattaagtattgatacatgatataacatgggtgaatcttgaaaacattatgctatgtgaaagaagtcagtcacgaaagatcacatattggatgattccatttatatgaaatgactagaataggcaaatccatagagacagtagattgtggttacaagggcctgcagacgggggagaatggagaatgactgctaatgggtgtggggtttctttttggggtgatgaaaacactctaaactagacgatagtgaagattgcatagttctgtgaaaacactgaaaatgactgagttgtacactttgaaagagtgaattgtatgggctgtgagttatatctcagtaaaactgttatttaaaaattgcacatatgcagagaaatgtatctagaataatgtccaccaaatgtatgcaatttgaggtgatttgttgtttttatatatcacttacattttaaaataaggaacatgaattaatttttataaaaataagtcatcgttttcaaagaagtaatggggcaagtgaatccagagtgtagaggtctcacaacttaaaggaaggggagtggctgttcagtggaaggtagagccgattttggggagaaaaactttgtgtttccagtggctgtgtctgctgtgcccatgcttgctgaagcaatgcaaatttcattagcatggttacaattagtaacataacttcatttttatgttctcctaacctaagctgagtaattttcaaatttaaaaaaaaactcaactcctcatgatacagttttactgaactcaaagaggatctgttgggacagataatttactgacagaaaagctactttcaaagaggcaagaacagaaatatctgctttccgtggtgtgcctctgatttattcatggaaaaaagtatgcctgcaaacattatctcagcaggccaagccataattagagctctaattttcctccaggcactttttcctgaattgtcctaaggacccaatgagaggtgcctatacaaccatgataatggttctgaacattttcagtgccagcacactacagggctgatgtgtgggaagtaggaagtcacatggtgcgataggagaaaagcaactgcctgaccgtaaggcttgcatccacattcctccctctcacaaaggttagctgggaagacccacagaatccccataaacctttccccaaagaaaagacccatttctttgaaccacccatcccagagagagtttgccttggtaaccagagtatctggaacaagatgaagataatcagtaaaaccaaaagggaactgagcaggctttgagaaatttcctttgcttcaaaaataataagagctagtagccaatgacaggtgaaaggacaagtgtctaaacatctgtcaccattgatttctgcatacctccaatatcttgatttcaagcatatctctttttattttttatttttgtttttatttttgagacagagtctcactctgtcacccaggctggagtgcagtggtacgatctcggctcactgcaacctccacctcccaggttcaagcgattcttctgcctcagcctcccatgtagctgggattacaggcatgcgtcaccaacacccggctaatttttgtatgtttttagtaggacggggtttcaccatgttggccaggatggtctggaactcctgacttcaagtgatccgcccacttcagcttcccaagtgctgggattactacatgtgtgagccgccacgcctggcctcaagcatacctctttttgattaccacctcctatccttccagtgcatttgctttagtaacccattccaacaattccttttttttttaaatttttaattgttaattttttttttttttgagacggagtcttgctctgtcacccaggctggagtgcagtggcgtgatcttggctcactgcaacctccgcctcccaggttcaagtgattctcctgcctcagcctcctgagcagctgggactacaggggcgtgccaccatgcccagctaatttttgtatttttagtagagacagggtttcaccatattggccaggctggtctcgaactcctgacctcaagtgatccacctgccttggcctccaaaagtgctgggattacaggcgtgagccaccgcacccagccctaaatttttttttttttttttttttttttttagagacaaggcctcactgtcacccaggctagagtgcagtggcatgatcatagctcacagcagtctcaaactcgggttcaaacgatcctgctgcctcggctgcccaaagtgctagggttacaggggtgagtcactgcgcccagccccagcaattctttaactgtcctgaaatctcagttcattgattttcattattgaacaacagcttcatgatttcacttcagttcttacccaaagcatctaagactcagggaagttgtgagtctacgtgtttatttatttttacttgatttcctttggttcacctacttctaaaaccagttgaagatggattgaaatagcaaaactctcatgacagaatagagtacagatttagaaaaggaaatcaaaattattaaaatgtcaatcacatgggaattggttatgtaattggaggaggaaacaccctttcttatcctctctttcactgtcagagattggagtgagggcaggccttggctaatcattgaacttggctgagggtaaagggaacgggcaactgtgaagacagtcctttgagtagctaagcagtggttcttgagagccagatagtttaacaatgggccttgatcttgggcccagaacactagtggtttgaagggagtgaagacctattgagagagtgaggtgagcaggaggaagtcacactgttctgcatagtgtcaggtgctctctctcccgtgactagggactgtcattagatttatgtgctactaacgacaaccttttctttggcaatataaatttggactgtgctgtctccaagaaattagttcttaccctttggaaatatatgtgagtgtgtatgtgtgtttcatcaatagttacctgtcggacatcggaagacaaaaaggaatagctacatctcaccccatgtggtggacagactctaagactcccatgattttcttgttcaggtatccacactttttgtaatacctcccttgagtgtaggtgggacctgtgacttgtttctaaccaataaaatatggcaaagatgatgggatatcatacttgtgacattatattatgtaaaactccatcttgctagccaactcacactgacagactccctgtcactggttctgaagaaggaggctgacatgtggcaaaaaactctgggtagcctcttggagctgagagaagcccccagcaagaaatcaaaaccctcagtcctgcagctgcaaggaaatgagttcttgcaacaacctgtgggagcttggcagtgggtcctttcctagtgagactgcagcctcagctgacatctggattgtagcctggtgaggcctggagacagagcaactcagctaagtcatgcccaggcttctgagctacagaaactgtatggtaataatttgtgttgttgtaagccactaagtttatggtaattggtcctgtagcaatagataactaatacatcctggatggtgggagcatttttggaaacatgggagaaattggttggagaagtggtacggaatcaaaagttctgttttagccacgttaaattttagcttcctaggaggtatctgagtgagatgtcaagcaagcaattagatatgtaaccctggagcttaagggagagacagaactagaatataattttgggagtcatcagaggatgacatttaaagtcatgaaactagatgagctctctacagagaaagtgaagggcaaaaaggaagagagcccagattgaacactggggtacaccagcatttagcagttgatcagaggaggaggagcaatcagaggagatgaaagattaaccagagaggttgaagggaaaccgagaaactgtagtgtcccgatagacaagaaggaaggaatcgtatctgtattgaatgttaatacggggttcagtaagatgaggatagagaagtggccacatggaggtcactggtgaccttgacaaaatcagtttcaggccaggcatggtggctcacacctgtaatcacagcattttgggaggccaaggcaggtggaccacttgaggtcagaagttcgagaccaacctggccaacatggtgaaaccccatctctattaaaaatacaaaaaattagctctgcgtagtggcaggagcctgtaatcccagctactcaggaggctgaggcagaagaatcgcttgaacccaggaggcggaggttgcagtgagccgagattgtgccattgtactccagcctgggtgacagagcgagacttcgtctccaaaaaaaaaaaaatcagtttcagtggaatggagaagtgaaataagaaccaggtgtgagagggatgaggagttaataagaggtgaggggctgctaagggtgaatataggcaatttgtttgaaaagttttattctgaagaaagagcattgggtcaatagctggagaaagatataggaacaagggtagggttttcaaagataggagatcataagacatgcttgtaagctgatgagaatagtttagtagagagggagaaattaataatgcaagagaacaagagaatggtttcaagagcaagctcttaaggcaaaaggggctgggattgagagctcaatggaagagttgaacttagatgggtttagggattcacccactgtgagagggaggaagtcagtgtatggatatagataggaggcggcagatctggtggtggaaaggggagaatattcctgtttgactgtcacagtgattagtaatatatgattagttgaggtcatcagctgagaaaagggggtggaaaaggtgttggagttttgaggatataggagaagctttaaagtagccttctttaggagaggtacccccatctcagtagagagatacaggattatcaggcagtatggatttacaatcccactttagatttgcactcatgaatttaaagtgaaaccagcccaaacagcaagacagcagacattggggagaatcaggttaaaccataattttcaattcctcaggtgcataaacatgcagatagtcgagcttgtccaggaagtgcagtggaagaagagagggttgaggaattaaggttataaatgagtctgtgtagtgaggagtgtgggtacagaggtttgtgagtaagacaagagggagggagagtgggagagggccagagtcactggattggaggtctcaatcagtcaaaaattgcctgaagtggggctactagaattaggaagctaaaaggatgggtggcagtcagagagacagatgcttgaaattgagatatttggaggcagcacattgatccataatgacagctatttggatacttgtcctttggccttatcgatcatctgaacctttgaaatcttaaactctcatgattcatatagaccacagcctccaactatttaagctcactcattctcttattcctaaaatagtggaatcacatcattcatgtttttaactaatttaaattcggttctacagtctggggaaaaaatgacagcgggtaagacagaacagtttaaatagcacagttgcctcactgactttcctttcaagagtgtgtgctggggagtgaggatgaggtgggagccatgagtctgctgtttcctaggttgacctcagttcccttgtacctcttatcgcgggattgtctcacggccttcactgtgattccagtgtttaaagagcacttcctcttgttgtcaatatggcctctaaacacatgccaactgctttatgggctgctcagccaatgacagagttctgggctaatgctggccatgtagttctgactggaaaacagtatattgggtgtcagtgcaaataatatattaagggtgatttaaggcatttccaaaggtaaatttgaccacctaaaatgtatgccccaagtgctggctaaattttgaatccaggccctccaatgatgcagctccactatattcccattttggccacaattggaccctccgtctcttgtccactacattttctcccaatgaaatccacaccctcatgctgtcatttctttcctattgaatctggacccccagaatgcccccttctgtcctttccatttcgccagaccctcttgcctcttgtcccaccagtgtgcttggcctccagaaccttaagcctcagttgattcaagcattcccttgctcagccgaatgctgctgaaaatattcatgtaagagtaatttccaatcatggtttccagcttcacctggcccctcatacctgctccttaattaatccttctgtatgtcagaaattatgccgttacccatctctccagtggttcatctaacctctttgcctttttctcacatcccctcccgtactaactccctctcgggtaaggacttgcctattccactgggaaaattgaggccatcaggcaagaactttcccaactagaaactaaacccacccttccttccttccttccttccttccttccttccttccttccttccttccttccttcctttttgagacagaatctcactctgtcacccaggctagagtgcagtggcataatcatggctcactgcagcctcaacctcctaggctcaagtgatcctcctgcctcagcctcctgactagctgggactataggcgtgtaccaccatgcctggctaatttttatatttttttttgtagagatagggtcttgctctattgctcaggctggtcttaaactcctggcctcaagcagtcctcctgccttggcctcctaaagtgcttggattacaggtgtgagccactgcatctggccccaaaccctccctcttgacccatcccttatcttttcctcctgcctcagtgaaaggtatttctctcccatctaggcttggaatgcctctcaccttctcaggaatctgctcactacagctcctgtctcctctgtatcttcagcctcttttcatacatgctgcgtttccttcagcacttgctcaagccaaatcctgtctcattcctatcctctaactctccattacttaatagccaggctatttgaaagagtcagctgcaagaactacttcactcaaaaaccactgcagctctgtgtctgtccccaccatgctggtgaaactctcttccttgagatcatccattacttccaggatgccaaattcaatggaccattatctttccttctccttctcttatgtgacttctctattactttattcctccttgaaattctctctttttttttttttttttttgacagagtctcgctctgtcacccaggctggagtacagtggggcgatcttggctcactgcaagctctgccgcccaggttcatgccattgtcctgccttagcctccagagtagctgggactacaagcatgtgccaccacgctcagctaatttttttgtatttttagtagagattgggtttcaccgtgttagccaggatggtcttgatctcctgacctcgtgatccgcctgcctcggcctcccaaaatgctgggattacaggcatgagccaccgcgcccggtctggttttttggtttttatcataccacactgtcctgattttccttccccctgtttgaaccaactaagcatccgtaaaaaccttcctcctcactctgacaatgaaggaaagggctcccattacaatcttaaaatccataaagctgtggccaattataccaatttcttaggccaatgactctcaaactgtggtcctggaccagctacctcagcatcacctgggaacttgttaaaatgcaaattactggactccaccccagaactactgaatcaaaaactttggggatagggttgaacaaactgtgttttaataagctctccaggtgcttctgaggcaagctcaagtttgagaactactaccttaggtctcttgacttctagctctcaactcacaattaaaatgtctttatttatgtggtgatacttcttggggtatttccaactagtatctcggaatgcaatgattaaaagatgaactgctagatgactttgaccattttggctttgccacaatgctacttagtttaggtagggctttggatatttttcttggcacttaattgtcagcattttgaatgtccatagttcatccagatgatagccttatgcattcaccaaaactgatgtctcagtagtcacgtattccacgcaatccttgctgcctgctaaagtgctttctctggacaaatctggggaagtcggtgtctggtagttcaagatacagttccaggggtcttgttctgtccagctaagaagtgagaaaaaggggtgatttggagtggatcctccagtgtcaaagtcaaccagagattcctgaagattgccagattctcttgatggtttattcatgacattttcatcttcattgtcccgatattggattatcttaagaagggaggtgccaacaatttgtgcagccaaggtgcttattacatgaatctatctcctggttaattttacaggtaatcccttttcagaggagacagagtctcactctgtctcccaggctggaatgcagtggctcaatctcggttcactgcaacctccggctcccggattcaagcaattatcctgcctcagcctcccaagtagctgagattacaggtgctcaccaccacacctggctaatttttgtatttttagtagagagcatttctccatgttggccaggctggtctcgaactcctgacctcaagtaatctgcctgccttggcctcccaaagtcctgggactacaggcgagagccaccgcgcccagcctcttccctgctgttaaatattgctttgcctcttactaacccggcgaccttggccaagttgcttaaccaatctgtcccttagtttcttcatctgtaaagtggacataaaaacacctacttcttgcggttgtgaggattaactaatgttatttagcatcatgtctgacaaatagtaggtacttcatacatggggtcaagttgatcatcattacttaaaaaaagaagtatctaccttgctttatgtctggagattttgtggctgtgtctcttgtagacagaaaaatgaaatctaaatgctggaaaaaatatacagagaaacagaatgattttaaatccaagttccagcaagacactgggaagagaatattaggtgcagagagatcagatgggagaaacaagatcacacaacaggaaagctgggaactgaagccatttttgagttttagtttaatgctttacatagtggattgattttgccattctgcctgattcagatgccatggggagaataacagattaatttgatagaatcagagaatacataaaagagtgtttaatggagtgctaacaaagctctgaatagattttctttttttaaactttttaaaaacataaatagaacatgtttattgtagaaaaatagaagaattcagaagaacaagaagaaagcatcaaaaactttagcaaccttgtaatcacactactcagagaaaaccattattaatattttgtagtgaatcctttcagttaacacttatttcaatgtgtgctcatgcacacgtgcatgcacacacacacacacacacaccagaaacacatatcccatacactttttttttttttttttcagacagagtctcactctgtcatccagactggagtgcagtggcgccatctccactcactgcaacctctgcctcccgggttcaagcgattctcctgcctcagcttcccgagtagctgggattacaggtaagcgccaccgtgcctggctaatttttgtatttttaatagagacggggtttcaccatgttggccaggctggtttcaaactcctgacctcaggtgatctgcccaccttggcctcccaaagtgctgggattacaggcatgagtcaccatatccggcctccatacactttttaaacaaatcaatttgcaccgaatataccattttgtaacctggtgtttccattcaacaaaatgttatgaaagttttccatagaccttatttcatatgataaaatatttcatttcatggatacaaccaatttatttaactaatactgtaactgatgaatctaatgacattatgtacaatgcaatgataaacattcctgtatgtacatatttttcattaatacaaccttaattgagatatacatacaaaaatacagaaatcataagtacatagcttgataagtcatagaaaaatagatatagccaggcaatacctcccaactcaagagatagagcaataccagcatctcagatgctccctttgtgcccctcccaatcactaccccttttctcttccctagtcttaactactattctgatttctgtctaccactatagattattattgcctggttttgtacttaatatacttggaattctatgatatgtattattttgtgcttggcttcctttgttcaacattataagtgtgagattgattcatgatattgtagctttggtttgttcattttcattgctgtatattatttcatggcacaggtataacataatttattcatccacttttattgttaatggacatttggattgtttccagtttttatttattaagaaaaatattgttcgtgaacattcttctacatttatttttgtgcacatatgtacacatttctggagtataggataggattgttgagtcatagtatattttcaactttcaatttattttcacttctggagtagtatcagttaaactccccccagcaatgtgtgagaattcacattgttccatattttctgcaatactatagtgtcagtcactttggttttagccgtgctggtggatgtgaggttatatctcatggttttactttacatgaggttgagcacttttaaatatggttattgaatgtttggacatctcttttgtaaagtgcccattcaagacttcatcatttttccattgggttgctgtcttttttattctaacagttctttgtacagtctggatgtaagtcctttgtcagttatatgttgcaaatatctttgccagtgtgtgacttatgctactgcactctttgtggtatcctttggtgaacggtagtccctaatcttaatgaaatccagtttattaatcttttcctttttaaataagtgcttttttttgtcctatttaagaaagttttgtctacctcaaggtcatgaagatagtctcatgttttcttctaaatctgttgtttcaccttttgcatttagatttgcaatccacctagaattgatttttaggtatggtgtgaggtggggtcaaatttcatttttgggtgtgtatattccattgatccaataccatttaatgaagagaatgcctgttttctactgctctgaaattctacctttgtcactaattgagcatctgcctatgtgtgggactgtttctgtactctctattcttctccattggtttatttgtctatcttttcacacagaccaccctgcctcaattactatagttttatgataaatattgatatctgattatgtctgttatcccaccttgttttctttcttcagcaagtattgataagactaaagggaaaaaggaatttttatatactgttagtggaatgtaaattggtacacatactttgaaaaacagtttggcagtacatgatgaagattcatatatgctatgactcagaaattccactcgtatgtacatacccaacagaaatgtgtccacatgtacaccaacaggcatgtataagaatattcttagtgacatattttttggaacaaaaatctgaaaataacccaaatgtccttcaacagtagaatggactaatgtattgtggtgtattcatctacacagccacgtaaataaatggatccagtgtaagagactgtgttcctctgcaactacagtgcctgtggggcggctcctccatagcttcacctcttgctgggattcagtaacacatttctttctcttgcttttcaggcttagggggtggtaacagtttctcactgtttcaagtcagtgtgctctgacagtaatcctgtccacacctctgtgcacagctccttcataacatctcttaattggatcatacatggtgaattctgtttggtgccaggaccctgaatgacgtacacacacacacacacacacacacacacacacacacacacacttttcatgtggatgcatacgcatgatcgtattatacatacaattattaaaaagttttatacaattgaattattctaatacaattctatttccttttcttagattaccaggttctgaaactcacagggacttcaaaagtcaacctgtctaatcccaactttgggtagatcaattgttttcaccatgtaacagatgggccactatgggccagagaggttaaaccccctgttcctggtcacacagctggatgggtggctacgtgaagactagaacccaatcccctgatacggagcacagtgaagtgccatttctcttacacgctggctttcaaacttggctgcactttagaatcaccagagagctttaaagaatattgatgcctgggtcccacctccagagaacttgatttaattgattttgatttaacattttaataaaacatattttaaagatatttgtattataaatattttaatttttattataaaataaaacatcacaatagaatatcacacaaatcaaatgcctggtttaatgaattattttaaggtgaataccacccagattttttaaaaaatagaactttgccagccactctgaaagccccttcagtgtaacccatcccaaccacaatctctcctcctcccctaaataacatcctgatttgttttatttatttatttatttatttattttagacacagagtctctctctgtcacccaggctgaagcgcaatagcaagatcatggctcacggcagcctccacgtcctaggctaaagtgatcctcctgcctcagcctcctgagtagctaagaccacaggcccatgccaccattctcagttaaatttttttgtttgtttgagatggagtttcgctcttgttgcccaggctggaatgcaatggtacgatcttggctcactgcaacctccgcctcccaggttcaagcattctcctacctcagcctctcaagtagctgggattacaggcatgtgccaccaagcctggctaatttttgtatttttagtagagacggggtttcaccatgttggccaggctggtctcgaacttctgatctcacatgaaccatccgcctcagcctcccaaagtgctgggattacaggcatgagccaccatgcccggtcctcggttaatttttaatttttaatttttttctagagatgagatattgctatgttgcccaggctggtctcatatttctggcctcaagcgatcctcctgctttggcctcctaaagtgctgggactgcaggtatgagccaccgtgcccagccatcctaacttttatagtgattgcttccttgaagttttcctttagttttatgacaggaatgtgcatccatagacactgtagtttgctcttgcccattagaagatatatctttttaagtatctttgaatctgtgggttcctcctccaccctttattttgcatacaagctttgtgacctgtactctcccacagtctggatttcattatttgaattctcttggtacagttcaatgtgtttctctgtcctctgtatttcctgtaaatagacaggtgatccagaggtggattagatacaggttcgatccctttgacaaaactatggacactgtatggcaggactgtaggaggcacagattgtcctgttgtcattttttaatgatcttaatagctgcaaagtgtcagtatctatcattcactgcagagttgtcaactgctgatattctatcatttctgttttatttgagttggaatactttcataaaaacacacattctctcatctactgttttgttttccaggagtaaagttcatatagaaaaggcaggtactatagtctaaatgtctatgtctccccggaattcatatattgaaatcctaacccccaaagtgatggtattaggaggtgtgaccactgggaggtggtcaggccatgagggcttttagtcccttagaccatgtgaggacacagcaagaaggtgccattctatgaatccagaggtgggccctcagcagaatccaaccatgatgacatcctgatcttggtctttccagactctagaatggtgagaaataaaattctgttgtttatttatttctttatttaattaaattaattaattaatttatttttgagactgagtctctctctgtcatccaggctggagtgcagtggcacaatctcagctcactgcaatgtccgcctcccgggttcaagtgattctcctgcctcagtctcctgagtaactgggactataggcacacgccaccacacccagataatttttgtatttttagtagagatggggtttcaccatgttggccaggctggtctcgaactcctgacctcgtgatctgcccacctcagcctcccaaagtgctgggattgcagacctgagccaccacgcctgacctatttattttttagagacagagtctcgttctgtcatccaggctgaagtgcagtggcacaaccatagctcactgtagcctggaactcctgggctcaagtgatcctcccacctcagcctcccaagtagctggcactacaggccgaggccaccattctcagctaatgttttctttgtttgtagagatagggtctcaccaggttgtccaggttggtctcaaacttctggcctcaagcgatcctcctgcctcggcctcccaaaatgttgagattacaggcgtgagctactgcgcctggccttctattgtttattagccacccagtttatagtgtcttgttatagcagcccaagtggactgaaagagcaggataaatgttctatttattccttttatttaccagttttctttttttttatatatatatatattttattatactttaagttctagggtacaggtgcacaacgtgcaggtttgttacatatgtatacatgtgccatgttggtgtgctgcacccattaactcatcatttacattaggtatatctcctaatgctctccctccccgctccccgcaccccacaacaggccccggtgtgtgatgttccccttcctgtgtccaagtgttctcattgttcaattcccacctatgagtgagaacatgcggtgtttggctttttgtccttgcaatagtttgctgagaatgatgacataggcatgggcaagaacttcatgtctaaaacacaaaaagcaatggcaacaaaagccaaaattgacaaatgggatctaattaaactaaagagcttctgcacagcaaaagaaactaccatcagagtgaacaggcaacctacagaatggggaaaatttttgcaatctactcatctacaaagaactcaaacaaatttacaagaaaaaaacaaacaaccccatcaaaaagtgggcaaaggatatgaacagacacttctcaaaagaagacatttatgcagccaacagacacatgaaaaaatgctcatcatcactggccatcagagaaatgcaaatcaaaaccacaatgagatatttaccagttttcaaggcaaaatattggttccctatcatcctttgaaggtgactaattccttttttcaaacaaatatcagatactttgatcttagatttagacacatttgatgagtttcaagctattacagctattataattattgaagcttttcagtgacaactggacaatgtgtgcctactatagtcctgtcatacaatacccatagttttgccatagggattgaacctatgtctaattcacctctggatcagctatttgcaggaaatacagagtttagggaaacacactgaactgcaccaagagaattcaaataacaaattgttgacctatggccaatgggagcctcctcaggttggcctctgagtccttttgacataactctagtagtctttgatggcttgcttgcaatctggtattacacgatactccaggttcatcttgtatttttcctgccccaaacttgggatcaactattgctttgggtaaactcttaaaccatgcttttcctctgctctcacaccacaacaatcaacacagaagacttctgtgaccaaatatatgggggttttcccccaactacaagcagtgaacaccagctgggtgtcttccaattcaactccgactctatgtacctgcagataacatcggattccacaggtcgagggctcagaccccaagattgccccctccttcagacactagttgcaagtttgggcctctgaaagttctgactgaccagcttcaagttggggttcccatgaccccctctttgggcttgattaacttgctggagtggctcacagaactcagggaaacacatttaccagtttattataaaggatattacaaaggaagcatatgaagagatttgcagggcaaggtatggttgaagggttgtagagcttccaggcccttcctggccatgccaccctccaggaacctccacttgttcagctgtccagaagctctctgaatcctctccttttgggtttttatggaggcttcattacattgacaactgtgtggaaatgtgattggacaaaaaggatatgatctcatactaatagactgagtggggaaatccagtaaggtctgtttgttcggattcttcttggcctcttggtaaagtatgggacgaaaccctctctggattgagggccttttgacccttaatcagattagagtcctgactgggcaggtaaaaggaggacaggacaaggtgagagagattctgtttcctgaggcctgctcctgaggcctaaagcaccccaacattataacaaaagactgtaacaagggctatgggagttatgagacaggaactgtggacgaaaacatagattttatatatatatacataaaatcatagtatcacgcccatttcttgagaagtcctggtttcttttagtgagaaacaatacttcaaggccccaatctgggtgctaagagatgttcaatggtacatcattagttattgtctttatgctttttttagtggacaaagtgaaataatccatgagtttaatatttacagttagatagtttttgtatcaataggacttttgtttaaccccttctctattatacctgtgtattctttcttttctactgagaatacttgttcttgaggatgatagaattagaatatgttgtaattgtgtatttgttttatcccatattacccacccaagagtatcagaataacagtgctaatactcccactgacataattattgaaaacattttaaactttgcatatattttctctgttctctctcatcatttaaaaatagttgtattattgggacaactagatacccacatgcaaaagaatgcagttggatccctacctcacaccctagacaaaaattagctcagcatggatcaaaaacaaaaatgtaagagctaaaacaataaaactcttaaaagaacacataggtgcaaatctttttgaccttagattaggcagcagtttcttagatatgacatcaaaagcacaagcaagaaaagaaaaaatagacaaactgggcaaagttaaaattaaaaactttcatgcttcaaaggataccatccatcaacaaaatgaaatggcaactcacagaatgggaaaaatatttgcaagtcatccctctaataaaggacttatatctagaatatataggaaacgattacacctcaataataaaagacaataatccaatttaaaaatgggcaaaaaatctgaatagacatttcttgaaagaaaatgtacaaagagccaggcgcagtggctcacgcctgtaatcccagcactttgggaggccgaggcaggtggatcacctgaggtcaggagtttgagaccagcctggccaacatgatgaaaccccatctctactaaaaaaacataaaaagtagccagtcatggtggtgtgcgcctgtaatctcagctacttgggaggctgaggcaggagaattgcttgaacccaggaggcagaggttgcagtgagtcaagatcacgccactgcattccagcctgggcaacagagcaagacttcatctcaaaaaaaagaaaaagaaagaaaggaaggaaggaaggaaggaaggaaggaagaaagagagaaagaaagaaagtgtacaaaggacagtaagtacataaaaatgttcaacatcattagccaatagagaaatgtaaatgaaaaccacaatgagatgctatttcacacctactaggatggctctaataaaaaaggatgtagagaaattggaaacttcacacagtgctggtgggaatgttaaatggtgcagctgctttgaaaaatagtctggcagttcttcaaaaagttaaacagacttaccctacaatccagcaatttcactcctaggtatctacccaagagaaatgaaatgtttgtgtctacacaaaaacttgtatacaaacgtttatagcagcgtgatttgcaatagccaaaaggtagaaaaaacccaaatgcccatctactgatgaatggataaacaataaacactgtatatccatatgatgggatattgtccaaccacaaaaacaaagtgaaatgctgatacatgctacaacatgaataaatattgaaaacattatgctaagtgaaagcagccagccacaaagaaaacacataatatatgatttcctttgtacgaaatatccacagtaggcaaatctacagagacagaaagtaaagtagtgattacatagggctgggggcaagggtgaagggtttagggggtgtgggatttcttttgggggtcatgaaaatgttttcaaattgattgtagtgattaatgcacaactctctaaatatatgagaacccactaaattgtatgcttcaaatgggtaaactatatgatgtggaatatatatcagtaaagctgtgtttaaaaagtaaaaaattttaaaaagttgtattacatttgtcttgtcagaccacatagtcatatatatatatatatattcaattttttttgtctcttagcccttatttatttgtagttctacaggtaattataattgaatgggccaggtgcggtggctcatgcctgtaatcccagcaccttgggaggatgaggcagatggatcatttgaggtcaggagttcgaaaccagcctggccaacatggtgaaaccccatctctactaaaaatacaaaaattagctgggcgtggtggcgcatgctgtaatcccagctactcgggagcctgaggcagaagaattgcttgaacccaggaggcagaggttgcagtgcagtgagctgagattgtgccactgcagtccagcctgggcaatagagactatgtctcaaaaaaaaaaaaaaaaaaaagaatgcccatcaggtatccttatattgatatatttatgtcagtctggttattttgctggttggaaattctgtgataaatttctcaggaaggctcatgaaaacaatattcccgagtttttacatgttgataacagtttatatcctttatacttgcgaattatttttgctggatataaaattattgtgtctttccttgaaagtcttaaatatgttagttctttttattttctttggcattatgcattgttgtcaaaaattctgatgataatccaattttctattcgctaagagtcaaatgagcctttttctaggtgcccagaaaatgtttctttctttcttttctttttcttttctttttttttgtagacagagtttcacttttgtcgctcaggctggagtgcagtggcgcgatcttggctcactgcaacctctgcctgtggggttcaagcgattctcctgcctcagcctcctgagtagctgggattacaagcgcccgccaccacgcctggctaatttttgtgtttagtagagaccgggtttctccatattggccaggtttgtctcgaactcttgacctcaggtgatccgcctgcctcggcctcccaaagtgctgggattacaggcgtaagccactgtgcccggccccttttttctgaagtctatttatttattttcagacaggaaactttttttaatactttttttttgggagtcgggggggcaggaaacagagtctcgctctgccactcaggctggagggcagtttcgtgatctaggctcactgcaacctctgcctcccaggctcaagcaattctcatgcctcagccttccgagtagctgggaccacagtcatgcaccactacacttggctaatttttgtattttcttgttggtagagaccgggtttcactctattggccaggctgggctcaagtgatccacctgccttggcctcccagagtgcttggattacaggtgtgagcctcccaaagttctggtattacaggtttggtgaaacccatccaggaaaactttttatttttcaaatcacaaacttcccaagtgtcaatttcctcgtttaaaaaacatatgcggccgggagcggtggttcacgcctgtaatcccagcactttgggaggctgaggcaggtggataacctgaagtcaggagttcaagaccagcctggcctacatggtgaaaccctgtctccactaaaaatacaaaaattagccgggtgtggtggcacgcgcctgtaatcccagctacttgggaggctgaggcaggagaatctcttgaatctgggaggcggaggtggcagtgagccactgcactcctgcctgggtgacagagtgagactctgcgtcaaaaaacaaacaaaacaacaacaaaacatatgcttggattaaatttgcaagtgttataatacctaaaattctataaaagaaaataaatgtttaaaatttcaagccagttatccttgtgggaatttatgtggatgctgcaaatagaaagagcaaagcaaaggaaaatcttacaagtaaaatcccacttttgtaatttcatattttcctaaaagtagagattaagctatgtttgaaagatcttaattttcatcccacagaataaatagagcaataacattttattttgtaaccaatcaattttagtttctctatcttctttacataatattgtatttgaaaactttttttatcatatgtggtgcaaacacataagcatacatatttaagtgcacagatattgtttccagtaggatttaaaattgcaatatactgtggtgttgactgttctgggaaaatactatcagctatgtggtgtgctctttcaatatgcaattaagaatcttttttattttcaggaaagtgttttttttttttaaagtaaatgtatacagtttaatatttgtcctatgcccttgctttggttttcttttataggaatttctattacctgtgtgtttaatttactttacctttctcttttccttttgatctctcttcatttctttttgatttaaaaagttttcctctttttcactttctagaaatttctctacaggtacttgttgcatttacttgcttgtattctttctggtttagtattcatttatgaactgattttctcctcttcttattcttattctttcttgagctgtgtcaccttgtttctgaggcttcctgattctgagttaggttgttgttaatgtctcttagctcatcttgaaataataggctacaatttttctttcttttgtgggcatgtctttttggcatactttcattatccttgggatgttatttagcttcccattttcttttttcttattataattttgcatgtgatttgaccttgatactactgtgtttttgtttttgtgtgaaatctgtttccctgtactttgaaaaaataaggtggaattccacagagcttttctaatttcacagagctccctcttctgttgcttttgtgtagtgttcaaaaatatggtggtttgctttctgggattttctggctccattcccctctccatttaggtctagcgtttctatttccatgtaatatctgagacatacatccactaaaaaattatttagtgtttatttaaaattcgcacttaactgaacatcctgtatttttctttgctacatctggcaacactagttgtaaatattttccatgtgctttggttttgcaatctagttctgctgtttttatgtagagatttgcagagattgaaaaactatgtggttgctgctgttgccactttcccagaatatgccacataattttcaaatgtaatcttctttcctttcttttaccgccttggtgtacttctcgcttcccttacacatatttcaccctgtatctcctcagataatccatgttctaaacctagtgtgcatttttctataatttttattacatgtatttaatcatacatgcctataagcattacatatggttttctgttttttaattttttttaaatttattattattattattattttaattttgggggacagagtctcactctgttggccaggctagagtgcagtggcgcactctcagctcactgcaacctccatctccgggttcaagtgtttctcctgcctcagccacccaagtagctgggactacaggtgtgcatcaccatgcccagctaatttttgtatttttagtagagatggggtttcatcatgttggccaggctggtctcagactcctgacctcaggtgatccgtccgccttggcctcccaaagtgctaggattacaggcatgagccaccatgcccggcctgttttttcttttctcttctttctttttttttttctttttaaacaaaagatcacatagtatacacttttcttcatcttgattttctctgtcaacaacatcctgcggaaatccctccaactcatagaactctatttccctctttttcataatattcagagatgtgcatgatctgtgattagttcaaccatgttcttagtgttggtctttgtagattttttgccactacaaatgcatctgtaataaacacccttatacagatccttataagtgattgcttttgtttctgtgagagagacttctgggagtatatttagtgagaaaaatgtgtttctgattgttgcaggaatggagattggggcattcaacatccatggcatcccccttaaagcacactttcccaaagaagagtgggcgtaaagctaaaaaagcaacaccccagactccaattagttcagccaagtaaatagcacttgtgcaggacttagttgctagaagtgagttatgtgataagtcactgaagcatttgacttttttgcacagaaggtgtagtgaagtttctccctactgtcttgtccttaatctcattcatgtctgtaaaaagtagtggtgtgggaccctccatggtgggattgggtgtttctcctggctgcatcatcacccaaaattgcctggaaattattaagataatattctgtaacaaatcctgttcattaaaacatgttacagtagattctgttggctaaaacaagtgcccttaacccctaccagctgtgattgcagtaaacagtacttcagtgaaatggggaggttggattggttatctgccttcgttgcaattgaaagctgtgacaatctgtttgacatcagagaatggttgctggcagtgcatggtacacagtggtggacgattacttcaactatcatctgtggatacctggagtgaagtacatattgaagataatgccttggcagtcaaagtagctgctgtgacagatgatgtgaaggccataaggaatataaaggttgtgacttgggtgggctatttctaactgcttaaggaaagaaaattaaagcttcagggttttaaattcttgcctgaagacatggttagagaaccagggaattcctatgactgccctgaaataatctctccatgtagtcctacatgactgaggttaactggcttgctggattaatataggttgaactcatagtcctccctgatcttttaggtaaaaacttagggcatcaattgggaaaggatggcaccatgagaatttggggaaatttgggaagatctgaggataccaagtacctcaaaccccagtgagtttctcttgatagcagaagccctctcacccttaccctaccccatcttatatgcctgttcctgtcctactttaagcccttcttgtgatctcacctaagatagttttcttgtcagcagatgacaattctcattatccccctcctgtccctgctcattgtactgaaacatacaagcagtcatattgtcctgtgtcccaggggaccaatcacaaagttaaaccttagcaaaataattgcaaagttttgctaatttatttcaacaagacatgggtaatatatatatggacatggagtctaagagattagggacacacttttacattggcctgaatttacttaattttagaggacctaattgcttctttagtttgattaaaacttggtgtggcctatgtgatgggaaattgagattccagaaactcataccagaaacacatcaaagtatgatataaagaaatcagctgggtgtggtggcttacgcctgtaatcctagcactttgggaggccgaggcaggtggatcacctgaggtcaggagtctgagaccagcctggccaacatcgtgaaaccctgtctctactaaaaatacaaaaaattagctgggcatggtggctggtgcctgtaatcccagctactcaggaggctgagaccggagaatcgcttgaactcaggaggcagaggttgcagtgagctgagatcgagccactgcactccagtgtgggcaacaagaacataactctgtctcgaaaaaaaaaaaaaaaggaaatcaaaagacatgtttttgaaattttggagtggatttatcaaatgtggatttgtcattcactcactcccttccaaattaaattacccaaaaaggctcagagaatatttctttcaccatgacatggaaaagtacaaatgtaaaaaagtggaaatggagaaggaaaatttggatgagggattttgctatagaaatgtatacctggattttggtgaaatgatcctatcccacagtggcggagaccatgaagtagcacttaaccagtggagaaaggcagcatggctacacaataggtatcagtgatagagtggttctcagaattgtctgacccataggaaactttgatggtggctaacctgaggtactcagggccaaaatacatagacagaccactaagattctactcaactgtaaaactagaagaaaaaaatttgttacgtgtatggaaagagtcctgacttaagctatcccaatgaagagtcatgtcctctcaccctttttccatttctgagtcagttcaatgacccagcaccccttgaatgaaggagagatcaggaaccctggggaggaaccctgtggtaacttagatatatataactcttattcctcctgtttcccaagaagactcaggcctatttaccatggaaactgagccccagggaagacaaacattcagatctctcaggaatgattagctactgggtccaaagcaagcttaatccctgagaaaactgaattccactgtcgtcccccagtcagggtgggatttatgagagctaagtgatgaattttgggctagcattcttctcacagtggcccagtgaatcttatatccataccgtgtttattttcctagttctggagttcatagtggaagtagaaatattcagctactggtagaaaaataaagattgttctctgacccatggaatcatgtctattatggtagaaagggccaagtgtaagtacatggagattcctctccctaccaaagttataaactaaagacaatatcatatccctttggaattatggtgatgaggtaccatcaaacacttaaaatacttataagtagtgattcttaccacatctccatttcactcacctgtttggtgtgtgtctttgagaattttattggattactataagcttaatcaggtagtaatttcaattagagttgctgattctctttactggagaaaaattaacccagagcctcatacctggaaggtgaaattgtctttttttctatctcctaataagcagataatagtagaggaaattgcttttacctgtaagggtcacttatctatctctattttcttggcttaaggtggtcatactttggtccctatgggcatgagtgtctcactgtcttactggatattactttgagccaatatattgatgacattcattctgataggaactgagagcaggaaatagtaggtattctcaattccctgatgagatacatgcctgctgaaaggtggttgtgaaatcccacaagaattccaatgtgttccctcatggaaatgtttgggaatctaatgtcctggggaacattagctgacaccttcaaaatgaaaaacaagttgtaaaatgaaaaacaccctgcattcctacctttaagaaaaaggtacaacacatggcagattattctggatttttgaggcaacatatggtgcatttactgagtaacctttactttagcccgttgactaagtaaactgtaagggggccagacttcaatgaactgcagaataagagaaattctggcttgttgaagctgcttggcacccagcttttatgatccaaagtatgaaatagtcctcgaagtatttgtggcagattggggtgatgtataaaggttgcaaaaagcctagatagaagaatcacagcatagccccttaggattttggagcaaaataaaattctattaggaaaatcactattctccttgcgaggctgtggcttgctagaggctgatagagactgagcacctgaatatagtacactaaatgaccatgagatctgaattatcagcataaactgggtattatctgatttaccatgccttgacatagagtatgcctagcaggagttcatcatcaagcagtagtagtatgtatgaaattgggcttgaacagctctaagaggcacaattcagttgaatgaataggggttgttcagtatttacagtgctcttatttttgacattctatagaaatttttaaacatacagaacaaaagacttgtccagtgagcatccatatacctaccacgtagattctaccattaacatattcatatatatatatatatatatatatatagagagagagagagagagagagagagagagagagagagagagagagagagatgaagtcttgctctgttgccgaggctggagtgcaatagcacgatctcggctcactgcaacctctgcctcctgggttcaagtgattctcttgtctcagcctcccaaatacctgggattacaggtgttcgccaccacgcccatctaattttttgtatttttagtagagatgttggccaggctggtctcaaactcctgacctcaggtgatcctcctgattacaggtgtgagtcactgtgcctggcccatattcttatatatatatatatttttgcacattatctgttcattcctgtatgtatccacaaattcatcttgctttttggatgcatttcagagtaactaacctagttctctacttctattgcatatccacctatccctcaagccacacctttggactcttgggttgttccttgtggccagctgactgaagaggaaaacccttagcctagtttatagctggcaccagacaatattctgtcagtagcgagaaatgaaatcagcagcattagagccctgctcagagtgggccttcaagacagtggtgaagagaaatctgcccaatgtgcagaacttatattagtacattctatttcctctttgcctggaaggagaagtatgaatgtacagcagttaataagtagtgattaattgtctggccagccagacaagagacttgggagaaacagctccagagggcaggggacaagggagtctgagaaagaggtgtggtttcagaatgtgaggatatttgtgtaaaacatgaatgctcaccagaaaacctccacaggggatgagactctcaataattgagcagacaagattagctgctctgggtttcagtcagcctttttgcccagcttgcacaatgagtttatgaactaagtggctatgacagcagagatagaggctgtgcttgtgctcaattctatggtctctcatcatcaaggccgacctagcttccacttgctgagcacccaatctttcagcagcaaaatccaatggtaagcacctaatatagtatcataatctaagaagatcagccagccacctggtggcaggttgattttattggatccctgtcaatgtattgcccatactagaatagacatttattctaaatatgtatttgtcttgcttgcttaacgaacatgcttctgttaaaaatgccatccttgaaattactggataccttattcctggccatggtattctgtacaacattgtttctggctaagaaagaagcaaggcaatgggctggcccatgcagaaattgctgatcttatcacgtgcctcattacccagaaacaactgagtatatagaatggttaaatggcctaataaactcaggtactgagacagctggaagacaactggggaagttaggcgaggggagtgggtgcttcaaaagtctgaacaagtaactattaataatatatggtgctgtttctcctacagtcagaactcatggatctgggaaccaagggggtgaaagtggtagtgttattttaaaatattatgtctaatgacccatgctatggttcaaatgtgtcccccaaaagttcacaagctggaaatctaattgttaaatatgttttagcaattaaagaggtgatttaggcagttgcctttaagaggcaattagtccacgaaagctctaccctcattaagggattaatgctgttattgcaggagagggttaattatcatgagagtgggctcccaataaaaacatgaagttcagcctccatccctctctgcctcttgcatgcatgtctcctcatcatatgatgccttccagcataggatgaccctcaccagatgctggcaccttgctcttggacttcccagcctccagaaccatgagtcaaataaatttctcttccttataaattatcctgtatgtggtattctgctatagcagcaaaaatggactaggacaacttaccaaatgtttgcttcctgttctttcaatttttggctgtattgatttagaggtttctgtacccaaagaattaatgtttccaccatgggacaaagcaagggctccactgaatgggaaactgaaactgtcacctgatcattttgtattcctcatgcacctggattaacaggcccaaaaggggattaccatttggctggggtaattgatccagataatcaaagggaaataggaatactgccacaaaactgagtgaggaaaaaatatatctgaacacaggtgagcctctagaacactccgtaagaatggaatatccagtaataaaatctagtgggaggctacagtacccctatggtttggatcaccccatggataaaatggctcaactggctgagacacagactgagaaaaagagaacatgatgttgacatcacagcatttcagtaatgttctgtaacttgtgcctagctgcagaaatgaggactttaggagctatcttatgattcctcaaattatgcatatgcatatgtaattacacatatatattaaatatttattttcaattatgcattttaatattccttgtatctttcttccactattttatataaggcagatgtttttgttatctgatgatgtataacaaaccacctgaaaatttaatgtgttaaaacaacaatttattagctttcacaactctgtgagattactgggtactcatgggagctagagtacatctttccaataaattttttaaaaaattaagttgaccagaggagtaattctattgcctacaaccaaagaacactaattggaacagaaattggtatctgagagtgtgttctaagcaaaaggccctcaaagaaatatggtgtattttgaattgattatcaggccaagataagacaagaggcaattacactccaatttctaactggggcaaggaaatgaatagctcatggaacccggtggagaaattccaaaatttctagggaacttgggagcagttgcctacaatacaccccagagcctgctcaaatgggcaccttaacaaagtggccctggtggcagggatggatactatatatgaactaaaaattatgaacttcctttcactaaatttgagctcactaccacctctgttacataacaattgctgacaacagcatccagctgtgaaccagtgatgatagcaacaacgccttgagaagataagtgagctgagatcatggggtcccattacaccatggatgagacagcaattttggactcacaggaatgggaacttattgcatatttcatgttgtgctcactggttcattgtatactaaatatattacgtctatctagctgcctatctagctatacatacacaaatatatacatacacatatagatatataaaacaacatccctggctcacacctgtaatcccagcactttgggaggctgaggcgggcggatcacctgaggttgggagttcgagaccagcctggctaacatggtgaaaccccgtctgtactaaaaatacaaaaattagctgggagtggtggcaggtgcctgtaatcccagctacttgggaggctgaggcaggagaatcacttgaacctgggaggcagaggttgtagtgagctgggatagtgccactgcactccaacctgggagataagagtgagactctatctcaaaaaacaaacaaaccaaccaacaaaaaaactcacaagatttctctctacatatgccatatattaacttttataattttttctgattataaaatcattataaaaatgaagctattattggatcaacttagcccttagtggaaatgactcatcctaaggaagactttgccaccaggagtcaatggcaggttataatcagtttccctgccacccactcaagcacctggaaatgcccttcactttctgcccacctaatgcccccagaagccaactgggagaggaaattaaaagttctccaataggcagacatctatatcagctccagtgaaaccacatttattccagtgttctattcagaatcttttgatcagccagttcaatttgaagtccatttatttctttcgacaatgttaaccaagcacctccttatatgccagaggcttttttagggactaggcatgcagtaatacaaaaaacacaccaaatcccttttcacactacctagagaggggtccatacagtgttgttctggattgccatcataacttaaaggaaaatgttcacaatgtctggagtccttaatgttctgcaaatgaaagaggaggatgtcatcaagttccttgcagcaggaacccatttaggtggcaccaaccttgacttccaaatggaacagcacatctataaaaggaaaagtgatggcatctacatcataaatctgaagaggacctgggagaggcttctgctggcagctcatgccattgaaaaccctactgatgtcagtgtcatatcctccaggaatcctggccagcaggccatcctgaagtttgctgctgccacgggagccattccaattgccggccacttcatttctgaaaccttcactaaccagatccactggcagccttccgggagccacatcttcgggtgtttactgatcctagggctgaccaccagcctctcagaggcatcttatgttaacctgcctatcattgttctgtgtaacacattcttctttgcactatgtagacaatggcattccttgcaacaagagagttcactcagtgggtctgatgtggtggaggctggcccaggaagttctgagcatgcgtgacaccatctcccatgaacacccatggaaggtcatgcctgatctttacttctacagagatcctgaagagtttgagaaggaagagcaggctgctgctgaaaaagctgtgaccaatgaggaatttcaggttgaatggactgctctagctcctgagttcactgctgctcagcctgaggttgcaggctggtctgaaggcatgcagctgccctctgtgcctattcagaagttgcctactgaagactagagtgcttagcctgccacggaagactggtctgcagctcccactgctcaggccactgaatacataggaacaaccactgaatgggcttaaagctgttcttccacaggctcttaagcaacatggaaataaggctggtggaaaataaacatcattttctaaaataaacaaacaaacaatcaaacaaaaagacaaaaatcgcttctcatggagcttgcattctcatggtgggaagtgagacaaacaataactaaataaataaatttgggtggtaataagtgcaaagaaaaaataagacatgggcaagggcatgggtatgttgggttatcagagaaggcctctagtagggcagagactctggaatatgttctaggcagtgagaacagcccatgcaagggccctgaggcggcagtttgctctgcacgttgaggaacagtgaggaagccagcatggctggagtggggtaagcaaggggcatgagtaggagtttagatctggaggtatttcaatggaagccagatcatgtaggctcctgtaggctttggttaggactctgaattttattgtgagtgtggttgaaaacccttgtagcattttgaatagagacatgaaatgctctgtttaatgatttagtgggatcactcttgctcccctgtagagaatggtcttggtctttgatgcaaaccaattccatgcctctacatttcttcatcttcagccatataaaaatgcaagctccccccatttcctctcactgaaaggctttcatctccctatttctgcaagtctttctcttgggctcatcttccactgctgacctcctttctccaaacttgtgttgcaattgtaggcaaacttccctgcctaaatagcctttcaccttatcccatccttaactgacacctctctttcccctgaggacacaactgtccttgcaacctctgcagggcaggttgacatgttcccatgttctacttattatacaacagaaccagcacttagacttgcctgctcccactgacccactggaagcttctacactgtttggtttaatgggaagctgcacagactttgtagtcaggctggacttgaatctccactgcacccccttttactggctgggttacactgggcaaattatttaacttaatatgagccttggtttcctcacctgtaaaatacattggacaatttttgtcaagattacaggaagcgatgattgtaaaatacccattatagtgcctagcactcaaaaaaccttactcttctcttccctcctgtccctttgataaccatatgaccctcttgtcacttatctgtatagtgtgcaggtagaatgtggcatttgactcacctcgctaatccagacttcatccccatttgtgatggttaatcttaggtgtcactttgagtagattaaggaatgcctagaaaattggtaaagcattattctgggggtatatctgtgagggtgtttccagaaatgattagagtatgaatctgagtagactaggtggagatctgccttcaatgtgggcaggtaccatctaattggccgggggacctgagagaacaaaaacagaggaaaggctaatgtatctatctatctgcttcagcgggtatacactcttccgctcctgtccttggacaacagaactccaggttttccagctttggactccagaacttataccagcagtcctccaggttctcagatcttacacctcagactgagaggtataccatcagcttccctagttctgaggcctttagacttggactgagctatgctaccagcatcccagggtctccaacatggaggtggtctgttgtggaactcatcagcctccataagctcatgagccaattctagtaaatctcctctcataaatccatatatctacatctatatagcctattggtgtgtattgctggagaatcctaagagttttcataaccacatgggagacttgtttcaggaactcattcctgtagccataccttgagccatttaaactccagtattttagtttctggtcaaaatctcccatctctccattcatccttaatcccttttgagccatcttgtgcactctagtctccaagtgctccttctccaaggtctgacagctgcatccctgggctctacctccttccttatttgacctggacttcctcctgtgaatcagcgagagttttagagttcaagtgatagaaactgactctggctaacttcagcagaaaaggaacctactggaagcatattgaggggtttcaagaaccagtgggaaagttggaaaccaggcttggaaaaggggtggcatgtgttaggtttgtcattcggtaaatatttatttcctatcctccatcaacctccatgggagaaatacgatgctggattttcccacatgattttctttggacattgaagcattggaggaagagacagtatgcctgtagccttagaagcatttgtgtttccactcatcctgagagctttggacctccatcataaaagaagcattctctgggtaacctgctagtcccagaagaatgaaagactggtaaagatatcctagccaatttgtacactgcagcctgaaacagatatccccaggtaactggcttgtgtataagcaagaaacaatgctttttattataggtccttgagattttatgattgtttattacacagcattgtgtgagtagctggtgaaccaagtagtcctggtagaaccaaggatgggcggggagcaggacccatgactaagcatgccataagaactaattgataaggatatgtctccttgctcctttggctgttgctactgctggactctcaattccaccagtatgaatgtgttctaagccagtgcttttcacactttaatatgtatgaatcatgtgggatcttgttaaaatgcagattcagtacatctcggtgggccctgagattctgcatttctaaggctcccaggtgatgccaatgctactggccccagacagacgggaatattcaaatgggtgaatctaggtcacatgtccatgccttgactgttaggtgacaggtagagggatgacccttctgtgatgggtcagggagcactgtatcccagtagtgctattcaatgggaaataagaagagcagctaaaaatattacaaatgtctatccaggctccctattaaatcactttaacccttctcttgctcttggtcctgctattgcacttgttctgcaactcctgatcttggatcaatcccctggctaccttcatcacttttaccaacttattcattcatttaaaacatattttctgaattcccataatatgccagccatcagggataatttggtaaaaaaaaaaaaaaaagaataatgaagattctacctcttgtggcttatattctagtggaaaaaaacagacattctcaaataaaaaaataagtttaatgaatccagggtgctaaatgtgctatgaagagaatatcatgatgagtaactaggggcagcttacatgagataagtcaagctgaagtctgaattatgagctcagagaatagctgaaagaaaaatgttcaaggctgagggaccaaatgcaaagtcttgaggcagggaaggtcttggcttgaaaaacagaaaggaggccagtgagactagggttattgagcaagagggagggaagtgttgaattagggtggagaagtagtcaaaggccaagcaacagggaaagctaagaaatttagattttattctgaacataacagggagcattggaggctatttaagtaacagcagatgaatccatacatattctgagagctcacttaggccactgaatggagaacggattgtaagggcaagaatagaaagcaggagaccaacaaggagagtgtcagtagtccaggtgagagatgcttgtggcctggcacctttcttttttctgctgtatgagaaagccacatacttctcctatttaagagtaatccctccacctgaatcactcatcatcttctctgtttttattaattctccctgttttctaccttcaatctctttccttcctttgggtctatttcctcagcatactaatatattcagatctcttacacataaaaaagtaacagtattaagaggtgggacctttaagagatggttaggcttcacccttcactccttctgccatgtaagagtggagtgagaaggtggctgtctatgaggaagcaggccctcgttagacactgaaactgctggtgccttgatcttgggcttcccagcttccagaacctcacgaatggattaatgccattattgtaggagtgagttagttatcttgggactggctttctgataaaacaatgagttcagcctaatttcctctctctgcctcatgcacttgcttctgccttccgtccatcctctttgggatgaccctcaccaggtgctagcactatactctcggacttcccagcctctacaatagtgagcaaaataaatctcttttctttctaaataacctagtctgtggtattctgttacagcagcacaaaatagactaagacacctacattctacaataccactctttttgtgttctcttcctatctctctggcagtttcttttcagtttgtgttttcatctttcttgtccgtgccactatccactcctgtcactcatcatctgagtggggttgcaacctccttactctgacctcccttcctctaccagagtcctcctccacatcattgtgtactttggtttctaaggaggatgggttaagacatatagtgacatattgatgtaattcagaaacacaactatttgtttaaaggatgatgtgttttaagaatgattcctggttactctctggaaggctaagcctcaggagacatcagtttggcattcagactgtccacttgcaatataagagctgctggacagcttaagtattttcagctgtagacactgggattgtctgcaccagttgatctaattactaccttacctatacctaactttataccttggtgaagaattatcttggaataaagtcattcctgcatccaatcacagcaggaacctcaggtaaacaattcattcaatgagagtaatggctctgccatatccaccaatcccaatcttggttcatacactacagagaacagcagcaattaaagtgacattttaataaccaggcataagatgtttttattataaggattgctgaaattcctcaattcatgattactatggtttgaatatttgcattcctccaaaattcatatgttgaaatcctaactccaaggtgattgtatcagaaagtgattagtgtccttattttaaaaagcctaagagagttccctccctccctctgccatgtaagagtagagtgagaagatggctgtctatgaagaagcgggcccttgttagacactgaatctgctggtgccttgatcttggacttcccagcttccagaactgtgagaaataaacatctgtcatttataagccacctagtctatggtattttgttataccactctgaacaggctaagaaaataattttactactggcttcatgaaaaatattcccagggttgcctccaggcagatattctaggaatccaatgtaagcaaacctggtcactgatttctaaggggaaactctgtctatgtctccccatgagactgtaaatcctgaaagcaggaagatgtcatttaatttctgttactctagcatctggcaaagtgactggcatttagtagctacttaataaatatttgttgagtatttgtatatttgactgaatatacaaactaataacagccagatcacatataagaatagaaccctcagctgcatgtggtggctcatgcttttaatctcagccctttgggaggtcaaggtgggaggattgcttgagacgaggagtttggggctgcagtgagctatgaccatgccactgcagtccagcctgggtaacagagcaagatcctgtctctattttaaaaaagcagaactttgatccacaaccagtagtaatcttcccaggaaatcaacccatatctacaataaccagtccaggaagctagcctgctatgccagacttgtaggaagccagattgccgtctctaataaaaatctaagaagccaaacaataatccttataataatcagccccaaatggctaggactttaataacttgacagcttccctaagttttgtctttgcttgcaacttaggactaaccagagaaaaccagatattcccccccaaacaactcctgtaagatgcccagtttccagttagccctccaacagcttcctgtgccaacagcctccaatcagggcacacctgaagccttcctttcttttccactataagattttcccactcctctgcctgcctttgagtctgccaaaacacaaataacagtggctgactcccttgctacagcaagttcagaataaatagactttgcttgttctcatttgattagtcttcatttattttcacaagtaaatgcctttggagacttaggacagacctggacccgccatggtaacacggcagaaccttaggattggaaaggggttacggagaaacgtaatctggatagctgttggaacctgactttctcaggttatttgcctctacaccctcttttcccattcaaaaaaaaaaaaaaatcctgaaccttcaaggcttgggtttgaaagcagaatcttttgacaagagacagccagctgactctgcagttcccagtggccccctgagcttcatctttcacaggtagtaggctcaatttgatgactcctgtcactgccatgctggtccatgttcaagggtcctcagagactccttccctgggttgcccttagcacagctccagtaagctggagtttcataatttttttttttgagatgcaggcttgttctgttgcccagactggagtgcaatggcacgacctctgatcactgcaacctccgcctcccgggttcaagcaattctcctgcctcagcctcctgagtagctgggattataggcatatgtcacatgccctgctaatttttgtatttttagtagaggaggggttttgccatgttgaccaggctcgtcttgaatgcctgacctcaggtgatccacccgcctgggcctcccacagtgttgggattaccggcgtgacccacagcgcccagcccataatgtttttacaaattaaaaaatgtcaccagaacctgcactttggcttcaggtaagtctatgttaatagtaacctctcggccgggcacggtggctcatgcttgtaatcttagcactttgggaggccaaggcaggcagatcatgaggtcaggagtttgagaccagcctggccaacatagtgaaactccgtctctactaaaaatacaaaaattagccgggcatggtggtgggcgcctgtagtcccagctactcgggaggctaaggccggagaatggggtgaacccgggaggcggagcttgcagtgagccgagatcgcgctactgcactccagcctggacgacagagcgagactccgtctcaaacaaacaaacaaacaaacaaacacataaaagtaatcttccagttcattggctctggttcttcttcaaatagggcctcatctctcctttactccaggtagtggtgatattaaaaatatttagcacctggaatgacaagtgcatggaccaatcaaaacaggggctcaagccgtaccaggcattaaccctttagtttttggatcctggggaggtctgggtagaaggtaatgggggtgaagaggggaagggtgcttgggggcattggctatttaccaatgaatatgaaagtatctcagtattttaacaaccagaacagcattcgtgcacaccagatgaatgtcagacctgccttcacatatttctgggagttgtcagtttgttgtattcaacataggctgccataacaaaattccttagactgggtggcttaaacatttatttctcatagctctagaggctgggaagtgtaagatcagggcactagcagactcagtgtctggcgagggccctcttcctggcttgtagagggctaccctcttgctatgtctttacatggtgaagagagagcaatctctctctcctcttcttaaaagggcactaatcccatcactgggactctacccttatgacctcatttatatctaattatctcccaaaggtcccacttacaaataccattacactgggggttaggccttccacatatgaggtttttcttttgttttgtttttggaatggagtcttgctctgttgctcaggctggagtgcagtggtgcgatctctgctcactgtaacctccacctcctgggttcaagcaattctcctgcctcagccccccagtagctgggactacaggtgcatgccaccaaacctggctaatttttgtgtttttagtagagatggggtttcgccatgttggccaggctggtcttgtactcctgacctcaggtgatccacccgcccagcagtggacagcctctcctgcaggtacactggcctccgctgggcatgactgaggtttctcatgtgtggttttatctctgaagcactaattcctccattagcctctacaggtgcttctgctcttgcctgacagaccttatgttcttctgtggatgtccaagctgccttgggaaatctctgggtaccaccaccaaagtcttagagccctcaaagtgcctggattttccagacatttaaggtaaagtgggggaggagaatgcctctttttctctttaaatctcttttatgagaggagtaccaaaagggcatttatgtatttattcattggacaaacattgagggcaactatacatcaggcataattccaggcgttagagctaaagtgaaaaagacaggctccctgctttcatgacacttacattctagtgggtggagacagaatatagacaaacatatgtaatgtcagctaataataggtgtaggaataaagcagagtcatgggttggagaatgatggagtatgtcgggtatgtctgctgttttatatcttagactggatgatcagagaagacctctgacatttgatcaggaaacttaatcaaataaaagcgtgagcaatgggggatatgtgggaacatagcaaatgcagactctgtagcagtgtgcacatgctacagtgttttatggttttaatgaccagttccttcactttttcctgtcgtctgccttttgtcttatgcttgccttctcaaacctcaattaagatttttttctataactgacaggttttttcatctatcatctttgtttatccagaaacagagagaaaagaagaagaaagacaaggaggggaggaagagaagcaaaaggaggaggaggagaagaaggtggcagcaataacaatggatgtaggagggggaaaggagggcatgctgcccttgctccttctttcattgccttttggcccctagatgaagaaagggctgcaccacgagagaggcaattctagtctgagcagtctcttgggcctgtgggcaagttagatgtggggcagcaggaatctgctccattttgagttgtctggcgtttttatgtactcatttggtgcagtcctgctatggggattgatagccaggaacctgcaccatgagtcccaggtataggatcctctgttcaaagtatatgggtaggtctcatgttccatatgggcctccacctatcttttcccattctcaggaccctcagctccttttggcagcagcatcttaatacctgctgctactagagggaaacatcattaggagcacttgcttggccatagtaggttggtaccagcaggcaacattcctgtttcctagtacagtgcctggaacctagaaagtactcagtaaacctttgatcaggaggtactgtgcttcaggacagaaaccaggattctggtaactaggaatacattccactcatgggaagtttaaaactggcaagctgtgtgtaggtatgtgcctgtagtcccaactactcaagaggctgagtgggaggattgcttgagcctaagagtttgagtccagcctgggcaatagtaagaccttatgcctaaaaaaagataaaaaattaaaaccagggtagaactagaacttacataattgcttcctttagtttaatgtgtcaatgttctctagcaacccagtgttggtttcactattcattgcaacaagggagaacgcacaccatgggagatcatgagagatctccatattgggatgttactaggagttataggatttgccttgtgttaggtgattttagggggcaagttttaaaggccagtgcttttctgtgtattggatgctgtcaagatgagggagtaattctatgattgggcatcttaataaattttattcagaggaaaagaatacagtgatgggctgggcacagtggctcaagcctgtaaccccagcactttgggaggccaaggcgggtggactgcttgagcccaggagttcaggaccagcctgggaaccatggcgaaactccatctctaccaaaaaaaaaaaaattagctgagcacggtggcacacacctgttgttccagctactcaggaggctgaggtgagaggatcactggagctggtgaagtcgaggctgcagtaagccatgatcgtgccactgcactccagcctaggtgacagcaaaaaaaaaaaaaaaaacaaaccacagtgatggtagagctataattgatttaaaaaaacagccatcaaattaactggaaaaggagggtgtttgatgtgcacagtaacgttgtttttgtctgtgcttaggcaaggttatgaaatggtcttgtcatgtctcgtttcttcagtcacagaaagaccatgtgtgatgtcggtgttctgtgagattatttatgctcaacaggagaatattgagggatgatggtgaatgccaggccagcatgtgacaaccctgaggcttagttacctgtgtcagaccagttcccaggcaccctagatttatttagtgagtcctccagttggtcaagggtagacaaagtcaggccacaggaccattaatctatgatattcagattttcactattgctaaatttatgctgatctggaggatgcagtctgtagttgggttaattccgccttctgttgtaggatttgttgaatcatatgatatgacaatggctgtgcagaaactagggctgtggataggatacgttgaccaagtgcaaaagaagcaattaccagaaacaaaatgattatccatctttgtaaaaagtcttgaagacagggacccagattataaagtcctggcaatgaaaacatgccccagaatccagctgagtctgtttttagggagccaggaggattttctcttttcagtttactcatgttctactgactctgtagtatgttctactggcccagtagtatgtgagtatggtgacaagtgttgctaaatacaaactcctccttggccagctaagaagaaatcaagggttatataattatccatgaaaactctggctaaggaattcaaatttaagtggttttctgagctgctatagcagaagttttgctatctatggggtctgctaaggtcaaatacctctcttggatctgatctctttctgttgtattatctttgttgtaggaatttcattcacagagtacacatgaaaaatgagtcagttatccctcctgggagttctcccaagtagtctgtgattgtgtgcttgcttcattttgttggtttctgggtgttcttttcaaagaaacacagtctactggaggggtggtaattttaagcactggagggtctctaacaattagcttcaatataaagggtaagttagtgggcaagccagatgttcccataaataagtagtaccctaggggagtacagactgcattgggaggataagggctatttagcacatcagggacaagccaactttctttccctcaggataagcagatttcttggatgaagttaaacaggtttctccaaagcaggctggaatgttttggaagagaatgacctgagtagtgtataaagaatagccccaatattagtttctattcctgaagggctaggtttttctcataggattgtaatgacttcagggaaacattctgttgaagtgtaggtcaaggtccaatttttagaagactgaggtttgggatgaactttgtggggaatgcttaagtaccagaagtgacctacagaagaaaatctacttgatgaagtctatgaaagcacagttattaaattttgtcaacaattccttaaaagcagtagctacggtttgagaaaaacactccagaaagtgtctcttttcttttctttttttccttccctttcctttctctttccctttcccctttcccctcctttcctttcctttctctttctctcttctccctttctctctttcttttttagattgggtttcactttgttgcccattggcacgatcacagctcactgcagtcttggactcctgagcttcaaggaccctcttgcctcagcctcctgagtagctgggactccagatacgtgccaccatgcccagctaattttttattgtttttagagatgggggtcttgctacattgcctacgttagttagactcctggtctcaagtgatcttcccaccttggctttctaaagtgctggaattacaggtatgacccactgcacctggccaaataatgtttactttaagtagtagaacatagggagtcaaaagtaacaggatgaagataagggttatctccccgagaaatcaatttgttagttaatcaagggtgtcaaaatgcaataagcaagattagtggtagggtaagagatgccaaggaatcagaaattaatcttgtcctgttgtcttgggtataaactgcccatttctgcagtaagcagcttctggggaagtcccaggaatcttcagtttgaggtcttccaatagtatgggatgtccggtagccaggagaaaatgatgcatgtctttttagttgtgtaatatgaatccagggatcaatgtcttctagttttactcctgggtcagttgttgatagtacctgacatttttttttcttgcattgaggctcaagggaaattgttttctaatgctttttcagaagaccaaatctctaacttcagaagctgcttaggtgggtgctttgagaatgcagcttccatctgttgttgataaagcaggagttaatcttagaagtctcctgcagcaattagtgataacagcttatattagtagcatctagtattggaagtaattcacaaatacatgaggaagcctgttattaactcagaaggagataacttatgggccttagaatggactgaccttattgccatcaaggcaaatagaagtactttgggctttggaagttagatggaagttagagaactttgaaagctattttttagaattctattagttctctttatctttcctgaaatttgtggatgcaaaaagatagcggagtttctaaaaaggtaaagctttacagttatttaatatcagtgaaatgtgtgtcctcttactggagataaacattgagatttcacattcaggaatacaaaattaataaattcttttctactattacagtaataggtctccggcaaggagaacccctaaaccccttaagaataaataaccctaatgtactcatagtccatttagaggtgttcaacagaactccaagactgggctccagttcatgttctacccttaaagttttgccaggattgggtcattgacaggtaggcaggacatacatccttggcaataaccctaaaattgccccaccaacattggttcagcatcattatcaatgtatctctactattgtgggtaataccatgaagactttttgctggactccactttaattatggctatttcctttggtaacagtgaagcatctcaagattataattattatttaaaattttaatttaaaaattttccccataggttattgggggtacaggtggtgtctggttacatggcatcttaagatactttaatttgccattcatttcgtagaggttcctgaagcggtcaggaaatctttgtttttaaagcactccaaagtcattgacgaaccaccaaaacatatgtactatcagtataaatatttgctctgttatcctttaacagctggttggtctaggccagggcaattcctacactaacagaggacctgggatggcatactgagtcagccacttactaggtaagaaactttgggcaaattaatttttttagcctcagattcttcatacatgaaatagtaatacttgtctcacagaaccgtgagcatctaatgagttattgtacaataaccaacctatcactaagtccttcacattgattatttcctttaattatcacacccatcctatgcgatggcctattatcatcgttttataaacacaaagtcacaccactttaagtggtgatgccaggattcaaactccgatctttctgatgccagtgcccagttcttaatccctctaacaacccgtgacacttttccacggttaggttcaagcgcccaccatacagcttgacactttaggagatactcaacctttaaaactttataatttgcatgtgtgattataacgggtacgtcctagagtttgatctcttctctgtgcaggggcgaaactcgaccccagccgagaactacactgtccttgcgcggggccctctgcgccccggaaggcgcccagtcccggtttattgtgagcttacagtacagttcgcaaggtcccggccggtgacggcggcgaggcggcaacgtcgcctgtagcaaacctccgccctaaggcgttcccgccgggcctcgcgcggcgtccctagcaacgcgcgcgcggtcttccggccccgcccaggcgggcggcactgcgcctaagctgggccacaaccgccagtcagggctctccccttcccctccctccccccctcctcctcctcctctgccgcccagagcgagacaccaacatggagcccgaggatctgccatggccgggcgagctggaggaggaggaggaggaggaggaggaggaggaggaggaagaggaggaggctgcagcggcggcggcggcgaacgtggacgacgtagtggtcgtggaggaggtggaggaagaggcggggcgggagttggactccgactctcactacgggccccagcatctggaaagtatagacgacgaggaggacgaggaggccaaggcctggctgcaggcgcaccccggcaggattttgcctccgctgtcgcccccgcagcaccgctactcggagggcgagcggacctccctggagaaggtgaggcgggccggggaggggtgtggagccgcggcgagttgggggtggaggctgggccccgagcgctccgcccgcccgcaggtcacgccgcctgacggagaaaacgcgcgcagctgaggctagtaggcggcccagactccagcccaggtgccggccggctgctccgcgtctcccaggctttgtggcaaagacttctcgtttcctccccctcggtgggtcctgggccttttgaactcccgtagacgttcaggggagcctgcattccccgtgagaaacgctgagagaggaccacgggtgtgtcgagccttgaaggagggaaaaggcggtgtaggaccggagcgagtggttgcctggggagcatcttgttggggagacagtgaacagatgagcttgactgaattgcaggactcccctgagggagtaatgtggataatgtacaccgcgcgatcacttagtctggagaggctccccgcgggttttcctcccctggaccatcatttgtagcaataaaggggttccccagccgataggaggggcttggaatgcaggaggcttgtgatgcggctccaaacagtctgcaaaactacaaagcacagataatacacaatggagaaagccgatttcatggtagacccccgaagatcattttgaaaataactctccatttaatttgctgcccttacccccctttttttaacttactgcttttcgaggagcaagacctgccctgctcagtacagtagccactggccacgtgtggctttttacatcttaaagagaaaactattatagttttgagtaagagttaaaaataaattaaatgaaaaattccatgcgtctgttcttaccggttttcaagtgctcagcagccacactaggctagtggctaccatgttggacagagcagataatggaacagttccatcatcacagacagtgaaggtgtagagttttgtcactaatgagcagtagttgttgttttgtctggtgaatattccagggggtacttaattcattgaacatgtattattagaattcaccatgtgccacacattgtgctgggtgctaaggaaagcttggtgaacccaaacatttttggtctctgccttccttgtgttcatggtctagacagggaggcagacagatttcagatgatcacataaagtgaaactaatatatgtgctaatattctatgaaagcataaagtaagataatctggccttgtcagtgaagttgggaaagccttcttgaggaggttatgatagagctgatatctgaagacagtttaaggttaatcaggcaaaggggagggaaagagccgtctagctgagggaaaaacacatgaaaaggtactgttgatgagagagagcatggcattttccaggacccagaagccaacgtggtttgagcacagactgcaaggctttgtgtgctatgccaaggatttaggggctttatctctggagctttggggagttgaagaaaatatttgaaacgaggggatgtatgataagttaggtgtttcagaaaggtcaggtgtatagcagagtgtggaacagctgtgatagcaaggtgggatggggagcagtagccataggtcaagggcataaattaggagaccattgcagcattgcccggcaaaagatgatgaaagtttggaatagggaagtgttggtggatacagggaaaagtggactgatttgagagatatttaagaggcaaaatcatcatacttaataacggatgggatgtgaaaggtgaagtagccaggtgttaggttattgtttgaatggttgtgccaatattaagatatggccagatttatggtggaggtggtggctgtgagtgggtggtggaattggttgggtgagtcttaagattatgagtttggttttgaagattgaaggagaaggctgggctagagatttcaagttgtaagtcatttacatagagaccataattaaagccacacttgttgactaggtcttctaacttttattttgtcctgtgacttcttgcttgaataactattaatattcaaactttatcttgaagcctagaggaggagttttttaaacaaaattttaatttaattttagatttgggggtacatatgcacgtaattaacatggatatattgcataatggtgaggtttgggcttctagtgaacctctcacccaaataatatacctttgtacgtgatagtgtaacattgtacttgataggtaatttttcagcccttctctcctccttctggagcccccagtgtctgtttatttccatctttatgtctgtgggtacttattgtacccatttatctttatgcctctgtgtacccattgtacccacttgtcttaccagtgagagcatgtggtatttgattttctgtttctgaattgtttcacttgataaatagcctccaggctccatccatgttgctgcaaaggacaggatttcattctgtttatagctgcataatattccacaatgtatattaatacatactacattttctttacccattcagttgttgatggacacttaggttgatttcatgactttgctattgtgaacggtgctgtgataaacatatgagcacagatttttttctttttttttaatggcgtctagctggtgtagaacacctgggctcaagtgatactccgccttggcttcccaaagtgctgggattacaggtgtgagccagtgtgcctggctcagatgtctttttgatgaaatgatttaatttcctttggagagatacccagtagtaggattgctgggtcaaatggtagttctgttttgagttcttcaagaaatctccatactgctttccataggagttgaacaatttacattcccaacaacagtgtatacgtgttcccttttcttctcatccttgccaacatctgttttttgactttttaataatagccattctgactggtgtgagatggtatctcattgtgattttaatttgcatttctctgatgcttagtgatgttgagcattttttcatgttttttggccgctcgtatgtcttcttttgagaagtgtctgttcagtcttttgcccgctttttagtggggttatttgttttttcttcctgttgatttgtttgagttccttatagatgctggatattagtcctttgtcagatgcatagtttgcagatattttctcccatcctgtaggttgtctgtttactctgttggtagtttattttgctgtgcaggagctcttctagtttaattaagttccatttgtctgtttttatttttgttgcatttgcttttgaggtcttaatcataaacttttttcctaagtattctttaggattattataatttcgggccttacatttaaatctttaatctatcttgagttaatctttgcatatggtgagagatagggacccagaggaggaatttttaagctaaccctcttttactacagccaatgtgagaaaattattttcctgtttaataatgaactatcttttttttcccccgccgagatgaagtcttgctctgttgcccaggctggagtgcagtgtcacgatcttggttcactgcaacctctgcctcctgggttcaagcaattctcctgcctcagcctcccgagtagctgggattacaggtatccaccaccacgcctggctaatttttgtagttttagtagagatggggtttcaccatgttggccaggctggtcttgaactcctgacctgtgatctgcctgcctcagcctcccaaagtgttgggattagaggcatgagccaccgcgcccggcctctaatcgtgaactatctatgggtaatagaaaatcttacttctgagagcccagagtattttttcctccccacagttttttctttcccctgagaaaaattagatattttcttatttacctgagagaatatatgataatatatagaatatatagatgattttctgtattaaaggtagatttacaatgatacaaggagtgttcagataatttttcctttagttcactggcatattgaaacttactggcataggttggaaagtgtttaaggaaaaaatgcttagtatgtttcttgctagaaatttatgaactatgaaattcttagatgtgttgtataacatctaatgctgcgtatttagaggatgagatcaaataataggaaacattaatttcatccagtttactctcaagttaaatgacaaaattgtgttattcaatttactctcatatctcactagataataagggagttatagtggtgttatataaggcagtgattaagtttgtagtcctgttactaaggctgagttaaactatgatactgtttaaatttgaagcaaaatatgaccagtcttataactaagtctttttaagagggaagtgagtgattcagagccttatgttgtttttatgctggtcctacagaccattcaggcacttcttgtaattccttaacctgaatttctgagttgtgctggggaagataattcagttctctgggttggcatcactagaaattcttggtctccaacttttaattgggtcatcaacagtgcttggaaatcttttttttttttctttttaagatggagtctctcgcttttgtcaccaggctggagtggcagtggcccgatcttggctaactgcaacctccgcctcccgggttcaagtgattctcctgcctcagcctccgcagtagctgggactacagatgcgcgccaccatgcccagctaatttttgtatttttagtagagatggggtttcaccatgttggccaggatggtcttgatctcttgacctcaggttatccacccgcctcggcctcccaaagtgctggcattacagatgtgatgagccactgcgcccggcccaacagtgtttggaaatctttagtttcctctctgtcagcttctttccccatcttccctcttaacttctgtctcccacccagagttcaacatccccccataagcaggttacagtttcaaagaagagaacacaggctgaacctctcccgtttctgctccccatctccaaatttatctacagtgcacaatttatttttttaacctattcatatacgttttattctttttttttttttttttgagacggagtctcgctctgtcacccaggctggagtgcagtggcgcaatctcggctcactgcaaactcagcctcccgagttcatgccattctcctgcctcagcctcccgagtagctgcgactacaggcgcccgccaccacgcccggctattattttgtatttttagtagagacggggtttcaccgtgttagccaggatggtctccatctcctgacctcgtgatacacccgcctcggcctcccaaagtgctgggattacaggcttgagccaccgtgcctggccttgtatacgttttattcttaatattaatcctgttttgtgttcctagggtaaaaactatttagtcgtagtgtgcttttctcttgaggaactgctagattttatttgcagatatttgataatggttttaaatgtttttctgtttgttttttgttttcttggccagtatttatcaaacttgacatcaatattatgccattttcataaaaggtacttggatgatttttgtttttctggaacagtgtaagtacaaaggaatttttctttcttgtatgtttgaaagatatcaccatttaaactatctctgtattcctttcagggttgtagtctatatatgttctcctttcttgggttaattttggtggttttcctaggaaatcattcttttcagtaagggtttctagttaattttaatggagtgctacacagtattctttattttttcatttgcctctgtggttattttcacttctcattcccaattttgtgtaattatgctttatttttctcctgatttgactaggtaggggtttatttaatttatttttcattcaaggacacagctcttgatttaagtttctgttgctgtttattttttaattattaaaaattattaattttaataataagttcagttttcatcttttactatttccttaggtttactttgatgtgtgtgtgtgtgtgtgtgtgtgtgtgtgtgtgtgtatttccttgtgttgagtgcttaattgacttacttaaaaacattttaatagcttttttgagatatcattcatatactgtacaattcacctttaatttaaagtgcacaattcagtcattttctgtatattcatagatatgtgccatcatgatcacggtcaattttagaacattttatcacttctaaaagaaaccccatacactttagctgatatcccccgtcttccattccccatatgcaactattaatctaccttctgtctatagatttacctattttgggcatttcatataaatggaatgatatgtgtttttgtgactgggcttctttcacttagcttaatggtttcaggattcacctgtgatgtggcatgtatcagtacttcattactttatatggctgaataatattttattatatggatatgtgatattttgtatatccattcatcagttgatggacatgttttttttcctcctttttggctattatgtataatgctaccgtagacattcttatataagtgtatgtgtggacatatggattcatttctcttggatgtatatctaggagtagaatttttgggacatatggaattctgttaaccttttaagtttctgccagattgttttccaaagtggccttcctattagcagtgtatgagagttccagtttctccacatccttatcagcacttgttattacctgacttttttgtagttttgatttgcatttcccttatggttcatgcatctattcatgtgcttcttggccatttgtgtgtcttctttggagaaatatctattcaggtcaaacttttgcccattctttaattgggttatttgtctatttactattggattgtaagaattctttatatatatgcatattacatatataatcccttatcaatcaagggattgacaagatacagtcccttgacaaatgtgaggtcatgaggacctactatgttttctttgagattttatagtattgctattacatgtaggtctttgatccattttgagttaatttttgtatacgatatgaagaaagggttcaaactttattctttcgtatgtgactatccagcactgtttttttttttaaattttattgatttgtgtaaatttatagagttcaagtataattttgttacattgatatattgcatagcggtaaagtcagggcttttagtgtatccatcactggagtagcctacgttgtactcattaagtaatttctcttcatccaccccactctcacactccccacccttccaagattccactctctgtcattccatactctgcttccacatgtacttgttgtttagctcccacttggaagaataggcagtttgtctttctgcagcaccatttgttaaagaggctgttcttttccccattgattggtcttagtacccttgtagaaaatcagttagccatagatatatgggtttatttctggattcccaattctatttcattgatctatatgcccaccttattccaggaaacacttttttttttttttttctgagataggtgtctctttgttgcccaggctggagtgcatgcagtggtgcaatctcggctcactgcaacctccacctcctgggctcaagtggacctcctatctcagcctaccaagtagctggaactacaggcacatgccaccatgcctggctaatttttgtaggttttgtagggatggaatctcaccatgttgcccaggctggtctcaaactcttgggctaaagccatctgcccacctcggcctctcaatgtgctgggattacaggcgtgagccaccatgcctgactaggagacactcttttgattaatattgctttatagtaagttttgaaatgggggagttctccaactttgctttcctttttcaagatattttagccattctggagccccttgcaattccatatgaattttagattagcttgtcagtttctgcaaagaagccagctagtattttgatagagattgtgatgaatctgtagatcaatttgggagtatggtcatcttgatattaaatcatctggtccttttttttgttgttgttgagatggtgtcttgcccaggctggagtgcagtggtgcaatctcagctcactgaaacctctgcctcctgggttcaagcagttctcctgcctcagcctccagtaactgggattacagatgcacaccaccatccctggtttatttttgtatttttagtggagacggggtttcaccatgttgaccaggctggtctcgaactcctgacttcaagtgatccacctgccttggcctctcaaagtgctgggattgcaggcgtgagccaccgtgcctggcctaatcatctggttcatgaacatgggatatcctttcatttatagttgtgtcaattaatgatgaggatacattctgagaaatgtttcattacagtatttcattgttgtgcaaacatcatatagtgtacgtacacaaacctagatggcatagcctactacacacctaggctatatagtctaactgtttgctcctaggctacaaacctgtacagcatgttacagtactgaatactgtaggcagttgtaacactgtggtaagtatttgtgttggatacgtttgtgtatctaacacaaatacttagaaaaggtatagtaaaaatatggtattttacatatatatgtgtatatatatatgtgtatatatatgtgtatatatatgtgtatatatatgtgtgtgtatatatatatatgtgtatatatatatatatatatttttttttttttttttttttgagacagagtctcgctctgtcaccgaggctggagtgcagtgacacaatctccgcccactgcaaccaccgcttcccaggctcaagtgattctcatgtcagcctcctgagtagctgggattacaggtgtgtgccaccatgcctggctaatttttgtatttttagtagagatggggttttgtcatgttgcctaggctggtcccgaactcctgactcaagtgatctgcccaccttggcctcccaaagtgctgggattagaggtgtgagccaccatgctgggccggtattataatcttataggaccaccattatgtatgtggtctgtagttgatggaaatgttctgatgtggcatacaactgtaattagatcttctttaaatttcaataatgttttatagttttcagaattgcactttttttcttaaattcttcaaaatactgtttttaatggtattgtcagtggaattatttttcttaattgaatgtttgggttattcgttgtagatgtttaaaaacacttgatttttatatatattaatattgtttctgaatctcttctgaacttgtttattcatcctagtagattatttgtgggttctctatgattttcttgtaaagatcatgtcaagggcaaatatatatagctttattttttctttccaatctggatgccttttatgtatttatttatggctaattgccctggctagaacctttagtaaaatattcaataggaggggtaagagtgtatatgcttttttattcctgatcttagggcaggggtgtccaatcttttggcttctctgggctacattggaagaattgtcttgggccacacataaaatacactaacaatagctgatgagctaaaaaaaattgcaaaaatataatgttttaagaaggtttacgaatttgtgttgggccacattcatggtcctcggctgtgggttggacaaacttgtcttagggagaaagcatcctgtcttttacccccgagtatgatgttagttgtgggtttttaatagatgcccttatcatattgatgaagtttctatccctaatttcttaagtgtttttaataataattttgtcaaattattaatcgatgtattacattaattgatattaggttattaaaccagccttgtgaaaataactcactttgtcacagtgtataaacattttatgtgttgcctgtcatagaaatacacacacacacacacacacacacacacacacacacgcttgtattttgtttagagttattccatctatatttgtaagatatattggtctgtagttttcttttgatgcttttgtctggtttggtatcagggtaatattgacctcataaagtgaattgaaagtattcctattggttttggaagagtttgtgaaggactggcattaattctttgaaagttcgataggttacatcaaatgggtctgagctctttttttttttttttttttttgcaggatggggcatagtattttaattactaaatcactcttttttcttcttatgggtctattcagattgtccattctttttttttttttttgagacggagtcttgctctgtcgcacaggctgggggtgcagtggcgcgatctcagctcactgcaagctccgcctcccaggttcatgccattcttctgcctcagcctcccaagtagctgggactacaggcgcctgccaccacgcctggctaattttttgtatttttagtagagacggggtttaaccgtgttagacgggatggtctcgatctcctgacctcgtgatccaccctcctcggcctcccaaagtgctaggattacaggcttgagctaccgcgcccagcctggttaggctctggtaaaatagtttgagggcaagttttctaagaagaacaaaatactctgggaatattcaaaatggttatccccaaccccctccccctaactggggggtaggtatttttctctcatcttaactgtggtaacatgctaggggttgtggagataaaacttacagaagcatggaggcccttcataagactggtttccctgcggcttttaattctcaaagttgtcgcattgcacctccagcaatttgtcaattacactagatttttcttccctagtactagttagttcccacagtggtttctgttcttgggattctgctctggtaagttgtgattctctgtgtatgtacctctccaatttttggaacaatagtttgcccttcatctcagttctctgatggatataagaagagttttgattttttagtttgttcagctttttttttttatgatggggatgggagtgattacttccaagctctttatatgcttcaccagacctctgttttggggtttttttgttttgttttgtttttgagatggtgtttcgctcttgttgcctaggctggaatgcaatggcattatcttggctcactgcaacctctgcctcccggattcaagtgattctcctgcctcagcctcctgagtagctgggtttacaggcatatgccaccacgctcagctaattatttttcttgtattcagtagagatgggatttcaccatgttaggctggtctcgatctcctgacttcaggtgatccacctgcctcggcctcccagagtgctgggattacaggcgtgcggttttgcttttaaacagactttatcttttagggcagttttaggttcacagccacattgaatgggcaatacagagttcccatctgtgccctgcccctctccatagcctccctcactgttaacatcctgaatcagagatgtgtcttttgcaaatattttcttccagtctatagcttgtctttttattcttttgacagtgtttttcacaaagcagaataagtttttaattaaatccagcttatcaattatttctttcatagattatatgcctttagtattgtatccaaaaagccatcaccaaacccaaggacatctagattttctcctatgttatcttctaggagttttatacttttgcactttgcatttaggtctttttatacttttgcactttgaattaatttttgtgtgtgaaggatgtcagatctgtttctgggttcattgttttgcatgtaaatgtccagttgttccagcaccatttgttgagaagactttctccattgtattgccttcgttttttggtcaaaaaatcaattgaatacatttatatgggtctatttctgggccttcaattctgttttattgatcaatgtgtctatttcgttgatacatgctgtctttattactgtagctttatggtaagtcttgaagttaggtagtgtcagtcctccaattcggagtcttttgcccctccatataaactttagattgaattcattgatgtgtattaaaaattgctgggattttgattgggattgcgttgatcttaatagatcaagttgggaagaactgacatcttgacaatattgagtatttcatgaagttgaaataccttctcatttatttagttctttgatttcattcatcgagttttatagttttcctcatatagatcttacatgtattaagttagatttatacctaaatattttatttcttgggttgttaatgtaaatggtattattgtacttttaacttcaaattccacttgattgctggtatacaggaagtgattgacttttatatattaacctatattctgcagttttgctagttttacttattaattccaggaaagttttgtcagttcttttggattttctttttttttgagatggagtctggcactgtcacccgggctggagtgcagtggtgtgatctcagcttactgcaacctctgcctcccgggttcaagcgattctcctgcctcagccttctgagtagctgggattacaggcgcctgccaccatgcctggctaatttttgttttttttgtatttttagtagagatggagtttcactatgttggccaggctggtctcaaactcctgaccttgtgatctgcccttctcagcctcccaaagtgctgggattacaggcgtgagccactgcgcctggcctggattttctgtatagacagacagtcatgtcatctgcaaacagagttttatttcctccttcccagactgtgtaccttttatttccttttcttgtcttattgcattagctatgacctccaatacactgttggacagtggtgagagggtgtattatgtcgtcattgtgttgctatgaagaaacacctgaggctgggtaagttacaatgaaaagaagtttaattggctcatggttctgcagagtatgagaagcttggtgccagcatctgcttctggtgagcttacaatcatggtggaaggtgaagtgggggcaggcgtctcacatggcgagagcagaagccagagagcaagggaggaggtgccactcttttaaacaaccaggtcttgtgtgaacttagagtgagcgctcacttatcactaagggaatggtgttaaactaatcatgaaggatctgcccccatgatccagtcaccttgtaccagaccccacctccaacactggggattacatttcaatatgagatttgaaagggacagatatccaaatcatatcagagagggaatccttgccttgttcttgatcttagaggcaaagcttcgagtttcattaggtatgatattagttgtaggattttttgtagatactctatgtcaagttgagaatattcccctctattcctattttaatgagaatatcgtgaagaggttgtcaaatgttttttctgcatctgttatatgatcatgtggtttttcttctaaagcttgtcaacatgatggattatattaattgtttttttttttttttttttgagacagggtctggcgttgtcacccaggctagagtgcagtgttgccatcttggctcactgcaacctctgcctccaaggctcaagccgtcctcctacttcaccctcctgagtaactgggaccacaggcatgtgccaccataccaggctaatttttgtattttttttagaggtggcagaggggggtctcactttgttgcccaggctggtctcgaactcctgagctcaagtgatctgctcaccttggcctctcaaagtgctggaattacaggtgggagccactgtgtccagccttaactgacttttgaatgttgaatcaagtttgcgtacgtgggataaacccacttgtcatgatgtacaatacttttgtacattgttaaatttgatttgctaatattttgctgagaatttttacattgatattcatgggagataatggtctgttgttttcttttttggtaatatgtttgtctggtattggcagtagggtaatacagacttcatagaatgattaagaagtattctctctgattctgtcttctgagagattttagagaactggtacaatttcctccttcagggtttggtagcattaccagtgaacctgtctgggtctggtgctttctgttttagaaggctattaatttatgattcagtgtttcgaatagatataggcctcttcattttgtctatttttgtgtcagatttgacagattatcttttttaagaaatcgttcattttatctaggctatcaaatttgtgggcatagagttgtttatagtgttcctttattatccttttaatgtccatgagatctgttgtgatgtcctctctttcttttttcttttttttgagacagagtcttgctgtgtcaccgaggcaggagtgcaatggcgcagtctccctcactgcaacctctgcctcctgggttcaagcgattcttgtgcctcagccacctgagtagctgggattataggtgtgtgccactatgcccggctaatttttgcatttttagtagatttagggtttcgccactttggccaggctggtctcaaactcctggcctcaagtgatctgcccaccctggccttccaaagtgctgggataacaagagtgagctaccatacctggcctcatttttgatgttcataatttgcatcctttatcttattttattagcctggccagaggtttattgattttattgatcttttcaaaagaaccagcatttaagcttttactgattttctctctttatttgctattctcaatttcattgatttctgctctactttttatttattttcttctcctcactttaaatatgatttattcttctttttctagtttcctaagttggaagctttgataattgatttcagatttttttcctaatatatacattcggtgttataaatttcccagtaagcactacttttgctgcatcacaaattttgataagttgtattcttactttcatttagttgaaatatttttaaatttcttttgagagtttttatttgacccacatgctatttagaaatacactgtttaacctcttaagtatgttgggattttccagctatttttctgttattgatttctagtttattttttcctttattgtatatatttatacatcatgatgttttgatatacatatacctagtgaaatggttgccatagtcaagcaaattaacatattcgtgatctcacatagttactgcctttttttttttgtggcaagagcttctaaaatccttttagcaagaatcctaaatataatacaatattattaactatagtacttatattttacgttgaattgctaaacttgctgatccatcatacctgcaactttgaatcctctgacctgcatctccccatttcctcccctgttcctcacttctacttctttgtacttgactttaaaaaatagatttcatatgtaagtgagatcatgcagtatttttctatcttccaggtttattcatgtgacaaatggcaggatcaccttcctttttaaggttgaataatattcctcccccttcccctcctcgtgtgtatgtgtgtgtgtgttatgtgtgtgtgtgtgtgagtgtgagtgtatgtgtgtgtgtcttgtagtttattcattcatcaatggatgctgtagttgtgttcgtggctattgtgataatgctgcagtgaacatgggagtgcaggtatctgtatgaggtggtggtttcattttctttaggtttttacccagaaatggaattgctaggtcatacaagaaaagtagtccttttctttcttttctttctttctctctctctttttttttttttttttttttaagacagaatctcgctctgttgctcaggctggagtacagcggtgtgttctcaactcactgcaacctccgcctcccgagttcaagcgattcttgtgtctcagcctcctgagtagctggggttacaggtgtgcactaccctgcctggttaatttttgtatttttaatagaggcggggtttcgtcatgttggccaagctggtctcaaactcctaacctcagtgatcctcccaccgtgtttggattacaggcacgagccaccgcgcccagtcgtagttctatttttaattactttagaaaactttatactgttttccgcaattgctgcactggtttacattgccaccagcagtgtacaagggttctcttttttccacacccttgccaacacttatctcttgaatctttttgttttttaaataatagccatcctaacaagtgtgagatgatatttcattttggttttaatttgcatttccctgatgatagtaatattgaatatcttttcatatgctgattggccatttttcttttctttttttctgttttggagaaatgtttgttcaggtccgttgcccattctaaaattaggttgtttgtttttcttctactgagttgcacaagttctttatatattgtggaaactgactccttatcagatatatggtttgcagatattttctcatctgtagtagactgtcttctcattttgttgattgtttacttagatgtgaaactctttagcttgatttagttctatttatttatttttgcttttgtagcctgagcttttgttgtgatatccaaaacaacattgccaagacaagtgtcaaggagtttttcccatatattttcttctaggtttatggttttaagtcttcatgttgagttctttaatcaactttgagttgatttttgctggtggtgtaagataaaggtccaatttcattcttttgcatgaggaaatttaattttctcagcaccattatttgaagagatcttctattccctattgtgtcttcttggtgcccttgtcaaaaattagcgaatcatatatgcttgggtttatttttctctctctattctgttccattggtttacatgtctactttttatgccagtaccacactgtttgggttactataactttgtaatataatttgaaaccaggaagtgtgcttcctccaactttgtttttgtcttacagaattactttggctctttagagtcttttgtggttctatatgaattttagatttttttttctatttttgtgaaaaatgccattggcattttcataaggattgtgttgactttgtattgctttggatagtacatacattttaacaatattaattatttcagtctgtgaacatggaatatctttctatttatttgtgtccttaatttctttcattagtattgtagttttcagtagacagatcttttgccttcttggttacatttatttctaagtctttttattttgttttttgatgctatcataagtgggatggttttcttgatttctttttcatataagtagttatttgtgtacagaagtgcaactgattttcatatattgattttgtatcctgcggctttactgggtttattcttttttctttttgagacagagtctcactctgttgcccagggtggagtgcagtggctcgatccctgctcactgcaacctccacctcctgggttcaagtgattctcctgcctcagcttcgcaaatagctaggactacaggcgcgctgccacacctggctaatgtttacatttttagcagagatggggtttcaccattgctggtcagggtggtctcaaactcctgacctcaagtgatccacccgccttggcctcccaaagtgctgggattacaggtgtgagccactgtgcctggcctattcattctaacagtgtcgattttagtctttgggggttttcagtatataggactttttcctttcaatttatatttctttttcttgcctaatcgctttagtaggatttccagtgctatgttgagtagaagggtgagagcaagcatctttgtcttgcttctgatcttagaggaaatgttttcagtttttcaccattgagtatgatatgagctgtgggacatacagcttttattatgttgaggtagttttcttctgtttatagtctgttgactttttttttttttaaataatgaaagggtgttgaattttgtgaagtgctatttcggtttcatttgagatgatcatgtggtttttgtccttccattaatgtggtgtattacattgatcaatttttgtatgttgaaccatccttgtattccacgaattccacttggtcatggtgtgtaatccttttaacgtactgctgaattcttattggtaatattttgttgaggatttttacatcaatagtcgtcggggatactggtttatagtcttctaatgtctttgtctgactttggtatcaggataatgctggcctcatagaatgatttggaatgttccctcctgtgcaattttttgggattgtttcaggaggattggtgttaattcttctttaaatgtatggtagaattctccagtgaagctttcttgtcctggaccttttttttttttttttttttttttttttttgagacagggtcttactttgtcacccaggctggagtgcagtggtgcaatctcagctcactgcaacctctgccctccaggctcaagcagtcctcctacctcagcctcccaagtaactgttaccacaggcaagcaccaccatgtccagctattttcgttgtatttttagtagagacaggatctcaccatgttgcccaggctggtctctaactcctgagctcaagcatttctcctgccttggcctcccaaagtactgggattacaggtgtgagccactgcgcctggccatgggcttttcttttgtgggaggttttggattattgattcaatttccttgttgttggtctgttgagattttcttattttttcatgttactcagtcttggtaggctgtgtgtttctaggaattgatctgtttcttctaggctgtccaattagttggagtacagttgttcatagtattctcctatgatcctttttatttctatggcattggttgtcatgtcccctctttcatttctgaatttagttatttgaggcctttttttttctcagtcagtctagctaaaggtttgtcagttttgtttatattttcaaataactattggttttcttgatttttttcctactgtttttctattctcttttctttatctttgctctaatcttattttttccttctgatcactttgggtttagtttctttttctagttcttcaaggtgtaatgttagattattgacatgaatttttttttttaaatataggcatttacgactgtagattaccttcttagtcctacttttgctacattcaataagctttggtattttgtgctttaatttttgtcttaatgtattatttcctttgttacttctgtcgtgacccattgtctttttaagagtttgttgtttaatttctataaatttcttgattttccacttttctttctgcagctaccttctagtttcattctgttgtgatcagaaaagatacctaatactttgtggccaacatgtggtgtatcctggggaatgtttcacgtgcacttgagaagaatgtatattgtgctgttgttgggtggagtgttctgtatatgtatgttaggtccaattggtctgtagtgctgttcaggtcttttctttccttattgatctcattctatccatcatggaaagttggttattgaagtctctcactgtttttggagagctcccttcaattttatcaatgtttgctttaagtatttaggagctccaatgtctgacacatatataattattatatcttatttgtgcattgaccccatagtgttctcctttgtctatggtatcagttttgtttaaaaatgtattttctctgatattagtgttgccgctattgctctcttttggttaccatttgcatggaatatctttttgaatctttttacttagagcctgtgtgtgtccttagttccaaagtgtcttttaaacagcatatagttgggtcctgttttttaatcctatgggtttttttttttttttttggtaatttctttgtcctttcatagcttgtttttcatttcctccccttctttctttgtgtttagtcaatttttttggtagggatatattttgattctcttatttccttttgtgtatattctgtagctattttcattgtagttaccatgagaatatcataaaactccttaggaggataacaacctgtttaaactagtaacaacttcacttcaatcacatataaaaactctagtcctttatagctttgccccccaacctccacctcccgggttcaagtggttctcctgtcttagtctcccaagtagctgggactacaggcacaggccaccatgcctgactaatgtttacatttttagcagagatggggtttcgccattgttggccaggctggtctcaaactcctgacctcaagtgatccacttgccttgcctcccaaagtgctggattacaggcatgagccaccgcacctggccaaagttattagtaccctgttaacaacattttactggtgtcacaagtaacatgtttatatattgtttaaccattaacatactttcttagtttgttttctcttgcttataacaatacttgaaactgggtagtttattttaaaaagttatggaggctgagaaatccaaggtcaagggtgagggcctgatgagggccttcttgctgatggggactctgcagaatcctgaggtggtgcagggcacatggtgagggggttgggcatgctagctcaggtctctcttcctcttcttataaagctaccagtccactctcatgataagccattaatccattaatccatcaagtcattaagccgttcagatctgccctcatgattaattcattcatgagagcccagatccctgatgacccagtcacctctgaaaggttctacctctcagtactgccacattgcggattaagttttaacatgagttttagaggggacagatactcaaatcatagcatgtagatttatagttaatttttatgcttttgtcttttaaatcctataaaagaattaaggtggagttactcaccagaattaaaataatgtttttatatttgtccatatatttacctttaccagagaactttatattttcctatggcttaaagttactatctagcatcctttaatttcagcttgaaggactgccattagcatttcttccaggtgaggtctagtggtaatgaactctctcagcttttgtgaatgttttaattttcccttttacaggtgcatgccatcatgcccagctaattcttgtatttttagtagagacggggttttgctatgttggccaggctggtctggaactcctgacctcaggtaatcctcctgccttggcctcccaaagtgctgggattacaggtgtgagccactgtgcaccttgtatctttctttgtctgttcgaggatctgagatactattttaaagtcctttggaagtatagtttgtaatcggatttttagtaagagggttattttttttttctctttgtgtgcattcttctctacctgctctttttgtggttgtttctgctttgtcctttatggcatcttctttagaaggaaatcttatattggaagctctgggttcttattcttcagggttattgggaaaaatctcaagaaccagtccctgaaccagtgggtggcttgaccaggtcatttttgttttattgcctgtaatctgtccatttgtgttccttcagcctttggctatggtcacaacgtttattgccttctttcactagtaggagagcgctgctccaaccccttacttcacaaaatgattctgacttttgttcccacttcttgtgctatgagttgatttcagtccctattattggggttgatttttaactgtctcaaccagtaagatttaaaaatagacaacttgcttgtttctgtttgctgcttttacctctatgggtaaaggaggttaaggaactgctgtggtttatgcaaaagtatgctgtgtcctaactgagtaaccttaaggataatagcttgtataatggttgtgaaatcaaaatgtcgtatatgtgaaagggctttataaactgggaagtatggtctaaatattagtttatttttgttgtataatcatagtgattgtgttattactctatttaagcctgcttttgattttcagattgttccattgacctgtcatgtatggcaacagatagtatatcaaggcaatagtagaacacaaatttctgatactaatgtggtctgtttggaaacaacagctcagcggggttctggggatgatcaggtatgtcttctgtaactggctaactttttttttttgataagcagcacaagaaattctgatttagctatgaagaatggaaaaataaattaatattcattaatattatgttttcttgtcttttttttttttttttttttttttttaagacagggtctcactctgtcacccaggctggagggcagtggcatgatcatagcttactgcaccctccaacttctaggatcaagtgatcctcctgcctgagcctcttgagtagctgggactacagacatataccatcaaacccacctaattatttttaaaaaattttttatagatggagtctcgctttgttgcccaggctggtcttgagctcttggcctcaaatgatccttcgccttggcctcccaaagtgctaggattacaggagtgagccaccactcttggccaaatattctatttttctaatagattgttgtaaactttatgtatatgtagaagagcagttcactttttttgaaggcgggtctcaggactcttatttttatttttagtttaaaaattttttatagagatgggagtctctgtgtttactgggctggtctctcattcctggccttaagtgatcctcttgcctcggcctcctaaaactatgagattacaggcatgagccactgtgcctgtcctcaggactctttatattcttcacaagtattcttaagagcttttgtttatgtggattatctatatagatatttagcatacaagaaattaaaacatttaaaactattttaaaatttattttgaaagaacaaacctattacatattaccataaatagtatattttaaagtaaaaaatgactgttttccaaaacaaaaaaattagttggaagaatagcattgtttaaattccggcttactagaactcagttggtatattattcagggttctccagagaaacagaaccaataggttggagatagatacatctatctatttatacacatctgtgtgtatgtgtatatatatatctttattgatatttattatatagaattggcttatgtgatcatgaagactgagaagtcccaagatctgcaagctggagccccaggagggctgatggtatagttccaattcaagtccaaaggcttgagaaccaggagcaccagtgatgtaagttttgatctgaatctgcaggtagaataccctccagctggaagaccatcaggcagagagaacacattctctcttgctcagccttttgttgtatttaggccttcaaggtactggatcaggcctacccacattggagagggtaacctgctttacttagtttccagattcaaatgttaatctcattcatacacacctcacaggccaggaacagtggctaacgcctgtaatcccagcactttgggaggctgaggtgggtggatcacctgaggtcaggagttcaagaccagcctggccaacatagtgaaacactgtctctactaaaaatacaaaaattagccaggcgtggtggtgcacacctgtaattgcagctacttgggaggctgaggcaagagaatcgcttgaacccaggaggtggaggttgcagtgagctgaggttgcgccactgcactccatcctgggcgatggagcaagacttgtctcaaaaaaaagcaaaaaaacccaagaaaacaaaacaaaaaataccacacacatacacacacctcacagacgtatccaaaattgtgtttaaccaagtatctggacactccatgatccagtcattttgatacataaattacctatcatagctggattctcagtctgctctgcatttcatttgttgggatgtattgttttggttgaagtatatgaagacaatcttgccttacatagatgaaaaaggagttagggaaaggaggctgtcattcagtccctgaaagggtgtcagtgaccctcagggatttttggactacactttgagaaccacttttctagaacatagttataaactttgaagaaatgaaacgatcatgagcagataggaaaaaagctcatgtagagttattggaggaaaaatgttattagtgttactaataatggttaaatatttaaggttgattaataacttgagtatcagatttctcctattaccttcagttttctttaaattgcagtgattacctgttagatgcaagatatggaggaagggagatacactatcactgaggcaaagtagttaagacaatccctgaccataaaaaacatagtacctggggccattaaatggaagaggattgttttgggtcgaattgtgccctcccaaattatatgttgcagtcctaatccccaatacctcagaatgtgaccttattggtgattgcaggtgtaattatttaagctcacactggagtagggtttgctcttaatccaatctaactggtgtccttatgaaagaggaaagcaccatgtgaggagaaaaacatacaaggagaagatggccgtgtgaagatggaggcagaaattggaattatgctgccacatccagggaatgcctggggcttccagaaggtagaagtagcaaggaaggaccctcccctaggggcttcagagggagcatgaccctgccaacaccttgatttcagacttctggcctccagaactgcgagataataaatttctgttgttttaagccacttagtttgtgtcactttgttatagcagccctaggaaactaataccgggtcatatttctaaataatattctctgagggcatatttttggtagagcacagtccaaaaattgatgtcttcctttttttcctgtttcacacaatacgaaccatgatggaccctaaagcacttcatttcatcctttacgcctgggttcgtcagaatagctggaatattatgccgctttctagttcacttccttcttattctttcgtcgttcatctttgttactgtcatctgagttacctttatttattcatggaaaactagctcttgctacattgtcttcctctcttttattggtttctttcacttcagcatacatttcccattcagtgcctgaagttctcatatatttcatctaatctccagtgatctttacttccgtacctccttccagtcttctcctacctgtatgtagttactcatcccatgatcacagtctatctagactttgtcatcaccagctactttactacaaatgtctcgtcctctcttcccttccggctactgcttcggtcaatattctgctccctgttacagtgaaattttcaagagttgtctatacattttctgtttctgtttcctgatatcctatgctcatttttaaaaagtcaggtttattgtaggtataggttaaacatatcaaaatttactctcttaggtgtacagtttgtcaagtgagctttgacatacgtatacagtcatgcagtcaccaccacatttgagatagacagcactgccttcacaaaaaggaacgtcacaaaaagttcctgtgtgcccctttgtagtcagtcacttccccaactcctagtccctggcaactacttatccaatttctttctctttagttttaccttttcagaataatacggtgtgtagacttttgaatttgacttctttcacttggcttagcttttgagatttatccatgttgttgcatgtatcaataatttgtccactttaatttttattactgagtagtattccatttgtgtgtaggtttttcggtagacatactgcttcatttctcttggcaaatacctaggaatggctggacgtggtggctcacgcctgtaatcccagcactttgggaggccgaagtgggtggatcacttgaggtcaggagtttgagaccagcctggccaatatggtgtagtagaaaccccgtctctactaaaaatataaaaattagccaggtgtggtggcacatgtctgtaatcccagctactcgggaggctgaggcaggagaatcgcttgaacctgggaggcagaggttgcagtgagtcaggatggcgccagtgcactctggcctgggtgacagaatgagactctgtctcaaaaacaaaaaacaaaaaacaaaaccctaggaatgggtttgttgagtgataaggtaattgtatgtttaactttataagaaagtaccaaactgtttttccaaagtggctgtaccattttgtatcccagtagtatatgagagttctgattgccctgcattcttggcagctgttggtatcatcagttttttctttttttcttttttttttttaattttagacaccctaggagatgtattattggtgtctcactgtgattttaacattcatttctctaatgactaatgatatatagcatctttttgtgtgcttatttgccctccatgtttcttcttcagtgagagatctgttaaaatctctgcctgattttttgtggctggtttgtttcattttttgagctttgggagttcttcatagctctagatgcaagtcctttatcagttgtttcttttgcagagacagtctgtggcatgtttttgtattctcctgtctttgaaaggcagaagttttaattttgatgaaacttgtttattgattttttttccttttttggtttatatttcttgtgtactatctgagaaatcttggcctaatcctatgtcactgagatttttcttctatgttttcttctagaccagggtttctcagccttggcactattgacattttgggttgaataattcttcatagtgggggtctgtcctgtgtgtcctaaaatgtttaacagcattcctggcctcttcccactagataccagtaatagtcccccaatttgtaaccatccggaatgctttctgacattgccaaatgttccatggttgaagggagaggcgtgtcaaaatctctcccaactgagaaccgctttttcagaagttttgtagttttacgttttacatttatgtctaagatccattttgagttggtttttgtatatggtgtgaaggaagcatccaagctgtattttcttacatatgaatatgtaattgttattctagcaccatttgctatctttttttccagtggatttcatgacatctttgtaaaaaaattaattgaccttgtatatgtgggtctatttctgcacactattccattgatttacatatgtatcctcatgccaatattacactcttaataactgtactgtcttaaaatcaggttgtgtgatttttcccactttgttcctctttttaaaaattgattatttcagattcttggaatttctatataaattttagaattagctcgcccaatttatattttaaaagcctgctggaattttttattaagatttcattgagtctataaatcaatttgaaaataattttgacatcttaacaatattgagttttctaatccataaacatgacagttctcttagtttattatttctctttaatgtcttcatcaatgttttgctgtcttcagtgtgccaatcttacacgttttgttagacttactggtatttttagaaatttctatttcctattattcattataagtgtatagaaatataattgactttttatattgaccttgtgtcctattaccttgctaatctcacttactagttttagtagcttttttaaaaaaatatttcctttaggactttatatgcagagttatattaactatgctttgttttttacttttaaatagatatgtcatttctttttttccgtttttttttttttttgttttttttttgctttattacactggctaggatctgcctattcttctacttgatctactttgtgttctgaccccaattctttgaaaggaagtatcaccagcaatatttatgtagctgaatacagtgaatacttttctgtcctcctcttaggtacgataaatagcatttgacactattattaatcattacataacttctttaaattatttcctttcttgggttcttctggttttttttctactcatctggctgttctctctcagtatcctttgctgctttatcttcctttgttaaaccttagaatcagaatttgtaagctgtggacccaagtatgactcaaatgtggttttcaaaaaccattttttttggcataatgttttaacacttttgaaatagaatactttaaagtgaacatgcatagttagccacaaatcccattactctccaattccaggttggattacttcatttgtcttacccccatacccctatacacatttagttttcaacctctgcttgaaacgctggaatgttcgcggtctcatccagatccaggactttaaatacatctgtatactgataaatcccaaagatgtatctcaaactttttttctccctcaaaatttgtttgtatttgtcagaatgcttatatgttgtccctccccacatctgtcaaaaatatttagagatttgttggcctttttatattgatttaaaaggtataacacaaagaatatttgatgattgaaatgtatcaagaaaatacataatgcagaaagttaaaaaaggttgaacagaggttgttttaaaaattcagtttataatgggaaattataagaatatgtttgcataggagaggaatgattcagtaatgagagaaaaatgggtgaaggaaagtgggagaatcacataggaaggaaatcactgaaaaaaatttgaatgagataggaaggcctttgagaggcagagtgacttttttttattttcaagtcataagatagaaatctggtaatggggattgtagatctggtggttgaagacaagggagttcctctctcctgctctacaattattgaaggtcatcagttgaaaattaggagcgttcaatagttctgaggagggaaggtatgaaatctcaactcaccaggaaaatgtagtaggatttccaggcaatattgagtgcccttcaggtttagtgacatcaatttaaagaaaagccagtcagtttgggagtgtgatttctccagcaacgtgctgctgattggattcatgcaaggagacggttccatcagtgttagcttttgctgagcaaataggataatgggagaaaaacgcaagaaagttaagggtgtttgcaaggcagtgattataaggatggctctgaactctcagaggggcaaagaaggaaatacagataagaaaggaataaagaaaagtataaatgtgataggatagttgtctccgtgaagttaaaaaattattgcagtagaggaactagagagtaagtgatctgtggaggtcagaggatggacagaaagactaatggatacatctgtcactattagagaggagtgaactatgggatacaggctcaaaccatggtcagagaggctaagagatgactcgggaccagtgatttttgctttagtgagtcacaagatgattggaattctgctttaaaaccttttgttggctcactaacctgtcccctatagaaaggagtgccctctaaagaaaaaatgtaaaattaatcattctcatattccttttattttcaagtatcagaaaggtgaaaagcatgtttccagtagcaatttgatgttatagatctgattaatttattcagtgtagaagggattgaggtattttggaatgtcaaaagtctgctattccataaatgtagcattaatattctagaatatttctctccacatttttttctttgcatgtatttgtataactgatattatgtagtggatagtgggtatcttttttcttaaacttactatatgaacattttgttaatgaattttaaatattagaaatgtctcttcaaagacaaaagtcttgctggaaaatgtttgcgaaaatatttgtaaattgtaatacatcttctttttatggattctgtaccacccgggaattaaaagatacctatattctcttagaaacatttgagttgatcaaggaagaagagtccagtagtggaatacttacaaaataatgaaaatacattatatgtctaaagttgctgtcaatataatgccactttctcatgtttttatttaaataaataaagcaaattgaatgaattatttaaccaaagattttacataaagactcaaaaacttgtaatcccagcactttgggaggccaagacaggggaatcgcttgagtctaggaggtccagaccagcctgggcaacatagtgagacccctgtctctacaaaaatttttaaaaaattaactgagtgcctgccagtagtcccagctactcaggatactgaggatgacaggatcacttgagcctggaggcagaggttgcagtgagctgagattgtgccactgtattctggcctggttaacagagcaaaagcctgtctcacacacacaaaaaaagaaaaactgcaaatagaggaaacgtggtaaagagaaaaacaagttataaaatataatacagttgttaaattcaagaaatagagaaaaaatagatatattcctagtaaatagagttgcgaaaaaaatctcaatgaaatagactttctacaaaactatgagtatttcatagaggtagactaggattactgaggaggaaattgagaaagttatgttagtaccagctatcaaaaagatttttggaactaatagatttgctgataaatcttttacatttttaaagaaaattgaagttgcttaaataaatacagaacaacgttatgaaacaagaaaatgagggcattctaaaatactgattaatcttagttgtgaccatatatgtgtgaaaatgttaaagaatatgctagcaaacatttaaacgtctgatctaccactactgtgtaagaaatgtgaacatgtttcaatataaagaaattttgtgacttgggaggctgaggtgggaggattaagcgctttagcccagagtttgagtccatcctgggcaacatagcaagaacctgtctaaaaaaaaaaaaattattgctatcctttatcaggtttaattggggaaaatgatgctataacatagtttgagctaatttagtgttcatttctaatcaaattatttgaaaatgaaaatactttcatatcatgatagactatacttaattattaactaatattaaattcaaaaggaaaataccttttaaacttgtcaccattagtatttaatacgactttaattcagcattgttatttagtgtgatctgtagattggtgccagtccatgatttgcttgttactggtctgtcaagaggtaagtatagaaattgagaggaagtgtttagaaacttttatagtagttggatagagtaattttatctttttttagacctagaaatcaaacatttgggcttgtgttttatatcttgggcttatagtaagacttttaatttcatttttctagttatttttactggttttaaaaattagcttatttctcattaatttttgttagcattttagtaaagtgtcagtatgtgatgacttggaagtaggaaaaactggttcttccctataggtagtttgagaagcagttctctggatcttgccactaatttaagagtaaatatttgaaggagacaagtgtatcacattcatgggtgatatgataatgttatatatactgagaagagccagggatcgtttagaaactagtagagctaataaaaattcagtcaagtgaattcccaagctaaagctgaagtgaatgcttcagtaggtcccatgaatcggcccagcccttgcgtgcaccaagtttggcccctctagcatcgccgccacgaagaaagtggctcaacagctttggctgaaggcccggctcaactgtgtaaaaatttcccaggcagctgcagacttgaaacaattctgtctgcagattgcttaacatgactctctgctgactggagtatcttcaagtacaaattccttcataccccagaaagtctgttcctttttgtagtaaaatgaatctttcagaggtttcccaagccacttctcatgatacagtgaatattcaaaagagagctacatttgaagcctgtacaaaagcttatccccggaacacatgtgccataatatacaaacttctacttttgtcagtccttaatatctacctctctgaattttcatgaatttctatttcacaagggtaattgttttatatacactggcagcggcgtacaatacaacttagtataaaaggtttttttgtgttaaaaaaagatgtgtagtaatgggaagaggtctaaaatgtagttaatattgtgagaacaacagctttacattttctatactttgatgagaatgtcattaaataggaagctatataatacatgagtggacattttcatctaaatattaatatgtatggtaactcagttaatgacttagcatgttttcctttaacatttttctagaaaacagaatcttggcattgtcttcctcaagaaatggactcttcccaaaccttggatacatcccagactaggtttaatgtgagaacggaagatactgaagtgacagacttcccctctctggaggagggcatattgacgcaatcagaaaatcaagtaaaggaacccaacagagatctcttctgttctccactgctaggtaatgcctgtttattttaactagtagtaatacctcacatttgtaagttttgcggggactgcaagtcttgtaactttcccctttttgagttaaggagctctgtagagacctactcagagggattagcttatttctgttttgtttgtttgtttgttttttactggctagctgatctaattatattaagtgtgcagttgcctctcattttatcacttcccgttgctcacagaaggcagaaaggtgataactgtctactgtgtgtgcctcctcagtcttgaatttgcagagtttttgggatgaatagtatactttggttatctcagcaatggagactccatgatcccccacaaaaaacaaaaaacaaatgttcatatttacctttttgatttttagaattaattaaggcatatgttattatccttagctttcaaaccaaaaattgagataaagaggttgagtggcttgtctgaggtagatcttgctgctttcctctgtttagtatggaactgatttattagctctttataaaagataaactttaagtacttttaaaatcattaagggattcaattatgaagatggttttatagttttgtagagaagcatgaatagaacttaattcagcaagttctttgccaacataaactaatgctatccaaatcaaggtattagtgccaacgttttctatgatgtggattattaaagtcaattttaggtccacattttaagaaaggttgacaaaattagagtacctttgtgggaggagaggactatcttttgatagacaaaagagaagactttgctatggtcaaatatttgaagggctgttatgctaaaaaggaagcaacttattttttattcattcatgtaacaaatatctaagtgcctcctatgtgtttgatgctaggggtatatagatgaaagctacctcctcttctgggaaggcaatccagtaaacagaaaataacagtaccatgggatttgtattataatggtgtagagcaggagtctgcaaactatagcttacaggccaagtcttgattttgtactgtccatctgctgagaatgatttttgcatttttaaaggattgtaaaaacaaacaacagtagtatgtatgaccaacaaaaccgaaaatatttaccatctgaccctttattaaaaaaggtttgctaactcctagagtttaaaatggatacataacttaaagggaagctatttttcaaattgatgtaaaaaagagctttctaatagagccatttaatactaaaaaaggctatataacaacaacaaaaagtgagcttcatgtcactgcaaaatgtctagcaaaattctgtattaggattttagcactggggaggagattgtacttcgtgacctaaaatgttttctaatgtaaagttttataactctggcttctcatttactatagttagggcagaacattttaaattattggagtagattcctggatctcataggcagaggaaattttatcattgctgttttgttacgcataagtactaacattgtgataaataggctctcgccaatggcaaatgtaattgatgatggctggtgccagaatggttagatacctcaaaaaagagggatgcaagccagtaacagatatccacacttgaggaaacaagaagcagttgaaagctgtaagaaaaaggcatggcaagtaaaatacaccaccttcccatcacacacacacagataatgttgtacacttgagaatcattaactcttcagctttggcctgctcctttagcaaatacatttgcctatttatgtgatttaactgtccaactccaccctgagatattcttaaatatttaaggagatgctgaaatgctgcttgtaacttacttgttacttactattccttacttatacttaaatagctattttaggtactgaggatttctgtaggttctgaaacctagttagggttaactctaattttttttgagaaaagactgtcagattctttaaatactcagcctgcagttaactggaaatagtaaatttgctttacttgcccaaaggtcccccttgtagcatcagctctagtgtatttggggactttgttaaaaactcttcatgagttttagaggggtgaaggactgttacggaggtttaagactgcttctctgtgccaagaccaaagaggaactgtaccaattgttccttggtggcaaaggttattgttacatatcagtttttcatttttaattaattcattcagtaaataaataactctcttttgagtaccttttaagtgtagcaggtactttgctaggcactgaggatatagctatggataaagtagacacagttgacaatttagatgggaagtggatagactgaagattaaaacaagagggtgatgaattatatggaaggtatgcagggtatataagcagtgacagtatgtgttttgactaagtaaaggacagatgacaacatggtgagctctgtgaactctagatttgcagtaatttcagggggaacagtaatgtgagagaggagtcctcactttgtacacagtgttgcaggcattctgaaatcaagaaagtatactgattaaaataagaatgatttaagtcaacattcaggaaatcacaagaatttttttttcattaaagcagcagttcaaactttttgggtaacaggattcttttacacttttaaaaattactgacaactccaaagagcttttgtttatttgggttattgatatttcccacattagaaattaaaactgaaacatttaaaatgtatttattaattcattgaaaaagtacatattttataaaaaataacttttccaatacaaaaatgtttagaagtatggccatgttttacttaaacaattctataatgtctgacataatagaaaacatcaagactctcatgtctgtttctgtattcaatctgttgctgtatcatatattctgtagcttctggaaaatgtcactgtatctttttgagagaatgggagtgaataaggcatatgatatcttaatattattttgaataaaattttgacattaggtaccccctaagagtgtcttagggcccaggagctcctgtgctatgctttgacataacgggtgtagtctatattatgtaagatgggaagaaacactcataatagttaagagcgtttgctaaaagttgaaatgcttgggttcaaatctcactctggcatttcagctacttgaatgtggacaagttacttaatgctttgtaagttttgtaatcatgtttcctcacctataagatatatataataatacctatctttcaaggttgtcatgaggattaaatgaaatattgtgtgttaagcacctggaccttgtcagactctcaataaatggtagctgctactgctgttgcttttatattattattctaatgcttgtagaattctccatatggcagcatatgtaggtgctttaaagtattatatacgtaagtaaataatcaattttcagcattacccagcatttaatatttgaaactttacagtcatacaagatagctttgcttctcctgatttgcctttgctgacctgtttgacacaagaccaagaatttgcgcctgattctttatttcatcaaagtgaactaagttttgcacctctgaggtaggatgatttatttgcatgtaacctttctcacttcttgttctatgtttttactaatattagtgacttcaggctaaggtgggactttgaggtggggcttagatactcttaggacacgccctgaaggtaacaaagtcctgtactaagacttgatgcatattaatttggcagagtaacgtatgtgtagagatgaaccagatgacagtagtttatttaaaaataattcctaatatattcaggcatttcaggcatattttaatttagaagacacactggattttccttcttataaacctccccatctgctgcccatcatctctctcctccctctcacccttattctgcaaaaagcatttatttcttgttcactaacttgtttggcactctcattttgttcagctgttattgaatgagactagttagctctccttaccttataggaggtgtcaatatgaagtaagaagatttttatgaacagacatatccaaatgaacagatactccaaattcatgtcctttagaatagtcataatgggagaaaagtctggaattttcaaaacatatttggaattattcaagaatatgttttcttcttttagaattgaaaacccaccttaaattaaaaactatagatgtttagaaagtaaaaagtaacctctgtccttatactatatatttacctgatatcacttttatgaaaagtgatctatacacattagatcatcaactaatgattaagttactgttgtgttttgggaatagatcacttcttaagtggtttctttctctttctctttttgtttgtttgcttttttttttctttagatggagtcttgctctgttgcccaggctggagtgcagtggtgccatcatggctcactgtgacctctaacttctgggctcaagcaatcctcctgccttagcctccacagcagctagtactacaggcatgtgccaccatgcccagccaatttttaatttttctgtagagatagggtcttgccgtattgccaaggcttgtcttgaactcctggctttaggtgatcctcctgcctcagcctcccagagtgctgggatcataggtgtgagccactgtgcctggccttgcatggtttcaagagcacaataaatgatgactagtaaaaaagttaacgcaggttaaatgaaaaatcatggtaggcgtcattatgaaaatcacattctgtgtctattgttttaggagaagtctgagataatatcagggttatagggtggtagttcaccttttagttaaaaagaatttgctttactcagtcattagccatcttagaagagtctgaaattaggagagctgtgtttttcaatacacttttcaaataaacttgatttcagtgacatatgtatttttgtgttagtacttaaaaattaaatgtttttaatgttatttatttatttatttttaactatatattttcaggggaattcctgataagtctgaagatactgaatggtcttctcgaccatcggaagttagtgaagctttattccaggctactgcagaagtagcttcagacttagcaagcagtcgctttagtgtatctcagcacccgcttataggcagcacagctgttgggtctcagtgcccttttttaccttctgaacaagggaataatgaagagactatttcgtctgttgatgaactgaaaattcccaaagactgtgatcgttatgatgatctttgttcatatatgtcatggaagacacgaaaagatacacagtggcctgaaaacaatttagctgataaagatcaagtttcagttgcaacttcatttgacataactgatgaaaacatagctactaaaagaagtgaccattttgatgctgctcgttcatatgggcagtattggacacaggaagattcatctaagcaggcagaaacatatttaaccagtaagtaccctgattctttttcagattcatctgacacaattgataaaaataaaattcccaagggaagtaacaattttagtgtccccttttcctatgtgccatggaacaaagagggaactttgcaccacccagaaacgtgtgtaatcagtaaggactgtttttcctttccatgtgaagttgctgctcatcatcatcatcataaaatgtcatattctttcttgtgttgtttgttggaaagaagaggaaaaggggttcccttgacttctgatagtcattattttgagaatgtttatttaagggctccagctgagattgaggtgaaacaaaagatggattctttggagggttatgaaagaaatgaagaaggtttacagaaagaatcatctcaatgttttgaactaacggagactagaaataatttagtatcttcagaagtgcaacccagattcttagaaatacagtatattaggaatgttatagctcctgataattctgtaaaagtttcagaagcacatgtactctccaggggacataatacctctaaaatggaggcttctggttggattgttatatccaacttggatgagatatcagaacaactgtattttcaagaggaaagaaaccaaaagggaacttctctttttggtgagaaaaatattgatgctgctgaaaatatagcctttgaggcagttattgcctctattgagccttctaagaaagagagtacagtaaatgaaatccaagctgacatcaataaatatttaacagatgacagccgagagagagccaaaaaatccagatctttctccattaaattataatgatgaaacctctttatttgataggcttaaacatccatgctatcagtctactcctggggtgtttgaaccagcagcttcaaaaccattgttgcataaaaagggtaataatgatagcttcctatactggcatccaaatttaaaattaccccctaatgctcttcagaaaatgttcattaagccactttgtgttattccagagcttaagtcatctgcttctttgagtgaaaaatcttacaaccaggctgttggtcttggcaaaacacagtcagctttatttcagtgtgatataggcaatgaaccatttcttccctttgggaagataaagtctgttccttctcttgaccttgcagagaaggtaggatcttcagatgttatttatcatgtgaaagtattaacttcagaatctttggttttacaagatttaaagcagccaacctttgaagaagttgcagattactcaaactttaatttgggtaaggatgttaactgtagaaatgccattaaaggtccacctgcagctgtaggaagcagcttttcagcaaacttggtggcatgtgatgcaaagtcacaaggtgcttgtaccattgaccagtgatgcatcattaattgcagtcgcccaagagacattgctgaaagctgatataggccaaggtgaaagtccttcgtgtgtgggagctgagcctagttttactatacatacaattatgcaaaatgactcctattttgtagagggcctgcaggggaaggttgagtctgacgtcattactctggatggcctaaatgaaaatgctgttgtatgcagtgaaagagttgctgaactacaaagaaaggtgagacacaataaaatgatagttgtaagaaacgtggcctttttcagtattgtttcaggaaatggtattgtttgtttttattttactttttactgtttcctgggtacatgaccaatgtcatttgactggtgagtacattgagctagcagctttagagaaatttcatggtgatctagagatgcatgacagctccctgcactggcagcctactttacaactaccatctgagaaggaaagcaattttgaaaagcctgtaagtttttctatttgacatttttttctgggcggggagggaatgttttatttgaaaaatttccaaattttatttgaaaatactttatttgaaaaattatatccttcagtaccagcttgtaacttgtacctgttcttcatctgatatccaggaatatgttccattagaggttggtataaaagaaatgtttatgtttcatagactgtaagacttagttctgtggtgtcttggaaaaggcatcccatggggctccaggcctttcatattgaggcagcctaactcttcctagttgtgccagcaagagtctttagacagtgttaagattaggcttattcatgggaacatccaaaagcttgtactttgaatgtttccctctggtcttcaatgcacaagccttgaaacagtccctggaatatttaagtatttctgtttagaagctcttgaaagacttgacttcagaggtgtcttgaaaattgtaagacccatgtttgaccaggaaggataatgaagtgcttaacgtccaaactggccagtgtctcttcaaagggaagtatctccttcccaaatgagcaacatactaatgtatatacatgtctcttccctctctctatttatatcattattaccattgtgtgtgtgtgtactagaaactggcaactctaggatgtcataaaaattttatatcagccttttttttttaaattaagttctggggcacatgtgcagaacgtgcaggtttgttacataggtatacaggtgtcatggtggtttgctgcacctatcaacctgtcatctacattaggtatttctcctaatgctatccctcccctaaccccctaccccgcgacgggccctggtgtgtgatgttcccctccctgtgtcattcttttatagaaggcttttatttgcttggatccttggatcccttggcattaatgttcctctggtcccgttgaatgaatatcctcttttgttgtctggtactgtaagtcccaaggtgggccctacttccttgtgatatagccttatatcctgttattcattaaatacagttagataaaatagtgatgtacttaatgttacttctcttttcctatgtataagattggtatgtttttgtaaagctctattagtttgggttacaaccacatttagacctttaggttcagattgttggtagaaaatgaacaaagatattatgatgaacttttatttataatttattacatttattaaagtttttttaacacagaatcactaggtttattttcttcagtattccaaactaggcattttttaattttaatctgataactggggatgaaaatgatagttaacactttgtgctcaagtactgtgctaagaggttacatgtatatatcaattcgtttttgtcctctgtcaatcctatcaaatagaattgttagagattattatcatccctattttgcaattgacacttagaggatttaggaacttgctcataattattcagcaattgggggaaccagtatttgaaactaaccatatctgtgtacttaaccaccataccataaactgttccccctccccccaccttttaactattcttggtgaaactaaggttaagattttgcattattttggcaagagtagaggttttgaaggagattgcctaaagttgagttcaataacaatgagaaacaaagtaacaacttttaaagtgttctaaatttttaatactacttttgtgacttacatcaacctttcatgtagataaaatgtattttaaaaataaagatatataggttcagttaatgtgcagagactagctttgaccactgctccaaattttaatctgtccttaaatatagcctgaagtattattctttcaatctaaggaagatgctattattccctgtattgtctttcagttgtagctctatctgtcttagtcttagatctaggagttatttaatgtcaatatcagaaaagcaaggagccagatgtggtagtactttttctgcatggcttagcttgctttcaggggcttgcttactccttggtttttccttatgctcctcatctgtgtcacagaagagtcattcatagttacttggttcacttttatatctactcctcataaatgattatttgggtatccacgtatgtatgatggagcttggaactgtgaaaagaacatagagattttgagtcaaaatccagttcagttactttgttagccttgtgtcttgagcaaatgacttcggaggatcactttcctgatctgcataaggaaataggaatagtaataaccttatacatcgtactattaaatggggtaagttatgtagcatttatatagtgcctggttgttagttctcttcactttgctgaaactagactactagagtctctggtggtttcctactttctatcattattttgtttttctttctcctttttcttggtagcagcacataattaatatgcttttttttttttttttttttgaggcagagttttgctcttgtcatccaggctggagtgcaatggcacaatctcagctcactgcaacctccatctcctgggtttaagcagttgtcctgctgcagcgtcccaagtagctgggattacaggtgcctgccactatgcctggctaattttttgtctttttagtagagacggggtttcaccatgttggacaggctagtctcgaattcctgacctcaggtgatccacctgcctcagcctcccaaagtgctggggttacaggcgtgagccaccgtgcctggcctaatatgctattttagtacctcttgttttggccatttagctctaaaaattatttaaggtttaagccttagatttcttttctctccccttcatatccccccacctttggagacctagttgcctgtctctgtgtggatgattatcacctctgtttttataccctaatatttgtctaatttattttgcatgttttcttgtgaacattgtcatcaccccaaattcaacttgctctatagcaaaataactcatgtttttcctaaaattagactgacttgtagagttcctgagtttttatgaaaggtattagtatttttctggtttcttggtcatctttcattctttcttcattattgattaaacattgggaagataaacgaatcttgatttctgttttctcctggttctttccattttcaattagcaatgaagatctgctggtttcttaaataaatacttctcatatttttcattttacattttcttttgtattacctactttttttttttttttttgagacagagtctcccactgttgcccaggctggaatgcagtggtgcggtctcagctcactgcaagctccgcctcccgggttcacaccattctcctgcctcagcctcctgagtagcttggactacaggtgcccgcccccatgcccggctaattttttgtatttttagtagagacggggtttcaccgtgttagccaggatggtctcgatctcctgacttcgtgatccgcccgcctcggcctcccagagtgctgggattacaggcgtgaaccaacgtgcctggccggtattacctactttaatagttctatattttgaaactttaacataatgaaatttttttgacgtcaagcttgtaataaaaatttttctttctgtgtttgaaagctgtagaagaaaaactgctgtaaactataaacacacactgtctctcacaagtgtctatatattatatatggtcatgccacataatgatatttcagttagtgacagactgcatatatgatggtagtcccataagattaaaatactgtatttttactgtacctttcctatgtttagatacacaaatgccactgtgttacagttgcctacaatatttagaacagtaacatgctgataggtttgtatcctggaagcagtaggctagaccatatagcctaggtgtgtagtaggctacacagttgtaggtttgtgtaagtcactctgtgatgtttgcacaacaacaaaattgcctgacagcacatttctcagaacatacccacatcattaagtgatctatgactgcatattatatatatgcatatatatttatatatatttatgtgtatgtatatatgtgtatatatatagcctgtacttcagagaagtttcctgtaggagtgaggtaattgaaaactatgaactttgtgctatgtggagagcagggcttcttagcagccttatcattttgagttggataattctttgttatggtgggaggcgtgtttcctgagtattgtaggatgttttgcatcatccatccctggcctctattcactagatactagtaacatcccctcccctcacaagttatgacagcaaaaaatgtttccaggcattgtcaaatgtcccttgatgggtacaaaatcttcacaagttgaaaagtagagaaacctgtatgggcagtagatgtagatatagaaatagatatggaatgttcatttccacacttcacatggcttcctaagaatgttcattactacacaatgaaaaattgatgaaagaggatagaaaaattcatgcttatgtacatttttagacttcatcctgagaagatgtgactgaaataatctttttagggaaataaagacaatctaaaaatgatatatatttttatgtgagggtgcactgtgaatttggtgaaaagttgtacatgtttaggtattaatgtcaccaatattggtttctaatttcttttctagcaagaacttgtgacttgatgaaacattaggaacttctttttgagcagttaccctttacttcactaccaatttatagtttccactacttttgttttaagaagtttgaggacttatgcttgttggtaaccgatcacagttatagttctttgatactctcatttttcttctaaacatttgtgagtttctagaaaatattcctgttttctttttccttcttactgtttcttgtccacagtgcacctctctaccttaaaaagaaatacctgagcttgtccagaataatcccatggttcaaattctgcgatgatcataaggagcaaacttctgagtaacagaacacttctaagcatactatctataatattttatctagacagatttctaagccctcaggatgatttagtcttctagagttgacccaataaaatatgacctatatatgaattcagaaggtttgagattatgatcttaagtaactagtataacttcttagtgttcctctttgtaaaaatgaaggattatttttagcagatgaggtttgaaattaatatcttattttcttcatttcttcgtaggtgggtcattctaatctgggcattaatgagtctttttcatttttattgccttcatttgttccacataagccaacaagagagtcggaatatcactcttcagatctcagaatgttgaggatgtctcctgacactgtgccaaaggctcctaaacatttaaaagcaggtacgtagaaaaaggagatagtaaatgtcctacttacggataccttgtgaaaaaatatcttgttaggtctatctaattttgtattttttaatatctataataattatacttcagattagatgtgtttataaatcctgtcttagtctctttagactgctattacaaaaatatcttagtactttaagtatggcttataaacaacagaaatttgtttcttgcagttctagaggctggttccaagatcaagtgctgggagattcagtgtcttgtgagggcctgtttgtcatagatgacatgttcctctgtgccctcccatggtggaaggggcagggggtctctctggagtcgttttttgtaatgggcactaatcccaaacatgtggcctgtgtcctcatgatctaattgctttccaaaagccttacctccttaatttcatcaccttggggattaggatttcaacctatacttttcaggggggacacaaacattcagaccatcacaagtcccctgatgttttattagaggaatcatttggtttttcatcgcattctacatggcagtgtttagaactgaattatatggaggagtctgatgttagaatctgtttattcctttgtgtgacttgaagggaaaaaggaattgattgagcccaagatgacaagagtggatgcagataagtttgtagagaggaaattgagagaaatcagttttgaggtaaaagcaagattatctgctgagggaggtgagtagtggtggaattaggttagaggcattgaggagagtggtttggtgaagacttacagtttatgttgaagtgaatttgagatggagctgaccaagggaaagtaaaaggattggagggcagttctgagaagtcagctcaggttagtgataatgattttataatattgtcagcctgtatagaatctctgatttgtttttcctgctaacactcaggaatggagatgatgaattacagtattgatccttggttcaggaatacattgaatgggtatggtagaaggatagcagtcaaagagagaagagttgatcacctattggtttcagcctacatagggaaagagaagccagaagagttgttattctaggagaaaatagaaggaccagagatttctttatgaagttgattagatgaggtgggaataagttactgagagcactagaagaataggtaattttggtcagattgaataagtagatttaaatgaggatcagtttgaagggtgatgaaatccattaaatagccctgggattggatggtagaaattgaataaaaattactggagtaaataaaattttgggtcaggtacagtgtcttatgcttgtaatctcagtactttgggaggctgaggtgggaggatcacttggaagagtctggaaaggattggtattaattgtttttgaatagaatttactgtgaaactacctgggctgttctttgtaggaagtttttaaattgctaatttcatctctttccttgccataagtctatttagattttatatttttcatgaatcagcttttgcaatttgtgtcttaaatagaatttgtccatttagctaggctatctgtttacatatatctgttcataatattcccatgtaatctctttcatttcagtaaggttagttacaatgccctctctttcattcctgattttagtaacttaagttttcccttcttttgttctccctttggtgaatctagttaaattttgtcaattttgctgattttttcaaataaactggtttctttatttttttctaatctttatttttcttttctgatttttgtttcatttattttcattctagtcattatttttccttccttccatttgctttgggtttagtttgtgcttttattctgttttttaaggtggacagttaggttattgatttaagctctttcttcttcttattacaaatatgtatacaaattgataaattcctaagcactgtgttagctgcatgtcctaagttttggtttattatatttttgttttagttcatctaaacttattttctagtttacattgtgatttttctacatttaccctttggttatttaggaatgtgttgtttaatttccttctgttattatgtttcatctggttcaatttttgttggaaaacatactttgtatgatttctgtctttaagttttattgaggctcgttttatgttttagcatgtagtttattctgtagaatgtacagtttgtacttgagaagaatgtgtattctgctgttgttaggtggtgtgttctatagatgtctttcagctctagtgggtttagctgttcaaattttctgtttccttgttggtcttctgactagctgttttctccattttcaaaagaagggtactgaagtcttcaactctttttgtttaattgtctatttctccccttttttctctcagatttgcttcacatattttgacattatgatgttagatccatatatgtttataattgttatatattcctgatgaattgacctttttttggagacagagtcttgttctgttgcccaggctgaaatgcagtgcgtggctcactgcaacctctaccttctgggttccagcgactctcgtgcctcagcctcctgagtagctgggactataggcgtgtgcccaccatgcccaattaattagaattgaccttttaattgttataaattgttcctccatattttgagtaactttttgttttaaagtctgttttgtcttaggttagaatagacacttcagctcttatggttgctgtttgtatggtttatcttttaaaatccctctactttcaacctatttgtattatcaaaaatgatgtatctgctgtaaagagcatatagttaaatcttgagtttttaattttaaaattctatctgtttttgccttgtgatgtgattatttaatctgtttgcatttaatgtcattattgatgtgattggattcacattttctggcttacgtttggtttttcatgtgtcttgtgtcctttttctgttccccttttattattgcatgaagtgaatattttctagtatgacactttcagtttaatttttaaaatgattttctaggtagttttttttaaagttatattcttagtgattactctggggcttataatatttatcttatcagaatatacttctgattcaatttatactaacttaattctagtatggtacagaaactttactcccgtatagctctattccctctttttttttggtgctattattgttatacatactactcacacacgtatataaatgcctttaagtttttgtttttttttttagtcggggacttgctctgttgcccaggttggattgcagtggcatgatcatagctcactctaacctgagctcaagtgatcctcctgcctcagcctcctctgttgtgaggcccacaggtgtgtaccactacactgagctaatttttcaaaaaatttttttggtagagatgggatctcactatgttattagtcttgaactcctgggctcaagcagtcctcccaccttggcctcctaaagtgctgggattacacgtgtgagccaccatgccagcctgctttatatattttcaaattgctctttaaatcagttaacagaatacaggaaatgaagtatgtctttttgtagttaccgatgtagttatttttactagcactatttgttttgttatgaaaatgttactactatctggtgttacttgtttttcagcctttggaacttcctatgttatttcttgtaagatggatctgcaagttccctgttttatgtatctggcgtgtttttatttcatctttttgaaagatagttttgctgggcttggaattcttggttgacagtctttttcttttcagcactttgaatatgtctcttcactatcttctggccttcatagttttggatgagaagtcagctgttatcttattcatgttctcttgtatgtaatttgttttctcttgttgcttccaagatatgctctttgtctttcactttcagtatcttgattatgatatgtctctatgtgaatctctgtgtttattctactttgagctcactgaacttgtcagatgtatagttaatatcttttgtctaatttgggaagttttcagccatcatttctttgaggattttttctgcttctttctcttttttgcccagttatcatctcttgcatcacagtccacatatatcagcacccctaatcttgtctcactggtctctaagcctttgtttgtttttgtttcttctttctttgcttcagtttgcatagtctctattcatttatcttgatgtttgctgatttttttcttctgtcttgtccatatctactgttgagcctctagtgaatttttcacttcagttactgtacttttcagccttagaatttccattttgttattttctaagttctgtctttcattctgtatttgatgtgactttgccatcatacctatctttaattctttaagcatccttttcttccctttgaatatgtttataatagctgcttttaattgttactggctaagcccaatgtgtgtgccctctgaaaggtaaatttgcctattttgttccccctgtgaaggggtcacactttcttttgcacatcttacttttctgttattgttctaaactggacgttttggataatatcttgttgcagctctggatattgatcccttctctgggctggtaacgtttgatatttttgttgtttcgttatttagtgtctggtttggattagttcttaattgctcaccaccaagatctccattgtttttgtcagtgtccttaggcttgagctttttatgctctgtttcaaacaaagtcattctccttatggagatgtgtggagctctctgttcttatggcttgcctctgctgggcagaagctttgtggcactgctctgtatctgggggcaggttcattggtctacttctcttggaataacacaccccttctctatgattgtgatgctgaaaggggtagtagttagtccctgttcttggattgcctctcctggagtggaccctcactccacctacaagtgagctgggggtagggatgattagagcttggtattctcaggttgggataaatcttccactctataagtggggactgggtgaaggtaggttgcttcttgtgttgtttctttttcacttattagctaacatcaacagccagtgttctttctttacagcctttctcccatgacatcccttccattctgccttgttactttatggatcgtatctactgatgtacccagttgtaccattttaattatttggttaaaatggtacatctcatttctaccagtaagatcctgagctcctcacttatctctcagagtagtattgtgctgtatgggctgacattaaagatttaatattatgcacagtcttgacatctgaaactgcgaggtcctcaaagggcctaaccacaagtttacctgccagatatgccctctactcagtggagaggcttcctacacagccagttcttctatcaacgagaccaactgtattctacctggtactcaacctcagatttccatcccatgctaacctgtgaaattattcaaacaagccaatcacaccctcctacagaaataaaaaaaatcacgctcttttcttgctactacaagcctgcctcctatggttcctgtttactcgttctgttcccatgtacaatccctgtgtggccctgcatgtcctgtgtcttccccagctgtgagtatatgtgactaataaactaccgtcagtctcctctgtgcagtgttgtatttagccaatccagatctatcaggcagcaatccctccctcatcaatagggtgaagaggaggcaaaagaaagaaatatttgtgtccatgtttgattcatattgtattgaaatggtcccttgatattagttgccctgatttcttatttttttattggcaaacctctgggtcctattttataacaagcagaaagtaactgccaagcttcatttcaaattggctccaaagtatgagtcaagataatttacatatcaggtaaagtagacacacttaacataatttgaatagtttagattacaagataaattggaaacaagtttggatctggattattgaaaaagaatcggtgacaccattagaaagtagggtagtgaatttagttttggacatactgagtttgatttgtcgggagatcatttggtttgaagtgtcttcaaagccagttgaacaaatgggaaataggaaatacaaatttggaagatcttaaataatttgtgattaaaattatgccgtaagagtagttaagctttctgaaggagcatgggtagagagaaaagcaaaatgtcaaggatggtacctcatggaatacaatagcagaaagaggccaatagaaaatatcaaaaaagacagatattttgtttaaattcttactctgatttagttcttacctcttcagtttccattccaatttcttagtgcctttactatatcacactggattttgttgcacgaacatttaggattgctttgtcttcttggtggattgatcctttacaattatgtaagaattctctttgtccctcgtaattttctttgttctgaagattatcttatatagccactcctatagaatagttcttttctttcttcttcttcctcttcttcctctcctcctcctcctttttttcttcttttttcttcttcttcttcctcctgctcctcctctttcttcctcttttttcttctttcttctttctttttctcctcctcctcctctttttttttttttttaaataaagataaagtctcactctgttgcccaggctggagtgcagtgatatgatcatagctccacagccttgaattcctgggctcaagcagtccttctgcctcactctctgagtagctaggactataaatgcacactaatatgcccagctaatctttttttttttgtagagacaaggtcttgctatgttgcctaggctggttttcagctcctggcctttcaaagcctgatcctcccatcttgacttcacaaagtgctgagattacaggcatgagccaccgtacccggcctttttatctgtcttttagtttcaaccttactatgtcattatgtttgaagtgaattttttgtagacatcatataattgggtcatttaaaaaaaatcctgtctgctaatatctgtattttagtttgtttgtttatttattttgagatggagtcttgctctgtcaccaggctggagtacggtggtgtgatctcggctcactgcaacctccacctcccaggttcaagcgattctcctgcctcagcctcctgagtagctgggactataggtgcacaccaccataccaggctaatttttgtatttttagtggagacagggtgtcaccatgttggccaggatggtctgcatctcttgaccttgtgatccacccgccttggcctcccaaagtgctgggattacaggtgtagtttgtttatttatacaatttatacctaaagttaatattggtctattatgccttaagtctgcaattttattatttattttcactttattcctgtttctcttgtcttgccttactatgggtttcatgaacatactttagaatttcactttaatttttaaaaattatttatttatttttgagacagagtctcgctctgtcgcccaggctggagtgcagtggtgctatctcggctcactgcaagctccgcctcccgggttcacgccattctcctgcctcagcctcctaagtagctgggattacaggtgtgtgccatcacgcctggctaattgttttatatttttagtaaagacggggtttcaccatgttggccaggctggcttcgaactcctgacctcaagtgatccacctgcctcagtctcccaaagtgctcgtattacaggcgtgagccaatgcacccggcccttggtgtggatttctttgggttttcctgtttggagctgacttactttctttaatctgtaagtttatgtctcatcaaatttgggatgttttcagactttgtttctttgaatttgactcatcttatttgcatcatttgatcgtcttctccctctctctcgcgttcgctgtctttctctctctctctctcttgctgtctctttctctctctcggctcactgaaacctctgcttccggggttcaggcaattctcgtgtctcagcctcctgagtagctggaatcatgggcgtgtgctaccacgcccagctaattttttgtatttttagtagagacggggtttcatcatgttggccaggctggtcttgaactcctgaccttaagtgatccacccacctcagcctcccaaagtgctgggattataggcgtgagcaaccacagccagccttgattgtcttttctcagtttgtgattttcctggttcttagtggtaaaaatgaattttgattatatcctggacattttggatattatgttaggagactttgattcctatttataatcttctgtttagcaggcagttgctctatttaggttttccatataggctctggcctatttatgtggctgtgatcccaaagataatttagttttcaggggagttgcattctggtctacttcagtggtaccattgaggctcccactcagtccctgttggtgatgcttatgtgggtggaatggtcttcctcaaggcctgctggtgctgtgtgggaagggggagatgctaacccacagagtagagaacatttccctgggcctgcccctggaccaactggtgtcagtgggtcacccacttgattcttgttgatggcattcatgggggtagaaaatgcttttctaggcctcctgatgcatctaggtaggatgctgggcccctgcgatggagaacatttccttgaacctactctctgggtttctcaatgctggtggacttcccattcaatttttgccagtgccactggggaaggaaagtgcctacctgagctgtcttttaccacaggagagttaggagatggtggacctaggtcttcttttgttactggctagaggacagggagatgccaaacctgttgggactctctgcctggtttttgctggcactacagttctggtggtgcaagcctacctgttgctgctgggttggagtagggtgtggggagatgggactgtctgctaagtctgctgggctgcccgtttcttggtccatggcccacagacagcaggcttttcttggacttctttctgccctcctttgttccctttctccttcccactattcctattcctattcctatttctggtcttagttccacactgcaggcctctccagggtccagcctcagatataagagagttaaaaagaaaactaaggaagtcattgccatgttgttctccagtctggaggtccttagctagtctgcgtttcactttctacctttcagttttttttttgtcttttggtttctagggaagtggagggaaaagcaaatttatatcattttgtcagggaccagaagacaactggagttttgaaaacttgacttagatgtgctattgtctttggttgttggtagaaataatcatatgggggaagtgaggtaaagatctttctcaggcaatattgaataaactcagggatctcaagccacagaagatggggacacctatgccatgaaactttcctaacttttctaaaatctgcagcccgtgtcctgaactattaataatagctgacatttattgcttgttcacattgctaagcattttatgtattattattataatgtatattatataatacaggttgagcatccctaatctgaaaaaccacaatccagaatggtccaaaatctggaactttttgactgctgacatgatattcaaaggaaatactcattagagcattttggatttcagatttttggattaggtatgcacaatatgttcttttatcatattattattatgccatattgttttatatttttaatctgacctatgatgatgggcactgctattattctatttgctatctgagcaaaatgaaacttagagtgacttgcccaaagtcccactttctctaagaggattttgatagcttcattccatattgttctctctgtttctcaagttttaaatgcctttataaccttaccataaacatatcacaccatcagtttttccaaattctttctcagagagggcaccaatttaatttctattatatacttaacactaatcttggcatatgataaaggcatgaaagagtctcgatgattgtttgctaactgaagagttcatcagttagtggtacagtactggctgcctcagcttgtttacagcatcaggtagtgacatagtaggaaacaatgctttgtgcaagaaaggattttaacttcttcctaggtaatatcaacacccagggcttcagttatttcctatgactgatgagttccaattgtaggactctatctagatatatctcatactccaaatgtttgtatctgactactatgaaatgtcttcatttggatgtcccataggtatcttaatgtcagaactttaaaaatggagccaatcatctctttaaacctactcttccttctctatttcttctcagtgaatgagtgtctacttattagccaagctagaaacttgtattcttctttcaccctcatccctgcccaatcacttaatctcgttgattctatcagactcccattctcttttctgaattactgcattagtaatggaaaactgatcatcttctctgccacttattttcagtattaagccaaaatagtctgtttacagggcagacttgataatgtcacttcctagtttggagttcttgctttccattgttaaccttggggtaaattctaaactccttagtacatagtagaaggtccctgcttaccttcttagccttatacttgcctcttttttcatagtgaactactctcatttcttcatgatgctagaccctcttgtctccttgatctcatagatgctattttctctacttggaatgcttccctattttttatatgcccatctttgcctaatttctggtttttatcctttcattctgagctttgctgtctttcaggaaaatgtttctgtttttcactgttcaccatgactctttaagaaagagccagatactccttacatatgttgctatagtatcctgtaaacagcctccctaatcactgcccttacactgaatttagtaattgcttttttttacttgtttctgtctcccacaggactttaagctctttgagggtagagctcgggctttgctaatatatatatttttgcacctggctgtgaaagctactcaataaatatctgttgaatgactttatgaattgatgaatattaaatgaattaacatgcctgggtagactgggggaggggtagaataagatggtaggctttgtattttggcagggatcagttctcaagaaagtggatcaaataaatgtaagtatcatatttcattgactatatgtcagttgtaatgaaggcctatggattagtgtcttatcactattttattgatctctggttaacatgaggctgtctctgtgacttgaatggcctatatgtctttctttatgttttttggtgggtattggggtttatgttccctgcctctaaacctaattgatttatcttttagttttcctgccataatttttattgacgcccagtcttttgtttaaccttactaccttaggttgttatttgttggatatcttctcccttgaagatgcaagttactcagggcagggagggaacctgcgtgtggctcagagtaagggaggactttagtttggggcaggactttgaagggttctcttaaagatgaggagcctggaagcaagatctgaaattacatcttctcctgtcccagagggaatttctttgagtaggggtgcacctatcagggtggtttgatgactgggtattactatgaccaggttataactttcccagggttattctgttaggaaatagtgtttgggatttgatgtttgagtacagagttcatgaacttttcacaacaccagcagttcattagctttttgaaacctctgcccacttaggaatgaggaagagtctataacgcactttacctaacaattcctgctttcgcattgaaaaagaacttagtgatagcaacaacaaaattaggaattaaattacaggctataatgttagctcgtgtatgtgatttttgtgttataaaacagtgccctattatcttttgaaactaagcaattttattacatttaacttgattttgtattgcaagttatttaaatggcatacatttatcaacaggacaacgttaatgaattatcagtatgcaggaactttggaagggttatatttacgtccactgtggtaataagggtatcaaaaatcctgtttgaactgtgatatattgtgttaagtttagttatgagagaatatagtttaagataatgaatgtagagataaacatctatgagttaactgagggattaacgaaatacaacacaattataattatttagttatcaataattaataattaggaacttataaaaatgatatagtaattaattagaaagcaagaataacaacaatgaagacaaagacccatttaaaatatcactggtgtctttttaaaaaaacatgaatttgagtttacttctctgtacacacaataggtgcttttgccagactttttaaaaaaaaatcttttttatttttaatatttatgagaacatagtaggtgtatatatttatggggtacatgagatattttgacacaaacatacgatgtgtaataatcacatcaaggtaaatggagtatctgtcacctcaagcattcattatttctttatgttgtgaaaattccagttatactcttttaatttaaaatgcgttataaataattgttgactgtagtcaccgtattgtactatatcatattctagagcttattcattctaactacatttttgtacccattaaccatcctcctcactaccttcccagccccgttacagacttattagtgactgaattagagggctcaaaatgagagctatagtaatttatttgtgtgtgctagtattaaaacccataataattgacttggaacgattactagtaagaggctcttagtcatatgtaaaatggaagtatggaattgtacctcattttgtctgtgagaaataaagggcttgcttattcttcttatgtcagaatatacttggggaaataaaaccgtgctgtcttcgtcacctaggaagccttctgtgttactaagcaccttagtatggagaagagttcttgagtaaattgtacccaggttgttagtcttaatgtcatctctaagaggcatacagttcccacccaatatacaactttggtgtagtatagtcttttctggcagccatcttcctcttcttggtcgaaacttacctcttcaactcacccctagttctgaaaatgaatctccaaagattatatcagtatgtagtaatattgttaacactcacacttagtgtattcttctttcccagggctgagagaataataaggactctaaaaacataccatttttcatcatgaactccttatgtacatagacacattttataaatcatagttaatggatttgtgatcaccattcttctgacagtttgacatatatgtccttaactttatatagaactgacaagaattcttctttaacatcgtgcatctcctccctcgcttttaacagcttaacctatagtcgttcttccagttttctttactaatcttttcctttacccctgaggttaaaaagaccttatcttatgtaacctggatctttcaaggacctgggctgtacatcttaactccttagctatttatctcatttgtccccattcatggtgtcactaataatttgtaatggccaaagggatataaaaactttgagaaatcaggaaatcatttagaaaaacaaggttttgttttgttaatctgttttggtttattagcttcttgagcctgttttgtttcctcacaaaaacttttcttattttctttagacccagcaggcaagaacctcacaaaattttgtaactcatattccttctcaggttacaatgcatgttacatttttaaattttttacttttcttgaagagttaatacttgatacttctagttctttactttgaaattcactcctcaacccatttcattctatattcagctcctttgaagctcgtcagtggccatagaaagccagtcagttgtagtggaaagagctcagaacttggatttagagtttaagggttaaagtaccagtttttgtcacttacccacctacaattctagaaaaactatgtatctttgcacttaaaatgtggtctgaggaccaataacattgggcatccctggggagcttgttagaaatgcagaatttcaggtcccacccagacctactgagtcagaatctgcaagttaacaagatcccaacattgagaagcactgttacatggtcatctttgcaactcggtttccctatacgtaaaaaatagataatacttgctctatatatcaatcatcttaacaccatctgacacatcatatgttttaccaatttattgtcagcctcctcctgctagaatacaaattagctctgtgaggtagggattattgtctgtttattgctgtatgtagaacagtgcctggaacatagtaagcattcatgtattttgaatgaacaatgaatgaagggacagcttgtcaaatcctgtcatttctgttgcagtcacttactttacactgctgtttaaaactgctttcatttagaattattgtattagtgcaaatatagaacatttctatagaaatgtttgttggattgtgcaacctagatttttacattcccttctcactcatcttgaaaactttaaaactgtaaacaacttgccatgctatacagaattaagttaaaactcttcagagtaacctttagagttttttcagttttatgtacttaaatatatatatattatacagggtgcatttgttgggagagtccttcaaactcactgcacgcatagcatccctctaggccatattgtactgcccctgtgggacttgggggcaggaggaacccgtgaaaataagctgatgagcctttgctgtaccagggatcatgaagtcttttgtctctgactgaagaccttatgtcttctgccagcctgcaagacacaataacagattgtaagtagggtgaaatcaaatcctagacccaatagcattatcccatctcattgaaaattctcttaaaaatccaagttgtaagatccatataatatttcatccactggatatactgttgtttataaccattactctgttgttgaagatttaaaatgtttttactttagttttctaattaatatctttgtgtgcaaatatttatctcctttgatggctgtttccttaggattcatttctagaagctttttaaaggctcaaagctggctttttttccagcacatagaattttaaaatagaaaattttatatactattaacaaatctctttttctttaggagacacttctaaaggaggcatagctaaagttactcaatccaacttgaagtcaggcatcactaccactcctgttgattcagacattggatctcatttatccttgtcccttgaggacctgtctcagttggctgtaagttctctagaaactactactggtcaacacactgatactctcaaccaaaagacattagcagatactcatctaactgaagagactctgaaagtcacagctattcctgaaccagctgaccagaagactgcaacaccaacagtactctctagttcccactcacatagggggaagcccagcattttctaccagcagggcttgccagacagtcatctaactgaagaggctttgaaagtttcagctgctcctggactagctgaccagacaactggcatgtcaactctaacctctacttcctactcacatagagagaagcctggtactttttaccaacaagagttaccagagagtaacttaaccgaagagcctttggaagtttcagctgctcctggcccagtggagcagaagacgggaatacctacagtatcctctacatcccactcacatgtagaggacctcctctttttctatcgacagaccttgccagatggtcatctaactgatcaggctctgaaagtctcagctgtgtctggaccagctgaccagaagactgggacagcaacagtactctctactccccactcacatagagagaagcctggtattttttaccaacaagagttcgcagacagtcatcaaactgaagagactcttactaaagtttcagccactcctggaccagctgaccagaagactgagataccagcagtacagtctagttcttactcacaaagagaaaagcctagtattttgtacccacaggacttagcagacagtcatctacctgaagagggtctgaaagtttcagctgttgctggaccagctgaccagaagactggcctaccaacagtaccctctagtgcatactcacacagagagaagctccttgttttctaccaacaggccttgctggacagccatctacccgaagaggctctgaaagtttcagctgtttctggaccagctgacggaaagactgggacaccagctgtaacctctacttcctctgcgtcctcttcacttggagaaaagcccagtgctttctatcagcagaccttacccaatagtcatctaactgaagaggctctgaaagtatcaattgttcctggaccaggtgatcagaagactgggataccctcagcaccatctagtttctactcacacagagagaagcccattattttttcccagcagaccctgccagactttcttttccctgaagaagctctgaaggtttcagctgtttctgtattggctgcccagaagactgggacaccaacagtgtcctctaattctcactcacatagcgagaaatctagtgttttctaccagcaagagttgccagacagtgatctacctagagaatctctgaaaatgtctgctattcctggactgactgaccagaagactgtcccaacaccaacagtaccttcaggttccttctcacatagagagaagcccagtattttctatcaacaggagtggccagatagttatgcaactgaaaaggctctgaaagtttcaactggccctggaccagctgaccagaagactgagataccagcagtacagtctagttcttacccacagagggagaagcctagtgttttgtacccacaggtgttatcagacagtcatctacctgaagagagtctgaaagtttcagccttccctggaccagctgaccagatgactgacacaccagcagtaccgtctactttctactcacaaagagagaagcctggtattttctaccaacagaccttgccagagagtcatctgcctaaagaggctctgaaaatttcagtagctcctggactagcagaccagaagactggcacaccaactgtaacctcaacttcctactcacaacatagagaaaagcccagcattttccaccagcaggccttgccaggtactcatatacctgaagaggctcagaaagtttcagctgttactggaccaggtaaccagaagacttggataccaagagtactttctaccttctactcacaaagagagaaacctggtattttctatcaacagaccttgccaggtagtcacatacctgaagaggcacagaaagtttcacctgttcttggaccagctgaccagaagactgggacaccaactccaacctctgcttcttactcacacacagagaagcctggtattttctaccaacaggtcttgccagataatcatccaactgaagaggctctgaaaatttcagttgcctctgaaccagttgaccagacaactggcacaccagctgtaacctctacttcctactcacaatatagagagaagcccagcattttctaccaacagtcgttgccaagtagtcatctaactgaagaggctaagaatgtttcagcggttcctggaccagctgaccagaagactgtgataccaattttaccctctactttctactcacacacagagaagcctggtgttttctaccaacaggtcttgccacatagtcatccaactgaagaggctctgaaaatttcagttgcctctgaaccagttgaccagacaactggcacaccaactgtaacctctacttcttactcacaacatacagagaagccgagtattttctaccaacagtcgttgccaggtagtcatctaactgaagaggctaagaacgtttcagcggttcctggaccaggtgaccggaagactgggataccaactttaccctctactttctactcacacacagagaagcctggtagtttctaccaacaggtcttgccacatagtcatctacctgaagaggctttggaagtttcagttgctcctggaccagttgaccagacgattggcacaccaactgtaacctccccttccagctcatttggagagaagcccattgttatctacaaacaggcctttccagagggtcatctacctgaagagtctctgaaagtttcagttgctcctggaccagttggccagacaactggcgcaccaactataacctctccttcctactcacaacatagagcaaagtctggcagtttctaccaactggcattgctaggtagtcaaatacctgaagaggctctcagagtttcttctgctcctggaccagctgaccagacaactggcataccaaccataacctctacttcctactcatttggagagaagccgattgttaactacaaacaggcctttccagatggtcatctacctgaagaggctctgaaagtttccattgtttctggacctactgaaaaaaagactgacataccagcaggacctttaggttccagtgcacttggagagaagcccattactttctaccggcaggctctgctagacagtcctctaaataaagaggttgtgaaagtttcagctgctcctggaccagctgaccagaagactgagacattaccagtacattctactagctactcaaatagggggaagcctgtcattttctaccagcagaccctatcagacagtcatttacctgaagaagctctgaaagttccacctgttcctggaccagatgcccagaagactgagacaccatcagtatcctctagtttatactcatatagagagaagcccattgtcttctaccaacaggccctgccagacagtgagctaactcaagaagctctgaaagtttcagctgttcctcaaccagctgaccagaagactgggttatctactgtaacttcctctttctattcacatacagagaagcctaatatttcttaccagcaagagttgccagatagtcatctaactgaagaggctctgaaagtttcaaatgttcctggaccagctgaccagaagactggggtatcaacagtaacctctacttcctactcacacagagagaagcccattgtttcctaccagcgagagttgccgcattttactgaagcaggtttgaaaattttaagagttcctggaccagctgaccagaagactggaataaacatcctgccctctaattcctacccacagagagagcactctgtcatttcttatgagcaggagttgccagatcttactgaagtaactttgaaagcaataggggttcctgggcctgctgaccagaagactgggatacaaatagcatcctctagttcctactcaaatagagagaaggccagtatttttcatcagcaggagttgccagatgttactgaagaagctttaaatgtttttgttgttcctggacaaggtgaccggaagactgagataccaacagtacctttaagttactactcacgtagagagaagcccagtgttatctctcaacaggagttgccagacagtcatctcacagaagaggctctgaaagtttcacctgtttctataccagcagagcagaagactgggataccaataggactgtctagttcctactcacattcacataaagagaaactcaagatttcaactgtgcatataccagatgaccagaaaactgagtttccagcagctacccttagttcctactcacaaatagagaagcccaagatttcaactgtgattggaccaaatgaccagaagactccatcccagacagcttttcatagttcctattctcaaacagtaaagcccaatattttatttcaacagcagttgccagatagagatcaaagtaaaggtattctaaagatttcagctgtccctgaactaactgatgtgaatactggaaaaccagtatctctctctagttcttattttcacagagagaaatcgaatattttcagtccacaggaattgccaggtagtcatgtaactgaagatgtgctgaaggtttcaacaattcctggaccagctggccagaaaacagtattaccaacagctcttcctagttccttttcacatcgagagaaaccagatattttctatcaaaaggatttgccagatagacatctaactgaagatgctctaaagatctcaagtgctcttgggcaagctgatcaaattaccggattacaaacagttccctctggtacttactcacatggtgagaatcacaagcttgtttcagaacatgtccaaaggctaatagataatttgaattcttctgactccagtgttagctcaaataatgtgcttttaaattctcaggctgatgacagagttgtaataaataaaccagaatctgcaggttttagagatgttggctctgaagaaatccaggatgcagaaaatagtgctaaaactcttaaggaaattcggacacttttgatggaggcagaaaatatggcactgaaacgatgcaattttcctgctccccttgcccgtttcagagatattagtgatatttcatttatacaatctaagaaggtggtttgcttcaaagaaccctcttccacgggtgtatctaatggtgatttgcttcacagacagccattcacagaggaaagcccaagcagcaggtgcatacagaaggatattggcacacagacgaatttgaaatgccggagaggcattgaaaattgggagtttattagttcaactacagttagaagtcctctacaggaagcagagagcaaagtcagtatggcattagaagaaactcttaggcaatatcaagcagccaaatctgtaatgaggtctgaacctgaagggtgtagtggaaccattgggaataaaattattatccctatgatgactgtcataaaaagtgattcaagtagtgatgccagtgatggaaatggttcctgctcgtgggacagtaatttaccagagtctttggaatcagtttctgatgttcttctaaacttctttccatatgtttcacccaagacaagtataacagatagcagggaggaagagggtgtgtcagagagtgaggatggtggtggtagcagtgtagattcactggctgcacatgtgaaaaaccttctgcaatgtgaatcctcactgaatcatgctaaagaaatactcagaaatgcagaggaagaggaaagccgggtacgagcacatggtaagaagaaagtttcaggcttataaacgttatagtttaataatgtgtttaaagttagatatttatgttttgaggaagcttagccaatgaaaaatacaagcaagatcaagaaaaaaaatgaagttagatatttggagttctaaatgtattttccaatagaattgttagaatgactatatttcttctaacaatccataaaaagcctatttaaccaataatgtagttaagttacagtattaatagtaccagcctgagtttacgttcttgttcttaggagaagtagattctgatgtatgacagagagtaaagaggtcgcttgggctcctgagctcctaaagctatcacgtttctctaatcctcccattatagtatccttattctgaaggcagaaagagcctccaatttgctgtgctctaaattgctttttgtcctaaaactacatatattttctctttcccttttctaatagaactcttggtgttgatctcagacatttgaaaaaaaaatcaatgtattttatactaaaacttgggttagaggtttaaaagagaatatttacaaatctcattaatttctaatagaattccattaaaaacaaaaatctgtaagacaatatggggaatatattttgggaggcaatttaaaagcaatttaggcctacctagaaatcgtcccttgaggttaacttcctacgctgttccctgcacatttttattctacaaaaaaacctccaagttgtatttattacttttaaatatcagctgtcatggaattatgagtaattgtagcaggaactaaagggagaggtaaagaaactcatgattaaaaggaggattaagataaatatgattgattacccgatttaaagtggcttttttgtagaattatctatagagcatgacttaagaaaatcagactttgcagtgaaaggatctctgtcaagaatttctaataggttgtcataattgagaagaagacatactaagcattgcagtgggtattaaattgcatatattgatgatcttctgtgttgcaattgttgacaaattatcacttttgcattgtattatctcaagtgtatgctttctctccagcctggaatatgaagttcaatttagcacatgattgtggatactccatttcagaattaaatgaagatgacaggaggaaagtagaagagatcaaggcagagttatttggtcatggaagaacaactgacttgtccaaggtataaaagaaatctggaaatgaagaaagtaaatatgaaagaatgggtgatggaattaggatctcttacttgggcatcagttgagttgctgataatatttggctaaagccttttttggttttgtttttgagacagtcttgctctgttgcccaggctgaagtgcagcagtgcaatcacaggtcactgcagccctgaactcttagactcaaggaatcctcccacctcagcctctcaggtagctgggaccacatgtgtgtaccgccctgctcggctaatttttaaatttttggcagagacagggtctctacgttgcccaggctggtcttgaacgcttggattcaagcgatcctcttgccttggcctcccaaaatgctgggattacaggcatgagccactgtgcctaggcaggcatcttaatttttcaaaagttaagatataagtattagattcttacagtctagatatgttgtttttattgattccaaattcatattttgaaaaatttatcttcttagaaaaataagaactgatttgcttgttttatcaattttttttttaagaacagagaagactaaatgtcacccttattttctcttgaaagtggtatcttgaaatccagctattttcaaatgtttattttaaatacttgagtacacagctataatagcttatgtataaatggctttcattttaatagtacatattttccttctattaactatttcatatgtagaaaagagcatatatgaatcatttatgtaaagaatagcagcaagtaaaaataattcccatatatttactatcgagcttgagaaatagaacattaccaaatactgttgaagttctttgtttctcctttcccagtcatatcttttttatttcttattttgatatataatgattttccttctcttcagtatggttttgaactctaagatggaatcaatatgatacatattcttttgagaactaggtgtttcacacagtgttagggttttgagctgcttctatgtagatgtgcagagttttcatttgttccttattactgtacagtattccagtgtgtgattacatgataatttattctttctcctgtggataaggcattttgtctgctattttgcatagtaatagggtataataatgcctatcgtagagggttttggtgtggattatgtgagataatgcctataaaatgcttagtagaacgtgtgatgtctagttagtactcagtaactggcagctattattactattattaatcctgaaagaattgatatagctatcatgcaaggatttcatggcctgattcatacaaagtttcttagaaaactattctccagtgatatagtttatttggattatctcaattttggaaaagtttgaaaagtacaaattagattggattttaggttttacgtatatgcttgtgaaaaacaaaattgacaaaataataagaataataaaatgggcaattggttgtcttagcaccatttgtatatatgctgccaaagtgagcactatcctagcatcatttgtttaaaattacatagttttctcagtgatctgtgtttccctatttttaaaaattattttcttgcgttcatatttccagatccctcagcaagcaagttgccttcctttgggtagactctggtttgtctgtgtttttcttaaaataaggtgtcctgaattagatgaaacacaatttcacttctgatctgaccagcatgaagtatattcagtaattctctttctgtcaatgtactgtgagatctttaatggtcacagcataattgggcattaaaaaaaaccatgtactgctttcaaaacttaaagctgtaggaaaaaagcaaagcaaaacaaaccaaacaaaaaccatgaactgctgccaagattcctattctcatttcttgtatctatataatacttttttaaaaatcttcttatttttgagacagagtctcactttattgcccaggctggactgcagtggcatgatcttggctcactgcaacttctacatcccaggttcaagtgattctcctgcctcagcctcccgggtagctgggattacagctgtgtgagtggcgcatgacaccacatccagctgatctttgtatttttagtagagatggggtttcactatgttggccaggctggtcttgaactcctgacctcaagtgatccgcctgcctcggcctcccaaaatgctgggattacaggccattacgcctcgccctatgtaatactttttaactctcctgtatttctttttattgcttttatatcagtgttcaagcctattggaatgatttttaatcaaatttgatcatttattttattaaaatatccctttcagctttgtgccatttatatatttgataagtatttttatccaaattagcaataaaaatgtttactaagggctatggataaaaagtctaaaacttttggctgggcgcggtggctcatgcctgtaatcctagcactttgggaagccaaggtgggtggatcacttgaggtcaggagttcgagaccagcctggccgacgtggtgaaaccccgtctctactaaaaatacaaaaattacctgggtgtcatggtgcatgcctgtaatcctagctactcgggaagctgaggcaggagaattgcttgaacctgggaggtggagattgtagtgagccgaagattgtgccagtgcactccagcctaggcgacagagggagactttgtctcaaaaaaaaaaaaaaaaaaaaggtctaaaacttttgaataagattactctcacacctattttgagtacttcactatactgtcattcaaccatcttgtgtttcaccatcttatgattgacttttttcagtctattaactgagtagttgcgaaatagtgtttgtctagatatgccttgtccttagggaacccatattggccacactgtgtacggttttggaccttttatttttttaacttaaatatattaggatttccctttaaaacttctctaaaagcataatttcacaggtatgtaatatcccattgtatgaacataacaatttattcactaccctactgttggacatttattcattcattccaaaaatacatattgaatgcctgatatgcgccatcctcatctatagcctgaggacacaccactgaacaaaatagacaaacatttcttcatcatgagcttacattaaggaagagtatatgaataacaaccaaacaagtaaaataaatgtcagatggtgggataagagccagggagacaaagcagaatggtcaacacagaacctgagaaagaggtattgaattaaatatctgagtaaggtgagaaagtaaggcagttaacagtgacaagagcattccagacagaaatagcatgcacacagttgctgaggtaaagtgtgtgaaaggaaagggagtgttaaaggaaagggaggaggccagtgtgtttggagagtgagagtgaaagggagaagactagattgtggaaggcagtggttcttgacgggtagtaaatgcctcccaaagagatattttggaaaattatgtgatttttcttgggtcatcactgtaattggggattgtttctggaatgcagtagataggtgtcaggtatgttactatttctataatgtacaagacagttccacacaatgaagagttgtcccttgtcacatacagcttttgaatgtccctgtagatattaatgttgatgaaaaacctgttataaattacttcagtgtttgtcatgttttaaatgattttttttttagtatagggtgtttcttctgttttttctttctttcatgactgacatttttgaagagtacaacctagttactttgcagatttccctcaaattgaatttatctggtgttttatcatgatttgattcagatttgatttttatgtaggaaagccacagaagtgatgtgcttttcatgcttgtcaggaggcacattatgttgatttgtctcaatacttgtaatgttaactcataccacttagttaaggtggtatctgctggtttgtgtgttttacatattaattaataagttaataaatattagggagagattctttgagactgtaaatatctctagtttaggatctagtgtttatacttgcctgaataatttattaataaaatagctttaatttggttattgaaaaaaattccatcattgcttcaacatttcattggagttctgtggtaagttaaaagtgtcccttctccatttatttatttatatcattatggactcttggatttttcgtttactcaaagggtcacaacctatcactatcattgtttattttgatactcaaattatcccacacttgcccactggaaccattttaagctggttcctgtgtgttcttatgatcccatcatttcatgagcattcacccactttctgagctaggaagacactctaggaatatcttatattttcttttcaccaaccctagagctgtccatttctccaaggggcgctggttctttttagcagagagtggcatgtagaaactaagatctgtatgcttggtgtgcttattgatactatgatgctgttgcctttatgccctttcagcagacaatgtgtgtgtacataattacacgcacccccctctcccctgtaggcctgtttccttatctattacttttattataaaatgagagctgagtttatactagtacatccaattccattccagtactacagggttattctagcctttttttctttctttatttgtaactcacttccctgatagtaagaaacttggctcccattatcctccatatatttacttgtttaaactcccttgtatgtttctccctcagccccactgctcttcatgcctgcaacagattgaatttctttcccagccccaactctttcaacccacaagttttatatgtaatataattattattataccattccagatatttttaaatttccattatgatttctttcaacaatgaatggtgatatattaggatgataccttcagtgtgctgaaagaaaataaattatatctatctagaattgcatgtcagtgaaaatctgttttaagaatgtggtaatataaaaatgattttagacaaaaacagttcattcagcactcattaaaaaattcgggtggagccaagatggccaaataggaacagctccggtctacagctcccagtgtgagcaacgcagaagactggtgatttctgcatttccatctgaggtaccgggttcatctcactagggagtgccagacagtgggtgtaggacagtgggtgcagcgcaccattcacaagccgaagcagggcgaggcattgcctcactcgggaagtgcaaggggtcagggagttccctttcctagtcaaagaaaggggtgacagacggcacctggaaaatcgcatcactcccacactaatactgcgcttttccgacgggcttaaaaaacggcacaccaggagattatatcccgcacatggctcggagggtcctacgcccacagagtctcgctgattgctagcacagcagtctgagatcaaactgcaaggcagcagcgaggctgggggaggggcgcctgccattgtgcaggcttgattaggtaaacaaagcagcctggaagctcgaactgggtggagcccactacagctcaaggaggcctgcctgcctctgtaggctccacctctgggggcagggcatagccaaacaaaaggcagcagtaacctctgtagacttaaatgtccctgtctgacagctttgaagagagtagtggttctcccagcacgcagctggagatgtgagaatgggcagactgcctcctcaagtgggtccctgacccccaagcagcctaactgggaggcaccccccaggaggggcagactgacacctcacacgaccgggtactcctctgagacaaaacttccagaggaatgatcaggcagcagcatttgcggttcaccaatatccgctgttctacagtcactgctgttgtgcagccaccgctgctgatacccaggcaaacagggtctggagtggacctctagcaaactcccaacagacctgcagctgagggtcctgtctgttagaaggaaaactaacaaacagaaaggacatccacaccaaaaacccatctgtacgtcaccatcatcaaagaccaaaggtagataaaaccacaaagatgcagaaaaaacagagcagaaaaactggaaactctaaaaagcagagcgcctctcctcctccaaaggaacgcagctcctcagcagcaacggaacaaagctggatggacaatgactttgaagagttgagagaagaaggcttcagatgatcaaactactccgagctacaggaggaaattcaaaccaatggcaaagaagttaaaaactttcaaaaaaattagacgaatggatacctagaataaccaatgcagagaagtccttaaaggagctgatggagctaaaagcaaaggcgcgagaaccatgtgaagaatgcagaagcctcaggagctgatgcgatcaactggaagaaagggtatcagtgatggaagatgaaatgaatgaaatgaagcgagaagggaagtttagagaaaaaagaataaaaagaaacgaacaaagcctccaagaaatatgggactatgtgaaaagaccaaatctacgtctgattggtgtacctgaaagtgacagggagaatggaaccaagttggaaaacactctgcaggatattatccgggagaacttccccaatctagcaaggcaggccaacattcagattcaggaaatacagagaacgccacaaagatactcctcgagaagagcaactccaagacacattaattgtcagattcaccaaagttgaaatgaaggaaaaaatgttaaggacagccagagagaaaggtcggattacccacaaagggaagcccatcagactaacagcggatctctcggcagaaaccctataagccagaagagagtgggggccaatattcaacattcttaaagaaaagaattttcaacccagaatttcatatccagcgaaactaagcttcataagtgaaggagaaataaaatcctttacagacaagcaaatgctgagagattttgtcaccaccaggcctgccctaaaagagctcctgaaggaagcgctaaacatggaaaggaacaactggtaccagccactgcaaaaacatgccaaaatgtaaagactgtcaaggccaggaagaaactgcatcaactaatgagcaaaataaccagctaacatcataatgacaggaccacattcacacataacaatattaactttaaatgtaaatggactaaatgctccaattaaaaggcaaagactggcaaattggataaagagtcaagacccatcagtgtgctgtattcaggaaacccatctcatgtgcagagacacacataggctcaaaataaagagatggaggaagatctaccaagcaaatggaaaacaaaaaaaggcaggggttgcaatcctagtctctgataaaacaaactttaaaccaacaaagatcaaaagagacaaagaaggccattacataatggtaaagggatcaattcaacaagaagaactaactatcttaaatatatatgcacccaatacaggagcacccagattcataaagcaagtccttagtgacctacaaagagacttagactcccacacaataacaatgggagactttaacaccccactgtcaacattagacagatcaacgagacagaaagttaacaaggatacccagcaattgaattcagctctgcaccaagcagacctaatagacatctacagaactctccaccccaaatcaacagaacatacatttttctcagcaccacaccgcacctattccaaaactgaccacgtagttggaagtaaagcactctcagcaaatagaaaagaaattataacaaactatctctcagaccacagtgcaatcaaattggaactcaggattcagaatctcactcaaaaccgctcaactacctggaaactgaataacctgctcctgaatgactactgggtacataacgaaatgaaggcacaaataaagatgttctttgaaaccaacgagaacaaagacacaacataccagaatctctgggacacattcaaagcagtgtgtagagggaaatttatagcactaaatgcccacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaattaatgaatccaggagctggttttttgaaaggatcaacaaaagaccgctagcaagactaataaagaaaaaaagagagaagaatcaaatagacacaattaaaaatgataaaggggatatcaccaccgatcccacagaaatacaaactaccatcagagaatactacaaacacctctatgcaaataaactagaaaatctagaagaaatggacaaattctttgacacatacaccctcccaagactaaaccaggaagaagttgactctctgaatagaccaataacaggatctgaaattgaggcaataatcaatagcttaccaaccaaaaaaagtccaggtccagatggaatcacactgaattctaccagaggtacaaagaggagctggtaccattccttctgaaactattccaatcaatagaaaaagagggaatcctccctaactcattttatgaggccagcatcattctgataccaaagccaggcagagacacaaccaaaaaagagaattttagaccaatatccctgatgaacattgatgcaaaaatcctcaataaaatactggcaaactgaatccagcagcacatcaaaaagcttatccaccatgatcaagtgggcttcatccctgggatgcaaggctggttcaacatatgcaaatcaataaacgtaatccagcatatacacagaaccaaagacaaaaaccacatgattatctcaatagatgcagaaaaggcctttgacaaaattcaacaacccttcatgctagaaactctcaataaatgaggtattgatgggatgtatctcaaaataataagagctatctatgacaaacccacagccaatatcataccgaatgggcaaaaactggaagcattccctttgaaaacaggcacaagacaggggtgccctctctcaccactcctattcaacatagtgttggaagttctggccagggcagttaggcaggagaaggaaataaagggtattcaattaggaaaagaggaactcaaattgtccctgtttgcagatgacatgattgtatatctagaaaaccccgttgtctcagcccaaaatctccttaagctgataagcaacttcagcaaagtctcaggatacaaaatcaatgtacaaaaatcacaagcattcttatacaccaacagcagacaaagagcgagccaaatcatgagtgaactcccattcacagttgcttcaaagagagtaaaatacctaggaatccaacttacaagggatggaaggacttcttcaaggagaactacaaaccactgctcaatgaaataaaagaggatacaaacaaatggaagaacattccatgctcatgggtaggaagaatcaatatcgtgaaaatggccatactgcccaaggtaatttatagattcaatgccatccccatcaagctaccagtggtaatttatagattcaatgccatccccatcaagctaccggtgactttcttcacagaattggaagaaactactttaaagttcatatggaaccaaaaaagagcctgcatcgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccacatatctacaaccatctgatctttgacaaacctgacaaaaacaagaaatggggaaaggattccctatttaataaatggtgctgggaaaactggctagccatatgtagaaagctgaaactggatcccttccttacaccgtatacaaaaattaattcaagatggattaaagacttacatgttagtccgaaaactgtgaaaaccctagaagaaaacctaggcaataccattcaggacataggcatgggcaaagacttcatgtctaaaacaccaaaagcaatggcaacaaaagccaaaattgacaaatgggatctaattaaactaaagagtttctgcacagcaaaagaaactaccatcagagtgaagaggaaacctacaaaatgggagaaaattttcgcaacctactcatctgacaaagggctaatatccagaatctacaatgaactcaaacaaatttacaagaaaaaaacaaccccatcaaaaagtgggtgaaggatatgaacagacacttctcaaaagaagacatttatgcaggcaaaaaacacatgaaaaaatgctcatcatcactggacatcagagaaatgcaaatcaaaaccacaatgagataccatctcacaccagttagaatggcgatcattaaaaagtcaggaaacaacaggtgctggagaggatgtggagaaataggaacactttacactgttggtgggactataaactagttcaaccactgtggaagtcagtgtgtcgattcctcagggatctagaactggaaataccatttgacccagccatcccatttctgggtatatacccaaaggattataaatcgtgctgctataaagacacatacacatgtacgtttattgcggcactattcacaatagcaaatacttggaaccaacccaaatgtccaagaatgatagactggattaagaaaatgtggcacatatacaccatggaatactatgcagccataaaaaatgatgagttcatgtcctttgtagggacatggatgaaactggaaaccatcattctcagcaagctatctcaaggacaaaaaaccaaacaccacatgttctcactcataggtgggaattgaacaatgagaacacatggacacaggaaggggaacatcacactctggggcctgttgtggggtggggggaggggggaggggggagggatagcatttggagatatacctaatgttaaatgacgagttaatgggtgcagcacaccaacatggcacttgtatacatatgtaacaaacctgcacattgtgcacatgtaccctaaaacttaaagtataataataataaaataaaaaaattctaagtggttacctcagactaaacggaaataatctaatatgggaaatttgtgatgtaggaaatattaaagagcaaaagatgtgttaagtatgtggataaataaagctaaatacttaagtatatgaaacaaaaatactaggaccttcatgactgagatgaaatgttataaacttaaaatatatgacaacaatatcttacaggttagaagctgcataaatggagttaattttttccaagtttcttaagtgtttgggaaaaagctgttatttactttagcttgataactatatgtgttataatttctataataaccctgtcccctacatcataccttactacaaaattaatttgagatggatcatagactttactttgaaaggtaaaataaagtttcttgaaaatctcttcatgatgttgggttaggcaaaggtttcttaaacaagaacaaaaatacacttcataaaagaaaggattgctaaattgctcttcatgaaagttaagaatggtaatcaaaaggcagcatgaagagtgaaaagtcaagctacacagtggaagagggtatttcggatacatatatttgataaaggacataaattcagaatatataaggaagtcatgcaaatcaattattcctacccaaagacttcactaggtgtctcataaatgaagatatccaagtggacagtaagcatttgaaaacattgtcaatatcactgcctgtttggtaatgcaagttaaccataatgagatactgcttcatatcagagtagctaaaattaaaaagaacaatgctaaatattggtaaagttatgaagcaactggaatagtcatttattgctgatgggaaggtaaactggtacacaactcttaaaataatttatagttatttcataatggtaaacatgccttacctgggacacttcattcctacttctttttaggtacgtactcaagaactatcagtacatatgttcagtagaagacatggtcaataataattatagccccaaactgaaaactacataaatgtatatcaagagtagaatagatgaatcaattatgctttattcatccagtgaaatcctgtgtagccataaaagatagcaaactactactgcctcaacaaccttgaaagctgtcacacacgttatggttagcaaaaaagccagacacaaagagtcatactgaataattccattcatatgaaattcaaaaactaatcaatggtgagaaaagtgataagaatggttacctctggtggagtcaggtgtgtggtattgactgggaagacgcacaagaggtcttctcaggtataggaagtttttgtatatctggatatgggcagcatttacattgctgtcatcacatataaaaaataaactacctgacttcagatttgtacaccttattaaagatttattatatattaattaaaaggaaacattgaacaagagatacaattgaaatcatagaaaaggaactttaagaaggcaaccatttctaatcatagcaaaaactttataaaaagcatcaaggcataaatctaacaagatttatgagatcttttattggagaatagtaaaatttattttgaaaggctagtagaagtcctaagtacatagagaaatacactgtatttattaataagactctatatccacctagcacaaactgcttcaatgaatcaaggcagatgtttctttaaattttttttatcatggtaaaatacacaacacaaaatttacctcaactgtttttaagtgtacagtttactggtgttaagtacattcatattgttgtgcaaccaccaccaccatccgtctccaagactcttttcatcttgtaaaactgaaacttatacccattaaataataacttctcattctgtcttcccatctgccccaggcagccaccattttcctttttgtctcatatgattttgattactctgagtacttcctataaatggaataatacaatatttgtctttttgtgactggcttatttcacttagcctcatgtggttcatccatgttgtagcatatgttataattttgtaaagtgagtctgttgtagacagtatatagttggatcattctgccaatctctattgattaaatttatttacttttaaagtaattaacaataaggaggaacctgcttctgtcctttttctatttgttttctacatcaaggtacaaattttatgttcagatacaattccaaaccacgatgaggatttatcttaactgtcctctaccccacagaaatgcagaggccaacaaatcacatttgtaggctcctaaaggaaactctcttagattgcttgtcaaaaactgttgagatcattttggccccttccatgctatgagaacgagatccacactgtacccaatgatttactgacttctctcggcagaatactgtctcttctccttctctcttctgtttggtaatgaagagaagaaaagtttgaagtcagtactttatatcatttagtatatggaccaataactcaacaagtttagaaaaaaaaaccatgaagtaaactttacttatactctaaaataaaccaaaccaaactacgaaagtagtaaggaggaaattataaaataaaatctatcagttaatgaactagaaaacagaaaaatgatagaattgagcattaaaatctagatttggaccttggaaaaattccaaatcagcagacaagttctgactagctagaagatgagaaggaaaaacatacaatgtggtaaatgaaaaggagcatgccagatataccacttagaaaatttgaaataataggagaatataatgtataacatataactctcgttaggtataaaattttcatgaaatgactgattttcttagataatataattaaacagaagcgtgtaacagattaaacattatgagagagaaaaactttattgatcaaacatttggcttccaagaaaagtacctgacctagctcaatattaggtaacattatgaagtatttcctgggcatgataatgacattgtggttatatgataggtgaattatttagggtcgaagtgtcaaatcagtgtctttgtggagggataaagctttgtggagggataggggaaaatgttcgacaattgttgaatctaaatagaaggaattcacatgtttattttaacattctatcaactttttggtatactcttaacaaaaagaaaaaaacaatgcctgacttagatgtttatatgggtgactcaagctatcaagtaatggataactcctgtgccatataaacagttccaatacactaacaaaaacttcctaatttacttgaggacattaacaaaaacctcagaagacatacctgaaattatgaatcttttgcatcagtgtgatgtctccacttttatttctgattttatttatttgagtcttctagttttttcttagtctagctaaatgtttgttaacattgtttttaccttttcaaaaactaacaatttagttgatttcttcctgtttttctattttttatttctgctctaatttttattctttccttttttctgctaactttgggcttagttttgttcttttttttctagttccttgagatgtaaagttgagttgcttatttgagatctttcttcctttaaaaataggcaattatcattagaaacttccctcttagtcctgattttgcttcatcctgtaagtgttcatttgttgtgtttttgtcttttttaaaattttccttgatttcatctttgatccactggttgttttaagagtgtgttgttaattcccacataattgtgattttcccagttttccttctgttactgatttctaattttagtctattgtgcttgggaaaaaaaggttggtatcatttcagtcttcttaaatttgttcagacttgttttatgacttaatatgtgatctatcctggagaatgttctatgtgcagttgagaagactgtgtattctgcaactgttaggtggaatattcagtatatgtgtgttctgttcattattgaaaatggggtactgacatcttctactgtattgctaattgctatttttcccttcagttctgtcagtgtttgctttatgtatttaggtactctaatgttgggtgtatatatgtttatacttgtgatatcttcctaatgaattgactcttttatcatatgaccttctttgtctcttgggatggttttttatttaaagtctatatcgtctgatagtgtggccacacctgtgctcttttggttgctatctgcatggaatatattttcctattccttcacttttagcctgtgtgtccttaaatctaaagtgagtctcttctagatagtatatagttagatcttatttttaaaattaattcagccactatatgttttttgattagggagtttactccatttacattaaagtaattattgatagggaaagacttactagttccattttgttaattggtttctttcttgtagttgttttgtcccttttgtcctctcttgctgtcttcctttgtgatttgttgattttgctttgatccttccttttttttttttctttcgtgtatcttctataggtattttctttgtggttaccaaagtcttaacagaaaacaacttatagctacagcgaaagcaatttaaaaaaatagacaaacaggactgcatctaattaaaaagcttttgcacagcaaagaaaacagttaagagactgaactggtaatctatggaataggagaaaatatttgcaaaccatgtatctaatgaggggttaatatccaaagtatataaggaacacctgcaactcaacagccaaaaaaaaaaaaaaaaatccccaaaacaaaaaacaaataacctggttaaaacatgggtaaaggacttgaatgaacatttcttaagacatacaaatgtgtaaggtgctgtatatgaaaagatattcaatatcgctaatcatcaggaaaatgcaaatcaaaaccacaatgactgaacacttcatatctgttaggatggctgatatcaaaacaaaacaaatggtaacaagtgttggagaggatgtggagaaattagaaccctgtacactgtagatggtcatgtaaaatggtgtagctgctgtggaaaatagtatggagattccttaaaaaataagaatagaattaccatatgatcccacttctgggtacatatccaaaggaaatgaaatcagaatcttgaggagatcccatgttcacaatagcattaatcacagtagccaagatatataaacaagatgtataaacatctatagatgaatggataaagaaaatgtggtatatacatacaatggaatattattccaccttaaggaagtcctgctgtatgtgacaacatggatgaccctgcaagacattacactatgtgaaagaatccagtcacagaaggacaaatactgaatgatttcacttatatgaaatatctaaaaatagtcaaattcagaaaacagagagtagaatggtgattgccaaggactgggaggaaggggaaattgggaattgatgttaggtaggtatcacaggcaaagcagatagttcaatattgggaaatttataaacaagtttttacattagtagatctaatggagaaatgattatttcattaagctccaaaaagtatttgggaataaaaataaaatctgtaattaaaaatatttctttagagtagaatatttctttatagtagaataatacctcaaatatgataaagaatatgtatttgaaatttgcaatcagcataatcaaagtcaaatacttttgttatgcatataaaacatttgtgagaaaagtcatataatatggtggagtagggaactctagggctcttgcaaaaacacctaataagcttccagaaactatcagaattagttttcgtagaactctggaatttagttaacattaaaaaccaggaaaagtagggagctgtagcgctgcagaaacagaacactatggcaccttaaattgctcacctaccattccttgtgcctagatgaacaatgactttgaagacagcagcttatactcatggtgcaggctgttagcacgagaaggattagtacagatcttgctctcaaaaaattgtgatcgttttaacctgtctgaagtatctgaaggaccagtatagagcttccttttgttttacacagctcagagctttcctgggactggggtggcttgtctgtcatcacgtgtcaaaagtatttaaaagccagagtatcagctgtagaagcctgagtctggggataaaaattaggacaatgaataggtagacaaaaagccaggaaggaacggttgaaaaggagatacggggtggtataaggacttttaaatgcttttttgtatgctgagaaattgagaatgctacgtacatgcctaggattggacaactcctcagagaaggcttgagaagaccctaagctttcaccactagctgaccttcaggctctgcctaactaagtaggatgtgaaggctaagatatagttgtaaacagcctggcttagtgttgatggaatgccccaacacagagcaaaaaagactgggagtgttttttttctttcctattaccttttttctttccttggctgcaggtgtttaaggaacctgtcaaaatagtagttgaccctgaagctaagtgagcacagacttcagttccatgcacaacaaagaatgtaagctttatgaaatagtcactaaacaacacttacaacaagcagtaacagcaaactccagggaagggggagaatccagtatccagagttgtcactttataatattcaatatgtccagttttcaacaaattataaggcatgcaaagaagtaaaaaagtgtgactcattcaaaggaaaaaagagaaattaatgaaactatccctgaggaagcccaccttaaatgtactcaatgagctgaacaataccatcagcaaagaactaagggaaaccaagaaaaggatttcccaccaaaaagaaatatcgacaaagagaaattataaaaaggaaacaaatagaaattctgcagctgaaaagtacagtaacaaaaattctttagaaggattcaatagctgagtagtctgaacaaagaatcagtgaacttgaagaaaggtcagatgatttatttattcgaggagaagaaagagaaaagaataaagaaaatgaacagggctatgggacatccccaagcataccaacatatgcattatgggattcttagagggaagagagagagagagagagagagagagaaaggggcggtaataatatttgaagaaataatggttgaaaacttccctaatttgttgaaaaatgaatttcaaatgggttaaactcaaagagacccacaccatgtcacattatagtcaaattgttaaatatttagcatagatgttggttctattagtagaaatatactttccttaatgaagtcagctttaacagattttttttttaaatctggttttgaagtcataaaatacaaaactatgagcttcttacaaagttattcctgccgtaaatactgtggtaatttatatatttaaaaaattgagaatttttttaaaacaagtgtttccaaaagcttattgatttttaagtaagttttgtatcacatggtaatacgtttcttttatttgtagaaagtagatttttactttttagtgcttgttgactctgtgtgtgtgtgtgtgtgtgtgtgtgtgtgtgtgtgtgtctattggtttacatgacggtgaagaactacttatttcttttttggtttgttacatgaagtctttgacattgtttgtttgtttgtgacagggtcttgctcagtcaccgaggctggagtgtagtggctggatcttagctcactgcaatgtccacctcccgagctcaagctatccttccacctcagtctctcaaagtgctggtattacaggcttaagccactgcacctggctgatgtgtcattaagctggatgatgagagaataatataacctgataaaactcatgtagatcttttgagaagagataattttaattgtgaaatagcatctggaaatcagtaaaagacatattttagctaaccagtaatttcataacatctttatctcagtataatttactataatacataaggtttgcccatttaaagtgtatagtttatttatagaattttacaatagcataattttagaactttgttgtcattagcccctctcccccaattccctagccctaggcaaacaccagtccactttctctcccaattcttttttgccaattctggacattttatataaacagaattgtaatgtatgatcttttgtgactgccttcttttacttagcatagtgttttcaagagtcattcatgttatgttgtaatgtgaatcagtacttcatccctttttattgccaaattacatttcattgtatagatactttgtttactcattagttggtagacatttggattttccactctggccattgtgaatagtgctgctatgaacatagtgtttatgtgttagggtaggtgtttcatttctcttggttatatacctaacagtagaattactgggacatacagtaactccatgtttaacatgttgaggaacttctaaacttttttctgaagtagctgcactatttacattcccatgagcagtatatgagggtttcagtttctccacgtcctttccaacatttgtcattttctgtctttttgattatagcctggttgggtgttaaggggtatttattgtgttttttacctatttctaacaactaatggtgttgaacatcttttcatgtgcttattggtcatttatcttttctgatgaaatacctattcaaattctttacccatttataaatttatcactgtacaaatttgttgagttgcaagcattctttttatattctggatattagacctttagcagatttatggacttaccaatattttctcccattctgtagacttttttactttcttgatggtgacctttgatacgcaaaagttttaaattctgatgaactctaacttatcagacttttattctttgcacttttggtgacatatctaagaaatcagtgcataacccagggtcacaaagatttagtgttttcttctgagagatttagttttagtttttatatttagatctgtggttctatttgaggcaattaaaatttttatttttttaattgtgataaaatatacataaaatttatcatcttaaccattgttaagtatatggttcagtggcgttaagtacattcatattgttgtttaaccaatctctatagctttttaaatcttgtaatactgaaactctatatccattaaacaacaactccccattttccccttccccttaggccctagcaaccaccattctactttctgtctctatgaatttggctactctaggtaccttatagtagcagaatcacacagtatatgtctttttgccactggtttatttcattcagtagtatattcttaaaattcacccatgttgtactatatgtcagaatttccttcttttttgagattatattattccattgtatgtgtatgccacaaacaaaagtttatccattcatctgttgatagacacttgagttgcttctgccatttggctattgtgaatagtgctgctgtgaacatgcttgttataattatctcttcaaggtcctgctttcacttcttttgggtacagacccaaaagtggaattcctggatcatacagtcattctacttttaagtttttgaggaagctccgtactgttttccataagggctacactgtttacattcccaccagcagtattcgagggtactgatttctccacatccttgccaatacttgttattttctgtttttttttgtttttttttttccttcttaataatggctattctaacaggtctgaattgatgtctcattgtggtggtgattttcatttcccttatgattagtaatattgagcttcttttcatttttggttggccagttgtataccttctctggagaaatgtctattatttttgcctatctttgaatcaggttttgttgttgttgttcctgagttgtaagtgtcctttatatgttctggatattaaccttgcatcagatacagtgagtctttgcctcacatctttgttaagttcttggaaactgacctgtaagtgaagtgacgtatagcatagcaaaacaaactgatataagttaattgataaaacaagagttaagttcctatggtattcagtacattgtttcacttaaaggtgcaatttctaagtacctattgatgatgttaagtgaagacttattgtacatgatttgcaaatattttctcccattttctaggttaccttttcactctgttgatggtgtcctttggtgaacagaagttttaaattttgatttagtccaatttatcaacttttacttttgttgcctttctcctttgagtcagtttttatgtatggcatgcaaattcgtctttgcatatagatattgagttgtttcagcactgttgaaaagactcttctttccctgttgaattttcatgacctctttgctgaaaatcaatttaccataaatgttagtgtttatttctggactgtcggttctatgccattgatctacatgactgtccttatgccagcactgtgctgtcttgattattgtagctttggagtaagttttgacatcggaaagagtgagtcctccaacgatgtttgtcattttcaagattattttggctgttctgggtttatgatctgcttgtcaattactgaaaaaaaaaaggctaggcttttaataagagttgcattgaatctctagattactatgggaaatattaccacttgaatctctagattagtttaggaagtattaatctttcagtccataaacatgagccagttttctagttattttgattttcttccatttcattctatgttttttagtatactagacatatatttattttgctaaatatattctcaagtatttaattctttttcttgctgttgtaaattgaattttttttaaaaatttatttttggattgtgaattgctagtgtattaaaatacagttgatttttgtatattgcactggcatcctgcagctttgctgaacttattagtcttaattggtttgtcctggtttccttgggatttcctgtatgaagttcatgtcatctgcaaatagatatagttgtacttctttctttccaatctagatgtctttaatttctaacctggttgccctggcaatatgtaacatccaatatgatactgaatagaagaggtaagaatggccatacttgtcttgttcttgattgtagggagaaagcatctagcttttcattattcggtattatgttagcgctgagtctttcataggtactcttcatctggttaaagaaatcccttccattcctagtttgctgagaaattttatcacaaaaacgtattgggttttgttgaactgaacagtagcttttaagagcctgttacatatctaatgacatgtggttggtttgttgtaaaattacttggaaatggttatgtgaaaatactgaaatttacttggcaaatactaaagctacaagaatttttaaaatggaaaggttattatgaaagcttgaaagatgcaatgggaggaggtagagcatgaagtgttaaaaaatttgtaagatataattgttggaaaggaagaagagaaatatattttgggatgagtataaatctactcatgcatacttttaagaggctggaataggtttatgtttctcaaattcggatatttgcttcccactgatatgtcaggaggtatcaaggcaataagaagccacagaataaaaacaacattttttttgcccaaataaaatttttacatctatttttatcagctaatgacgaatatttttatttatttagtaatttattcgtttatttatttatttatttatttatttattttactttaagttccaggatacatatgcagaacgtgcaggtttgttacacaggtatacatgtgccatggtggtttgctgcacctatcaacccatcatttaggttttaagccctgcatacgttaggtatttgtcctaatgctctccctccccttgcccctgaccccaccgacaggccccggtgtgtgatgttcccctccctgtgtccatgtgttctcattgttcaactcccatttatgagtgagaacacgtggtgtttggttttctgttcctgtgttagtttgctgagaatgatggcttccggcttcatccatgtcccagcaaaggacatggtcttattcttttttatggctgcatagtattccatggtatatatgtgccacattttctttatccagtctatcattgatgggcaaaaagtggcatttttaaagtgttagttttgtccagtggtatttaattttttttaagtcaacattttgttttgaaataattgtagtttcacatgtgataaataatatagagatatcacatatacccttcacccaatttccccaagtggtaacatcttgcataactgtggtgatatatcacaaccacaacgttgatattgatacagccaagatactgaacatttacatcactacaaaggtgcctcatgataccttataatagctacttccacttctctcctgccccaccttttactgagctcctggcaaccactaatttgttcctttgtttctataactttgtaatttcaagaatgtcatataaatgataacatatagtatgcaaccttattgtattggcctttttttcactcagcatacttatctggagattcatccagactgttttatgtatcagtagtttttcttttttattgatgtatagtattccaaagtatgaatgcaccacagtttaaatattcactattgaaggacatcttggttgtttcccagtttttggctttaccagtacagctgctgtaaacattcatatgcaagtttttgtgtgaatatgtctttgtttctgtggggtagatgcccaggattgcaattgctgggtcatgtggtagttgcatgtttagttttttgaaagacaggccacacctttttccagggtggttatatcatttacattcccaccagcagtgtgtgagtaatccgtctactccatatcctcaccagcatttgatactgtcactatttttattctagtcattctaataggtatgtagtggtatcttattaagtttttaatgtgtatttccctaatggtttttgatgttgcacattttttaatatttctgtttccagtctctttctcctgttaagtgaaatgtctgtatcttttgcccattttctttttttatttttatttttatttatttatttttattattatactttaagttttagggtacatgtgcacattgtgcaggttagttacatatgtatacatgtgccatgctggtgtgctgcacccactaacttgtcatctagcattaggtatatctcctaatgctatccgtccccactccccccaccccaccacagtccccagagtgtgatattctccttcctgtgtccatgtgatctcattgttcaattcccacctatgagtgagaatatgcggtgtttgctttttgttcttgcgatagtttactgagaatgatgatttccaatttcatccatgtccctacaaaggacatgaactcatcattttttatggctgcatagtattccatggtgtatatgtgccacattttcttaatccagtctataattcttggacatttggattggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaatcgccacattgacttccacaatggttgaactagtttacagtcccaccaacagtgtaaaagtgttcctatttctccacatcctctccagcacctgttgtttcctgactttttaatgattgccattctaactggtgtgagatggtatctcattgtggttttgatttgcatttctctgatgtccagtgatgatgagcattttttcatgtgttttttgtctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccattttgtaggtttcctgttcactctgatggtagtttcttttgctgtgcagaagctctttagtttaattagatcccatttgtcaattttgtcttttgttgccattgcttttggtgttttagacatgaagtccttgcccacacctatgtcctgaatggtaatgcctaggttttcttctagggtttttatggttttcagtctaacatgtaagtctttaatccatcttgaattaatttttgtatacggtgtaaggaagggatccagtttcagctttctacatatggctagccagttttcccagcaccatttattaaacagggaatcctttccccattgcttgtttttctcaggtttgtcaaagatcagatagttgtagatatgctgcattatttctgagggctctgttctgttccattgatctatatctctgttttggtaccagtaccatgctgttttggttactgtagccttgtagtatagtttgaagtcaggtagcgtgatgcctccaggtttgttcttttggcttaggattgacttggcgatgcaggctcttttttggttccatatgaactttaaagtagttttttccaattctgtgaagaaaggcattggtagcttgatggggatggcattgaatctgtaagttaccttgggccgtatggccattttcacgatattgattcttcctacccatgagcatggaatgttcttccatttgtttgtatcctcttttatttccttgagtagtggtttgtagttctccttgaagaggtccttcacatcccttgtaagttggattcctaggtattttattctctttgaagcaattgtgaatgggagttcactcatgatttggctctctgtttgtctgttgttggtgtataaggatgcttgtgatttttgtacattgattttgtatcctgagactttgctgaagttgcttatcagcttaaggagattttgggctgagacagtggggttttctagatatacaatcatgtcatctgcaaacagggacaatttgacttcctcttttcctaattgaatagcttttatttccttctcctgcctaattgccctggccagaacttccaacaccatgttgaataggagtggtgagagagggcatccctgtcttgtgccggttttcaaagggaatgcttccagtttttgggcattcagtatgatattggctgtgggtttgtcatagataactcttattattttgaaatacgtcccatcaatacctaatctattgagagtttttagcatgaagtgttgttgaattttgtcaaaggctttttctgcatctattgagaaaatcatgtggtttttgtctttggctctgtttatatgctggattacatttattgatttgcatatattgaaccagccttgcatcccagggatgaagcccacttgatcatggtggataagcttttcggtgtgctgctggattcagtttgccagtattttattgaggatttttgcatcaatgttcatcaaggatattggtctaaaattctctttttttgttgtgtctctgcctggctttggtatcagaatgatgctggcctcataaaaagagttagggaggattccctctttttccattgattggaatagtttcagaaggaatggtaccagttccttcttgtacctctggtagaattcggctgttaatccatctggtcctggactctttttggttggtaagctattgattattgccacaatttcagatcctgttattggtctattcagagattcaacttcttcctggtttagtcttgggagagtgtatgtgtcgaggaatttatccatttcttctagattttctagtttatttgcatagaggtgtttgtagtattctctgatggtagtttgtatttctgtgggatcggtggtgatatcccctttatcatttttaattgtgtctatttgattcttctctctttttttctttattagtcttgctagcagtcttttgttgatcctttcaaaaaaccagctcctggattcattaattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtgggcatttagtgctataaatttccctctacacactgctttgaatgtgtcccagagattctggtatgttgtgtctttgttctcgttggtttcaaagaacatctttatttgtgccttcatttcgttatgtacccagtagtcattcaggagcaggttgttcagtttccatgtagttgagcggttttgagtgagattcttaatcttgagttctagtttgattgcactgtggtctgagagatagtttgttataatttctgttcttttacatttgttgaggagagctttagttccaagtatgtggtcaattttggaataggtgtggtgtggtgctgaaaaaaatgtatattctgttgatttggggtggagagttctgtagatgtctattaggtccacttggtgcagagctgagttcagttcctgggtatccttgttgactttctgtctcgttgatctgtctaatgttgacagtggggtgttaaagtctcccatgattaatgtgtgggagtctaagtctctttgtaggtcactcaggacttgctttatgaatctgggtgctcctgtattgggtgcatatatatttaggatagttagctcttcttgttgaattgatccctttaccattatgtaatggccttctttgtctcttttgatctttgttggtttaaagtctgttttatcagagactaggattgcaacccctgcctttttttgttttccatttgcttggtagatcttcctccatccttttattttgagcctatgtgtgtctctgcatgtgagatgggtttcctgaatacagcacactgatgggtcttgactctttatccaatttgccagtctttgccttttaattggagcatttagtccatttatatttaaagttaatattgttatgtgtgaatttgatcctgtcattatgatgttagctggttattttgctcattagttgatgcagtttcttcctagtcttgatggtctttacattttggcatgattttgcagcggctggtactggttgttcctttccatgtttagtgcttccttcaggagctcttttagggcaggcctagtggtgacaaaatctctcagcatttgcttgtctgtaaaggattttatttctccttcacttatgaagcttagtttggctggatatgaaattctgggttgaaaattcttttctttaagaatgttgaatattggcccccactctcttctggcttatagggtttctgccgagagatccgctgttagtctgatgggcttccctttgtgggtaatccgacctttctctctggctgtccttaacattttttccttcatttcaactttggtgaatctgacaattaatgtgtcttggagttgctcttctcgaggagtatctttgtggcgttctctgtatttcctgaatctgaatgttggcctgccttgctagattggggaagttctcctggataatatcctgcagagtgttttccaacttggttccattctccccatcactttcaggtacaccaatcagacgtagatttgttcttttcacatagtcccatatttcttggaggctttgctcatttctttttattcttttttctctaaacttcccttctcgcttcatttcattcatttcttcttccatcgctgataccctttcttccagttgatggcatcggctcttgaggcttctgcattcttcacatagttctcgagccttgtttttcagctccatcagctcctttaagcacttctctgtattggttattctagttacacattcttctaaatttttttcaaagttttcaacttctttgcctttggtttgaatgtcctcccgtagctcagagtaatttgatcgtctgaagccttcttctctcagctcgtcaaagtcattctccatccagctttgttccattgctggtgaggaactgtgttcctttggaggaggagaggtgctctgctttttagagtttccagtttttctgctctgttttttccccatctttgtggttttatctacttttggtctttgatgatggtgatgtacagatgggtttttggtgtggatgtcctttctatttgttaattttccttctaacagacaggaccctccgctgcaggtctgttggaataccctgccgtgtgaggtgtcagtgtgcccctgctgggaggtgcctccctgttaggctgctcaggggtcaggggtcagggacccacttgaggaggcagtctgcccgttctcagatctccagctgtgtgctgggagaaccactgctctcttcaaagctgtcagacagggacatttaagtctgcagaggttactgctgctttttgtttgtctgtgcccttcctccagagatgtagcctacagaggcaggcaggcctccttgagctgtggtgggctccacccagttcgagcttccaggctgctttgtttacctaagcaagcctgggcaatggcgggcgcccctcccccagcctcgctgccaccttgcagtttgatctcagactgctgtgctagcaatcagcgagactccgtgggcctaggacgctcggagccaggtgcggggtataatctcgtggtgcgccgttttttaagccggttggaaaagcgcagtattcgggtgggagtgacccgattttccaggtgcgtccgtcacccctttctttgactaggaaagggaactccctgaccccttgcgcttcccaagtgaggcaatgccttgccctgcttcgtctcgcacacggtgcgcgcacccactgacctgcgcccactgtctggcactccctagttagatgaacccagtacctcagatggaagtgcagaaatcaccgtcttctgcgtggctcacgctgggagctgtagaccggagctgttcctattcggccatcttggctcctgtccctcttttgcccattttttaattggattgtcttttttttccctttttactgttgtgaattgtatatatatgttagtatatatgttagatgtaaggctagcagatacaagacctttgttggatatgaggtttgcaaatattttcttgtaacctgtagctgggagagagaggggcattttgttttgctggatagtctaggtaggcttcgctagactttcactgacgccgcaaggaagggatagcactttatcagccagcagagatgaaagtaagtcctgggttcctcacttggcctttgctggtgttggtgtgagtgggtctagttttttctgtgatggttggctggagtagaatggttattatttaaaagttctctcttttgctaggcttcacctctactgggtttttagctagtgagaaaagctcttgttagggttttttgtctgtgtccattgttcctgggtagctgacttcttcagctccaagtctgtatatgcaagagaaaaagaaaacccagtgattgcaccacagcataattcctcaggtccctagatctctagctaatctacgttcttctctacatctttcagattcttcttacatttgttttatatatattttccagtgggctgaattatacttagcaggagaaatagggaaaagtacatctggccaggcgcagtggctcatgcctgtaatcccagcactttggggaggctgaggcaggaggatcccttgaatccaggagttggagaccagcctgggcaacaaagtgagacccagtctctacaaaaataaaaataagaaaattagcccggtgtagtggcatgtgcctgttgtcccagctatccaggaggctgaggtggcaggatcgcctgagcctgggaggtcgtggcttcagtgatagtgccacagcattccagcctgggtgacacagcaagaccctgtcttaaacaaagaagaagagaaaagaaagaaaaatgtcacaggatcctcagggtgtcagtttgcaagctggaaaccactgtggccagcagcaccttctgcctgagtattgttcacactgctgggcacattctccgcacttggcctggcaggctgtgcttggctcgcactactatgctatgtcttacacctgccaagcatgagccaggtgtggagtggtgaggggtgtgtgggcaagcaagtgtggggtctagccactgtacacagccaggcatgctgacggctgtggtagggcaggcagctccagtgctggcactggtgctggcgccacgcaaggctgcagctggaccagatgtactgtgcgtggcttctgctgtgggcacccacatctgggtgagggaaatgtggtggtggcaactggaagcttggaagacaccaggaaccacaaaaccccaaagagggtgtcatagtcctggctcagggaccccctaggtctgggcttcccgaaaggccacagcttgtctcttcttcttgttccctgcaacatggcaggcggggagggggcatgtttcagccctgtttgtgttacagctcttttagtcccaccatttggtggatcctgagctcttgtcctgcatccatgaagaatgaggtatgtggacaactggagggtgagcaagatggagaggagtttcatcgagtggcagaacagttttcaggagacccaaagtgggtagctcctttctgcaggcaggtcatcctgatgactgtccatctgtcagtggagaggagaacctagagtgggtagctcctacctgcatgcaggtcatcccataatctgcatctttcagcagagaagagacccggagtggctagctcctatctgcaggcaggtcattccattgtctctccaagtctggctgagtccagggtttttatgggcttcagaggggaggagatgcatgccaattggtccatgggcaggcccggaaaaagcactataagttctcctttctgcgaaactggcagcccttcaacccatccctggcttaaaggtggagcttcactggtgacctgcccctttctgcccaggagcctgtctgccatctctgccatctacggtacccatgacacacccaggctgtttatgtcagggggcgcctgcaggcccactctgagctgccctcagccctccctgcccctgcaacctccctcctgtgcttgtgggtgcccagtgtctggaggggcggcagggggctggtgtgacagtgctgtcccaagtgtgcgcacacctggctgggtcgtaacagtgcctggacttggcctcaactttgctgtgtaattggagcgggaaccaggagtggggagaggcctggcagtgggagcaggcacttctgagcctgcaggggcagggggagttcccagatccccgaaagtgcagtggtgcctgggtctacagctgtgactgggcagctgcagctgtgcccaggagggtggggttcctgcccctccaactcagaagggagtggggcttctgcctgttcctggctcccaccacctcagtacagcgagtagccctagctgttccttccccactgcagctggcatcattgcagcagccactccagatgggatgccgctgccatcaaaaatatatctactccatcacctggggagatgcagcataatttaaaatatttttatctgaaatcagatgttgctatatcatcaaatagaatgccaagttataatgactgtataggtaaaatacagaaatctcaaagaaatgttatatttatggtagctttgagtactgatcctatgcgccaggaaacaaggtcttggttttttccccccattgttaggggtaaaatattaggatgtaggtcaagcttctgtaatagccaaacatagagtggctcaaataagatagaattttattttttcacgtcattgttcatgttaggaaagtgagctctgttccttgatgtcatccagggacccaagctggctggacaactaagctgtcttacacatgacttccaaggttgttccatgcattaccattttcaggaggcaagaaaaatagtgatgatagtggtggtgatattgggggaacatgcaggtcaagaacttgattttaaggacaggaagcagaagttgtacttgatgccttcattcatatcccattggtgtcagtctagtaatgtggttacaactagctgcagaagaaacaggaaatgtctcccacttggtggctatgtggccagcaaaaatttgagtaaggcatttctattattaaaagaaaggtgagaatacttactggtggacaatagtaatctttgctgtttactttagtggaaattttttagaagaactctctgagtggtcttaatcattgtgagccattttattctgtagttaactgtcttttttgcttgctgtgctgtttgtcctgttatattataaaagaatgacatgaccttcatggtctaatcttagcgtgggtataaaactgatttgtataaaactgatttgtttataactacttggactacttcaaataagaacctgtttgtttgtatcttctagggtttacagagtccacggggaatgggatgcaagccagaagctgtatgtaatcacattattattgagagccatgaaaagggatgtttccggactctaacttctgaacatccacaactagatagacacccttgtgctttcagatctgctggaccctcagaaatgaccagaggacggcagaacccatcatcatgcagagccaagcatgtcaacctttctgcatccttagaccagaacaactcccatttcaaagtttggaattccttgcagttaaaaagtcattccccatttcagaactttatacctgatgaattcaaaatcagcaaaggtcttcgaatgccattcgatgaaaagatggacccttggctgtcagaattagtagaacctgcttttgtgccacctaaagaagtggattttcattcttcatcacaaatgccgtccccagaacccatgaaaaagtttactacctccatcactttttcatctcaccgacattctaaatgcatttccaattcctctgttgttaaggttggtgttactgaaggtagccagtgtactggagcatctgtgggggtatttaattctcatttcactgaagaacaaaatcctcccagagatcttaaacagaaaacctcttccccttcatcatttaaaatgcatagtaattcacaagataaagaagtgactattttagcagaaggtagaaggcaaagccaaaaattacctgttgattttgagcgttcttttcaagaagaaaaacccttagaaagatcagattttacaggcagtcattctgagcccagtaccagggcaaattgtagcaatttcaaggaaattcagatttctgataaccatacccttattagcatgggcagaccaagttccaccctaggagtaaacagatcgagttccagactaggagtaaaagagaagaatgtaactataactccagatcttccttcttgcatttttcttgaacaacgagagctctttgaacaaagcaaagccccacgtgcagatgaccatgtgaggaaacaccattctccctctcctcaacatcaggattatgtagctccagaccttccttcttgcatttttcttgaacaacgagaactctttgaacagtgcaaagccccatatgtagatcatcaaatgagagaaaaccattctccccttcctcaaggtcaggattctatagcttcagaccttccgtctcccatttctcttgaacaatgccaaagcaaagcgccaggtgtagatgaccaaatgaataaacaccattttccccttcctcaaggtcaggattgtgtagtggaaaagaataatcaacataagcctaaatcacacatttctaatataaatgttgaagccaagttcaatactgtggtctcccagtcagccccaaatcactgtacattagcagcatctgcatctactcctccttcaaatagaaaagcactttcttgtgttcatataactctttgtcccaagacttcttccaagttggatagtggaactttagatgaaagattccattcattggatgctgcttctaaagcgaggatgaatagtgagtttaactttgacttacatactgtatcttcgagatcactggaaccaacctccaaattattgaccagtaaacctgtagcacaggatcaagaatctttaggttttctaggacctaaatcttcactggatttccaagtcgtacagccttctcttccagacagtaacactattactcaggacttgaaaaccataccttctcagaatagccagatagtaacctccaggcaaatacaagtgaacatttcagatttcgaaggacattccaatccagaggggaccccagtatttgcagatcggtgagtctcattgtgataacaagcaagctggatggactttgtaataaagggtttcaaatacttaagtagatatcggttcttgggggaaaggccgtgtgatttctctgagtggaggttgtgtctggctaattactagaagtctttcaaggaataaatgtgttaagtaagagagaacatgtgacagtaatgcatttatatttcagagggcttttggcaagacttgacatcaagtgtttttaaaaattgaatgaccttgaaaagaatctttgatcatccaatgatacaattacgaatttatttactgtgttggacatatgctgaagatctcctttggaggttgcacacacatacatgggtgcttggtcactctctctcccgctctccctctctctctctcttctccagctctctggttccatttcatgacttccctcaaatatttagtatttacaaagaaagtggctttacagtggaaaatcctggcagataccagtttagtcaaatgatgaagattagcatcgccagtaaaacatctgtcaacataatgtgctccctgatatgatgccccacctcatagagtattgtaggaaaggaaacccaattcagggataagttcgttagatttatattattcagcccaattttagcacattgccattaattcagttttacttaatgagcctcttatgatatggtgaagcagaactacctgtatgtttagtattttaccttatagttgaagaggcatcgtaccacagtggagagcgctaggcttggaaaggaggaagaaagaaatgtttattcataatctacatgtggtagtagtcatggcaataaggaacttagatatgctatcctacttagttctcatcatgatcctatgaagcaggtgatgatatctctattttcctactgggaaatcttttgtgccaaaagatgttgtttgtgggcaagagaaagagcattcaaatgcattctgtttaatcagagtaaggatttgttcctctacacagtactctgtcctagaatttgaaccctaattccaacacttccaaccatgatgctggacaactgatttaatatccttggcactcatatgtagaaggaaagggttggaatgctgaaaagtcccttatagctctaaaagttagggattgagataatagattggggcaaaaaaacaaggtcaatctctgatttcaatgttaaaaatagtagagtgccatagagatcagtaatgatacctttaaaatttttttctttgagacagagtctcgctctgtcacccaggccagagtacagtgatgtgatcttggttcactgaaacctccgccccctgggttcccgtcccgcctcagccacctgaatagctgggaccacaggcgtgcaccaccatgcctggctaatttttgtatttttagtagagacgaggttttgccatgttggcaaggctggtcagtattgatacttaaaaaaaaaaaaaaaagtttagaaattaaaacattgacaatttcatatacaatttttaaactcttcaaaggttaaatgctaaggtattgtactgaataccttaaaaatgagataaagctatgaatgagaagaaagtaatagatgattatatatatgaactaacaataatgccatttgttcagggtgtacgtttgtagtggatgcttatattatacttagtgtaataatcagaaatagatattattacctcagttgtggatttaaaaaacctgagacttagagaaaatattttgcccaagatcacatggccattgagcattggaggtggcatttgaacctaggtttatcaaactccacagtccataaataaggcaaactacacacacacacacacacacacacacacacagatgcataaaacatatatatataagtaaatatatatataataccttataaaatatttataaagggctgggtgcggtggctcacacctgtaatcccagcactttgggaggccaaggtgggcggatcacctgaggttgagaatttgagaccagcatgatcaacatggagaagccccatctctactaaaaatacaaaattagctgggcatggtggcacatgcttgtaatcccagctactcaggaggctaaggcaggagaatcgcttgaacctgggaggcggaggctgcagtgagccaagattgcaccactgcactccagcctggcaacaaagcgagactctgcctcaaaaaaaaaatttataaagtatgtatgtataaaatatttataaagtatatgtgtataaaatatattcaaactttttcattttgagataattttagacttaggagttgcagaaatagcacagagagtttctgtattatctattgctgtgtatcatgttaccctgaaatatagcaacttaataaacgtgaaaatttatgtaggtcagaaatctaaatgtagcttatttgggtacttctggcttaggttgtctcagaacgttgcaaacaaggtattggctgcaaatgtggtctcatcacagggcttgcctgagaggatatgcttccagcctcactcacatggccatggctttggccagagacatgggctttttgattctataaggcactcataatatgacacctaagcttcccttagtgaggcagtgagagggcaagagagggtgcccaaaacaaaagccacagtctttttgtaacctacccttggatgtgtcatctcgtcacctctgccacattctcttcagtagaaatgagtcactgtctaaagctctcacccaagggttgggaattccacaaggtcctgaataccaggaggtagatgtcattgggggatatgttagaggttgtccatgtaacctttactgaagcttccatttatgttaacatcttaccgtaacatcataaccataaaatatttatcaaaataagaaattattattggtgtaaaactatatagtctaacattgatttcaccaggtttttccacaaatgaaccttttttgtttcagatccaacccaggattccgtcttgcattttagttgtcatgtcttagactcctccaatctatgacagtttcccagtctttttttgtctttcatgaccttgacgctttgaagagtgctaatcagttattttgaagaagcttcttcaatttagcattgtttgatggttcaaggttagattagggttatgcattctcaacacgtcacaacagagagcacattatgttgatatacattactggtgatgttaaccttgatcacttggctaaagtggtatctgctaggattctccactgtaaggttattgtttttcctttggagatactaaacatttgagggagattttgttagactatacaaataacctgtttctgcttaaactttgactactaattttagcattcatgagtggatcttggctgtagcagttattactctggtgttttaatttttttatgtctgtcatctcttttacatttgttaattggaattcttctgtaaggagaattgttcttccctcatttattccgccctaggttatttatttacattactgtagactcattagtgtttatttcattatttggattataatccaatgctaccattactgttttgttgctcaagttctctttttagctctttagagcacttttaggttggctcctgtaccctgccccagagctggaatcaactgtttcattaaaaatccctctttttattgttgttgttgttgttgttttctttctttttgagccggagtcttgctctgtcacccaggctggagtgcagtggcgtgatcttggctcactgcaacctccacctcccgggttcaagcaattcacctgcctcagcctccagagtagctgggattacaggtctgcaccaccaaacccagctaattttctgtatttttagtagagacagggtttcaccatgttggccaggctggtcttgaactcctgacctcaggtgatctgcccacctcggcctcccaaaatgctgtgattacagacgtgagccaccatgcccagccaaaaaaccctcttatttttgaagggaggaaggagtacagtatttagaaagcaagatgtaaggacctggcatgctcattgctggtgggatgttgctgtgtctagcctttctcagtgaataaagctaagaaatataggtagatttgtaaatttatgtgtatactcatatctgtatcttttaacgtgtgtgtgtgttttaaatcatgtgtccatattgatacctctgactctagtacagatccacaaggttgattctagcctttcccctttctttatttgtaacttctgtctctaatagcgataaaactggctctcattatctgtaatatatttaccctaatatataaatagtttcagaattcatagcccctacccacatgaaaaacaaattctgcatctagagtacagtgtttatctgcagtttcgaagttatcattaaaatatctagacaaaaatgctgtttgctaaagtttcttaagtcagcatctcccttccccatattcttcagtatgagtacgtgattcatttataatatagattcattagtcacattctgcattccatctttgcctcatatcctggttcacctttgaaaatttccgtacttataaatttcactttctgtgctttacaattctatccattgctacagtttcatatgagcagttccatcaatccccaaattcccctatgccgttcctttggagacagtctttcctctgatccctcacccctggcagtccctggtctgtattcttccttagagttttgcctttctagagtgtcacacaaatggaatcataaaatatgtaacagccttttgggtctgaattttttactttgcaaaatgcatttgacacatccatcttgtgtggatcagtagtgcttttttattgctttattaaatgcagtaatattttattgtaattgagtttgtttatccattcactcactgaaggatatttgagttgtttctagtttgggaccattaaaattagaactgctgtaaacatttatatacaggtttttgtttctctacagtaaatactaagaaagaggattcctgagttatatggtacaggtgtatatttaactttgtaagaattattcagagctaaactaatactggtttttcagagtgtctgtatcattttgcattcctgctagcaatgggtgagcattctagttactccacatcctcaccgatgtgtaaattgtcaatatattttagccatttttatagttgtacagtggtatctcaacatagttttaatttgcatgtccttccttaatggctaattattatgaacaccgtttcaagagcttatttgccatcctcttcggtgaagtgctttttaaatagggttgtttgctttcttattggtgagttttgagagttcttaatatattctggatacgagtcttttctggatatgttatttgcaaatatctcttcacagtatgcgacttgtcttcttatcctcttaaaaaaggctttcaaaaaacaaaggtgtttccttttgatcaagtccaattaattgatttttaaatggatcgtgtgattgggttggcaatactttcctttcagcacttaaaaaatatgctccttccttctggattccatggtttctggcaaaaaattcatagtcatttgagtcattgttttctatgtaagaacaatgtgttatttttctctgattattttaatttttttccagcacattgattatgcttctagatatgggtttcttcttttttttaatttttaaattttcatttcagtagtttttgggataaaggttgttttggttacatggataagttctttagtggtgatttctgagattttagtgcacccatcacccgagtagtatgcacaccatacccaatatgtagtttttttaagcccataggtttacaggcatacctcagatatattgcaggttcagttccagaccactgcaataaagccggtcacctaaattttttggcttcccagtgcatgtacaagttatgttaacactatactgtactctatgaagtgtataatagcgttatgtctaaaaaaatgtacctaccttaatttaaaaattgtcgttggttgatgggcacttaggtttttatgagttacttcacttagaataatggcctccagctccatcaaagttactgcaaaagacattatttcattcctttttatggctgagtagtattccgtggtgtatatataccacattttctttatctactcattggttgatgggcatttaggtttttatgtctttgcagttgcaaattgtgctgctgtacaagtgtgtgcacgtgtctttttaatgtcttctttttctaggtatagatttctttggtacatcttgtttggggtttgctgagtttttttaagcctataggtttacaggcatacctcagatatattgcaggttcagttccagaccactgcaataaagccgatcacatgaattttttggtttcccagtgataatacaagtgatgtttacactatactgtactctattaagtgtataatagtgtcatgtctaaaaaaagtacataccttaatttaaaaattctttattgtttaaaaatgctaacaattatttgaaccttcagtgagttactttttgctggtggcagtcttaccttgatgttggtggctgctaactgctctgggtggtgattgctaaagattgaggtggctgtagaaatttctttttttttttctttttaattttcattggtacatagtaggtgtatatgtttatgggtacatgagatgtttgatacaagcatgcagtgtgaaataaggacattgtgaagaatggggtacatcccttcaagcatttttatccattgagttgcaaacaatgcaattacactaagttattttaaaatgtacagtcattatttactatagtcacccttttgtgttacatagtaggtcttattcattctttctattttgtgtgtgtgtgtgtgtgtgcccattaaccatccccatctctccccaaacccctcactacctttctcagcctctggtaaccatccttctactctctatgtccatgagttcaattgtttggatttttagattccacaaataagtgacaacatgcagtgtttgtctttttatgcctggtttatttcatttaacgtaatgatctctagttccatccatattgttgtaaatgactggatctcatcattttctatgtctgaatagtactccactgtgtatatgtaccacattttcttacccatctattgatggacacataggctgcttccgaatcttagcttaccacaaacactgctgcaacaaacatgggattgcattgaaactgtagattgcttttgagtaacaaaatcagtcttccatatactgatttcctttcttttgggtatatactcagcagtgggattgctggatcatattgtagctcagtttttagtttttttaaggaacctccaaactattctccttagcaattgcactaatttacattccaagcaatagtatacaagggttcccttttctccacgtcttcaccagcatttgttattgcctgtcttttccatataagccattttaactggggtgagatgatatctcattgtagtttttagttttagtgttttgattggattattagattttttcctatggaattgtttgagctctttatgtattctggttattaatcccttgtgagatgggtagtttgcagatattctcttccattctgtaggttgtcttcttcactttattgattgtaccctttgctgtaaagaagctttttaatttgatgtgatcccatttgtccattttcgctttggttacctgtgcttgtggggtattgctcaagaaatttttgcccagaccaatatcctaaagattttccctcatgtttttctgtagtagtttcatgatttgaggtcttaggtttaagtttttaatccattttgatttgatttttgtatatggcgaaagatatagagttctagcttcattcttctgcatgtaagtatccagttttcatttatttctgctgttttatagtttttgtcagggaattctgatatctcttttatcctagccttggtgtctgttcccatgtgatttagtttttatatttgtctcttcaaaatctcatattgaaatttgatccccagcattagaggtgggacctaaagagagatgtttgggttatgggggcagatccctcatgaatggctcagtgacctccctgtggtaatgagtgagttctggctcgtagttcacccaagagctggttgtttaaaaaagaatgactcccctcctccccaccttccacttcttttctcttcccgtgtgatgtgggctctccccttacacttctgctgtgattggaagcttcctgagttcctcaccagaagcagatgctggtcccatgcttcttacacagcctgcagaactgtgagccaaacaaacctttttacttggtaaattacccagcctcaagtattccttaatagcagtgcaaacagactaagacaccatttgagctgagatttttctggttctaaatatgatgaataattttggattgtatttcataatgacgtgttatgaaatgacaagtcttgtttaaatgccatagagaatgttgatgtttttgttttagcagacaatgacccaactagtttcaggttaaaaattccagccagccttctgtaggttgttgttccaagtccattcagttttcaaagattttgcagtgctgttcacatctgttgtctgtgtgtgtgcctagtggcccttttctaggtacttttatcccttttctatgtactttctgtgactacattgtgtcagggctgagtggcacaggacagaaggagaaaaagccaaccaggagtggtcactcttgagaccacatttcctttggtgtaagttgaaggtttcccttcttcagaactttaggctcctgccagcctctgttactgcttgtaccattgccaccatgagattactgggatgctatgactaatatacagagataaggaaaaaggggattccacactctatatagagtgttgggagacccttctttcattcctgaagcctgaactagaaggcttcgctgtgaagtctttctctctatgctaatatccacttttaggtttcaggctatattgagttcaggccaaggaatgacagaggaaacaaaaatgatggcaaactcattaccattttgatgatatttcagattttatgatcttcaatctgcctgcttctgttattttccacagtcttccaatagctgctccatgtagtctatctaggttttatagctcatctcatctaggattagatacgctaacctggaactggaaccacttctactgttttttgttcccattaactgcatatgtgagtttcagttattccacatccttgccaacactgggtattaacagttatttttaatttcactcattctaaatgtgagtatgaaacagtatggtattgtaattttaatttgtatttccatgatgactaatgatgctgagcatcttttctgtgtctcttgcccatttgcatgattttatttatgaagtatctgttcacttttatccattggattatttgtctcagtattataggaatttttataatatattccagatacaaatcctctgtgtattgtgaatattttctcatagtctgtggtgtgatttttgttttcttaatggtgtctctggggaacagaaagctttaattttgatgaattccactttggtaatttttttttaatgattagtacttttttgtgtcctgattaagaaatgttgcctaatcaagtgttgtaaaagttttctcctgtattttcatgtagaaatgttatggtcttaactttgtgatcaagtctgtgatctgtttccaaattctcctttgtggtgtaagttatgcagttgaggatcattttctttcctaggaatattctagcaccacttttcaaaaatgattatttccttattgaagttagttggaaccttggtcaaaattagttggccttatatgtgtaagttttagagtttctactctgtttctttgttctttttatttatctttacaccagtaccacaatgtcttgattactgtagctttatagtaagtcttgaaatcaggttatgtcctccaaccttgtttttgttttcaaaattatttttgctatttcaagtcctttgcattttcatacaaattttagaataagcatactgggatttttttggggattgtatggaatctgtagatcaattttgtagattgacatttgaattttcagtcttcctatgcatgaacatggtgagatttttcttccccacttatttaagtcatctttaatgtcccccggtgacatattgtaattttcgttgtgtagattttacacatctactaaatatattcctaagtagttaatatactatggtatatggcattttaaaatttaagttttctcattgttcattgcttatatatagaaatactgttgtcatgccaatttgtaggtcccaacaagttaattttaattttaatttattattctttgggtcagagctttcatgcctactgtgggatgttttagggcaatgagtgctgtagagaatttatacaaggcaaaagttccagtggttgacatatatctatttgttctctgttttatgttcagtaactcctctaagatttcaagagtttcttctttaaatttactgggatcctcaggtataaagtctattcttcatacataatcatttcaccagagaacagagcagttttactccttcctttgcagttctcatgccttttatttctttttcttgcttaatgcactggctagaacattgaatatgataccaaaacaagtagtaaaagcagacatcttgccctgttccctattcctggagggcaggagagtagagtagtgttttgtcattaagtttaatattacctgtagatttttaatgggtgccttttatcagcttgaggatgttttctaagtgagttttatcatgaatgggtgtttaatttttcagatgtttttcctgatctatggatatgatcatacatgttttctccttttttctactgatgtagtgaattacattggtccagttgatcaactgtattgttgaaattgtcatctttactgaatttttatctgcttgtttaatcagcatttgaaaacagcatacatctccagttatgtttgtgtgttttctgtttctcctttagatcttctttggagtattttgtggggaggggaacaataacaaagttatagaaaaggtaaatatggcacaaggaacttctctttctccttagttatttgggaatacgttgttgacctgctgcacaatcactttcgtgtaatatagtctcatttcctataaaaggacattctcctataggaccacaataaaaccatgagaattaggaaattcatactagtgcattacagacattatttagatttctccagttctcccagtaatgtctttttttttatttttatttttttataacactttaagttttagggtacatgtgcacaatgtgcaggttagttacatgtgtatacatgtgccatgctggtgtgctgcacccattaactcgtcatttagcattaggtatatctcctaatgctatccctccccccttccccgaccccacaacagtccccagagtgtgatgttccccttcctgtgtccatgtgttctcattgttcaattcccacctatgagtgagaatatgcagtgtttggttttttgttcttgctatagtttactgagaatgatgatttccaattttatccatgtccctacaaaggacatgaactcatcattttttatggctgcatagtattccatggtgtatatgtggcacattttcttaatccagtctatcgttgttggacatttgggttggttccaagtctttgctgttgtgaatagtgccgcaataaacatacgtgtgcatgtgtctttatagtagaatgatttataatcctttgggtatatagccagtaatgggatggctgggtcaaatggtatttctagttctagatccctgaggaatcgccacactgacttccacaatggttgaactagtttacagtcccaccaacagtgtaaaagtgttcctatttttccatgtcctctttatcactttaaaactcaataaagttgtggctgttaggtgcctttatcattttaaaactcaataaggttgtggctgttaggtaccttttcatcccacagtaggcatgaaagctcttaggcaccttaataccccaaacaagatactcttttcccttttgtaattaagtgttttatggaaggtatgtttaaattatgtaaacatcttattcatcagcaaacttacttattaatttgtatctgtacagaatcttccttattttattcagtgagttatgatcaatttctaccttttttctttttaacttgttatttgaaaatacatatagactcacaagaaaaatatgtgactcacaagaggttacaaaaatagccaagggaggtccccactcagcttccccattgataatgtccttacataactataatgcattatcaaaaccagtaaattgatattggcacaatacagtcaagtggactacagacgctaatcagatttcacaagtttttgcatgcactcagttattctaatgcatatatgtgtaattctgtaaaatttaaacacatgtatagctttatgtaaccgccatcataatcaagatacagaagtgctctgtcactgtgaaggaattcccttgtgccttcccttttctagtcacaccctgttccctcaagccagccttctttaacccctgaaagccagtgatctgtttttctactgtataattttgtcattttgagaagttatatagatggaatccaacatgttacatagatggaacataacatgtaaactttgagatgtgttcttcttactcagcatactcacctggagatccattcaacttgtgtgtatcagtagttaatttctttgtattggtcagcattccattatttggaatttatacagtatgtttatccattcacctgttgaaggacattgtttccaatgcttattactaataaagctgttatgaacattcatgtacttttttttttatactttaagttttggaatacatgtgctgaatgttcaggtttgttacataggtatacatgtgccatggtggtttgctgcacccatcaactcgtcatctacattacgtatttctcctaatgttatctctccccttgcttcctaccccacaacaggccccagtgtgtgatgttcccctccctgtgcccatatattctcaactcccacttatgagtgagaacatggagtgtttggttttctattcctgtgttagtttgctgagaatgatggtttccagcttcatccatgtccctccaaaggacatgaactcattcttttttatggctgcatagtattccatggtatatatgtgccacattttctttatccagtctaatattgatagggatttgggttggttctaagtctttgttattgtgaataatgctgcagtaaacatacatgtgcatgtgtctttatagtagaatgatttataatcgttggggtatatacccagtaatgggattgctggtcaaatggtattctagttctaaatccttgaggaatcaccacactgtcttccacaatggttgaactaatttacacttccaccaacagcataaaagcattcttgtttctccacatcctctccagcatctgttgtttcccaactttttaatgattgccattctaactggtgtgagatggtatctcagtgtggttttgatttgcatttctctaatgaccagtgatgatgagctttttttcatgtttgttggctgcataaatgtcttcttttgaaaagtgtctgtttatatccttcgcccactttttgatggggttgtttgtttttttcttgtacatttgtttaagttccttgttttcattcttaagtaaattaagtttccttgatttttgttcttttgcatttgctgaggagtgttttacttccagttatgtggatctcagccctttttcagatggatagactgcaaaattttctaccattctggaggttgcctgtttactctaatgatagtttcttttgctgtgcagaagctctttagtttaattagatctaatttgtcaagtttggcttttgttgccattgcttttggtgttttagtcatgaaacctttgcccatgcctatgtcctgaatggtattgcctaggtttttttgtagggtttttatggttttaggtcttacatttaagtctttaatccatcttgagttaatttttgtataaggtgtaaggaagggatccagtttcacttttctgcatatggctagtcagttttcccagcactatttattaaatagggaatcctttctccattgcttttgtcaggtttgtcaaagatcagatggttgtagatgtgtggtgttatttctgaagcccctgctctgttccattggtctatgtatctgttttggtaccagtaccatgctgttttggttcccgtagccttgtagtatagtttgaagtcaggtagcatgatgcctccagctttgttctttttgcttaggattgttgtggctatacgggctcttttttggttccatatgaactttaaagtagttttttctaattctgtgaagaaagtcaatggtagcttgatgaggatagcattgaatctataaattactttggacagtatggccatttcacaatattgattcttcctatccatgagcatggaatgtttttccatttgtttgtgtcctctcttatttccttgagcagtggtttgtaggtctccttgaagacatccttcacatccattgaatgttgtattcctaggtattttattctctttgtagcaattgtgaatgggagtttgctcgtgatttggctctctattattggtgtataagaatgcttgtgatttttgcacattgattttgtatcctgagactttgctgaagttgcctatcagcttaaggagtttttgggctgagatgatggggttctctaaatatacaatcgtgtcatctgcaaacagagataatttgacttcctctcttcctatttgaataccctttatttctttcccttgcccgattgccctggccagaacttccaatactgtgttgaatagcagtggtgagaggccatccttgcattgtgtcggttttcaaagggaatgtttccagcttttgcccattcagtatgatattggctgtgggtttgtcacaaatagctcttatttttttgagacatgttccatcaatacctagtttattgagtgtttttagcatgaaggggtgttgaattttatcgaaggccttttctgcatctattgagataatcatgtggttttttgttattggttctgtttatgtgatggattacatttattgatttgtgtatgttgaaccagccttacatcccagggatgaagctgacttgatcttggtggataagcttttgaatgtgctgctggattaggtttgccagtattttattgaggatttttgcatcgatgtttatcaggtatattggcctgaaattttcttttttgttgtgtctctgccaggttttggtatcatgatgatgctggcctcataaaatgagttagggaggagtccctctttttctattgtttggaatagtttcagaaggaatggtaccagctcctctttgtacctctggtagaattcggctgtgaatccatctggtcctgggcttttcttggttggtaggctattattgcctcaatttcagaacttgtcattggactgttcagggatttgacttcttcctggtttagtcttgggagggtgtatgtgtcgaggaatttatccatttcttctagattttctagtttatttgcgtagaggtgtttgcagtattctctgatggtagtttgtatttctgtgggatcagtggtgatctgccctttatcattttttattgtgtctatttgattcttctctcttttcttctttattagtctggctagtggtctatctattttgttaatcttttcgaaaaaccagctcctgaattcgttgatttttttggaagggtttttcgtgtctctatttccttcagttctgctctgatcttagctatttcttgtcttctgctagcttttgaatttgtttgccctcgcttctctagttcttttaattgtgatgttagggtgtcgattttagatctttcctgctttctcctgtgggcatttagtgctataaatttccctctacacactgctttaaatgtgtcccagagattctggtatgttgtgcctttgttctcattggtttcacagaatttatttcttgctgccttgatttcgttatttagccagtagtcattcaggagcaggttatttagtttccatgtagttgtgcggttttgagttttcttttctcttttcttttctcttttcttttcctttcctttccctttccctttcctttcctttcctttcctttcttttcttttctttccaccctgccctcctttcttcttcttctttttttttttttttttccgagtctcactctgttgcccaggctggagtgcagtggcgcgatctcggcttactgcaagctctgcctcctgggttcacgccattctcctgcctctgcctcccaagtagctgggactacaggtgcctgccaccacgcctggctaatttctttttgtatttttagtagagacagggtttcactgtgttagtcaggatggtctcgatctcctgaccttgtgatccacccgcctcggcctcccaaaatgctggggttacagatgtgagccaccgcacccggctgagtgagtttcttaatcctgagttctaatttgattgcactgtagtctgagggactgtttgttatgattttcattcttctgcattttctgaggagtgttttacttccaattatgtggtcaattttagaataagtgctatgtagtactgaggagaatgtatattctgttgatttggggtgtagagttctgtagatgtctattaggtctgcttggtccagacctgagttcaattcctgaatatccttgagctgagttcaagtcctgaatatccttgttaattttctgtctcgttgatttgtctaatattgacaatggggctttaaagtctcccactattaccgtgtgggagtctaagtctctttgtaggtcgctaagaacttgctttatgaatctgggtgctcttgtattgggtgcatatatatttaggatagttagctcttcttgttgcattgatccgtttaccattatgtaatgctcttctttgtcttttttgatctttgttggtttaaagtctgttttatcagagactaggatttcaacccctgtttttttttgttgttttgttgttgttgttattttttgctttccatttgcttggtagatctccctccatccctttattttgagcctatgtgtgtctttgcacatgagatgcgtctcctgaatacagcacaccaatgggtcttgactctttatccagtttgccagtctgtgcattttaattggggcatttagctcatttacatttaaggttactattgttaggtgtgaatttgatcctgtcattatgatgctagctggttattttgcccattagttgatgtagtttcttcatagtgttgatggtctttacattttattattattattattatttttgcagtgactggtactggtttttccttgccatatttagtgcttccttcaggaggtcttgtaaggcaggcctggtggtgacaaaatccctcagcatttacctgtctgtaaaggattttatttctccttcacttatgatgcttagtttggcgggatatgaaattctgggttgaaaattcttttctttaagaatgttgaatgttggcccccactctcttctggcttgtagggtttctgccaagagatctgctgttagtctgatgggcttccctttatgggtaacctgacctttgtctctggctgcccttaatattttttccttcatttcaaccttggtgaatctgacgattatgtgttttggggttgctcttcttgaggagtatctttgtggtgttctctgtatttcttgaatttgaatgttggtcagtcttgctaggttggggaagttctcctggataatatcctgcagagtgttttccaacttggttccattctccctgtcactttcaggtacaccaatcaaacgtacgtttagtcttttcacatagtcccatatttcttgtcttcacactttatttcatgaagttgatcttcagtctctgatatcctttcttccacttggtcgattcagctattgatacttatgtatgcttctcgaatttctcatgctgtgtttttcagctccagcaggtcatttatgttcatctctaaactggttattctagttagcaattcctctaaccttttatcaatgttcttagcttccttgcattgggttagaacatgttcctttagctcggagaagtttgttattacccaccttctgaagcctacttctgtcaatttgtcaaacttattctccatccagttttgttgctggtgagagttgtgatcctttggaggagaagaggcattctggttttggaattttcagcctttttgcactggtttttcctcatctttgtggatttatctacctttggtctttgctgttggtgaacttcagatggagtttttgcgtggtcgttctttttgttgatgttgatgcttttgctttctgtttgttagttttccttctaacagtcaggcccctctgctgcaggtctgctggagtttactgggggtccactccagaccctgtttgcctgggtatcaccaatgaaggctgcagaacagcaaagtttgctgccttctcctacctctgaagttttgtcccagaggggcacccgccagatgccagccagggctctcctgtgtgaggtgtctgtcgacccctgctggtaggtgtctccccaccaggaggcacgggggtcagggacccacttgtggaggcattctgtcccttagcagggctcgagggctgtcctgggagatctgctgctctattcagagctggcaggcaggaacgtttaagtctgctgaagctgtgcccacagctgccccttcccccaggtgctctgtcccagggagatgggagttttatctataagcccctgactggggctgctgcctttctttcagagatgccctgcccagagaggaggaatctagagaggcagtctgggcttcacccagttggaaattcccagcggctttgtttacactgtgaggggaaaactgcctactcaagcctcagtaatggtggatgcccctcctctcacaaagcttgaatatcctaggtcgacttcaggctgctctgctggcagtcagaatttcaggccagtcgatcttagcttgctggactccttgggggtgggatccactgagctagaccacttgccttcctggcttcagccccctttccagaggagtgaacggttctgtcttgctggcattccaggcgccactggagtatgaaaagaaaactcctgcaactatctcggtgtctgcccaaatggccgctcagttttgtgcttgaaaccagggccctggtggtataggcacccaagggaatctcctggtctgtgggtttcgaagactgtgggaaaagcctagtatctgggctggaacagtcctcatggcttcccttggttaggggagggagttccccaacccgttgtgcttcccgaatgaggcgacacccccaccctgctgctgctcaccctctgtgggctgcacccactgtctaaccagtctcaatgagatgaactgggtaccttagtcggaaatgcagaaatcacctgccttctgcgttggtcttgctgggagctgcagactggagctgttctatttggccatcttgcccgggaatccctcatgtacttgttattacatgaccataagttttttatttctctatgacagtgggtctcaactaggagtgattttacctctcagggaacatgggaaatgtctgaagacatttttgattgtaatgactaggaaagaaatgctactggtgtctagtaaataaaacatagaggtgctgtggaatatcctagaatgcacaagacagtcctccacaacaaagaattgtttcaaaatgtcagtgctgaggttgagaaactgcactaggattaagacccaggagtgtgattattgagttgtacgtaagtatatgtttaattttataggaaactgccagagtgttttccagagtggttataacattttgcatttccaataacaatgtgtgagagatcaagttcctccacatcctcatcagcatttatactgtcagtattttttatatttttatttttttgagatggagtctcactctgtcacccaggctggagtgcagtagcatgatctcagctcagtagcaggatctcagctcactgcaacctctgcctcccgggttcaagcgattctcctgcctcagcctcccgagtagctgggactacaggtgcgtaccaccacgcctagctaatttttatatttttagtagagatggggtttcaccatattggtgaggctggtcttgaactcctgaccttgtgatctgcccaccttggcctcccaaagtgctgggattacaggcgtaagctaccacgcctgtccactgtcagattttttttttttttaatttagccttcctgattggtatataggggttatctcattattgctttaatttgcatttctctactactgacaatgaacatttttaatatgcccatttactatctatatgttttctattaaaattgttttgctcattttctaattgcattttttttactgttgaatttagagaatctttatatattctggatacaaattctttattagatttcagacttgcaaatttttacattgtacaaaaccaagttttcttattctatttattgttactattgtaatttgttttgatgctcaaaatacagatttggtggccggtggcagccccttcaagctggctctatatcctttttgtatgtccccgtcatcttttgatcatgtctttgctttctggcacaagatatttcaggctcatcttatacttttctttctccagctctggaatcaactacttctccaagaagcctgttcttccagtggaatgtgctatttagaggttaggatttaaacactaggcatatgtgttgctcccagccctacacttatttacattaatatttatttctctatctgtatatattaaaaaccatatgttcatctcattttttacttttttctttgtttttgtaactttcatatctaatagtgagtaacctcacttccagtgtgctttatataacgaattctctcatttcttgccaacctctccccagcaaggatgaccctaacgctccacttggattcaaacactttattgtctgtctccttgggtggatgctcttttcaccctgcttgggctttggcattccatgctagaccactcctcaacatgtatagcacccctccctacttggtctctgacttccctgtgtcagaatgctcatcaccctcaacgtatggacactctccccaccccacgtaggttctgactccccatgctgaactaccccctcacatggatttcttcctcatgctgcataggctcatgacacctcacattggtctccctctacttggatgccctcatcttgtttaggatttgactcaagatgtcaggctacgccatttagacagcctcctcactcaggttctgacattcaaggtcagactgactcctgtcaccctgcttgagtagtgactcaccacactgcccttaccctgtatgacacccttctcatcctgcaggaaatctgacttcccagcctaggctatctctctgtgtaggtattttccttctctactgcagctctgccactccatgtaaggccaccccagtgtggagacacctacctcattctgcttgggctctaactccacatgtctggtggacctccctcttcatggaagccctccatatcctgatttggtgcttttattgcacactaagccacacgtctgcagagactgtcttcttgccctgcttgaattctaatactctgtgctaggacacttctttacatgggcaccttccttaccctaatgagattctgataccttgctctggcccgttgtcactccatttgctacagattcccgccttgttctgcctacctaatggttttaagattgtattgtttgggatgggaaagataaaagaaagggagaatagaaacacaggagaggggaaagagaataggaagcttattttttgttactttgtgattaatatatcttttctctccttttactttcaacctatctgtgacctcacagtaaagtgcatcacttgtcaatgtcatatggttgaatgttattcatttttaatcttgtctgttaatctttatcttataactgagatatttagttcatttacatttaatataattattgagctagttgattttgctatcctctattattttagattaattaattatttttattccatgctattttctctattggcttcttttacttttttttcttttagtggttattctagagattataatatacttccttgactgtatagtttaccttaaattagtacttttttcgctttatgagcagcaaagtatcttaggacagttgaactctatttactcactcctcctatttgtgcttttgttgtcatatatttctgttctacatgttctgaatcccacaggacattattattgttgctttgaacaaacaacattcttttccatatatatgtatataagaaatataaagcaacgtaaaatatgtatatatacacaagagtgtatgtatgtatatgtacatgtatatatttacatacacacatcctttaagtgttctgcattctttctagcagttctgtactatgttggatcattttccttcagcctaaaaaatttcctttagtatttcctgaagtcttctggggattatcttagtttttatttgtttggtttaagaaatgtatttcagttggttatagaattgtaggtttttatttcagcactttaaagagctactatctttgttgaaaggccaagtgtcgttcttatttttgcacctttgattttcagcagtttggctttgatgccccaagagttggtttttcttagtattactttgtgttcactgagcttcttaactcagtagattaagtcttctacctgttttggaaaattcttacccattaactcataaatattgtttctgcactcttccctcttctcctaggagtaagattacacatgtattggattatatgactgtgccccgtgagtctcttatgctgtcatccgttcgttctttttatgtttggcagttttctgtagactttttgaatttacaaattatttttacacagtgtctatttgcttctactacagttgaatcagattttagataatgtatttttattctgaaatattcatttctttttttaatagattgtagttttctgttgagattttccatattttatctatttccctttatcttttgctctcaatatattaataaccattttgaagtctttttgtgctaactagtagctaaatcccctgtaaatcagtttttcttgtctattttttcctctcaatttttgatcattttttcctgcttcatatgttacataatgttttgtgtgttggatattgtaaataagaggataatagagattgagggtatttgcatctagagggactagaaaaacaagagcaagccaaccccaaagctaacagaagaaaataaataacgaaaattagagctgaactgtatgaaattgagatgcgaaaatccatacaaaagattaactaaaccaaaagttgattcttccaaagaataaacaagattgacagaccactaactaaattgttaaagaaaaaagaagacaaaataagtacaatcagaaatgacaaaggtgacattatcactgactccacagaaatacacacacacacacacacacacacacacacacacacaaactcagagactattatgaacacctattcacacacaaactaggaaacctagaagaaatggataaattcctggaaatatacaatctcccaagattgagccaggaagaaatggaaatcatgaacagaccaataatgagttctgaaattgagtcagtaataaaaaacttcttcacgacaaaagaaaccgtcagcatagtaaatgaacaacgtacagaatgggacaaaatatttgcaaactgtgcatccaacaaaggtctaatatccagaatctgtaaggaacttaagcagttcaacaagcaaagaacaaccctatttaaaaaatgggcaaaggacatgaacagacgcttctcaaaagaagacatacttgcagccaacaaatatatgaaaaaatgttccacattactaatcattcaagaagtgcaaatgaaaaccacaatgagataccatctcatgttagtcagaatgtctattaataaaaagtaaaaaaaataacaggtgttggtgaggttgcagaagaaagggaaggcttgtacactgttggtgggaacataacatagttcagccactggggaaaactgtggagattccttaaagagcttaaaacagaactaccattcaactcagcagtcccattactgggtatatacccaaaacaaaataaatcattctactgaaaaaacacatataatcaatatcatattattgctattcacaatagcaaagacatggaatacaaaaaacaatttttgctttaagcctgacagatctttggtttttcaacaactaaatattgtaagaactcagttgaggctttcaggtgtttgaactcagctctttagactctttgccctcacaactttagaatatggtaaatgttgtaaggggaacaacagtcatgcatttcttgcagttaaactttctcaagggaaattcatttcattagtactcttgcaaataattttactgtttttctgactcagcccctttattggggttggcccttctgacttttgactgagagtctagtgagtctttaattccttacctttaaaagttctctccctcaagcattagttattaagccccctgccttaagcaggtacaacatttggcaaatgttttaaggaggagagctgatgtgtgtttgtcccaggcctctccatctagtgggacttggtttcttatagtgccatgagtatgaatgagatttcagtctattactctgactcagcccccagcctcccataccaccaaaaaaagggaaattgaccttttttttcttttctcataattgtccttttcaattagtgaatggtagacatacggtagagactcaaaagttctacttttgtgatatattctagatattactctaatacaatatgacattattctcttttcttgaaactttattttcaagtgattttttgaaagttttagtctttttttttttttttttcttatagagacaaggtgttgctctgttacccaggctctggagtacagtggtgcagtcacggctcactgctgcctcgaactcttggtctcaagcagtcctcctgcctcagcctcctcagttgctgggactataggcgcatgccactgtgcctattttttttatgttttgtagagacgatcttgctgcattgcccagggtgacctctaactccttggctcaagtgatcctcctgcctcagcctcccaaagaactgggatcacagatgtgaggcactgtgccaggccttattttcaagtaattttagagttacaaaaaagaaacacgtatagtagagttccttataccactcttctagcttctcctgttgttaacatctttttttttcattttatttttatttttatttttttgaagacagagttttgctcttgttgcccaagttggagtgcaatggcgcaatctcggttcattgcaacccctgcccgctgggttcaagtgattctcctgcctcagcctcctgagtagctgggattacaggcatgcaccaccacgcctggctaatttttgtattattagtagagatggggtttcaccatgttggccaggctggtctcgaacttttgacctcaggtgatccacccacctcagcctcccaaaatactgggattacaggcgtgagccaccacgcctgacctattttttatttttaaacttttaggttcagggggtacatgttccaatttgttctataggtaaacttgtgtcatgggggtttgttttacagattatgtcatcacccaggtactaagcgtggtacccagtagttttttttttttttctgtttgtctcccctgtccctccaccctcaagtaggccccagtgtctgttgttcccctctttgtgtccatgagttctcatcatttagctcccacttataaatgtgaggatgcagtatttggttttctgttcctgcattagtttgctaaggataatgacctccagctccatccatgttctgcaaaagacatgatcttgttcttttatatggctgcatagtattctatggtgtaagtgtaccacattttctttatccagtctgtcatttatggggcatttaggttgattcatgtctttgttattgtgaatagtactataatgaacatttgtgtgcacgtgtctttatggtagaatgacatatatttctttgggtatgtacacagcaatggattgctgagtcaaatggtgcatctggttttagctctatgaggaattgccacactgctttctacaatggttgaactaatttacactcccacaaacagtgtataaccactcccttttctctgcaaccttgacagcatctgttatattttgacttttttataacagccattctgattgatgtgaaatggtatctcattgtggttttgatttgcatttatgtaatcagtgatattgagcttttttcatatgcgtggtggctgcatatatatcttcttttaaaaagtgtctgtcatgtcctttgcccactaagttccttgtaaatgctggatactagacctttatcagatgcatagtttgcaaatattttcttccattctataggttgtctgtttgctctgttgataatttttcttttgctgtgcagaagctcttatgtttaattagatcccacttgtcagtttttgctttcgttgcaattgcttttggtgtcttcgtcgtgaaattgcctgtttctgtgtctggaatggtattgtctaggttgcatatgtggtatccacatcatcactggtgatgttaactgatcacttggataaggcagtgcttgctaggtgtctccaagatggaaataagttactatttcttccttcttatactctgttttttgaaatccagtcacttagttcagtgcatacacaaaggaggaggggaaggttaagcttaaccttctttggtgggaggtattcacatctgttatctggacttcttcttaaagcaaaatttgcttcttctttccaattatttatgtgttcaattatttacttgctccactctgttttaatattaattccctatgatctcaagatataactgcaaccttgaaaactcctagttctcccagaactccttgacagaatgctctgtttcttgtttatctttgtacactgtgtgtatttagcacagtcccctaatttatggtggacatctagtaatttaaaaaaatgtattttcaattcagctgaaaaatagcatttcctcaactaaattatataatttaacattctaaattaaaagtaggttttaagataaaagtaaacatgtgtgataacattgtatcaaacaaaataaagggtactgctgaaattttaccttaataacgtttgacattgatgtgtccacaatatattcctatagctacttaaatattccttgaaaccacttttggaaagagatttcagtctctaatggccaaggatataatctgctgtattctttctcttttttggtcagattaccagagaagatgaagaccccactttctgctttctctgaaaaattgtcatctgatgcagtcactcagataacaacagaaagtccagaaaagaccctattttcatctgagatttttattaatgctgaagatcgtggacatgaaattatagagcctggtaaccagaagctacgcaaagctcctgtcaagtttgcctcatcatcttcagtccaacaggttactttttctcgcggcacagatggtaagagaatgtgattgcattttagattgttagaccagctcttttgtgtagttatcttagaaatttgtggttgtgtctttctagtcagaaaacctaattttaatttaagaattagggtccatatgattatatctgtcttttatttgcatagctgtcaacagaatatgatttattatattttatcttaatttcaagcacagttaacatgtagaactcatgctgtttgtgttaaaacttagttttgtctcatcatatttgcaataaaaatgcaagtagttcataacaacttgaaagtaattgaggtctgcttttaaaatggcggataaattatcttgtattacagtaacagtaatgttattactatatagtgcgtatatagggagagtatgtatatactaaatactataacaaatagatgcataaacatgtgatagtgcaaacacaatagcagtttatctcttacttataaagcaatcaccagagacccaggttgtcttcttgctctgttttctttaaaatatataacccaaggttatgttggatttaccttcatggtcaacaggaaggaacaaaaagcattgaggagcacatataggagatgtttacaggtcaggtctggaagtaacccaaatcactttccactggccaggaatgtagtttagctgtgttttccaaaatgaagaagaaatggattttggtgaataggtagcagtctctaccataaggtgaacatttagagtgttttatatgctatagtttcagtgatattttgatagtacttttaccatgtaacatttagaattagaccaaattgtaaattattttctttttttgagtaagtagcaggaaaattaacaattaagtttccattcttccccaaatgaagatggagactttactaatttttaaaatatttactattttggacaagtctactcaaagtacatcttaagtttctctacttatacaaaaaacttgtccacctacacacgtagatggaatatacctaaatgtaaagaaaattgtttagttcagcatttaaattgcattgaatttaagtaagtgcatttatatactaagatggttataattcttgaacatttttcttgtgaaattaaagcataccaaaaatatatatatagtttaataaattatcacgaagaacacctttatgtgttggttacctgttgttacagtggtacagtataacaaacaacctaaaaaatctctatggtttataacagcagacatttgtttttcttactcacaggcttatgggtcaattggactttggctgggctggctgtaagctgcagaaatggtttgggtctgttccagatacttctttctgagactcaggctaatgggtcaatagctatcctagagtgtggtgttctcatggtggtggcagaaacacaagagaggaaacagctataaaagcagtgcgcggtggctcacgcctgtaatcccagcactttgggaggccaaggtgggcagatcacgaggtcaggagatcgagaccgtcctggctaacacaatgaaaccccgtctctactaaaaatacaaaaaaaaaaaaaaaaaaaaatagccggacactgtggcgggtgcctgtagtcccagctacttgggaggctgaggcaggaggatggcatgaacccaggaggcggagcttgcagtgagccgagatcgtgccactgcactccagcctgggcgacacagcgagactccgtttccaaaaacaaaaaaaaagcatattttaaacttctactgtcatcaaacggatcaatttatagaggccatgatttatggttcatataaactttttatcaaagaatcgatattatttggtttagtaataagtgttattttcaacactgtgggtcaaaagtaaaattttatgaggtgtttttttttgtttgtttgttttttgtacaaaacattttaaggtacaaaaaatcgtatgttaactacatgactgcttacccattagtctgaattaacaagttcctctacccctctccagtgcttttttcctttcccggaagcagccattatttgctatgtattcacttcttcccctagacaggcagtccagtgtaaccttaagactactgcatccaaacaaagttcttgagaacttcaaaaaattctttatttgtctcaggtccatcgccatgtgatagtcatttctgctctgggtccattgtaattttgtccaatcgttgctgttaattttctccaacctgtgaactgcatggcatattcagccattgctatcaagcttaatatataccgatggagaaagaaaattaataaatcagtattctaacaataactacgggaacgaccaaatagaattggaggctacagcttttttctttttctttttccttttttcctgatagagtggtacaaatattcctaagttgtctcagactaaggttgaggtcctttttttttttttttttttttgagatggagtctcactctatctccaggttggagtgcagtggtgcaatcttggctcactgcaacctgcatcttccgggttcaaggattctcctgcctcagcctcccaagtagctgggactacaggcgcgcaccaccacgcccaactaatttttgtatttttagtagagatggagtttcaccatgttggccaggatgatctcgatctcttgaccttgtgatccgcccaccttggcctcccaaagtgttgggattacaggcgtgagccactgcgcctggccaaggtcattttttattaatttcttgactggactttctttagaggtcactttgccatgcccagtgatagaaattaatgcctctaggtgttaatcatactccttttaaaaagtaaccttatttcttgtggataattcaggtctatcatagatttagtcagcactctgtaagctgctctatgttttgctttagtgatataaaaaatgttaagtagctaggtggctcctgtccctggtagaattgtccctttcttatttctaagatcttccatctaacaacactcatagtcacaaaggttgaaagcaaaaacttcttaaaaagcttgtttgttcccaaatgcatgcattctatctggtactcggtataggcaagtaccatctatggttcagagcatatcccaaatcccataagaaagcattgtagactcagaaagtggtgttccccagcttatgccataacacttttcagtaaatagcttgctcactttatttatttatttattttttaggtttagcatagttatggtccataaggcatggataaggacagtaaatccctttaggcatcaaccttttgtctcatttccttcatttggaagtgagtttcttccttagaagcaggctactaacaatgtggaattcattgccaaaatgtcatcagaatgatagagttgccaaagatggtgacacagttctaagtaatataaagattattgtttctggccaggcgtggtggctcacacctataatcccagcactttgggaggccgaggagggtggatcacctgaggtcaggagtttgagaccagcctggccaacatagtgaaacctcatctgtactaaaaatacaaaaattagccgggcatgatggcacatgcctgtaatcccagctactcgggaggctgaggcaggagaattgcttgaacccggaaggtggaggttgcagtaagcagagatcgtgccagtgcactccagcctgggcgacagagtgagactccttcccccttccccaccaaaaaaaaaaaaagattattgtttccagaggagagctcattctactccaggactatcagaatcagtctagaggattttgggttttattaactagagatgagcaactaggagtccatctagaaatggttacaggatataagagaaaaccgtggtctataagaataaagataaacggaaggagctggaggcattcggccatgagaaatggtagctacattgaaaattcataatcatatgagcaatacaaccctatgaaatgctcagaaataacctaaacaaaaattttaaatgacctaatgcaggaaacactaatcctttttgtaaagatagaaaataataatttactaaattaaaaggtatgattctagaatggaaggctgaatattgtaaaggtatcatttattcccacataaatttatactctaaagtaattctgcacaaagttctaatggaactgttcttttgaactgtgtaatttatctttatgaatttatttttatttttaatttgtctgggtatatagtagacatagatatttatgaggtacatgagctattttgatataggcatacagtgtataataatcccatcaaggtaaatgggatatccatcacctcaagcatttatccattctttgtgttaacaatccagttacattattttggttattttattttttaagtatttattttttttgagatggagtcttgctctgtcacccaggctggagtacagtggcatgatctcagctcactgcaacctctgcctccttggttcaagcaattctgcctcagcctcctgagtagctgggattacaggcacccgccaccacgcctggctaatttttgtatttttagtagagaaagggtttcaccatgttggccaggctggtctcgaactcctgatctcaagtgatctgcctgcattggcctcccaaagtgctggattacagatgtgagccaccatgcctagctgtcttttggttattttaaaatgtacagtgaattattgactgtagaccctgttgtgctatcaaatactatatcttactgattatatctgactatatttttatacccattaacaatcctcacttcccccacactgctcactacccttcccagtctctggtagccatctttctctctcatgtgtgtccatgaattcagttgttttaatttttagatcccacaaataagtgagaacatgtgaagtttctgttcctatgcctggcttatttcactttatataataacctccagtttcatccatgttgctgcaaatgacaggatctcattattttttttggctaaatagtattccattgtgcatgtactgcctttgtgtatatgtactacattgtgtatatgtaatagattaatttatccattaatctattgatggacacttaggttgcttctaaattttggctatggcgaacagtgttgcaataaacatggggtgcagatttatcgctgacatactgacttactttattttggttatatacctagcagtgggattgctggatcatatgttagctctaattgtagttttttgaggaatctacaaactaactgttctctatagtgttgtactaatttacattcccagcaacagtgtacaagagttcccttttctccacattcttgccaacatttgttgttgtatatcttttgaataaaaccattttaactggggtgagatggtatctcattgtagttttgctttgcatttctctgatgatcactgatgttgagcaccttttcataagcctgtttgccatttatatgtcttcttttgagaaatgtttgttcagatcttttgcccgtttttaaattggattattagattttttttttcctatagagttgtttcagttctttatacattatggctactaatcccttgtcagatggatagtttgcaaatgtttcctcctattcggtggtttgtctcttcagtgttgtttcctttgctgtgtgaaatctttttaacttgatgtgatcacatttgtccatttttgctttggttgcctgtatatgtgggatattactcaagaaatctttgccaatatttaatgtcctagagagttttctcagtgttttcttcctgtagtttcatagcttgaggtcttagatttaagtctttaatccattttgatttgatttctgtatatgatgaaagatagaggtttagctttattcttctgcatgtagataaccagctttcccagcaccatttgttgaagagaccgtctttttcccagtgtatgttcttggcacctttgttgaaaatgagttcactgtaggtgtgtggatttgtttctgggttctctggttctgtttcattggtttatgtgcctgtttttaagccagtaccatgatgttttggttattatagtattgtagtataatttgaagttaggtattgtgattcctccagtttcgttctttatgcttaagatagctttgtctattctgggtcttttgtggttccatataaattttaggattttttctatttctgtgaagaatgtcattagtattttgatagggattgccttgaatacagattcagtggaataccccactgctttaggtagtatggacattttaacaatattgattcttctaattcatgaacatggaatatttttccatttttttgttgtcctcttcaattatcttcatcagtgttttatagtttcctttatagagatctttcacttctttggttaattgctaggtatttgattttatgtgtggctattgtaaatgggattatttttaaatttccttttcacattgttcactgttgacatatagaaatgtgaactgatttttgtatgttcatttttgtattctgcaactttactgaatttattgattgtggaggctctaggtttttacaaatataagattgtatcatttgcaaacaaggataatttgacttcttcctttccaatctggatctcctttctatctttctcttgtctgattgctcgagctaggacttccagtgctatgttgaataacggttttgaaagtaggcattcttgtcatgttccacatcttagaggaaaggctttcagtttcttccccattcagtatgatactagctgtgggtctgtcatatatggcttttattatgttgaggtatgtgttctctatacctagttttttcagggtttttatcacgaagagatgttgaattttaagaaatgttttttcatcgtcaattgaaatgatcatgtggtttttgtcctccattttgttgatgaggcatcacattgattgattagcatatgttaaaccatccttgcatccctgggatgaatacttcttggtcatgatgaatgatctttttaatgtgttgttgaatttggtttgctagtattttgttgaggatttttgcatcaatattcatgagggagattggcctgtagttttcttttcttttttctcttttttttttttggatgtgtctttggtttactggtcttgtagaaggagtttggatgtattctctcctcttccatttcttggaatagtttgagtaggattagtattagttcttctttaactatttggtacaattcggcagttaagccattgagtcctcagcttttctttgctgggagactttttattacaacttttaccttgttgcttgttattggtctctttcaattttggatttcttcctggttgataggttgtatgtttctaggaatttgtccatttttttctagctttttcaatttattggtgtaaagttgctcatagtagtgtctcatcatcctttaaatttctgtgctataggttgtaatgtctcccttcttaatctctgattttatttacatgagtcttctatttttcttagtctggctaaaggtttgtcgattttgtttatcttttcaacaaacaaactttttgtttcattttgcgatgtttttctcatatcaattttatgtatttttgctctgatctttatttctttttttctgctaattatggatttcatttgctcttttctagttcattagcatacgttgttaggttgtttatttgaagtttttctatgtttttttaatgtaggtgcttatacctataaactttcctcttagtactggttttgctgtatcccgtaggttttggtatgttgtgtttccattatcatttgtctcaagaaatttgtaaatttctttcttgatttcttccttgacccactggtcattcagaaacattatttcatttccatgcatttgtatagtttccaaagttcctttcattattgatttctagttttatttcattgtgatcagagaaatgacttgatataattacaattatttttgaatttttgaacacttgttttgtggcctaaaatatgacctgtccttgagaatgatccatgtgctgagaacaatgtgtattctgcagccgttggatgaaatgttctgtaaatatctattaggtccattcaatgtatcttcgttgatttttctgtctggatgatctgtccaatactgaaagcggggtgttgaagtttacagctgttattgtattaggttctatctctctctttagctgtaagaatacttgctttatatatctgggtactccaccgttgggtccatatatatttacagttgttatatcctcttgctgaattgacctctttatcattatataatgacctttgtctgtttttataatttttgtcttaaagtctattttgtctgatatgagtatagctgttcttcctctcttttggtttctatttgcgtgggatatctttttccatcctgttatttttagtctattcatggtctttataggttaattgtgtttcttgtaggaaatagatggttgggtcttgtttttttttgcattcattcaaccactgtatgtctttggataggaggatttagtctactgctttttattttttattttagatttgcccttttgaggctattttctagatcctgtaggcatgctttattcttttttattcctttttttcttttgtcttctctgactgtattttcaaatagcctgtcttcaaactcactgtgtttaatcaattttgctgttaagagagtctgatgcattctttagtgtgtcaggtgtatttttctactccagtatttctgcttctttttaattatttcagtctttttgttaaatttgtcttataggattctgagttctttctctgtgtcatcttgaatttctttgagtttcctcaaagcagttttttttttttttttttttttgagatggactcttgctctgtcgctgaggatggagtacagtggtgcagtctcagctcactgcagcctctgccgcctgggttcaagcaattctcctgcctcaccctcccgagtagctggaactacaggggtgcgccaccacacccagctaatttttgtatttttagtagagacggggtttcaccatgttggccaagctggtcttgaactcccgacctcaggtgatctgcccacctcggcctcccaaagtgttggaattacaggcgtgagccactgcacccagccaaaacagatatttttgaattctttgtctgaaaggttccacatctctgtttctccaggattggtttttagtgtcatatttagttcatttggtgaggtcatgttttcatagatggtctagaagcttgtggatatttgttggtgtctgagcattgaagaattaggtatttattgtagtcttcacagtctgactttgtttatacccatccttcttgggagtgctttcaaggaattcaaagggagttgggtgttatgatctgagtttttggtcactgtagccatatctccattagggggcacttcaagcctagtaacactgtggctcttgcagacttgtagaggtaccaccgtggaggttgtattagtccattttcaaactcctagaagtatgctacctgagactggttaatttataaagaaagggagttcaattgactcagttctgcatggctggggaggcctcaggaaacttacaatcatggcagaaggtgaagggaaagcaaggcacgtcttacatgggggcaggagagagtgagatctcagggataactgccacgttaaaaccatcaagatctcatgagaactccctcactatcacaacaacagcatgagagaaactgcccccatgatccaatcacctcccaccaggtccctccctcaacatgtggggattacaatttgagatgagatttggctagggacacagagccaaaccatattattccactcctggcccctcccaaatttcatgtccttttcacatttaaaaaccaatcatgccttcccaatagtcccccagaatcaactcattctagcattaacccaaaagtccaagtccaaagtctcatgtgagacaaggcaggtaggtcccttctgcctgtgagcctgtaaaataaaaaacaagttagttagttccaagatacagtgggggtacaggcattggataaatgttcccattccaaacgggagaagctggctaaaacaaagggattacaggccccatgcaagttgaaaacctggctgggcagtcattaaatcttaaagctccaaaatttcctttgactccatgtcatacatccagggcatgctgatgcaaggggttggctcccacagccttgggcagctccacccctgaggttttgcagggcacagctcccacagctactgtcatgagctggtgtttagtacctgtggcttttccaggtgcacagtgcaagctgttggtggatctaccattctgaggtctagaggatggtggccctcttctcacagctccactaggcagtgcccaagtagggactattagtgggggctccaactcacattaccagtctgcatttccctagtagaagttctccatgagtgctctgcccctgcagcagacttcttcctggacatctaggcattcatatatcctctgaaatctaggtggaggctcccaaagttcaactcttgtcttctgtgcatctgtaggcccaacaccacgtgaaactgtcaaagcttggggcttgcaccctctgaagcaatgtcctgagctgtccttttagccacaggtggagctggagcagctgggacacagggcaccaagtcccaaggctgtacagagcagctgggccctaggcggggcccacaaaaccatttttccctcctaggcctccaggcctgtgatggaagggactcccgttaagatctctgacatgccctggagacattttccccattgtcttggctattaacatttggctcctcgttacttatgcaaatttctgcagccagcttgaattccttgaatgggtttttcttttctactgcatagtcaggctgcaaatttttcaaatttttatgccctgcttcccttttaaacataagttccaatttcaaaccctctctttgtgaatgtacataactgaatgctttcagaataagccaggtaacctcttgaatgctttgctgtttagaaatttctcctgccagataccctaaatcatctctttcaagttcagatctccagggcaggggaaaattgctgccagtctctttgctaatgcatagcaaaagtgacctttatgccagttcccaacaagtttctcatctccatctgagactacctcaacctggacttcattgtccatatcactgtcagcatttttggtcaaaacctttcagtaagcctctagaatgttccagactttcccacatcttcctgtcttcctctaagccctccagcctgttccaatctctgcctgttacccagttcccaagttgcttccacatgttcaggttatctttatagcagtgccccacttctggcaccaattctttgtattcattttcacactgctttaaagatactacctgagactggtaatttataagtaaaagaggtttaattgactaacagttccacatggttggggaggcctcaggaaacttacaatcatggcggaaggtgaaggggaagcaagacacatcttacgtggcagcaggagaaagagagctcaggggaaactgccacttctaaaccatcaagatctcctgagaactccctcactatcgtgagaacaaggtgggaaccacactcataatctaatcacctcccaccaggtctctgcctcaacatatggggattacaatttgagatgacatttggttgggtactcagagccaaaccatatcagaagtttctgataagatccagaagaattctctggattaccaggtagagactcttgttcttttcctttactttctcccaaagaaatggagtctgtctctgtgctgagctgcttggagctgctgtgacacaagcacccctgtggccaccaccagtgggactgttctaggttagacctgaagccagcacacactggttctcgcctgaggtctacaggaaccacttgctggatactgcctatgtttgttcaaggccctggggctctacaatcagcaggtggcaaagccagggagtcttgtgtgctttccttcagggtggcgatttccccctggccctaggcaggtccagaattgctgtctgggagccatgacctgaagtcagaaaccttagtgctttattctgctgtggctgaactggcatccatgccacaagacaaaaagttcttattctttcctcttttccacaaacagaggagtcactccccatggccaccactgccccaggcttgtggtgactgctggctgcctaccactgatattcattcaaggcccaagggctctttcgtcaccttgggtgaatgcttctaggcctggaactctcacttcagggcaatggcctcccttctggcccagggcaggtccagaaatgccatccataagccaacacctggaatcaggaactcctggagcttactttgtactctacccctctgtggctgagctggcacttaagctgcaaaacaaagtcctatttaatcttccctctccttttctcaagcaaagggagtctctcctcatagccaccatagctgggaaatgtgctgggtcacacctgaagccagcacatctctgagtctcacccaggtgagtactgcctggctaccactgctcactattcaggacccaagtactctttagtcagcaggtgatgaatcctgccaggactgggtccttccctgcaaggcagcaagttcctttctggcccagggtatgtctagaaatgtccaggaactaggtcatggaatggaggcctcaggactctgcctggcatctctatcattctgtggctgagctagtatccaggttgcaagagaaaggtctgtttaatcttccgtcttccgaagtgaaaggaaggaccctctcctggagcaagctgtgctgtctaggtttgggtatggagtggtgcaagtgcttcccttggccattttggctggtgtcttactaggttgcatacgcccaagtcagctggctctgattccagcacatcaccaagactttcctgggaattgtgtttcttattgctttaggctatggtatatgctattttatcacagaaaccctaaattacaggtaagtagtctagagcagatagctgagttgcatatctggtggctgtggcatccttccatctctggaagtgtgacttccatttctgggtctagagtaactgctacagctatcatgatctgcagtatctaggaagtggggggctttaatgggatacctttactggaatgcctggaagtttcacacattcttgcactcttcccatgggctgaacaaagttacccatatgttcacacatggctgtaagggagattggaagtgtatatagatgtataggtgtaaggaggtagggaagttggacacgaggtcatatttgacagtttctaccaatcatgaaagaaaaactagtatatagaaaaaaaatgtccataatagtaagatgagggcatatttttaaaatgagattccatttacctctattagcaattaatcatttcttaatgactatacacgtgctgttggaggaagagtaaattgtcgcagacatattaataagcaatttagcagtcagtacttttttgaaaacttcatgaatggtgtaatttttcactttgtaaaaagaatttaccttggatgggtgatgggggagaataagtataaacaggaaaatctttattcacagaattctttataattgaacattttaacacaacctgcattttcaacattaggctaagagttgactatatttaatctggtataaccataaaaatgtgaagaataggaagtaaaatgaatatttttattataaataatagaaaagctgaatttcaaattatgcatactatatgcagacaactttgtaaacaaataaaaaacaggaaaagatgggtaagttatacattaaaatgttaacaataattatccttgagtggtaggaccttgtaatttttacgtttatatattttcttaatattgaaaattcatactcttgcagtggaaactatgttttgaagccctgtcacatagatgagaattagacttattttctatgactctggaagcagaataagaccagtataagggcttaattatgaccaggttagggttcgatgtaagataacatgttttttagttagagtcatctatatatggaatggattgctaaaaagtcatctcaatgaaacatatccctaccatcctacttaaactgtttcccctgcatttaaaccccaatttctcctgtattcttaattaccttcagcatttctatattttccctgtatcacttataatgtattaaaatgctagataatttactcatttagtatgttaattatttactgtatattttcctcctctagaattctcattcttcttgaaggcagagatacatttctctttgtttactcataaattccaaaagtcatgaacactgcctgaaacatagaaggcattccatatttgttcaatgaattaacaaatgtttttcatattaattgttctgatttttacctccttaggccagcctttattattgccatataagccttctggtagtaccaagatgtattatgttccacaattaagacaaattcctccatctccggattccaaatcagataccaccgttgaaagctcccattcaggtattatgcagaaattattcgaagttttattgtttgatattttatttgtgtattggctagttgattctggagtgacaaaagtccagtgctctttaatttttcagtgaaatatggaacttttcataccttgatctctggttcagataggtgagttatttgatcagatatatacacataagcatttataaatatttgatgaaggaaattgtatggatcattatttgggttagtgggctccatctagggcaaagtttctaagctttcttggttcacagtacccttagttgtttcactgatttattttctccgctttatatataataagcaattatttttttcaaatagcaactaaggaattagaacataaatactaattgttttcctctgacaactaatttaacaaatttgttatttatttttggttttggtttttgaaaaaaaaatttgttcataaataacaatgatgacttactaatgacatatgtgtagctgttggacactgtgcaacttcccaaaccatgagatgagattggaccctcttattttctgccttttattacagcagctgccaaaaatccagcttttgccaaggcacaacagcattgaaaagaacatagcactatctgatgttgacactttgaccttgagctagtagtttatcactgtcctttccctagaattttaaactatcctgcagcatccctgtgaatccactacagaggaggtgcagagttaggaaatgaataataccccatggaggcactagataaatatttcctatggagattttaagttaagggtgcagagttagtgtggtgtagggttttgattggtgggacagtattttgagttgtcgacgggatatgacttgattcactttagacaaattgttttttccatgattgtctataaatgtcattatatatatagaaattacaaagaaaggagaagttgcccctggtcttaggcagacatttttgttactttgatatgaaagagaaccaaactagcttctgatagtaactgcactaaagcctctgatacaacctcgcctgatttcccacaatgatggcattgaaaaaaaagaaccacaaaaatactcaccacaagttaagattgttaagtttatagccataaatgaagaaatgtttactccttgcaaggctatggttttaaaaacactgttaatggctaccagaaagaaaacaagctcacttaattgattgtaggtaggaaaatcttctgtcccacttactcatagttcagaagacagaatgtctgtaaacagtagaaacctggtcagatgtgccagtactttctgaggcctccagtgtcctgctatcgatatacgtcactaacacttccatctggttggagagattaacttagagaaagactgaacagtgtaggtgatgggagaagaaaatgactcaggactggcacactgtatcctgataaatatagtttctgtaataaagtactgtatgcatcagtacgacaaaaagagatctagttgccatacacacttgagattgttttttcatgttttcatctattctgagagtgaggaagttgggaatagaaaataatcctttcttagaccagtagacctacactggatgaaagcctagaaattgttctaattcctagccgttatctatgaatatgttgtacatctatagctatgagaggagccaattagagtttggggccaagagcaagacatacatcattgttagcaaatatttgggccaaataactgttcttcagttgagaatcagtaaataatagaactgatgcaaacaacgacggcaaaaaactggggcaaaggaaacagccatccaaagaaagagataaatgtctgcaaataaaccagtcttaatagcaacccagggagtatttattcaagaaaaatggccaaatcccactaagcacagtaagcgttagcattttaacttgccctagtcccattcccagtacctcagctcagtgatagccttcagaaatatagctttcttttccaagaccagaggatgcaaaatggagctgggcctcttttaaaagttccttcccaaagaattgttaatatttgacctatctagtggttccgtgaagattccacacaaaaggcttatctttacttgacctgactgggagccatccagtgctaaaggcctctggtgaaggggagggaaagatgtttatcaaaaacatttacagacaagtattttaatatcataatggctgaggtgtggactagcagttgaggtaaacagtaaactaactggaaagcttaaaagtaacacccgaggataagatgttgatagattctttgaaaggcgctgacatattcctaagactctgggagaccccatgtatgcgtaggcctgtgtatgctcaggaaagacccaaggcgctaggtcttatctctggctgaccttgaggatctgtacaagaagaaaagggaaggctaaggtaaagttgacagctgcctaggtcagtgttgaaggtacatcccaacatatgtgtggagccatttgatgaaggccgggagatttattggttctaggcatttaaggaaatccctgttcaggcattagctgaccattaagctaattgagtagtaacttcagtggccatatataaaaagaatatagactttacagaattaattcagagaagttattaaaacaacataaagcaacaacaccaaaccctgagatggaagtagaatctggtttccagagttggcgcattgtattacttaaatgtccaggccgggtgcggtggctcatgcctgtaatgccagcactttgggaggccaagatgggaggattgcctgagccctggagtttgagatcagcctgggcaatgtagtaagaccctgtctctacaaaaaattaaaaatcagccaagcatgatggcctacgcttgtagtcccagctactcggaaggctgaggcatgagtatcacttgaaccctcgagtttaaggctgcagtgaactatgattgcgccactgcagtccactctgggcaacagagtgagaccatgtctcaaagaacaaaaaaacaaaaaaacaaaggcaacaaaagaaaaatatatatgggagactttatcaaaattcaaaacttttgtacacaaaacccagtcaaaagaatgaaaaggtgactcacagcataggagaaaatatttgcaaatcatttatctgttgagagattaatacctaaaatatgtaaggaacccttacaactcaacaacaataacccaaacaatctgattcataaatgggcacagaatctgatggacatttctccaaggaagacgtgcaaatagccaataaacacctgaaaatcttctctatatcattaatcatgagggaaataccaaacaaaaccatagtaagataccacttcacacccattacaatcactgttattttaaaaaataagcggagagagccaagatggccgaataggaagagctccagtctgcagctcccagtgtgagcgatgcagaaaacgaatgatttctgcatttccaactgaggtactgggttcatctcactagggattgtaggacagtgggtgcaggacagtgggtgcagcacaccgagcgtgagccgaagcagggcaaggcatcgcctcacccgggaagcgcaaggggtcatggaattccctttgctagccaaggaaaggagtgacagaaggcacctggaaaatcgggtcactcccaccctaatactgcgcttttccaacgggcttaaaaaatggcacaccaggagattatatcccgcacctggctcggaggatcctacgcccacggagcctcgctcattgctagcacagcagtctgagatcaaactgcaaggcggcagcgaggctgggggatgggcgccctccattgccgaggcttgagtaggtaaacaaagcggccgggaagctcgaactgggtggagcccaccatacctcaaggatgcctgcctgcctctgtagactccacctctgggggcagggcatagccaaacaaaaggcagcagaaacctctgcagacttaaatgtccctgtctgacagctttgaagagagtagtggttctcccagcacacagctggagatctgagaacggacagattacctcctcaagtgggaccctgacccctgagtagcctaactgggaggcaccccccaggaggggcagactgacacttcacacggccgggtacttctctgagacaaaactttcagaggaacgatcaggcggcaacatttgctgttcaccaatattcgctgttctgcagcctctgctgctgatacccaggcaaatagggtctggagtggacctccagcaaactccaacagacctgcagctgagggtcctgactgttactaacaaacagaaaggacatccacaccaaaaccccatctgtacgtcaccatcatcaaagaccaaaggtagataaaaccacagagatggggaaaaaacagagcagaaaaactgaaaattctaaaaatcggagcgcctctcctcctccaaaggaacgtagctcctcaccagcaatggaacaaagctggatggagaatgactttgacgagttgagagaagaaggcttcagatgatcaaactactccgagctaaaggaggaagttcgaacccatggcaaagaagttaaaaaccttgaaaaaagattagacgaatggctaactagaataaccaatgcagagaagtccttaaaggacctgataaagctgaaaaccatggcaagagaactacgtggcgaatgtacaagcctcagtagccgatttgatcaactggcagaaagggtatcagtgatggaagatcagatgaatgaaatcaagcgagaagagaagtttagagaaaaaagaataaaaagaaacaaagcctccaagaaatatgagactatatgaaacaaccaaatctacgtctgattggtgtacttgaaagtgacggggagaatggaaccaagttggaaaatactctgcaggatattatccaggagaacttccccaatctagcaaggcaggccaacattcacattcaggaaatacagagaatgccacaaagatgctcctcgagaagagcaactccaagacacataattgtcagattcaccaaagttgaaatgaaggagaaaatgttaagggcagccagagagaaaggtcgggttacccacaaagggaagcccatcagactaacaactgatctctcagcagaaactccaatccagaagagagtgggggccagtattcaacattcttaaagaaaagaattttcaacccagaatttcatatccagccaaactaagcttcataagtgaaggagaaataaaatcctttacagacaagcaaatgctgagagattttgtcaccactaggcctgccctaaaagagctcctgaaggaagcacgaaacatggaaaggaacaactggtaccagccactgcaaaaacatgccaaattgtaaagaccattgaggctaggaagaaacttcatcaatcaactaacgagcaaaataaccagctaacatcacgatgacaggatcaaattcacacataacaatattaatcttaaatgtaaatggactaaatgccccaattaaaagacacagactggcaaattggataaagagtcaagacccatccgtgtgctgtattcaggaaacccatctcatgtgcagagtcacacataggctcaaaataaagggatagaggaagatctaccaagcaaatggaaaacaaaaaaaggcaggggttgcaatcctagtttctgataaaacagactttaaaccaacaaagatcaaaagaggcagagaaggccattacataatggtaaagggatcaattcaacaagaagagctaactatcctaaatatgtgtgcacccaatacaggagcacccagattgataaagcaagtccttagagacctacaaagagacttagactcccacacaataatgtgaggactttaactccccactgtcaacattagacagatcaatgagacagaaagttaacaaggctatccaggaattgaactcagctctgcatgaagcagacctaacagacatctacagaactctccaccccaaatcaacagaatatacagtcttctcagtaccataccacacctattccaaaagtgaccacatagttggaagtaaagcactcctcagcaaatgtaaaagaacagaaattataacaaactgtctctcagaccacagtgcaatcaaactagaactcaggattaagaaactcactcagaaccgctcgactacatggaaactgaacaacctgcccctgaatgactactgggtacataacgaaatgaaggcagaaataaagatgttctttgaaaccagtgagaacaaagatacaacataccacaatctctggaacacattcaaagcagtgtgtagagggaaatttatagcactaaatgcccacaagagaaagcaggaaagatcgaaaattgacagcctaacatcacaattaaaagaactagagaagcaagagcaaacacattcaaaagctagcagaaggcaagaaataactaagagcagaactgaaggaaatagagtcacaaaatacccttcaaaaaatcattgaatccaggagctggttttttgaaaagatcaacaaaattgatagactgctggccagactaataaagaagaaaagagagaagaatcaaatagacgcaataaaaaatgataaaggggatatcaccaccgatcccacagaaatacaaactaccatcagagaatactacaaacacctctatgcgaataaactagaaaatctagaagaaatggataaattcctcaacacatataccttcccaagactaaaccaggaagaagttgaatctctgaatagaccagtaacaggctctgaaattgaggcaataatcgatagcttaccaaccaaaaaaagtccaggaccagatggattcacagccgaattgtaccagaggtacaaggaggagctggtaccattccttctgaaactattccaatcaatagaaaaagagggaatcctccctaactcattttatgaggccagcatcatcctgataccaaagcctggcagagacacaaccaaaaaacagaattttagaccaatatccctgataaacattgatgcaaaaatcctcaataaaatactggaaaaccgaatccagcagcacatcaaaaagcttactcaccatgatgaagtgggcttcatccctgggatgcaaggctggttcaacattcgtaaatcactaaatgtaatccagcatataaacggaaccaaagacaaaaaccacatgattatctcaatagatgcagaaaaggcctttgacaaaattcaacaacccttcatgctaaaaactctcaataaatgaggtattgatgggacatatctcaaaataataagagctatctatgacaaacccacagccaatatcattactgaatgggaaaaaactggaagcattgaaaactggcacaagacagggatgccctctctcaccactcctattcaacacagtgttggaagttctggccagggcaatcaggcaggggaaggaaataaagggtattcaattaggaaaagaggaactcaaattgtccctgtttgcagatgacatgattgtatatctagaaaaccccattgtctcagcccaaaatctccttaagctgataagcaacttcagcaaagtcacaggatacaaaatcagtgtagaaaaatcacaagcattcctatacaccaatagcagacaaaccgagagccaaatcatgagtgaactcccattcacaattgcttcaaagagaataaaatacctaggaatccaacttacaagggatgtgaaggacctcttcaaggagaactacaaaccactactcaatgaaataaaagaggatacaaacaaatggaagaacattccatgctcatgggtaggaagaatcaatattgtgaaaatggccatactgcccaaggtaatttatagattcagtgccatccccatcaagctaccaatgactttcttcacagaattggaaaaaacgacttttaaagttcatatggaacaaaaaaagagcctgcatcgccacgtcaatcctaagacaaaaggacaaagctggaggcatgacgctacctgacttcaaactatactacaaggctacagtaaccaaaacagcatggtactggtaccaaaacagagatatagaccaacggaacagaacagagccctcagaaataatgccgcatatctacagctatctgatctttgacaaacctgagaaaaacaagcaatggggaaaggattccctatttaataaatggtgctgggaaagctgcctagccatatgtagaaagctgaaagtggatcccttccttataccttatacaaaaattaattcaagatggattaaagacttacatgttagacctaaaaccataaaaaccctagaagaaaacctaggcaataccattcaggacacaggcatggtcaaggacttcatgtctaaaacaccaaaagcaatggcaacaaaagccaaaattgacaaatgggatctaattaaactaaagagcttctgcacagtaaaagaaactaccatcagaatgaacaggcaacctacagaatgggagaaaaattttgctatctacccatctgataaagggctaatatccagaatctacaatgaactccaacaaatatacaagaaagaaacaaacaaccccatcaacaagtgggcgaaggatatgaacagacacttctcaaaagaagacatttatgcagccaacagacatgaaaaaatgctcatcatcactggccgtcagagaaatgcaaatcaaaaccacaatgagataccatcttacaccagttagaatggcgatcattaaaaagtcaggaaacaataggtgctggagaggaggtggagaaataagaacacttttacactgttggtgggactgtaaactagttcaaccattgttgaagtcagtgtggcgattcctcagggatctagaactagaaataccatttgacccagccattccattaactgggtatatacccaaaggattataaatcatgctgctataaagacacatgcacaggtatgtttattgcggcactattcacaataacaaagatttggaaccaacccaaatgtccaagaatgatagactggattaattaaatgtggcacatatacaccatggaatactatgcagccataaaaaatgatgagttcatgtcctttgtagggacatggatgaagctggaaaccatcattctcagcaaactattgcaaagacaaaaaaccaaacactgcatgttgtcactcataggtgggaattgaacaatgagaacacatggacacaggaagggaacatcacacactggggcctgttgtggggtgggggaagcggggagggatagcattaggagatatacctaatgttaaatgacgagttaatgggtgcagcacaccaacatggcacatgtatacatatgtaacaaacctgcacgttgtcacatgtaccctaaaacttaaagtatagtaaaaaaataaataaataaaaaagcaaagaatggaaaattatcagtgttgtcagggacatggagaaattggaaccattgcttattgctggtgggaatgtaaaatggtatagccattacggaaaacattgtggctattcctcaaaaaattaaacatagaattatagtatgatccagtaattccacttttaaatatataccccaacaaagtgaaagtaggatcttcaagagctatttgtacacccatgttcataacagcattattcacagtggccaagaggtggaaacaacccaatgtctattgatctatgaatggataacaaaatgtgacatatatgtgtaatggagtattattcagccttaaagaggaatgaaattctggtacatgctacaacatggatgaactctgaagacattaggccaactgaaataagccagtctgcttatatgaggtacctagaaaagtcagattcatggagacacaaagtagaattgtgtttactaggggctggggctagggggagtgggaagttactatttaatgggtacagagtttaagtctgggaagatgagtaggttctggagatagatggtagtgatggttacataacagtgtgaatatacttaatgccactgaagtgtacacttaaaaatgattaattttttgtggtttttttttttttttttgaaacagagtcttgctttgttacccaggctggagtacagtggcacaatctcagctcactgcagcctccgcctcccagattcaagcagttctcctgccttagcctcctgagtagctgggattataggcacccaccaccacgcccagctaatttttgtatttttagcagagatggggtttcaccatgttggccaggctggtctcaaactcctgacctcaagtgatcttcctgcctcagcctcccaaagtgctgggactataggcgtgagccaccgcacgtggtcatggttacattttatgtatattttaccacagttaaaaaaacttaagaagttaccaaaaatcttaattgaattttaaaagtagatgtctatgttccaaatcagggttgagtatacgaaattagcaaagtaacagaacgagaaatacaggaaactttaagatagcataaacagttaaatcagatgagtggaattcggtgagatgtctttagctgtgataaataattttagcatatgttgatattgtctcatgcatgtatttaacttaagcaagttcaaataaatatacttaacaaaagaggagagaaagaattttaaaagtgcaggtgttttgtgcatgcactctaccctttatgaacatatgactacggaagaatatgtaatgaaagatgggaaacttcattctcaaaattaattgtagtcctcgtttgccttttgtagtgtaggcatcttatcttgcttatttgggtatgattctagagtttgaagaaatgtctttttggtacaatatcagatacttcatcaggtgctcagattcaggtgcagtgctatgttacaagcttggaggaaaaagtggaccctcatgggtttcttgagcagacacgatgtcaatttcaacatatgccttcctaaggtaccttcaagggtgattttgaccctctatagcaaggttttcacctcacatgctgactttaaaccctgaattccatttaaaatgtatctctactttagtgaaaaaatagtaatgagactgtatatggtatgatccctgtaaatgttcacttagtaggatatataaccaaggtttagttgggttttcttttattgtttccttcatactttcctgaattttctacaatgatcaggttgttttgtttgtttgtttgtttttaatgatgggaagctgtcagctcacgttcatttgtttattcaaacaataggtattaaagatgttactgtgttccacgcagatacactgtggtaggtgttacaatttcagtattgctcaaaataaactataatggagtttatgttcacgttggttgacagatattaaacaaataaaccataaataagtacgtaattacaagtgataaatgctaagaaaataagaagagttccaagataaacaagtagacccagttttgttttacgaacttatgaagatcttttccaggaagaaacttttgagctgaactctagaggatgggaatgaattaggtaggcagagagtggagtgtgagaatgttccggcgagagggaagaagatggggaaagcccctgaagcagaaaagagcttgatgcatttgacaaatggaaagaaggcagcgtgggttgaaattccaacaaatttaaagatctgattggtttttgtagtgattcatgaatcaggcaacatcccattctataatatggaaagggtgcactgggcatgggagagagttggttctggtaaggtagccagaccaggaacaaggaaacacaatacagacagtccccaatttacagtggttccgttgaactattttttgactttatgatggtatggaaatgatacacatttagtagaaaccgtacttcagtacagtatttaataaactatatgaactatttgatactgtattataaaatagacttcatgttagatgattctgcccacctgtaggctattgtaaatgttctgattatgtttaaagtaggctgggctaggttatgatgcttggtaggttaggggcattaaatacatttttgactaacagtattttcaacctatgatggacttaccaggacataactacattgtaagtggtggatcatctgtaataccaaaaaaagcatattgattaacatcaggttacttacctggtaaagattaaagcagagataagttcctttttacgccagctcagggtgactggcccctttttcagttagttgctgtgaatctcttgtttttagaaaaaactggtccattttggaactttccttctttcttaaagtttcagttggattgtatggcatttagtacaagtgactacattttggtttggtctcttggggcttagtgcaggagctcagtctaaaacaatggactcccataaattttatttaacagggtagagcatagtgatgggtagatatggttaaagataggtgggttcaagatcatgcaggatcttatcaactgtattaaagtgttcggattttattctagtactactgcaattatggtttgatttatacttgtaaagtttattcttgccactatatggagaaatggtttatagatgggaaggcggagagccatggcggggcgggttagagtgcaagtagagacaccagttagttagaaggctgttgctttagacagtaccagagatggtagcatgagtatcacagtgttatctatagagatagggagaaatggacagtcagcatccttttagaaatagtttctcctagatttggtgataggagtatggagagtgagggagaagagcgtgaaaaaaatgtctggcgtgagcaactaggtgaatagaggtgccatttactgaggtggagattgaggaaaaagcagatttggggaagatgatcaaaatgtctgctttggacatgttaaaaatgagattcctattatacatccacatagagatgttgggtctacaattgaatgtaggcagagttaatttacacatgttaaggtaatagtgccacatataaggtggcattattcacattataaatatatacttttatgaaaacatacagatgggcatgaatattccagatctttagttagagttctggagcaataatgttaactcttgtgctgccccaaacataaatctatactcgcacatactatagctatgcacaggtacaggaaaataagtatatgtacattcactgacacgcacacatgtgtggctgcactctctcagctgcctctcacctcctctgaggttgattctcttgcttattcacccactgctctcagggatgtaattgttcctcacctgactctgaacttgaaagggtcatctctcttggagaacgaggggtggtggtgcagactgtgcgtgcacatgctgctgctggtgtgtactccctgggagaatttgtgaaaagaatgaccaccatttacagttgtttgtgttgtactacaatatatagtaacttaaacattttgatttgaagaagacacatcttttctaatttccataaaagtgctgtaagagctagccgtgtccctgaatgctggagtcggatgctcagaagaaaggtactcttgggcaatggtacattatttgttggtcatcagcatataccttggtggtcgataagtttacctaggaagagaagagaggccagttccgactccttaaggaactccaccattgagagatttggtagaagaaaatataaaaaataagtttccggagaggtaagaggcaaataagagaaagtgatgttacagaagttgagaagaacccaatataatctatttggcccagaaggcttggtgttttccttgcagtctgtcctgcaaaactcttcagggcttactgtgcactgtactaatgatacaggtacttacccagctcttattgccatatacatactaggagctcaggaaatagaggatcttagaaaaatcatctcataggagattagtataagcctttatatgtccagaaatgttcttttgcctgttagatgtggcatggattatagattaatttattttatcaaggaatgtgggaatattgaaatatattttaaaagaaaattaaatactttcaaatcttttacttatcatctgtcattttccattcagtgttttgtcttttgtattgtcagattaatcttctggaaacatagctaaatatattattattttgttgaaaaatcttccgttatgttgcattttatatacaataaagtttatactcttcagcttgggatcccatgcactggtgaattttgtcccaaacaactattttttaatatcttctaccactgtccttaatgtattctgcaatccaacctgaccataaagtgtccactatttttaaatacttcttttctttgcttatgctataacttctgcctaaaatgcctttcttccttcctctctatatcaaaaaactactttttttccaagatacaatccattttctccttcctccaaaaccatcatgagctggaaatattctctctcctgaactgattctccattttacatcttcttttatatcatttgcatctctgccttgtattattagaatttattttacatggcttacaacttcagtttgactgacgtttccttctgattatcctacccattgtagtgtagtgtttttttgtttgttcgtttgtttgtttgtttgttttttaagaccaagtctcactctgccactcaagctgcagtgcggtggcaacatctctgctcactgcaacctctgcctcccaggttcaagcgattctcctgcctcagcctcccaagtagctgggattacacgcacctgccaccacgcctggctaatttttgtatttttagtagaaatggggtttcaccatcttggccaggctggtcttgaactcctgacctcaggcaatccgcctgcctccaccttccaaagtgctgggattacaggcatgagccatcgtgcctggcctgtagtgcttttttcatagaattggtctaagaggcaaatttttaaaaagtctttctcaatctcatgtcgctatttgtgttttgtattacttccccgtttttctgtaggatccaatgatgccattgctccagacttcccagctcaggtgctaggcacaagagatgatgacctctcagccactgttaacattaaacataaagaaggaatctacagtaagagggtagtgactaaggcatccttgccagtgggagaaaaacccttgcagaatgaaaatgcaggtaactggattggctttgtatactttgtagctttttctccccttttcatcctaaatgagattactatccaatctttatcctttttttctgttttgttatacaagcttttctccttgctgttatattgagcatatacaagaaggcaaaatttcataaaatcctttaataataaaggaagcataagcttcagagatttttgaaaattataaacttcattttcacaacggataataaaatatgatacaacttttgaaatgaaataatacccaatcaagtatgttttagatactaagatccacagaaaatcttgattaaatgccacattagtttatctctgtcagatattacaaagtaatgtcttataggctggattctgtagggagatatgtagctgacatgattttttttttttaatttttttaaagacagggtctcactctgtcacccaggctggagtgcagtgatgcaattatagctcactgcagtgttgaactcctggactccagtgatcctcccacctcagccttctgactagctggggccagaggcacacaccactatgcctggctaataatttttttatagtgatggggggttttttgtagtgatgggggtctcgctatgttgcctgggctggtcttgaattcctggcctcaagctgtctaccagcctcagactctcaaagtgctgagattacaggcatgaaccaccgcatccagcccccatgttgttttaaaatttttaaaattttccaccaacattcaaaactaagatttttgattttccttgaaaaatcagaaaatctgactgccctttggctcgcattcattcatggtggtaatcagctgcagctgagccatggctgtccccctgcaggcaggacatccattctcttgtttgcttccatctccacagctctgagacccagtctgcttcattcatccaccttatctgtctgctgatcactgtgggtatttgagtttcaatcttttttttttttttttttttttttttttgagttggagttgcgatcttgttgcccagggtggagtgcgatggcgcgatcttggcacactgcaaccttcacctcccgggttcaagcgattctcctgcctcagcctcgagtagctgggtttacaggcacccgcctccacacccagctaattttttgtatttttagtagaggtggggtttcaccatgttggccaggctggtcttgaactccagacctcagatgatctgcctgcctcggcctcccaaagtgctgggattacaggcatgagccaccgtgtctggcccccactgttttataaaagctttttttcttctgcacccctgccattatacttttgtagctgatagatgataatatttattaaatgaaaactccagtagccactgaaaattctgtgtgtataattgcctgtgtttgttacatttatacttttttattcctgtgtataactattagctaaataggtgctactgatcccagggcagtttttaaatgtagggtggctctatgttctagttttggcatagtgttaatgactttgtgtgatagcatcagtgcctttgtgagaaaaataaatgagatctgcatggttttttttattttattttattattgttatactttaagttttagggtacatgtgcacaatgtgcaggttagttacatatgtatacatgtgccatgctggtgtgctgcacccattaactcgtcatttagcattaggtatatctcctaaagctatccctccatttttttagataatttcgttctacttcaaaacacagtgaagtctttttataaatcccttttgaaataagagaagcatatctaccagctaacattgagactgccaagtaaataaaaagaaataattttacaaaatatagttcataaatgaaaactcttctgcaccagggaatccgttacgtgttttccactatccatatgctagtggcagtttggttgaaacagataagcctaagatcccagtgtcagatacttcaggtgagtgtagatgtggaaaaattggcaggaaagaagtgaactctgtcagaagtctgcacaaagaactcaccagctttacctcactcccaggcttcatttggtaaggatgatacatttgaaataaagtcaggctcagttatctgaggacctctacaatctcccatctgttggtctacttacagactttgcatcctaaggaaaagccctcctcctggcttagtccttagtagccatttcaccttgagagaagcctagcctcagaacctcttgccttagagaattaaagaagaatagaggagagaatgcctgcctgggaaactgaacaatgacctatgtaatagctaaatgagctcataaaatatcatatcataagtaaaattctcagtggaaggggtggaatataaaagtgagaaaatctcttcaaaaatagaacataaagagaagggaaaagattttttaaaattcaagattgtccccaaagtttcaacattcaaagcaggtattctgtaaagacaatagagaaggcaaaaaagaaagaaccaaaggactagtataggaataaatttcagaactgaaagatgtgagttttcatactaaaagggactaccatgtacataaaaatacacaccgaggcacatgaccttgacattttaaaactgatttgaaaagagattctaacagcttccaaaaagaaaacatacattccatataaagaaatggaaatgagactggcatcagacttctcaaaagtgataaacagagttagaaaccaaaggagtaataccttagaattctgagagaaaattatttcactaaaacaagggagtttatttaagttacaaaagaaggaaagaaagaaaacatggtgggattgaggaaatggagggggccaacacaggaaatgggaaacgaattctcactgtggcggtggtacacaaggcctagaacccattcagtttgaagcctgcaaggaaaaagtcgaatttgtaggataccttatgtgattgactatacttagaggaggttttcagttctatttgcagctatgaggatgaagtagggattggcacatagaaactcagaaaatgcaaaaacaaagcggttattaactcttcaggaaagcagaagtttgtaatagaaaataaatgtaattacagtatactagctggcttagctgtgaataataattatataaacaatcatatgtaaatactaaatataaacttaattgaatattgatttaatcactcagaccattatttatgtgacatataaacactgaatgttaatatagggctaaaacttttcagaagataggatgaaaggatatatatgggtaggagggttttataatgagataaatccatagatcttagtatatagtataagaataaatcccagaactgaaagatgtgagattgtaattgatcttagaaggaaatcaatagataatttctggaacataaaatgatatggtagtataaagcatgttattaggaaataaggatattgatcctagaataaatggctaaagaagttgaaaatggtagcctttagaaattggggatctgtagtggataggagagggaactgctgacttttcttgtaagcctcgtgaaactatttgacttttaaaacatatacattgttatttttattaaatcagaatttatattgtccttcacctcaaaaaagtaagaagaaaaaaatacatttggcaaatactgactaaagaaaagctagtgttgatatggacaaaatataattccaggacaaaaaaaaaaaaattaaaggtagatacaagagtttatctcatagtaataaaagggtcaaatataccaggaaagataaaattgtgagcttatatgtatattctcaaatatattcaggaaaaattacaggaatgtgtaggtaaattcaacagtacatttcagaaatgtgaaagaccaggaaatagagaaagtagggctgagcgtggtggctcatacctgaaatcccagcagtttggggggccaaggcgggtggatcacgaggtcaggagatcgagaccatcctggctaacacggtgaaaccccgtctctactaaaaatacaaaaaaaaaaaaaattagccgggcgtggtggcgggcgcctgtagtcccagctactcaggaggctgatgtaggagaatggcatgaacctgggaggcggagcttgcagtgagccaagatcacgccactgcactccagcctgtcgtatttaaaatcaggtgttaccctcagcatgggggagatggaaggaagaatgaggggagcgtgtaagtttctagccagattttatttcctaacctgggtaaggttatatacacttgctttttgtaattcactgaactctttatgtgcacgttttcccccatattttccactgtacaattaaaaagatattttaaaaagcaatatggtacttagaaatgaatctaaaagggtctgtaagataaacaatacggtacttaggaatgaatcttaaagggcatgtaaaactattttaagagaaaattataaaactacattgaaatatattaagacctaaatagttggaaaattacacaattgtaataataaagatgtcactaaattaatttctaaattaaatgcattgctactcagaattccagtaggatttttcgtggaacttgacaggctgattctaaaatatttatggaaaatcaaaaaacgtatatttaaagaataagataagggatcttgccataccagacatcaagacttattacatgtgtagcttatttaagacatcatttacaaaggaataaacagaaagaaaaatgaaacagaatggtgacccagaaacatatccatgaatgtggaaacctaacatacacagagatggcataacaatctgtggagaaaggatgaagaggaactattcaatagagtttgaaccaatgaatatatatctggaaaaaataaaatcagataaatcactgccacacactaaacagaaatgtatagtataccaggcatattaaaagtaaatgggataactaagtttttagaatttagatggtctgtccttatgaccatgtgataataaaagatttcttaatcaggacacgcaaagcacctaccctaaagggaaaatataataaatatgccattattaaactgaaaaactttttgcctaaacgatatcaaacaattaaaagacatgaaacagcctgggagaagatatttataatgcacaccacctgcaaagacatttcacaggaatgttcattgcagtgcttttcaaaataggaaatagccttaagttgatcaacagaggtgtattcgtatatattaatacagtagaataatacataacgttcagaatcaaagacatatattaattgactaactctcaaaatcataatattgagtgaaaaagtcatggtgcgtaaaggtacagacagtattgtacccttcgtataaggtttaaagaaacttgcaaaacaatgctgtacactgttggttaatatatgcaatgtggtaaagaattacaaaacatccttgatattgataaactgtaaatttggtgtaaaggctgctactgagaagtgagaatggcatcattgggaacactttacacaagaaacattatatttgtaatattttatttcttaagctctatggtaaatacttgggtgttcattttgttgcctatatttttggtatgcctgaaatattttattaacattaaaacattttttaatgatctaaaaatttcagaagacatttagagaaaagagattaggaggattgaagtaataagaagtaagaaaaatgtgtgaatttaaaagaggtagcaagagttaactggttctttgcatttttagcaatctctattgcctttctgatttctttgttgaaaaatatacttacagataaaaaaatcaaaaatgatcaaaaaagtagaaaagagaaatagtattagacaagtttaaaaggctataactgcatgaaatttttaaaaaattgtatgagaatgctgtatttaaaatctagatgataaaagtgcttttcttggattatatgaaatataaaaattgactccataagtggtaaaaactttgaataggccaaagaacttgaaagcattgttcacatcttttttcctttgaaaaaacatacctgatctttcctttttttttttgtttttttttttactgttgcatcaaaccatcaaggaacagattttttttagaatgtttttattttattttatttttttgagacagagtctcactgtcgccccggctggagtgcagtggcacagtctcagctcactgcagcctccacctcctgggttcaagtgattctcctgcatcagcctcctgagtagctgggactacaggcgtgcaccacgacgcctgctagtttttgtatttatagtagagttggggtttcaccgtgttggccaggctggtctcgaactcctgaccccaagtgatccacccgcctcagcctcccaaagtgctgggattacaggtgtgagccaccccgcccggctttagaatgttattttatttaaactgttgttgtacacagtaaagtatgaaaaatttttcaattcatgtcacagtttttacacaggtggagcctaagaggtacttttttcctaatatgtaaatgggtttggggttttgtttgtaattgtgggggaggattacttgtctgttgtgtttattatctttccttttctgaaatcaaatgatgtcgttattccagatgcctcagttcaagtgctaatcactggggatgagaacctctcagacaaaaaacagcaagagattcacagtacaagggcagtgactgaggctgcccaggctaaagaaaaagaatctttgcagaaagatactgcaggtagctaaactggattgtctgcgtattattttcttctggtcttctaaaatgagactattcccttaagaacagaaacagaaatatattctctattttcttcttgctgtcttcagctctcttctatctcatgtatatatgaaatagttaaggtatgtttagctatattttttatatactttggcaggtaaaattaacatatttaaagttctttgtgatatttctggagggttttgaatatagtaaacaaattaatctaatttccagaaattcatttaatggactttttacctccgctgataaaatctctcaattctgtgagacacggaccctctaataccatttttgtacataatcctttatttaggaattcagcttgtttacttcctacctatcagttactttacttcaaagggattatgctgatttaaataaccctaatagtgtatttacgttaggattagttttgactcagagaggaaaacaaaagcaaaataacataggcttgaagaagacagaaaactctttctcttttttgtaaaactccagaggtaagcatccagagttgacctggtactccagagtatcggaacaaagctgttgctctcttgttactctaacatttttggccttcattcttaagctgtagccatcagcccatattgcaaccagcaggaagaaaggaaaggaaaaagcacattccttcctttaaggatacttctgagaagctatatataccattccttttgcattctattggccagacttagacacatggtcacatgtaccagggaagctgggaaattgagttgttattctaataacaatgtgcccagctaaagttagggttctgtttagaggaaaaggaaagtggatattagaggacagatagtattgatagtttctgccacaattgtaaactaaaacattttagaacatgattagctgcttctcttctttaggtattgcttgtttaaaaaggttagattgtattctgggcaacacttctgaattttaccccattttactattactaaatgaatgttccaaaaacagttttgatcatgttaatctcctaatcaaaaactttagtgtttcattgcctgtaaggataaaatctaaactcttttagcactgtcttctagaactgtagagtccactctcaaacaactttctaacactagttctcacctcttttccttataaattctataatcaaatgctatatacttgctctgtttcttagatgctaagttactttcctgacaccattcttgtgttatgtagtcaccttttttttccatcttaatgtgtatcccatcttagctctcttctaatgccaaaatcagttgtctagagttctatggtactcccacctgcaattagtttttctttctttgaataccgctacctctttttctgactgtttcttacagtatatctgtttacgtatgtgattttttgccccaattttcaattattttctaaacgcatttctgagtcatcttttttcttcaatatcagtaacaaagcctttcacataatacgtacttgagagacatttaaaaatcttttatgtcaagttcctgtctgtatagtgtgttaatttccctttcgtagattccagtgctgctgctgctgcagagcactcagctcaagtaggagacccagaaatgaagaacttgccagacactaaagccattacacagaaagaggagatccataggaagaagacagttcccgaggaagcctggccaaacaataaagaatccctacagatcaatattgaaggtaatgggattgggttgtgtatgagtgtgtgtgtctttgtgtgtatgtgagggtcagtgttagtgtttccatttcattctgatgagaatatagcctcatgattaaacaaaaattccttttctttttttaaaactttcttgtgtaaagtactttgatgctactattaggttactatatatatatatagatttccaaaaagtaattgttactttttggaaaagatattagcaacttagaaacaggattataaataaatacctaaggctgatagttttagaagtgaagtatcttgtctcagtccccagagacaaccttaaaggccttccagaagactatagcaaagatagtgagaccatcaatacaaacttttaaaaaattacaataaaatcacatttatttttgtttgtccatgggtctctttttctgacataaaagcgtatagaacacaagacattactacctagccatcaagctttacctcttagaggaagatggaacagcctacataaggagagaaaagagtcaaggagctaaactacccatactggattttggaaatagtctgggttcaactgcttacatcaagaatatacaaaataaatgattttttgacaatagccaagactacacaatggggaaaggatagtctcttcaacaaatggtgttgggaaaactagatatccacatttaaaagaataaaggtggaccttttctttacaccacatacaaaagttaactcaaaaagggttagtgacatacatgtaagaccttaaaaccataaaactcccagaagaaaacataaggggaacacttcaggacattgaacttggcagtgatttctcagatgtgacaccaaaaacacagacaacaaaagcaaaaatagaccaatgaaactacatcaaacttgaaaacttctgcatgacaaagtagacaagagtgtgaataggcattctacagaatgagagaagatatttgcaaatcatggatctgataagggattagtattcagaatatataaagaacttatgcaactcaatagcaagaaaaacccaaacaacttatttttaaaaacacgtaacttattttaaaatgagcaaaggacttgaatagacaatagaagatatacaaatgccatatcaagcatataaaaagatgcccaatctctctaatcataagagagatgcaaatcaaaaccacaatgagatatcactcacacctgttaggatgaccactatcaaaagaaaaaatagcaaatgttcgtgaggatgcaaggaaattgaaacccttgttcaccattggtgggaatgtaaaatgatgtagctattatggaaaatattatggaggttcctcagaataataaaaaagaattagtatatgatcccgcaatcccactgccactgctgggcatataacctaaagaattcaaaacaggacttcaaaaagctacctgtatactcatgtttattgcagcgttatttataatcaagaggtggaagcaacccacatgtccatcaacagatgaatgtattaagaaaatgggaattatttgtcaataaagaaagcatgttacatacatatttgtattgtgcagccttaagaaagaaggaaatcctatcacatactacaaggtaggtgaacctcaagaacattatactaagtaaaataaatcagtcacaaaaagataaataccatatggtttctcgcaaatgttgagggatttaggaggatgagagagaccttgggtttaaacaggagagtcttttattgagtgcactcaggcccagcagactcacgtccaaaggctgggcccagaacaaagacagcacttgacttttatacacacttcacaaaaggggatgggctagcttgaagcaagcttacagtggcgtgaaagcgggaatacagaagcaggacaaagacagttaatcaaattgtaacaggttcataactcaggattgcacataaccgttgctatgcaacccagatgtccattatctaggtttgtctaggcacgggcttatcccataaccttcactatggtgcccaggcagctgtagttcaggcctactcaggcttctcatgaccttcattgtacttcttagataaaacagaatacttgaagtcactagttacagagaacaggaatctataaactcattccataaaacaaaggaaaatttgttttttcttctccctgtgttgaaggagtgctgggagagtctccagagcacattacataatattatcaagacttttcctgggtctgggctgtgcctgttgctgcctctgggacaagtcagcctaatgcaggaaaacttatttctctttctttttaattttatttttctttaatttcccacctcagtatgaaatatctaaagtagctgaaatcatagaaacagaaagtagaatagtgattgcccaggcttgtgagagggtggacagagaattagtgtttagtaggtgtagggtttcagtgttacaagatgaaagagttagagattgttgcacaacaatgtgaatatacttaacactactgaactgtacacttaaaaatggctaaggtggttaatttaatgtcatactatttgtcacacacaaaaataatacataagatgaagaaatataacattgcaactttgcaggggttagtgtgggagacatagcttgcaaaagaaagatggaaatataatctgtaaaagcatactctagaagataagaaaaatttttctaaatgttttacactctgaataattttagaagacatgaagtatatgaaataaaaattgatagaataatgaaatgaaaagggagatttaggggatagaacaaaatgaaatgaaaaaaaagtaccaattagaagaaagagatgaaagcaattaaaaaaaaatgatggaatttgaaggctgacagtgaaaatccaacacaatgatatctgttgttgcctctaaagaagataatagaaaaataaagatataaggaagaatggaacttacaattgtatgtatgtatgtatgtatgtatgtatgtatgtatttttagacatggagtctcattctgttgcccaggctggagtgcagtggtgtgatcatagctaactgtagcctcaaactcctgggctcaagtgatcttcccacctcagcctgtagagtagctgggactgtaggcacatgctaccacacctggcttgaacctgtaatttaaaaggacgtaccaagtcctaggaaaattttgtatatagtgttcaatactgagatgtatgtttgcgatagtactgaactttaaagaaaaagaatgaatcatgcaggcctccatacagaaaaaaacaagtagaaaatgctaaaagacagggggacaggcagaggacagggaggttaaaggaggtataaatgagcaaacttcacctttaaagttgtaaaatcaaaaaatacatgtgtaagtaattatagggttgaaaattataggccagacgcggtggctcatgcctgtaattccagcactttgggaggctgaggtgggtggatcacctgaggtcaggagtttgagaccagcctgaccaacatggaggatccccatatctattaaaaatacaaaattagccgggcatggtggtgcatgcctgtaatcccagctactcagaaggctgaggcaggagaagcgcttgattccgggagacagaggttgcagtgagctgagatcatgccattgcactccaacctgggcaacaagagtgaaactccatctcaaaaaaaaagaaaattataaaatttacccctaaaagagttaaaattaaactataaaacatccaaaatatttagaaaaacaggttagcttggaggtaaagggagggaaaaagaaaacagtaaatttgtaacaaaaacaaaaaattgccataaaatgaaagacagcaaagcatgcaagtctatatataagtaaactaaattcacctatattaaaaaaaaaaaaacttcttggattatattaaaaaacaaacccagttccatgctatataacaaacatatcaaaagtaaaattactgagaaaggtggaaagcaaaaagaaaacttagagatgtatcaaaaagaaatttggggacattaatatagatgaggttaaattcaccaaaagttttgcaggaattgaagaggtatatttttatactgatttgcaatccacaatgaggatattttgcataaatctttaaacatcaaataacttagcataaaaaattcataaaacgaaaaccaggagctcaatcaataaggaaatttatgttgccttaaaaaagaatgaaggagggcagggtgcggtggctcacgcctgtaattccaacactttgggaggctgaggcgggcggattaagaggtcaggagatcaagaccattctggctaacatggtggaaccccgtctctactaaaaatacaaaaaaattagccgggcgtggtggcaggcgcctgtagtcccagctactcaggaggctgaggcaggagaatggcatgaacctgggcggcagagcttgcagtgagccgagatcacgccactgcactccagcctgggtgacagagcgagactccatctcaaaaaaaaaaaaaaaaaataaaaataaaaaatgaaggtatggaattatatgaaaacaactcttaggatggattgtgaggcaaaaggtgcttaacagggggattggacggtggaatttgcttgtagggagaaaaaaaataaaacagacaaccctaggaaatcttcaaagagaaatggacaaatacaaaataatactggggagcttgaacacacataattagcttatgacagatcgtggagatgaaaattttttttgtaagtgaagtttaaaaacacctgggaataatacaagtaatgaggttgatgtggggtgtgtgtggctgtgtgtgtgtgtctgtgtgtgtgcatgcatgcataagcctatatgggttaaacttaaaaaaaaaaccctttccaaaagaccatagaaaattttttcatacagctttattcagatataatttacccatttaaaatatgtaaggctgggcacagtggctcatgtctataatcccagcactttgggaagctagggtgggtggatcacttgagatcaggagttcaagaccagcatggtcaacatggcgaaaccccgtctctactgaaaatacaaaatatagccgggcatggcggtgcatgccgataatcccagctattcaggaggctgaggcaggagaatcacttgaacctgggaggcagaggcgacagtgagccaagatcgcgccactgcacttcagtccaaacgacagagcaagactccgtctcaaaaaaaaaaaaaaaaaaaaaatcaaacatgtaattcaatgatttttactgtattcacagataagtgcaatcaaccatcaccataatcaattttagaatattttcttcacttcaaaaaagaaaccctgagacttaagcaaatactgatccactttttgtctcagtagatttccctattctggactttcatacaaatgggaccattaagttggggacataacatgaaacttagggcccgcccataatgaaaaatgttaaaggaaattcccatgtaaagtaggcctccagtgggctacactcttactataagaataaaagataaacacgtccatgtagaaggggacagtaagaaaactgcctgttttaaccttgataatgagtttggggaaagaagacaaaatttatcctcagaatttataaccacaaactagtgttcacacagatttacagtcattaattttgtactggtataatttattagtgcagtaaactacaggtgtgttctgaaaaatctcaagccgagaatttaacaatggcagtaggttggtattgcagactcacatgcctttcggatgcaaatgcacatctctctgaagaaacttcaacccagacctaaaataccctaaattccctaaaataccaagctcatagtcaaggcactattagtagaaacagtatcaacaacaagaaacagaaatgagacccacaattacaaatgtccaggattccttggcaaaatgcctgattctcagactgggacaggagatatattagatgagcctgaacccttttgtggtgccagaaagtaaagaaaaaaaaatgcacagggatggggaatatgtcaatggaacacagggaccaagtaaaaaacttcccagtggctaaagctgggacagtttctgtagtatagcaatagtattagatcataaccaaagtataaaataaatacctatgagtctgtactgataaaagtaaatgatttaataaataaatggggggaaatgacaaatctgtgcagaattctaaataatgtatgtagatattccaccatcagggaggtggaacataactcccactcttaactaatgggttgcacatagtaacttaacttccttccaaagagtgtatttttataggaagggggaaaaagaataactatatagtgaagaattctggcaaacactggctcagccgggtcatcaagatcatcatcaatagtggtaagtcatgttgatagtatgtacccttaatatgtaatgagaaatatattttaactttgaaatcttcttcccaaaaacccataaccccagtctaatgagaaaaacatgagacaaactcaaatgaaggacatttatacttgaccagtacttctcaaaactgtcagagtcatgaaaaacaagggatgcctaagaaattctcacagacctaagagaagcctaaggagacatgataattaaatgtaatgtggtgtcttggatagaatcctggaacagaaaaagaacagtatgtacaaaacaaggcaacacaaagtatggcctttggttaataataatgtaactttattgattgattcatggtaatatattgtattaatgtaaggtgttaataggggaaactgggtatggggtataggtaatcatctgtactgttgtcaccgtttttctgtaaatccaaattattctaaaattaaaagtttattttaaaaaattaatgcattctgacattaaaacaggtggatagataaatgataaataagaaatttcatcttcttaaagaaaacactttacagcttctctttggcataaccaatttgctggcgttactgctcttgcactttggggccattactaactacagttagggttacttgaacacaggcactgcagtatcatgacagtcaccctgataacccagacagctatgaagtgcctaaggggcaggtagtgtaaatgtacggacatgctggataaagggaggattcacatcctagggtgggatggagcaggatgacaagagatttcatcatactactcacaacgacgtgcaatttaaaaatcatgaattgtttctggaattttccattaaatatttttggaccatggttgactatgggtaaatgaaaccttagaaagcaaaaccacagataagggggactactgtattcacacctaatgtgtggggtttcccctcacgttaagcagttctgacactgacaacctgtacttagcacagtccccacaggttatgggttcagtcccacaagactgcccaccacttcagataccaattgcatggcttggcctcctatacttctgaccaactagctccaaattggggtggtggggagaatcccatgaccccctcctcaggtttggtaatttgctcgaatggctcagaaaacttagggaacattttcttacatttcctgattttttatgaaggatatttcaaaggatacgcacgaacaaccaaatgaagagatacatgggcaaggtatagggaaggtgcacagagcttccatgtcctctctgggcctgccatcctcccagcttctcctgtgttcaccagatgtgaagctcttcaaactctgtctgttagggtttttatggaggtgccattacataggcaatattgattaaatcattggccattggtgattggctcaacctgcagtccctgtcccctccccagagatcaggggttggggctggaagttccagccctctaaacatatggttgttcccttggtaaacaggccccccatcctaaggggctttccaaaattcacctcattaatgtaaactcaagtgcaatcagaagtgacttgttttgaataacagaccctccttgaacctttatcactcgctcttgaactgtttcaagaaccaagtacaaaaaccaaatgtcataataaaagatgcccctattgctgttatcccgttttcctaaatggaaattacaagagttttagtagttctggccaggagctagggttaaggcaaaatatgtatttattatatcacagtattacaatttacagggtatttaaatgaattatacagtgataatagtaaaaggatgctactgggacaagagactgatacatcagtgaaaatacttaaatacagtgggcacttactctatgataaaggtgccatttcagatccatttgtaaaaagatggattactcaacaaataatgccaagatgctttgctagccatttagagtatcgtgagaattttaagttgtagccctactttactcctgtcaccaaaatgcaagtataaaagatccaagtataaaaaagagatcataaaagttctggttttctgacttttctaataaatttgagcagaagaaggctttcaaggtctgacaaaatcttggagtcataaagccaaagatttgataaatgtatatattaaaattaaagtgtcttcatggtagaaaatatcataatgcaaaatcaatttataaatgaataatgggaaaacaattttaaaaaattgagaaactgagaaaaggggctaattttcttactatgcaaagagctttttacaaatcaatgacagcctaaaaggaaaacaaaggacctgaacaaaaagcttatagtaaaagaaatgcaaatggccattaaagatatgaagaaatacttaactgtcttaataaagaatgtactaagccacaaattttgttttacctgtcacaatagcacagatgaaaagatttgattatccttagtattgttgagtacatggggaaacattcatgtactcttataagactattattagaagtgcaaatttgttgcaacatttttgaaagtgtggaacaattggatgatatctaactaatgttgaaataaatattcccttttcacccagacttccatttaagggaataacagatacacagataaagttgtacctagaaaaataaaatgaagctacacataaacgcacaaacaaatgttcactgcagcatgctttggaagaataaactattggaaacaacctaaatatttagttaaattttggtaaagtaaatcagtttaatataaaatgtttgtagatgcacaaaatcttgctttgaacaagtagttttactctacttggcaggaatttttctataagaaaacttgtttaatattggttatcaccagaaagtggaactaggaggttcagagataaccgggagaactttactgttcatattaaatttgtctgcattatttgaatattttgcatgtttaatttttaaaagtctgcagttctaggtatgaaagaaatatttaaaaataaatgaaaagcagtaaatgatattttcatgtacttttatatattcggggagaattttgcaaatgttatgagtaaaattaattccattatcagaagtaaataactacttcaacttatttgacctttactcttaacaaagtgccatgagatgatggtgattatcaaatcctgttgttttataaagccctttatacttaaggaattcagcttgcttgctgctgcttctgcttctttttttttttttttttttttttttttttttttttttgagatgaagtctcgctctgtcgcccaggctggagtgcagtggcacggtcttgactcactgcaacctccaccttttggatttaagcagttctccttcctcagccaccacagtagctgggattgcacgtgtgcaccaccgcacctggctaatttttgtatttttagtagagatggggtttcaccatgttggccaggctggtcttgaactcctgacctcaagtaagccacccccattggccttccaaagtgctgggattacaggcctgagccaccgtgcccagccttcagcttgcttattttctactatcctttctgtattttacttcattcccaggctgggaggactatgttcattgaaaatactgatattagggaataaacattttagagcaatgttatctatttccttattgttttacttactgctttaatgaattctagataggccttcaaatttttgctttattctgttgccaaggtatccttcctgaaatacagccataattatgttactcgcctgttcaaaatggtatactggctcttcatttcttttaagaataaaatttaaagtccttggcctagtgttccaggccctgtagaatttggccccaaataacctttgctaccttactttcctctgctttataaatgaaataagccatacatactgaatatttgcactctcccttattgcctcagttctttcctacttttatgcttttgctcatgctatttgatgaagcaggggaaaggccagcacaactatagtatagttatcaggaagagtggtacaaaatgaggaagaaaagttatgtatacttgtacttcttgttaaggagtttgggctttgttcttagtgtaagagaaggtcattgaaaattgctaaatagaaaatgacaagtccagcttacattttaaaaagactactttgaccactatttgacaaatagattggaaaggtacaagaagggacaggagacctattagagacttgcagtattgtgacaggaaatagaaatagatgtgtgattgtttggtgaaggattaatgacaggaacaggaagaggagggagttggaactatcaagaataactcccagattttggcttgggcagtaggatgatgtgccattcacagagatgtggaagactagggaaggtacagttgtcaaggaggctagaatagagctcagttagatggctgtgacatccaagtggatatatcatgtatatagttggaactcaagaaaaatcgtagcttgaaatgcaaatttgaaaataagtaacagcatggaaatggtactaaaagccacagggagagatgagattacctagaaagagaatatagactgagaacatttcctggaaattggccatacattgaggtcattgatcatattagcaagagcacttttagtccagaagtatggctgtactctgcagtaatttgaagagggaattggggtagggagagaggtaaaggagaggaaaataggttcctctttagaagcttggctatgaagaggagcagagaagtgggccagtacctggaggaagatgtgaggattttctaaacatacatactaaatgatgttttcaagccaatggaaatgaccctgaaagatagaagagagatatgatagggaaaggaagcaaggtctggaagggaagatgagttgggttccagagcagcattggaggaacttgcctttaaaagaatgggagaggtggtgttgtctctgtatccagaaaggttatgggagcaggtggattggcataggtggcagctgcctcaggacatccacagaaagtgatccctcagcccagaatgttcttcttcctgtttgtttgtttagctctcatttttacttcacatctcatcctcctcagagaattcttctctagctaccatttcccagtaaaatttctcttatatcagcttgtttttgttctttgtagcccctaatttagaattatatatttgtttgcatttgttcagtgcagaagctgcaatctcaagaacctgtagaatctaaatgaaatctcccaatttttaaatgttgctagataattctaaaattgtatagtctacatactttaggtataaatgcaggccctgtgtgtcccactctactctggggaagtccgtagggattcctagagtatttcatgaaaactctgcttgccagtaatctatagtgtgggtgaaaaggatcccatcattgcatgtgtgtatctttgtttctgcccatgatgtagaactctaaaacagtgttttgatgtcatatcagagattagaattagggtttatgattgtgtcttagctcattcctgctgctgtaacaaaattctgcagatctaagatcaaggcaccagcagattgggtgtctggtgagagcccagtctctgcttccaaaatggtgtcttgttgctttgtcctcacgtggtggaaggcagggaaaggccaaacaggagccttcaggccaaagggcactaatcccattcatgaaggtggagccttcatgacctaatcacctcctacctaatactattatattggggattatgtttcaacatgaatcttagagggacgcaaacattcaaaccatagaaaattgtaagatattacatctaaaaatgtcaaaaaatgaagatagaaagtggtaaattttatgtgttttttaccacaataaaaaaaaatttggaaaaaaagtcaagatggtccacagaaaaggaatttcaaagggctctaaaacatgtaaaaagggttttttaaaaaatttatccatttaattactgactatgagaatgaagtgtcaggtttcttagctgaatatggggttaccaatatctgtcaggtttaatagcaggattaattaaaaaaaaaacagatgcagagcatatgtcataacaccctacacagaacaggtggtaaatacatatagcagttgcttttatgatctggagaactgaagtgccttcctccaggtcacgtagccggtcagtggcagagcccaggccaaaattcatgtctcttaaacttttactgtaccagcgaggctactaagaacaaggcatcagtcttttgtgcaggcagtgaattttctgataccttatataaactattctttcctttagtctttgttctgtatctaaagaattgtgaatagtatttctaaacagaatgctaattttttaagttctttcaaaatctttttttctccttttcagagtccgaatgtcattcagaatttgaaaatactacccgttctgtcttcaggtcagcaaagttttacattcatcatcccgtacacctaccaagtgatcaagatatttgccatgaatctttgggaaagagtgttttcatgagacattcttggaaagatttctttcagcatcatccagacaaacatagagaacacatgtgtcttcctcttccttatcaaaacatggacaagactaagacagattataccagaataaagagcctcagcatcaatgtgaatttgggaaacaaagaagtgatggatactactaaaagtcaagttagagattatccaaaacataatggacaaattagtgatccacaaagggatcagaaggtcaccccagagcaaacaactcagcacactgtgagtttgaatgaactgtggaacaagtatcgggagcgacagaggcaacagagacagcctgagttgggtgacaggaaagaactgtccttggtggaccgacttgatcgtttggctaaaattcttcagaatccaatcacacattctctccaggtctcagaaagtacacatgatgatagcagaggggaacgaagtgtgaaggaatggagtggtagacaacagcagagaaataagcttcagaaaaagaagcggtttaaaagcctagagaaaagccataaaaatacaggcgagcttaaaaaaagcaaggtgctttctcatcatcgagctgggaggtctaatcaaattaaaattgaacagattaaatttgataaatatattctgagtaaacagccaggttttaattatataagcaacacttcttcggattgtcggccctcagaggagagtgagctgctcacagatactaccaccaacatcctttccggcaccacttctactgtcgaatcagatatattgacccaaacagatagagaggtggctctgcacgaaaggagtagctctgtttccactattgacactgcccggctgattcaagcttttggccatgaaagagtatgcttgtcacccagacgaattaaattatatagcagcatcaccaaccaacagaggagataccttgagaagcggagcaaacacagcaagaaagtgctgaatacaggtcatcccctagtgacttctgagcacaccagaaggagacacatccaggtacatggctacagattccatctggcaatgtgactgccctcttcatggactttttagttaagctttgcacacaggtgaaaaaaacaagccatttgccctttcctctctataccatttcttaccaactggaaaagagtgagaaagtgtagtacagtgctattgtgagttgtcattattttcatcaacattcatttacgaattaccttttgaatgaagggctttgctgctagatccttcacagtctgcaaagtttacccctgaaatgggggagctttgaggttagaatgatctctatcctgaacaatattgctgggtttatgtttatttgggttttttttttttaattcattcctctctgtctttcaggaaatatatttgaggttcttttaaacacacacacacagacaaacacataaataatacagggtttttttttttgtaattgaagaatggagttgatttgaaaaattgaagctggggtgagctacaatatgattttttaaaaatgagtaaataaggaattgtgaataatggaaagacaagtcaaagccaggaggaagataaataagccttgtggatgtggtcgctagaaaagagctacaaatttggctttatgttttctagcacttagtgttgctagaattttgtgatcaataacattcactttttaatataaaaagagaacaaaatttttagaaacattacagatttctgataagagacctaaaataaatttctctcatctgtcttcttaaagaaggcaccacagtgcaacaaagagtatcctcctataaggccattatcacattccttagtttagggtgttaataagggagatctcaaggggtcaataaatgtaggccattgctttaacccaagaggaaaatttcaagtatcactagacatgctctccaaagaaggatgcttgcaaaatggttcagtggagtatggaacgaaaaatatttgaatttatattcattttccccaactttttaaaatttatatttttgtgtatattttatgagtacagtagtgacataacatataaataaatatctattttgacaacttgtgctaaaaaaatttttgtcagattacatgatcaaaagaaattggagaccactggtctagagaaagggaggcatgagatgttcagacaatttattataaatattatttctcctcattgagcttttggcagtgtttacatatataaataatagagtatgctttttttctaaagtaagtgaagaattgggaatggtacagagtaaggcttttgttccataggggagatagataaatggatccaggagagggtttgtgtgtgtgtagtaaaatacacatgacataaagtttacagttttgatcattttcaagtatacaattctgtggcattgagtatattcacagtgttttacaaccatcaccactatctatttccagaaatttttaattaacccagtggaaaccctgtacccattaagcagtgactctctgttccctcttctcaccagcccccggcaaccagtaatctattttctgtctctgaggattttcctattctggttgtttcatataaatggaatcatttaatatatgaccttggtatcaggcttctctctcttagacacagtgttttgaatattcatccatgacataacatgtatcaatacttcgttgctttttatagctgagtattattccgttgcatgtatataccacattttgtttatccattcatttattgatttgggttgtttccactttttggttattgtgaatagaattgatatagaacgtttgtgtacacctgtctgcaattcttttgggtatatatctaggaatggaaatgctgggccatatggtaattctgtggattagcttattgaggaactgccagactattccatagtacctgcactattttgcattcccatcagcagtgtataagggttcctccaaccctcctccttttttccacagcgactgctttagtttatattcgcaccagcagttcacaagggtttcaattttattgcatccttgccaacacttattttccattttttaatatataaactaaccatcctgttgggtgtgagttagtatcattgtggttttgatttgcatttccctagtgattattcatgttgcacatctttttatgtgcttatttaaccatttgtatatcttcttttgaaaaatgtcttttcaagtcatttggccattttaaatctggctgtttgattttttggtttttgagttgttgggattttctatatattctggatattgatctcttgttagatacatgatctgcaaatattttctcccattttgtaggttgcctctttcacagtcactctgctgaccgtgtcttctgacatatagaagttttaaattttgatgtagtccaatttagctattttttttttgttttttgtgcttttggtgtcatatcttagaaatatttgccaaatccagtgtcgtgaaagttttcttcttaagagtttaataattttagcttttacttttaggtctttgatccattttgagttaatttttgtgtatggtgtaagataagggtccaccttcattcttctgcatgtgaatatcccgttttctcaacatcgtttgttgaaaagactttttttccccattgaatgttcttggcactcttgccaaaaatcgtttagccatatacgtgagggtttttatctgggctctctattctgttagtctatatatctctctttatgccactactgcactgttttgatttactgtgtatttaaaatgagttttgaaagcaggtagtgtgaaacctgtaacttagttcttttttcaaaattattttggctgtttggtttcccttgagatttcatgaattttagtaaatattttattgtttttaatactaccatatgtgcaatttctttcttaatttcatttttggattattcattgctaatgtataggtacataaataatttttacatactgatcttgtatcttgcagctttgctgaatttattaactctttttttgtgtggatttttaaggatctctaggatttcatgttgaggaagttcccttctttttctattttgatgagtgtttttgtcatcaaagcattttggtgtttgccaaattcttcagcatctattgagattactagttttttctttttctttttctttttttttttttgagacagagtttcactcttgttgcccaggctggagtgcaatgacgtgatctcagctcactgcaacctctgcctcctgggttcaaccaattctcctgcttcagcctcccgagtagctgggattacaggtgcctgacaccatgcccagctatttttttggatttttagtagagacagggtttcaccatgttggccaggctggtcttgaacccctgacctcaggtgatccacccccctcagtctccccaaagtgctgggattacaggcatgagccattgtgcctggcctgtttttttcttttattctgttaatatggtgtatatttattgataatcatattttgaaccaactttacattcctgagataaataccacttgatcataaaattctgttcatatgctgctggattcagtttattagaatttgaagaggactttttacatctatattcacaaggaatatttgtctatagtttctttgtgatatctttactgcctttatatcagggtactactggccttatagaatgagaaagttttcccacctcttggtttttgataatgagtttgagaaggattaatgttaatgattgacgttaattctttaaatgttggtagaattcaccagtaaagccatctggccctgtgtttttctttggggaaattttttttcattactgattcaatgtcttttaagtctatttagattttgtttcttcttgagtcagtttctgtagtttgtgtttctagaaatttgtttatctttttttttaaagtctgtttcatttatctccattgaatctacattattctgcttgctttggatttagtatgtttttttttcttctcatttcttatgattcaaagttaggttattcatttgggattattctttaatgtaggcatttgtagctataaatgtctctttaagccttgcttttgtagataagttctgccttcattgcttgccataagttttggtatgtttgtcttctttttcattcaaaatgtttgctgatttcttctttgaccattgactgtttaggacagtgttgtttaatttctacttgtgaattccccacctttctttgttattgatttctggttttactccattgtgatcagagaatatattttgtattatttcaatccttcatttatgaccttgcatatgatctgtccttgagaattttatgtgcatttaagacgaatgtgtattctgttgctgtttggagtgttctctattgtgttaggtctagctggtttataatgttgttcaagtcctctatttccttatcagtcttttgtccagtttttctatggctatcagaagtggattattgaagtctccagctattactgtggaactatctatttctccattgaaatctgtcagttttttgcttcatatattttagggctctgttgtcagttgtgtgtatgttcataattgttatatcctcttcatggattgatccttttatcagtacaaaatcctctttttgtctcttgtaacaatttttaatgtaaagtctgttttgtctgatattagggtaactaccccagctctcttttgggtaactatttgcatggaataccttttttatccttttaatttctaactctttgtgtctttggatgtaaagtaagtctcttttagacataacatagatggtcattttttataagctattctgccctctctgccttttaattggcgagtataatccatttacatttaatttcataagaaaggacttactttccccgtgctgtttgctttctttatgtcatatcttttttgtttttcatttctactgccttctttgtatctaattgattttttgtagtgtaccagttttattcccttttcattttttttttaacatttcatagttattttcttacgggttacattgaggattacaattaatatcttaaacatgtaacagcctgatatgaaataaaaccaacttagcttcaataatatacaaacactgttgttaatacatctctattcttccccttaactttaataatacacaaaaactgtgggtagacatctccatcccttcctctttaattgttatagtcacagattacatcttcattttgtccccactaaaatagatttataattattttatgcattcgttttttaaatcagataagggaaaagggaagagttacaaaccagaagtataatagggactttatttttacctatgtaattgtcttacccttatttcttatgccttatttctatgtatatgccttaagccttatttctatgtatacttcaaggtattgtcaaatgtcctttcatttcaccttggagtactctctttatcattccttatagggaaggtctacttgtaacaaatttacttttgtttatctgggaatgtctttaatttctcctttattctttttttatttatttttttttttgagacggagtcgtcctgggctgtagtgcaatggcacaatctaagctcactgcaacctccacctcccgggttccagcagttctcctgcctcgacctcccaagtagctgagattacaggtgcctgccaccatgcctgggtaatttttgtgtttttagtagagatggggtttcaccatgttggccaggctggtcttaaactcacgacctcaggtgatccacctgccttgacctcccaaagtgctgggattacaggcatgagccactgtacctggccttcttttttttttctttttaagatggagtctcactctgtcgcccaggctggagtacagtggcgtgatcttggctcactgcaacctccacctcccgggttcaagtgattcttctgcctcagcctcctgagtagctgggattacaggtgcacaccaccacacccagctaatttttgtattttagtagagacgtggtttcaccgtgttggccaggctggtcttgaactcctgacctcaggtgagccacccacctcggcctcccaaagtctcctttattcttaaagttttgctggaggtagatttcttgtcttacactttttatctttcatcatttcaaatatgtcatttcactgcctattgtcttccatggtttctggtgagaaattagcaattaattttaccgaggatcccttctgtgctcgcccttattcttactgctttcaagattctctgtctttggctttgacattttcattataacatgtctcagtgtggctctctttcagtttatcctactaagagttcattgagctacttcgatgtttagattaatgttttccccttatcaagttggaggagtttccaaccattatttctccaaatattccttctgcccctttctttctctcttcttttgggattcctaatatgcatctgttggtaagtttgatggtatcctacaagtctcttaagctctgttcatttttcttcattctgttttctttcattttctatttttttagagtagggtttcacactatcccagactggagtacagtgatgtaatcatagctcactgcggcgtcgaactctgggctcaagaaatctttctgcctcagctttccgagtagctgggactacaaggcatgcaccaccacactcatctcattctgttttctttctgtccctcagagtacgtaatttcatttcacctatcttcacctatcttcaagttcactgatctttcatctgtgtgatcaaatctggtattggactgctctagtgaatttttcattttagctcttgtacttttcagcttcatattttctatttgattcctctttgtcttcattgatattctctttgttgagacattgttctcatttcctttagttctttatccatggtttcctttagctctttgagcacatttaaaacagttgatttaaagcttttgcctagtaagtctactgtgtgttcttcctaagggacaatttctatttatttctctcctctaacccatgaatgggccatactttcttgtttcttttcatgcctcataatgttatgttaaaattttgacattttgaatattatgatgtgataactctggacatcagattttcctccccttctctagggtttgtagttactgcttgttgtaagttaaagttgttggtttatttagtgacttttctaaattatgtttgtaacgactgtactctttgttgttgtgtgatatttctgttcctttagcttagtagctggctacttgtttgatagagattttcttaaatgcctggggccgaaaaaagcaaaagaaaaaagaaaattccccaagtctttgcagattttctgcattcggacactccttcaatgcttagccaggccattcacaactctctgccttagccttgcttcctacttgtactgagcctaaaggtcaaccagtggtgaaagtgtctggtcttctcaggtcttttctgaacatgcatcttgctctgtgcatgcactttctagtttccctggtatacatgggaaattttccaagcccttatgccccaaagattctcagtttttctttcctgcttttagtatgtctattgtttcccttgaactgttaatttttgctccaggcagtagtgccttgttcatttggttttcagtattttcaaggaacattcttcccctataaaactactttttcactctaagagagttatgttaggtgaaactaaggcaggtcccttgtgtcagtccacggctaatactcagacatgtcaaaacaaacacaattccttgggaacaaggtactactccgtgctgtctttgggagcaggtacctacatcaggaccaagggctgccgtctttaagaccaccatgctgggtaaggggatagcagaaggctaagttaaaatgctacaaagttcttctaccttgtgctgatggcctttctcctggtttagcaaacacttggttactataaacctttgactatcttccaaagttcagataaggttggctttgacagttttccgtattttttggtgtttctagagaaggcactttctcccttggaggtctctactctaccatttttgcttgtcgttcccaggtggagttgtgtgtccctctgaccatcaaaataactagagtgacatcaattattttttcttttttttttttttgagacgtagtctctattgcccagcctggagtgcagtggcacaatctcagctcactgcaacctctgcctcctgggttcaagcgattcttctgcctcagcctaccgagtagctgggactacaggtgtgcaccaccatgcttggctaatttttgtattttagtagagacagggtttcaccacgttggtcaggatggtcttgatctcctgacctcgtgatctgtccgccttggcctcccaaaatgctgggattacaggcgtgagccactgtgcccagcctgtgttacatttttaatatctttgcctttgattttacttcatccagtacagttttttttaaatttgcatttgcactattgctctttgcccatccttttagccttttgagtcactttactcaagtgttagtaacatctgaatttaacttttttcccacccaatctgaaggtctctcttttgaaacaaatttattccatttaagtttactgttgtaaatgtttggtttaaattttattattttggctttttttcattctgatgtatttattaaataaaggtgtgttcaactaaatttcatttcaaagtaaggagaggggtgaatttcttgtgcgtggtaaaaaacacataccaaaaaattcccttcttaactgttcttaagtgtacaagtcaataatgttaaatgtattcacactatgttgaaaccaatctccagaactttttcatattgcaaacctgaaactctatacccattgaacaacaactgcccttgtccccgtcccccagctcctggtaaccactattctgcttcctgtttatgaatgattaccttagatacctcatatgagtgtaatcatgcagatttgtctttttgtgactgacttgtttcacttagcataagatcttaaagattcatccatataatccatgtagcatgttataggatttcctccctttttaattctgtatgatattctactgtttacatataccacattttgtttttctgttattcattgatggacatttgggttgtttccaccttttagctatggtaaatagtgctgctgtgaacatgggtatgtaactgtctttttgagaccttgctttcaattctttgggatacatacccagaagaaggattgccagatcatatatagtagttctatttttaactttttgaggagcctcaacactgttttccctagtggttgtgccattttgcaatgccaccaacaatgtacaagggttctaatttctttacatccttaccaacacttgttttttttttttatagtagccatcctcatgggtgtgaggtaatactgttttgatttgcattgctcttatgcttagtgatactgagcatttttttatatgctttttggccatttgtatgtcatctttagagaaatgtctattcaagtccacttttacttttttttttttttagaggtggggtctcactgttttgtccatcctggtctcgagctcctgggctcaagcagtctttccccattgacctccgaaagtgctgggattataggcatgagccaccatacctggcctgcccatcttttagttaggtatttgatttttgttgttgttgttgcgactgctttatattttctgtatgttaaccccttctcagatacatgatttgtaaatattttctcccattcaataggttgtctttcactctgttgattgtgtcccctgatgtacaagtttttaagtttagtgtggtcctgtttgtctatttttgctttcgttgcctgtgattttggtgtaatattcaaaaaattattgccacattgaatgtcatgaagctttttctgtaagagttatatgggtttaggtcttacatttaggtctttaacccattttgagttaatttttgtacatggtataagataagggtccaatttcattcttttgcatgtggatatccagttatccaagcatcatttgaaagctatccttttcccactgagtggtcttggcaccctcgtcaaaaatcatttgaacatatatgcgagagtttatttctgggcatgttttcttatttcattggtctgcttgtttgtctttatactaatatcacactattttgattactgttcctctggtaatatgttttcaaatcaggaagtgtgaatactccaacattgttcttgtctttcaaagttgttttggctattcaggatttcttatcatttttaatgatttcttgctatttccaatcttttttgttgattgctatgtaatgcatgtttcttcgagttggttttttgataacttagcaactatgtaatctaattaactaatatgcttcaaccactttctcaatctgtcagtgtcagatatgaaactatctattggctctattatatgaaaaattaagaaactgattttacttcttcccacctatcccccacataattttccccaactaaccaatagtggttatattttgtgaattttgtctcagctcatttatagtagtagtattactatattgactagcttctgtttcaagaattagaacattttacttacattttataagatcatgagttattttacattagttgtatttaaatatagggtcagtatttaccaggagtcttttcacatacctatttatgagtttgttaatttcaatctgttgtttgactagactgttaaatgttcaggagtgatcatcttttatgttcttgcaaatttgagactattacctttatgtaccagaacaacaactgagttggatataaaagttttgattaacagttcttaattcaaaactgttaattttgctcctcttttctggattaccagtatttaatgtttcatatgcttgcttgctttgtttctttctttattatttccatagtttggagggaacaggtggtttttggttacatggataagttgtttactggtgatttctgagattttggtgcacccatcacctgagcagtatacactgcacctaatgtgtagtcttttattcctcaccccgctcccacccttcccccctaagttcccaaatccattatatcatccttattcctttgtatcctcataacttagctccccgttataaatgagaacatacaatatttgattttccattcctgagttacctcacttagaagaatggtctccaactccacattgctgcaaatgccattattttattcctttttatggctgagtagtattccatggtgtatatgtaccacattttctttatctacacattggttgataggcatttaggctgcttccatgtttttgcaattgcgaatcatgctgctataaacatgcatgtgcaagtatctttttcatataatgacatattttcctctgggaagatacccagtagtgggactgctggatcaaatggtagttctacttttagttctttaagggatctccatactgttttgcataatggttatactagtttacattcccaccagcagtgtaaaaatattcccttttcaccacgtccacaccaacatctatttttattttttatttttttatttatggccattcttgcaggagtaatgtggtatctcattgtggttttaatttgcatttcccttacaattaatgatgatgagctttttttttttttttttttgagacagagtctcactctgttgcccaggctagagtgcagtggcatggtcttggctcactgcaacctctgcttcccaggttcaagagattcttctgcctcagcctcccaagtagctgggattacaggcaggtgccaccacgtctggctaatttttttgtatttttagtagagatggggtttcactgtctgagccaggatggtcttgatctcctgacctcgtgatctgcctacctcagcctcccaaagtgctgggattacaggcataagccaccacgcctggccgatgatgatcatttttgcatacacttcttggcctttttttttttttttccccgagacggagtctcgctcttgttgccctggctggagtacaatggcacaatctccactcactgcaaccactgcatcccgggttcaagcagttcttctgcctcagcctctggagtagccgggattacaggtgcctaccaccacgcctggccaattttttgtatttttagtatttaattttttgtatttgtattttgtattttagtagtggtttcaccatgttggccaggctgatcttgaactcctgacctcaagtgatccacctgcctcggcctcccaagatgctgggattacagacatgaaccatcgcacctagcccatttgtatatcttcttttgagaattgtctattcttgtcctttgcccactttttgttgggattatttgttcttttcttgatttgtttgagttccttgtagattctgcatattagtccgttgtcggatgcatggtttgcaaatagtttctcccattttgtgggttgtttgctgattatttcttttgatgtgcagggcccatctatttatttttgttttttgttgcatttacttttgggttcttggtcgtgagctctttgcctaagtcaatgtctacaagagtttttccagtgttatcttacagaattgttatggtttcaggtcttagatttaagtctttaatccatcttgagttaatttttttgtaagggtgagagatgaggatccagtttcattctcctatatgtggcttgctaattatcccaggaccatttgttgaatagggtgtcctttccttactttatatttttgtttgctttgtcaaagatcagttggctgtaagtatttggctttatttctgggttctgtgttctgttccattggtcttggtgcctatttttataccagtaccatgctgttttgttaactatacccttgtagtatagtttgaagtcaggtaataaggaggcctccaggtttgttctttttgcttagtcatgcttcggctgtgtgtgctgttttttggttccatatgaattttaggattgttttttctagttctgcaaagaatgatgatggtattttgatggaaattgcattgaatatgtagattgcttttggcaatatggtcattttcacaatattaattctagccatccatgagcatgggataggtttccatttgtttgtgtcatcaatgatttctttcagcagtgtgtttctcattttccttgtagagatctttcacctccttggttaggtatattcctaaggttttgtgtttttttttgtttgtttgtttatttgtttgttttgcagttgttgtaaaagggattgagttcttgatttgtttctcagcttggtctttgttggtgcatagcagtgctactgatttgtgtacattgattttgtatcctgtaactttactgaattcatgtgtcagatctaggaactttttgaatgagtctttaggattttctaggtatacagtcatatcatcggtgaacagtgacagtttgacttcctatttacctgcttgggtgctctttatttttttctcttgtctgattgctttagctaggacttcgagtacagtgttgaatagcagggatgaaagtgggcattcttgtcttgttccagttctcaggggaaatccttttaaccttttccccattcagtataattttggtgtgggtttgccatagatggcttttattaccttaaggtatgtcctttctatgccgattttgctgagggttttaattataaagtgatgctgaattttgccagatgctttttctgcatcttttgagatgatcatgtgatttttattttaaattctgtttatgtggtgtatcacatttattgacttgtgcatgttaagccatccctgcatccctggtatgaaacccacttgaccatggtggattatctttttgatatgctgttggatttggttagctggtattttgttgaggacttttgcatctgtgttcatcagggatattggtcttttggttctttgaaaagataaacaaaattggtagaccattagtgagattaaccaagaaaagaagagagaagatccaagtaaactcaattagaaacaaaatgggagatattacaaccaataccacagaaatacaaaagatcattcaaagctattatgaaaacctttacatgtacaaactagaaaatctagaggagatgattaaattcctggaaatatacaacccttctatattaaatcaggaagaaataggaactccgaacagaccaataacaagtagtgagattgaaacagtaatttaaaaaactgctaacaaaaaaaagtccaggaccagatggattcacagctgaattctatcagacattcaaagaagaattggtacaaatcctactgaaacaattccaaaagataaagagggaatcctccttaaatcattatgtgaaaccagtatcaccctaataccaaaaccaggaagggacataacaaagaaactgttgactcgctctttctgtgtttctctctgccccgtgctgtctcccctgcagtctctgcagtccaggccctcttctctctctatgatctctttgcactctgtctgtttctctgttctgtctgcctctgctcacccacacgtgcacttgtgccccacatgccccaccctcgttctctctggttcccactgtgtctgttgtgtgtctctattcttcttctgattactctttcctcccccacccttatctctttaatgttattgctatgtttgtctctgtgctttctctgtgtgttttcactctttttctttcctgtctcccttctgcagttctgtttcttggtgtctggttgtcttctggtgggggagaagggatagtttactattgtcagtacaaaaagtgaagaaaaaaggtcattcagtagttttcctttatgaagaaaaagctgaagatggtgaatgatttctctgtattttagatctctccttatttatattagctcatgtttccagcaggtgggcaaataaacaacacaagcaaactaccaaacagagagacctccatgagaaaagaaaatgagagctttatgtccaactaaatgtttattaatgtttcatatatttttgtaaaatggagttcatatcagactttacatgcctatatacgaagagaaattacactcaaaagcagcatcctgaaatccaaagcgtaaatttttttatacgtttacagagaaagagttaagatataaaactatgtatacatattataatccaaatatacatacctatgtgtatatatatatacgtatacatatcagtatcaccggaacaaaatatatgtcatacatagaacaaaaaactgaaattattataaaaatatgaattatgattttttcttttatatttttgtctattttttacaagaattatgttctaataagaaaccataaaagttgtttttgtttaacaataatagaataaggtaagccagattgataaacacaaaaagttataagcttagaaggtgaattcccaaatttctcattgacttctattttgggtgtaaaagaccattttgagcttcccaaactagtcataatgaacccttctagaagtatagtgaaataataggattagagaactagtatacatggtttctgtgtcatgataaagtacagttttcttttaacttccagtgccaagggttgagttaaatgtactcctcagttcatgacccctctttttaaaattttatcaccatatgccatcctcatttctgtctccccataggaaaccagtcttatgtaaatatctttcaggttacatgagttcttacaaaatacatattttttaagtatgcatgaactttcatcatatataaaaggtgtaccattatttttgttatgttggctaagttttgtattttcaaagtctgcctacattgctgtaagtatatatctacattgctgtaagtatatctaatcacataatgctccatggagtgcatctacccacttacgcatgaacacccaggagcacccaggttcctgtcagttccccagcaccacagaacatggcagtgctgtctttgtccatgtccccttatgcgtctgtctgaaactgtctttagcatatatacccagaagtggaattcttggcttacaggctcgtatacttaatttcctacattagtaccaaattgctttccagttcatattcctacctgcagtgcatgaaaatttccatttctacttctctgccaattatttctaatttttatcagaacactagtaaaacaagtttctataccatgggaagataggtgttttttttttttctttttaatgagatgatacttgtcttgaacatctagagtatctctttaatttccgtgggaagagagtttttcatataatttacagtgctttcaaccagaatacattcgtttgttcattcatccaaacataattatagaattgacaaaagcacagagtgacatactttaataagcttttctcagatttatatatatgagtgagaaaatatatggaaaatttttataacaatatttaaaaacttgatttaaatatctggtaaaacttcattgtcaaaacacagcatccacatttgttccaaatacccatggaatatttacaaaattaattgtacgttagacaaaatattgatgaattctcaaagcagaagttttataagtaagcccatattctgtaactgtaatgcagtaaaattgaacttaacaactataatttttaattttgctaatgctttaaattgcatttctattaggtttcagatttgccatttacatttcttactttgagaattacctttgtgtatcctttgcctgtttttctctgaaggtttttttcctattcatttttgtaaacatatttggagaccactgtagttgctgtttttttaaattctgttttattacttttttttttttttttttttttgagatgtctcgctctgtcacccaggctggagtgcagtggaaagatctcggctcactgcaacctccaccttgcgggttcaagcgattttcctgcctcagcctcctgagtagttgggattacaggtgcgcaccaccacgcctgactaatttttttgtatttttagtagagacggggtttcaccatgttggtcaggctggtctgaaactcctgaccttgtgatctgcccacagcctcccaaagtgctggaattacaggcgtgagccaccgcgcccggccccttttttttcttttttagagatgtggtcttgctatgttgcccaggctgatctcaaactcctggcctcaaccaatcttcccacctaagccttctgagtagctgggactacaggcatgtgccactgtgcctggcttcctattgatttttaagcttaattacattgtgttaatattggaataaataatattgttatgtatttctttatttctgtgttcatgctaaaaaagatcttccaacctcaagatttgtttttaaaaaattatcctgctttacttttgatactttttgagggatatggaagctttaagctttaatttaagcttaagcttttaatctacatggaatttatttttgtgtgtataggtataaatttatttccaaatggtagctagttgccaataaagatttttaaataaagtaatgtcaataaattaatttattagaaaaatctaatgttcttttgttaagctagactccaatcagttagaattggtaaataattggcatggttcttttgaaatttccgttcaaaacaactgaaataacatatggacaccttataaaaattgacagacctgaagaggaaattaattctgactgaaacatgaaggagttttatttattaaacttgaagttggatcagatcatccaagtgaaaatatctaacaagctagatggatctgaccccatgaggatatcatgagtgagatgaagattttagagtcatccatgtaaaggtggtggtaattcttatagttattaaagccaagaggaaacatatttgggagaatgcaaagaataaataaatgtatagggctacttccaggctgtagtttttcaaagctgatagaaaaacctttgggaaacatgctgtctcgctctatcaccctctacaaaattgtatgatcatttgtcaaccttctggttttcctgtgagttaattgagtgttttagctcttgattcattttctttctgtccaacccaaagccctaccattatctgggaaattttattatcccattagagttttttttaccttagttgtaatgatcattgactgtaataatttccactgcattttagccatacattctcttaactattgtttgaattttggattcactcaaaattatttgttctctgcaatctttacttcttcacataccaaaataaatttttcaagaacctgttatgcaggcacagtacattgttaattttctgaggaagattttccatagttttctgatcaaccacactggcagaaatagataactagttctaatttccaaaaggtaagtaatagatagttaccacctgagtttctgcttctgcaaccttttttcactaggaattaaatgtatgtcacagcattattttttggacagacacttggtcattgtgtcaccccttggtgatgagtaaattttattgaaaatatacttgctgtttgattgacacagttctataaacttttcacaagaaaagacagtcttttggtgccatagactatatttctcattttgactctgactgttttatcaatgtgtgtctctggtcctaagcctgagtgattataaatgaattagcgtaaacccatgcctctaaaggaaaagcatctcccagcaggcacctggctagctgaaggccagcagtaccaatcctcctgcctgaagaatagcagtgtagggtcatatcagaaaacatggcccttttcctatgttggatatgtttttctaagatcagtttccacctaacaagtaattttttaattgaaaagcttttatttctaggtctgtctcatgtctttaataaatacgggaatgagtctcatttagttcttaagctaaagagtactacaaaaaccctgaaacatctctgagatggtacatttactttccattgttactcttgtataagacagcaacaggtgagtttgctctcatagtggaaattaaaacagaagcagccagccagcatatatttccttccaagattgtgtacaagagaaagcctaacccatggtggctgataatgccatttaatatatagtcacctaaatctttatgtgtcagctccattgcagaggactcagtgggctactccacacccaagcctgagggatgaggagatcattagagatgcgtgggactctcccacggactaaggaaagtgaactgttttggtttactgaccgcatatataaatggattcaggaaaaaaaaaaaaaaaaagttccatcccatgatgggtcctcagcacaaattgtgggacatgagtgcagatgtgtgcattgtgattattgtgaatccgtggatggattcaacatcacatggcacggttctgacagtttgtgcttggcccagctttagagtagtgctccatcactcctatagcatccagtgcccacctttttttaggattgtttgttgtttattacatgaaggcaacactgtttatcactttccaaggttgatctttttctgatttcggtaacataggagatgcagaactatagagtataaccttttttctccaaacaagacttcatagtccacatttttttcagttggcatacaataaataagtgcattttatgcctttcttgacatattttccatgccgtaaactttacctgtttaaagtgtacagtttaatgggtcttgatttagtcacagaattgtgcaaccatcaccgtattctgattttagaatattttaaacaccctccaaaaaaatcctgtaaacattaacaatcaatcttcatttccccagcctcccacctctgccacagcctgcaggcaaccactaatgactttctgtttctatggatttgcctattctggacctttcatataaatggaatcacagtctttcgtgactgacttcattcacttaacatagtattttcaaggttcgtacacctcatagcatgtgctcattccttttaatggccaaaggacattccattctatgaatataccacattgtatctattcatcagttcatggacatttgtgttgtttctgttttttgtctattatgaacaaaactgctatgaatattcatgtagaagtttttttgagggcatatgttttcatttctcttgggtatatacctaggagtggaattaatcatatgacaactctatgtttaaccatttgaggaactgccagacttttccaaaatgactgcaccattttacattcccaccagcagtatgtgagggttccaatttttccacatcctagtgaacagttgtttttgtctctgtctttgattgtcatcttcctactgggctggataattatattgggttgagagagaatttattcttagacttttctatttttaaatgtttacctttttataggcaagcattactaaatgaaaaagaatttgaaatgaaaacggaaacattccaaggctcctcatataatccagtgtaattgtgtgttttttcacaccctgtggtgcacaccatgactacttgtgtcctttggaagtgcaggatgttcacagaacctcagacttggaagggaccactggagtcctgtgaatcctacccactcccatcaacacttctgctgcatctgcaccaaatgacaggccaccctctaatagctccacctctcacgtgctacttatggggcagccagttccatttgcctaaggcttcagttatcaaaggctttaacttaaatccaatatctccttgcattggaatacctggctctgctcccagagctgctctctgaaggcacaggaaacagacctagccctctgcctctcggacccccacctcctttcctgatgtgttttttctcaggtcttcactccatgaaccgttccacatgggctgtggtatctactctcctttctgagtttggcagctttcctgggtataagtttgttggtatcttgtttttaaatatgtccctctcagctaagcttgttgtgcttcaggtagtgagagcgtattgaccaacacaacttaccaccttccattcagtgactggaccatgtctgtggcctaacgtatgttattccttttgaactccttaaatcttttctgccacacccttccccacaagtttttgtttattctcatttcttgaactggtaatacagtacaaccacatgatacaaaatatttgatcaagagtgtccttcctactcctgttccccagcacccagccctccttcgcagaggtatacaataaaatttcatccattttaagtgtacagttaacttttagtaaatttacagagttttgcaatcatcaccaaaatccagttttagaacatttctgtcgttaccaaaaggtgcccttgtacctctttgcagtcaatccttgttcccagccccaggcaacctctcatctgccttctgtctcttctggagattttatgtatatagcatcagatgtggtcttttgcatctgcctttttttgttaacatcatgatttcaaggttcatccgtgctgtagtatgcatcagtggttcctttttgttgctgaatagtgttccattttatggatataccacattttgcttatctttcagttgatggacatttggattgtttgatttttttggatgtgaatgctgctattaatacttctgtaaaagttttgtgtgggcatacattttcatttcttctgggtttttactcaggagtgaaattggtaggtcatatgataggtttatgtttaactttatcagaaattgcctaagtgttttccagagtggctgtatcattttacattctcgcctgcaatgtgtgagggtttcggtttctccacttccttgtcaacacttgctattgtccacctttttatttatagccattctagtgggtgtgaagtggtatcttattgtgagtttaatttgaatttccctaatgactgatgatgttgaacattttgtgtgcttattggccactcttacatcttctttgatgaagtgtctttttaaatctttttgcccattttaaaattaggttatcttcctactgagttgaaagagctctctatatactctggttacaagtcctttatcagatgtattttttgtaagtaacttctcccaggctgtggcttttgaattgcaaatggttttaatacagtaatgatgaagtcccatttaaactgtttgtcttttatggattgtggattgtacttggggtgttatattcaagaactgtttgcctaaccaaagtcatagagatattctcctatgtttttcattaaaagtttattgttttaattattatgtttaggtctgaaatccatttttagtgcattttttgtgtacggtgtgttgtctaacttcatctctttgcatagggatatttaattgtcccagcatcatttgttcaaaagagtattattttctccaattagttgctttggcccttttgttgaacactaattgatcacatatgtaaggcttcatttggaattttcattctgttccattgatatgtctgtatctttttttttttttttttcttaagacggagtctcactctgtcatcaaggcgggagtgcagtggcacaatctcggcacactgcaacctttgcctcctgggttcaagcgattcttctgcttcagccttccgagtagctggaactacaggtgcgtgccaccacacccagctaatttttgtatttttagtagagaccgggtttcaccatattggccaggctggtctcgaactcctgagtttgtcatctgcctgccttggcctcccaaagtgctgggattacatgcatgagtcactgtgcctggccaatctagatctgtccttatatcaatgccacaatgccttgattgctgtaactttataagatttgaaatcaggaaatataagccctctacctcttttttttttttttttttcttttttttttttcagtaactttggcccttgtggtccttgccatttctatataaagttgagatcattttgtagatttcttgaaaaatgcttgctggaattttgatagagattgtgtagaatcaacagattaatttggggattattgccatcctaacaattttgagacttccaatccatgtatggtgagtgtctcaccatttatattgacttctgatgtctcccagcaatgttttctagtttttaatgtgcaaaccttgcacttcttttgttaactttattcctaaatattttattctttttgatgctattgtgagtgagatagttttcttaattttattttcaggttgtcacttgctagaatgtagatatacaattgatttttttatcttattgatcttatattctgcaacctgggtaaccttggttattagttctagttctttgaggattccttagtattttctacaaagaaaatcatgtcatttgtaaataaaaccagttttacctcttcctttcaatctggattcttttctttcagagacattttataaataaaaaagcaaacacctacatatacttttttagtacctaacatgctttcttccccttttttatagctgcagagaaggtataatgtatttcatatcagtcacttcttttttatgttcccttttttaaggagaaaataaaaatatattttttaacatcattttagtctctagttttatatcagtgttttatattttaccatctaaatcatctgttatttctaaaatgcagatttcgtttccttttctagatggggaaactgacacaaagaggttatggcttgcctaaatagagaccgctaataaagggaagacctgaaactggcctagctgtttggaatcctgacacagagcttttttttttttttccctgcatcaaagttgctgccattcctcctgcctaatgagcaggactttacaattgcccctactaaacatgctctgatttagcccatcattcctgttattatttgggatcctgacagaggccctctctctgatgagtattgtatgtaaattctcctgctttccatatctaagacatttatgtctaaaatcttaagaaggaatagccagcaccaaacccagtgattgcacacagtaattgcttagtaaatatttgttgtcaaatgcaagatgtaccactcagtaaagacatctgttttgaaattatatttggataaggttggttcaatcagttatgattctatcaacttgtattatcatccaacccatataactccatcttgccctcaaagatgctattaaatgttttggcattttgttcaaatctaggtatggtatgctatgtattcttttggttagccagtaaagtgaccttatgtactagagaaaacaaggctaacctggcagggcatgctctgcctgcctgactgagcctggcttaaatctgctcatcttttttttcaccagttccttgcaaattatctgattcgtgatgcatcctaaattttacatgtcatcagtgttcagcccactagtttgcaatgcctacctttccgaaaatcaaagtatttcctttcttcaagtgtctaacatagatcttcctccccaaaattctgttacaagtttctttgtgactctggaatatggtttatctagttcaagggatcagatatgctcagtttcctatccttaccctggctttaatttacctccaccagacctgcctaagtagaaaattccctaacatttattcaaaatatagattaccagacccctcccctagagattccaattcattattctgggttagcctccctctgtccttattaacgatggagatattaacaatgaacaagtttaagaagcactattttacactatagtatttgttcagctatatcttgttaatcattttgtcatccaccgacattccaccatcagctgcaatccctgaaattgccttcattgctctacttgctctgaacgtaatttaaaaggcctttgggactctctaatactttttgcaatgctctactcatcaggacgtctcacgagccttgtaggtctttggtgtttgtcttagtatacgtgtttgagtcagctcttaattggagagcgctctcttcagttcatcactctttttagctatctccaggcctctttatctcacatgaaaattatgtgcaggtgtcacttcagaatttcattctgaagatgcatcagactaatgtttggctagcattctcttcagagtttttccaagcccagtgaaaatagccaggcttttttttgtcttaccaccttataacagcttaagtcctatccacctacaacattctccctatcacagatacaaggcagcaagttcacagtatctcaagcttcctccaaatgcatctcaacagtttgaattggattagaaagaggacttgttggcttgcttatcctgtggataactgtgacattgactgcaggtaataataacaagatctcttttatttttctaggtagcaaaccatgtgatttcttctgactctatttcctcttctgccagtagtttcctgagctcaaactctactttttgcaacaagcagaatgtacacatgttaaacaagggcatacaagcaggtaattacttgaatctaaactttttcattgaaatacattgaaatggctcttaaacatgtaagatactcaacctcaatcataataaaggacatgaagataaaactatatccaactgccaattttcatcttgtcagattggcataaattcaagtttgatggcaagtttgtgggcaagagtgtagggaagcgggttcccttgtatatttcagtgggagtagaagttgattcactgtgaaaggcaatttgaaagttcctatcaaaattactttcactcagcatttttacttttgaaatgtctgctatagatatactatgaatatgtggaatttaacctatagatatacttgcatatgtgcaaaatagtgtacaaaattaaccactaaaacattgtttatagcaggagattaaagtgaatttaggtgttcctcagtagctgaatgattacataaattattgtatgtctagataatgaatgaaattctacatagaatgaggaagctctctctgtactaacatggaaagatttccaaggcatgtaattgattttaagaaaaggtacagaacagtgtatagcatactacaatatgaattttttttaataaaagaaagaaaggagggaagagaattgcttaaatgtgcaaaacgaattctctggaaccatatggggttgtgaacagaataaatggagaccagaagtgggaaggaggatttttagctattgcctttctataccttttggtttcataaaataaaaataaataaactttcatcttgatttgtttcttcccctcaaatcctgtcttctgtcagcctcttccttacctgcgtgactaccaaaaggactcttaagtatttattgtctttctaacttgggatcagagttgaaatcctttaccctttgaacccatattcattcttctttccctctcatacctaaaatgagttacaaataaaaccctttcttttttctttgccctctttttccctcctactctcccctgtccttctttctcttcctctctcttcgcatccctccatcccacacaaagggattgtatttttgaaacattaaaaagggttcacgtactcacttgaataaatctgtcaactcagcctcaaggttactcccagagacacctatgatccttcccctcaggtaacttggagattgtgaacggtgccaaaaaacacactcgagatgttgggataactttcccaactccaagttccagcgaggctaaattggaagagaacagtgatgtgacttcttggtcagaagaaaaacgtgaagagaaaatgctctttaccggttatcctgaggacagaaagttaaaaaagaacaagaagaattcccatgaaggtcagtttctcattccagatcttgtagtagagaaactagtgaatttcaagtcccctgcgatgctgactctgtgtttccaagtgactctatgtggtccccacctacccccagccacaaccttgtcaggttttcagagtccagcatggcagttacacagtcatccctgcagcaaaaccagactcaagggcaccctgtggcctctgacagctgagacccctgagaacctgtattatatgcatatcctggataagagctgggtggggctgtaaaaaagtgaaaaatctgtgttccttctaaaaactgtttcctgtaggagtttcctggtttgttcctgtggaaaatgtggagtctagatcaaagaaggaaaacgtgcctaacacttgtggccctggcatctcctggtttgaaccaataaccaagaccagaccctggagggagccactgcgggagcagaactgtcaggggcagcacctggacggtcggggctacctggcaggcccaggcagagaggctggcagagacctactgaggccatttgtgagagcaacccttcaggtgcagtgacgttgacttaactttaatgctacgtgtagggagaagaagggcaaggcgcagagaagctggctctgtgacttggtgagctgaggtgtaggcctgagacgctcttttccagcacctccgcagttcacctgttttcacgcacgagggttgggtttctcatcatctgtcaaaggctgccttactcctaaccatagatggtgcatcaccgtcgggatctggggacaaggccgcaggagggaggatttgcatctctgcttattgggcctgtgggtccctgtgtgtggagggcccggaggcctgacgcttgtgcggcctcatgtgtgttcctccatgtccgtagctcctgtttctgctccatgtcggatttaaaggaaaccaggtctcttctgatgtgtcccttgggggataaaggaaatgttattaatgatgtatttgtctgaatagatctttaaaatagcactgtgggaagagctgtattgcactgacatttcaaagagttgtaatcgccatagtcttagttcctggcctatgctgaaggcatgtctttgatccctgcggtttccaagctgcagctctcccccatagagagatgtcagaggattcttcaacgctagatactttctcggcatttgaatcagacttccccaaacctcttgggcaggtggtgtgtctccaggcatatggagagtagattgcatcattaatctgaggctgggcattttctcttttttttttcttttaggaatcgcttcagtttcacagacctgacttcatctcccgctctggggagcggataaagcgcctgaagttaatagtccaggagaggaagctgcagagcatgttacagaccgagcgggatgcactattcaacattgacagggaacggcagggccaccagaatcgcatgtgcccgctgcccaagagaggtacgccctgcccgttcactttcctgtgagtggaatagagaaggcaaggtctgctgctctgctgcagagccctgctaaaggccagcccaggccagttacctgggcagacagtgaccttttgcttgccaagactcaaaccttcttagctagcagaatctccccaacttagccgtcagcacattcatgatccaagtaagcccagttactctaagccctgtcactgcatcgtaaaccagctctgtgtctgtgagcaagttagttccaaaagtatattgatttttcctgaaaagatggggctgattccaggcttgcctacctcttaggatggtaataataataataataaacacatagtccttacatcttgcccaggcactgttcacattagcgcttacagcactctcagtaggtactattaatatatctactttatagtgagaaaactgaatcaaagggagattaagtacctctcccaagatgacatacctagtaagtaaaagctaagatttgaacccaggtgtgctcctctggagtctgcattcttaacaattatgataagcagatcttgataaaataatgaaagctgtgaaaactacaaagcactacctaattcttttatccaactttagaaaccggccagatttttaattcaaagcctagtctttgctacagagtgttaggtccttagggccttaggctgctgtccctgttaccctagtcttacactggcttgccttggtcattgctccccactcagtcttgccagctcagggtaggggcaccaagtcctagcagctccctctcccagctcttgcaccacaccctctgggttaagtcactgtccactgaaaaccctttcttctcttgcctagtcttcctggctatccagaagaacaagcctatcagcaagaaggaaatgattcagaggtccaaacggtaagaccaagaaaacaagagtacgtatacaagtgtaaaccaggccaccaagtggtcgggagctctggcttgcacccagaataaatgtattatactcaagtttaaacattatgagaaagttgtgagagtcatttctcacttatggcactgaaaaaaaaaaaagcacatcatggtatgaatgactcatatgcaaaagatgaaaaacttctcactaatttgcatgtcttaggctcctctttattagaaacctaaatgtaaataaagaatattttaggccaggtacggtggctcatgcctgtaatcccagcacttagggaggccgaggtgtgcagatcacatgaggttaggagttcgagaccagcctggccaacatggtgaaactccatctctactcaaaatacaaaaattagctgggtgtggaggcacacgcctgtaatcccagctactcagtaggctgagccgggaggatctcttgaacctgggaggcagaggttgcagtgagcttagatggtgccactgtactccagtctgggcgacagaacgagactccatctcaaaaaaattaaaaaaaaagattattttgaaataaaattaaatatgtatttagaccacatcttttattctagcaatcccatttctaggaattgatcccatagggtgagaaagaacagtgttctccagaacacagtaagttaaacttttttaaaagattaaaatttcgaagcatgtgaacactgtttccatttgtggtctttaaaaataagtatttgtgcctatgtagatgtgtgtacttttatatgcatagaatatttctggaagacagaaaaaactggtaatactgattgcctttggaaaggataacagtagattttttgactttttataatttttgaattttgtgccatgatgaacgaacattagctactcgttaattaattcatttgagtgttgaatgtggtggtacatttctgtaatcctacctactcaggagtcttaggtgagagaatctcttaagcccaggaatttgagactgcagtgagctgtgatcacaccattacgctccggcctgggtaacagactgaaactctgtattcaaaaaataataaatacaatttttaaaaattaattcatttcagatattcatatattgatcatgagaatgccagagaataaggttgacttttttaggcattcattggacacttgctttaataagaatagtttcttagttggcataatgcttctgaagtaatggtactttaaaataatttatctcatgaactttaagcattcctcttgaaatcttgtttttactctatctagtcagcccttataggcaatccaaagggatgctttggatgctttagtccagtggttctcagagagtggtctgtggagtcctggaagttgctgagaccctttcaggcaatttgcaagctcaaaactaatttcagaatgacaccaggatgttctgtgccttttttgctgtgttggctatttgcactgatgatgcaagagaaatggggaggagtaaaatcactggtgtcttaccactattcaagacagtggcaccaaactgtagtagtctagtaggcattgtatcctgcacagcctcccgccaggagtgtgatggaagaacaaggaagcagtgtaaatggaggtacacgggaagcacttcctttgcaccgtgaaggatgatgactgttttaaggaaaagcacttatgccatggtttgtgtcgtgagctgaaccagctgcctttttcatggaacatgacttttacttgaaagagtaactgacagaaaaccaattattctgacttgtgtttgtagcagacattttcttgaaaatgaagaagtgggtctgtgacttcaggaaaatgtctgacagtatctgttgccaaagaaaatgtgagttttcaagccaacaatagaaatctagaaaacttgtattcacctccatgggcttgatgacctcttagtacttagacctttctgacgagataagcggtggcattaacaaatgtgatgtttttcatgttatctaaatacatttgtcaacatttggaagatctgcacaactccctggaccaggattttccaaatgattgttgctttttgttacaaaatcagggaatagatagaagattcattcaaataatgaaagatagactgatggatttttatgtaacagaataggaaaagtttatgacatgttttcagattccaccttgcaactaatttttaagaagctaccacttgtcagccaggcacagtggctcacgcctataatcccaacactttgggagtccaaggtgggcagatcacttgaggtcaggagttcaagcctggccaacatggtgaaagacttctctactaaaactacaaaaattagctgggcatggtggtgggtacctgtaatcccagctactctggaggctgaggcaggagaattgcctgagcctgggaggcagaggttgcagtgagccaagatcgcgccactgcacaccagcctgggcaacaagtgcgaaactctgtctcaaaaaaaaaaaaagaaagaaaagaaactatcacttatcaagtttgggtgtagtatcaaagaagatcaagagttacctgaaaagagtattgcagtaacttctctcgtttccaagtacgtggaaaatctatatgtaagctggattttttaatgtatactttaaccagagcaggttgggacagcagacttaatacagaagcagatacaagtccagctgtcttccattaaaccagacattaaagagttttctaaaaaatgtaaacagtgccactcttctcactaacatttttttgttttcaaaaagtctttatttcccattaaaaatacaggtttcttatgttagcctgtcacaggcttattgttatttcaagtgaataaaaaaataaagttttgcttaatttttaattttaatttttgatacagtatcaatagataaaacctacattgggatcttcagcatttgggagtgtgaagggggccctaagtctaaaatatttttgaaccactgcttcattctaactcagtcacaaaatggaattaggggaaatcatacagggtttgttgtggggcagctactaatactctcacctcagcaagagcagtccctgctgtaagggactaggaagcttgcctacaggaagacagccaggacatctcctaaaatgtatcaatgctttcgacgagaaatatatgagtggatttagtgcccatcttctggagagactttttaaactgtttttaaactggtacattttcattaggttgcttctgttcatgtttatttcttgtgtggcctcttttcaagggagtgatctgtttccatctgagaatcttcaatatcatctagtaactcttaatatttaggaaactacaacccagagaggggaagtaacccagcaagttagggtcacagctggagttagagcctggtgtttggccttcaattgtgtcctattaaggatttgtgtctctggagcctgtgttactcatcttttgttatcacctatctctaccaaagatatttaaaggttcccgtgagaaggaaagatttcaagaggaaagaacatagctcttttatttggaagcacttgttccagagtggccttcaggagacaccttcaccttctctaatgtgtttcccgcagagccctccatgaatctagtgattttttttccagattatagaaaactttaggaaatatagagaaaagtagaaaaaagaaaatgaatatcacttatcatccttccatctaatggaggatgggaatttttggtgggaggcgtgggaattttgcagagacctgcataacacacacttttttccctcaaattgttaatcatactgcatatacaggtttgtgtcttgctttattttattcaacattatgttgtgaacctttcctatgacactaaaaattctgaaacttttgtttggtatacatgaaacatacttgtaacatatacattccattatacttcataatctatttaactgttgttttgttggaaacgtatttacaatgagagtgcatttatcaaatgcacatttgtgtttctcccaacttaaaatcattgccacccaacccctgctagaacagatcccatgtttttcacatggccacaagatcctctctgcagcccacctcaccctcacccccatctgtttcccacctattcctttacgctcctttttgggaagcctcgagtccatttttaccaacactcattttccccaccttgagtgtcttctgtccttctccccattccctttctgctcttatccatcattattatttttttattattattattatttttgagatggagtctcactttatcacccagctgtagtgcagcagtggcttgatcttggctcagtgcaacctctgccacccaggttcaagcgattctcctgcctcagcctcctgagtagctgagattacaggcatctgccaccatgcccacctaattttttttttttttttttggtgtgtgtggttttagtagagacgggctttcaccatcttggccaggctgctcttgaactcctgacctcgtgatccacccacctcagcctcccaaagtgctgggtttacaggcttcagccactgcacccagcgctcctatctattcttaagacccaacttacctataatttcctctctggagcctttccgaacctccttccaccagaccaagtttcttctcctctagaacattttataggtgtgaccattttagtacttttccataatttcacttgtgtttccaaacacaatagtaacgctgttcattatcttatattgaaacagtccatttttgcatgtagagcagaaagtagaagttcagctctccttcatttatctcactcctttcccccctttatcccacttctttccacatgtaagcactattaggattttattgtgtttgtatttatctccccttccagattgtgagccccgggctagggttttctgtgctggagtatctaacggggcccagggcctttctgagagggatgagctcctggagagtgggaggtatagggagggatgatgagaggagatctcatgactctgcaccctggtaacctccccgatttctgtcctttcactggtgccatttctaaggatttatgagcagcttccagaagtacagaaaaagagagaagaagagaagagaaaatcagaatataagtcataccggctgcgagcccagctatataaaaaggtcagtgggtcctctgtctagagtgggatggatcaggtttattggcggaaagagaaatggagaaaacacgttgcctgttgagtgtgaagtcagaatgaaccgcatttgaggaatgaacttagaatgcactggaccagtgttgaaatttggaagaaggcatctgtaattagacttcaaatcaggtttaactccccatccataatggtgtaaggccctttccatgcattcgtttgcagtgaaaccaaatttcacacacaggagttcattgacatatcatggtagaaataccagaattatcaggtttccattccagagtcccaggtggtgatttcatgcccctcattggtgagtcagatacctttagaactgttaaactggaaaatgtgtaactgcggcataagagttgtccacttggatggtcagaggcctccaacttgtcaccatgccctgcccattgtcccagtagagtcagcgttgctgcaagtccaagaacctcacggggcagggttggtgttaccagttaattctctgagaaaccagaccggctcctatgcctgaagggccatatacttcactgtagttgtttccttctcaccctgtcagtcaccagactgatgtgggatgggagctgcttctggagcaggccccagaacccaggcgggtgcctcaggtggaggccccaggcaccacaccacctgtctggtgtgtcagcatcgcagggccaggcctctccttatgctactgtatgtgggaactgatggattatctgtgtctctatagaaataatagatattgattacagatcctctttcctgaacctttcgtgaagtattttttatatgacgaccatagtttctgaagcagagtaaaatgaacaagatttgaataggaccacactgattctccttggtgacatggatgcagggaggagaggcatctgcctctgatggcagtaatatctaacttctttcctgcctttcttttcttctacagagagtgaccaatcaacttctggggagaaaagttccctgggactgacacaagtttattttcctcagagccttggaattctattttatgaacctagagaagcagaatccttacttttgtgagtctggttgaataaagcttattctttgtccatgtgtattttagaaatagtaacttctaaagagtctggaacaaagtggtgattaaaattcctaatggtttgggagcaatactttctgcatagtggccttgtccaatggcctgtgtgttacaatgatatgatcatttctcaagaataagtccctttttgtatgtgtttttatacttttagaaaataaaaactttagattaactcatagtaaacaattctttcccatctggagagatgcaaggagctatgcattgtctccaccgaggtgtctaaatgcatcagttgagggaaatgggctcttagaaggaaatgtagcctgtatctttagaaacacaatgtgctgtagtgggagcactgttgtgggctttgaggagacggtgtctggttagatgcccaccgggtgcacccagagactgcagcaggagaggtgtcttgggtccagagcctcctttcagccccagtccagctcccccagggactctctgtgtaggctggggagaaaggccactagccttggctgatggatcttcctccacccacactcggttttgggattaaagcctcagaccatttatcattacacgtgaaaccaacacttacaacataaaggacaacttgtgaaaaagatgagtcaatgaatgagataaagcatttaacccagtaactggatcagaatgtaacaggattttttttttttttttcatgtggcctggaattatagctaaggcccaaagtaatggaggaaggttttcaacagcctcactctaagttcagcaaaccccagtacaaagattggcaagggccagtcatccaccgagaaggcttcctgggaggctggatccaagagcaggccctgccctcaagcacggttcagagctttcagatcctgccccctgggctccctgccatgaagaggacactgggaaaagaacccatggtgcatgaggtcaactcatagttcttccctttcttgctgggcaaggtaatgaagcacttttggccaatttacctgtaaaatgagagggttaatacctctaagcccctctttacataaactctaaaacaatgtgactatttccacctcttgtgagacattattattcagaatagaaacaaattgttgtctcaaacctgggtctgcaaagaacccagggcaagatggagtctgatgaggaatgatgagcatgattctctctaggcccaaggctgtggcatccagacaccttccaagggcagttagatttggtcctgggaggcccaggtggcaggtgccagccagctcctcccattcccagccagggcgcacctcctcaggagaacttgttagcatcttcactgggttcctttaaaccagaaatctatggacttgctttcacattataacagtattcccccaggccaccagccatgggcatgttcccaaggcaatgtcaacgcacagaaggtggcaggaaggggacatattgtggagagtctcacaactgcagtctacctgcttgtcctatgccaggagacctttggctgagtgcaggctgaagaatccaggcatcaacctcacaatcccagggactccctcagtgtccgttctgccccagagctcaggatggcttcccacactgcacagttctgtgggggctgagagcacctgctacaaaccacagagtcactacctcgtgcagccactcctgcagccatcagacctccctgtgtgtgggaggaccttgagcctcttattttgttttggtttggtttttgttcttgcacaggtgcgggaagcccaagcggagcctgcatcattgcccctggttcttaactgtggtgggattcacttttgaattgcttcatcctaggcctagatcatacctaacctgggaagctccgccctcttccctcctggaggtgagactccggcctattcagtcagtcctcttccctgagggcagcttctgtttcagcccctcccagcaaagaccaccagtgcaagtattttcaggggagagaatatatgaagaaagcagtcttttaactgtaaatcacaatgtgttttgtatgttcccaatgagtgtttggaatgatcaaatgaatgattgtttggaacattgtctggatgttatcaaaacaaaaacaaaccaggatttatacccctggcaagtaacaaatgactttccatgagaatgcaggttttgaccaaaggagttttattacttctcacaagtaaggagggcactgggagtgttctccaaagcaatgtttcctgagagaaaatgacaggaggactttatgtggtgttggagaggggagagggtgcatcatcgcatgtagaggagggttcccagtggcacagatgcagtgagtcatggtgccagcacataggttgcatgttatggtaatgaatctacaggtctcctgaggtgcaggctttagcttggtcatgaggaaagttcactggggttcatctgtaagttgtcagggtctttcaggagctggttccaaccaactaggtgaccacatttcacacagggtttgggataaaactggcttcaaggcaagaggctgtaaaacaggctgactgcttgaattgacaaaattcctatactccctggattccctccctgttggcctacaaatttccttccctggagatatgtcactcctcattcttgagaggtccttcttctgtaacttcatgctgattagggatgccgtgtccagagatattaggggagtgaaaatctctagacactaaatctaaatgtgttttgaggtctcccaagataggctgcaaaggctggtattgttggaagctgcagttgtaatttttctaatctggaagatacaaattttaccaagagatttaagatccagggaccaaacaacagtagtacaaacaataaagttagtagttctactaggggcagaatattaccaagtaattggtattgcccatttgatagcttcccaaatagtcagaactatggccctgtggattaaaagccgtgtaaccaagaggcttgggcatagaccttcttaatgtcaacttctatttcccctgaggtgtttactcagggacagcaagaggaattagtaactgcacacactcctccctgttccactaaaagattgtctaatactgtaccattacctagcaccacagtaaccaaggagtcaactgaggatgctaaagactcaagttttctagtcaagttttttacggtggtttcatgataagcaaatccccaaagagcagccatcctggatgctgctcccatctcccccaaaattaaactccttgctctttttttttttttgagttggagtctcgctctgtcaccctgtctggattgcagtggcgggatctcagctcactgcaagctctgcctcccgggatcacgccattctcctgcttcagccttctgagtagctgggaccacaggcgtccaccaccatgcccggctaattttttgtattgttagtagagacggggtttcaccatgatcgagccaggatggtctcgatctcctgacctcgggatccgcccactttggcctcccaaagtgctgggattacaggcgtgagccactgcgcccagcgccaactcatttctctttttgacctgacttgtagtagacccgcctcaacagcctcaatatcagaggctctcgcaggccaattgtacattgtccttccatccaaacgtcctccgtgcagggatgagcgatgtcaagtggggtaaagggaaggaacctcaaggcaaacaggcgtggggtagatatttgtgggggacccacagatgaacacattccccctgaggggcacatactcatttggggataaagatatttagctgtacacagtggctgatacctgtaatcccagcacttggggaggccgaggcaggcagatcacttgaggtcaggagttcgagaccaacctggccaacatggtgaaaccccgtctctactaaaaataaaaaaattagccaggcgtggtggtgtgcacctggaatcccagctactcaggaggctgagacaggagaatcgcttgaacccaggaggcggaggttgcagtgagctgagattgcgcctctacactctagcctgggtgacagagcaagactccgtctcaaaaaaaaaaaaaaaaaagacatttaatagtggtgctatttggctccaggtgccattagttttacataaagggagaccggcccaccccgcaggtggtccctgtccctgcagtcagctgagacattattcttataaggtgttggcctgtttacctgggtgtaccagaaactggggcttggtattcctcaccatgaactgcatttactgctctgtcctcaatattttttagtgatgttcatgtcaggacaccagtaagtaatattggggtggcaggtggtaggtagtgttcaaggagaccggttacctcccttttgtgtctggtcctgtgtcagtgtagtaagtctgccctggaggaattgcacagcttctgcatacttgctgtatagctgagccatttagcaaggttgacacctaggtggcatttgctatagagatgctagtcaaggtgatgcaagggatttccccactctccaggaagagcgtaaccacagggaaggggagcttgctccagttctgtgggtgaaagtgaaatgtgaagattgccacaagttctgtcacttgggcagaccaagttcatgggtgcctatagcatatccagcattggaacaggctatttcccttcactgtgatgtggggaaaactgactattgtattgtcttcccggagaccactggccatggtaataaaagggactaaccagtagagagagaccaagttcattgtatatactacatggtgatttagcaccagttaagagtttttacagaatgtaaaagagcatagagattcctctaagaatgaagaaattcctgatacatcgagagcattgcaagggcacattttacttatctatgtcctttctctggaagaggagcttaagatcagaggccagttcataggaataagttggatctaatgaagcctggtctgagggcgctggtgcaggcttcattcaggaatgacagatccagggtgtgagtccctgcaatttaagagaagaatgagtggttatcaccacagggtagggcccagtccagacaggatttagctggtcttgaggtgatttagatttccaagtcttaaggtgacctcagtctcaaggttgatataggtgaagcttaacatcagtgggaactggaagacaggttcacatattagattgagagcttgtgttagttttcctaaatacacaatatgttttacatccctctcagtctcaccatcagggcatgtcaaaggttccattgtgccagggtaagctcttccatatagtaattcaaaagggctaagtctcactttcccttttggtgccagctgaagtcatgttagtgtgatggagaggcacttcagccagctttcatgagtttcctggcatggtttagctaaagtgacctttatggttctattagtatctttgtccttttcagatgactggggcctccagtctgcatgtagtctccatttgatttccaaggcagagaataccccctggctaactttggaaatgaaggcagctccattgtcactctggatcaatcaaggtaagccacacttcaagaggatgtctttaagtaaagctctggcatcatttgccttttctgtgtgacaaggtaaggcttctatccagccagtaaaggtgtccacacaaaccagctggtacatgaggtttccaggtgcctggagcatcactgtaaagtctatttgccattcttctcctggtatgcccgcctccgccccttagttgaggcacagagagcctggtctgagcattattctgtacatatttgtggcacttgttagagatttagtctatttgaccacttggatcgggtacctttattacactcatcaaccaattgtgtaaggttttgatatggttaggctttgcgtccccacccaaatctcatcttgaattgtaatcctcgtaatccccacgtgcctagggagagacctgtgtagatgattgaatcatgggggaggtttctcccatactgttctcgtgatagtgaattctcacaagatctgatggatttatgaggggctctttccccttcgctcctcactcttctctctcctgccgctgccgtgtgagaaagcctaagcttgcctcccggttgccttctgccatgattgtaagtttcctgaggcctccccagccatgcggaactgtgagtccattaaacctctttcctttataaattacccagtctcaagtatttctttacagcagtgtgagaacagataccaaaatgtgatgattcatggacatatttaattacagcccagcctaggttttctggaatctggagtcttcctttgtcattgatagctcatccttccaaatccaaatgtgggagatacctttcccatttggatttttctaatttctgagtagtagggccaaaacttgtggagtgacaaatctggggatcagggtcatttgcaaactttgaactgttcatgctgccttcttagcagctgtgtctgctaggttatttccttttataacctcagaattcccctctggtgtccccagcagtggaccgccactaaccttttaggtaactgtactacctgtaacagctgtaatatttcttatgcacgccttatctccttattgtccacagtcatggttccaagggggataatcccagaggcatacttctcatctctctaggaatggggatggattttcattagggtcttgttctgtttcctggagttttccccaattgattggcttacttccctcatgcctctgagtagcatattgctacaatgatccctgttatcactggagtcccagtttgggtcggctgttgggactgacacctcagcagccaagtagatttggtagcccatgttatcagcctacattttgtctgcctcttccttagctttgcccgtgatggtagatttcttttccaccacgagcagagttgccattaaggtctgcatgtctgaccaggtgaggttatgtgtgaagaatattccccttctgagagttctgaactctcatgggctcactggtaagcctttaatattggatttctcattgtacaaatcagtagttgtaagaggtacatagacccaaacaaaacctccctctccatcacacacctgcctgggtgggaaccatcccactctggggttccctctcagggaacttgttatgctggtaacttgagctaccaaatctattgactactgaggtgtcagtcctaacacaacccaaagtgtgaccccagtgataatggggatcaacaacagccagatcattatgggaatatgctgctcagaggcatgagggaagtaagccagccaccagtcagaaggggagtggtgagtggtgctgcctggcaaagagggatacatctgcaagcctcataaaatgagttgggatgaggtttttcttttttattctctagaagaatttaatgtagtttattgcttaaatgattaggagaattcaccagtgaaacccatctgggcctgaactttcttttgtggaaatcatttttttcagtagctattcagatttcagttttccttttatgccaaggtttggcaagttttgtttttcaaggaatttgtccatttcatagttacaaattgattgatccaatgttataatatccttttgttaatgtctggaggttctaggatctgtaatgatgttccatttttattcctgatattatttttgcctcctgtcatttttacctgatcactctttccaaggggtgtatacatttcactggtattgtcaaataacccactttgggttttattgatttttctctatcatatattggtcttatattgattttttgttgtttattttggggcctgctgttgccatagcaggagccacagccagctccacagaattcggaaggcctggcaagtatggaggaagacccttgctttcccctggaggtgctgagagtttaattccagaggaagtaggggagttatttccctgtattggcaaaggttctagtttttgagtaggactaaaggacctcacctctcttgaaggagaagtagagaggcttcttctcattcccttccctacaagtccccacatgactggttcttcctctccaggggcttctagaattagcaacgttgttccctttggtcctttgaccaattacatatcctgcacttcttctgtaaggctgggtgttgatagagggccataagacattatacataaggtattttatcccatttaccttgtttcttacaaaataagtcccagttggagaattgtattactaatgtatgtaagtaactcatgttcagaccatctctcctgattccctagggaacaatggggccacatggcattacggtaatatatcatttgctttttggtcatggatatgggcatccaaatagttttcagtgccggaggagccactgtaacgggctctcctccagtattgagacctggttccccatattgagacctggttccccatattctagggtctttgcccagaaggctgattccactctccaaggagcttccactggggattggtgatgacacaaatcctgtaactcttagcctgtaggatatttctggtcctttcagcttgccactacacccaggccaccatggagtggaggtactggggagctaggagaggataataggggttgcctctccctggatcatgttcagtctgggtagcttcgctggagatctcatggagattccagagtagttctggatccagcttgagtggtgggaggtatgtaaatcaatcaagtaagcaaggtgttctttcgggagagggtgcatgctctgctcatgtaaactctacatcactgtattttggttccaggaattataatcccacccaaactgcaccattctaaattaggcctcttcaaatcaataacagttcttctgtgactgctggaaaatggaagcccactattttagggattaccacagactaaggggaagggaatccagacttcacatgcattcaggtcacaaagatgcataagtattgccaaaagtgacaagcattcagcctacagatataccagacagggacaaagtcttgaagatgagactcctcgtctatattagggcttccccttgttaaaatggatgaagatgtctctcaactcctagacctgaaggggagtcctgcccgttcccaacaatcctggatcccttgagctaggtcctaaacctgacaacccagagtcctaataatcaaaaccaaaaccactcttttagtcagttcacagaaccaccaccacttactgcctcaaagttgcaccaaatgaaaggtccaagcagccacttctgctctcctggtgggggcggaagggggtccctgagccccttcaccaatctccctctcggcaacttaggcaacttaacccactcatgcttcctcagactgctgcttcgcctgtgagcaagcctataagagagatcgagtcagggctcagtccatagtcccatctggatcaaccagatttgtttcccaggccctcccaccccaccacctccaaaaaaaaagcaaggtttgtttgcctggtgagtaacaaaagttgctgagaacacaggttttgatcaataggagttttattacttgtcgcaagtaggaagagcactaagaatatttgccaaagcagtgtctcttaagaaaaaatgacaggaaggctttatggagtgatcagtgatcgagaggggagaagatgcatcatcacatgaagaggaggggtcccagtgacacagatgtagggaatcatcgtgccaccacataggacgcatgctatggtaatgaaactatagctctcccaaggtgcaggatttagcattgtaatgaggaaagttcactgtaagtgagttcatctataagttgctggggtctgtcaggagcaggttccaactgactaggtgaccacatttcacacaggatttgagacaaaaccgtctgcaaggcagaaggctctaaaacaggctaattactcaaactcattaaattccttcactccctgagggcccgccctgtcttgtctgtacagccagtccttagaaagtgaggccaagctgaactgttgacccagtctatgtctttacgagtctgggagctgggtgattttacctccaatactagacttatttttactggttctgaagccttgcttacagccctttcccagtttctcacgaaatactcatgaaaggggatggtggggagtgggcatgggaaaaagggaatggtattaaaattaaagctagcatcattggcctccagggggcactcatggctcatctccatagcccttggctcttgcagctgcccaagatgagggtcctgatctaaggagactccaccaccaccaaggctggggtggtggcaatgggggcattattttccaaacctgactaggccacattttaagtcgatcatagtcacaaggaacatagatgtggactattaattttctttgcctgagcagccgtagccctatgtgctcctctccagcctttcagagagctatgatgggccaaaaaatgctcagtagcccaagccatctccctgcatagacactagagtaaaatctctgcctatgtgcgtgtgtgtcttactctgtcctggacttgtttcctacagtgctggtggctttactttggatactctgattgttgcggagatgagacccaaggctccactctggagaaaatagagccctcttgagaggtcagccaaacttgctagacttcttatgcggtttggggttcaacgttctggcctgtcccccgaaagtcatggccaaattcacatattcctttgatccctgatgccatctcatcacagtgttcgtctggcctcaccattgctacactaagtccaggaacctcaaggaactatgagacaggtagggctagacttaacaaaatggctatcacagtcttcatttcatgagacagaggtgaactgagaaaaggtggaaggtgcttcagagacttcgccgtattcatccctaacttaggcagagagccttggccacacaggaaactggggaagacccttctcatactcaggagtcccccacactggtgccgtcttacaataggcaggcatcagttccagagccaatcaaacaattaggaataagtaacattgctccagaatttgttggggtttttcttcccctgaatttggtagtaagctccacaaaggcaagaatgacaaccatcttgttctccttgtttctttggagcataacacaacatgtagcacgtggttggctcaataagaatttgttgaatggtggccggatgtggtggttcatgcccgtaatctgagcactttgggaggctgaggtgggaggatcgtttgaggcctggagttcaagatcaacctgtccaacatagtgagacccctatctttttttttttttaagtgggttttcttgaatggcacacagaaaataaacaagcaaaatgagagatcagagatgctggaaaagaccagggagatctgatggttaaatcagatcatctaaatgttgaggagacatttggacatctgatagagtttggcattgaataagtgataatacacagaaaactgaacaatggcaaaaagaaaagtatcaattctaagggaaaagattcaaatcaaaagccttcaggaaaggataagtcatcatggtttactgtatagcttagcttaaaatgacacagaatagtggagtagcccaaagggccatctaaccacactgggaagctgagcaggggaggagtgcgtatatgcatggaggagacttgtcttcaccctcagcagctgagagcagacaggctagacctggacaatggagcaggagccacacagcctgtgaaacatgtgccggggaatgtagaggctgctgtttcacatgacagagccgtcaaaccctggctgtttaaaaggggtacatacataattttaatagaaataaaaaccaaaaagaaaaaaggcttaagtgagaaaggcaaaaccattacaatttttagaggaaaacatgagagtaagaacagaaaaatgtctaaaggggcaaagtgctaaccataaagaaaaagactgataaattcatcttgataaattttttatcaaaaggcaccataaagacagtgaaaaaacaaggctcaaaatgggagaagataattgcagcacatctgaccagcaaagagtttgtatccagaatatataaagaacttctatgagtcagttttttaaaaacacaatttatatttaagtctctttaaaacaaatagataattcatagaaatatggtgatatggtttggctgtgtccccacccaaatctcatctaaaatagtagctcccataatttccacatgttgtgggagggacccagtgggaggtaattgtatcatggggtgggttttccccatgctgttctcgtgatagtaaataagtctcatgagatctgatggttttataaagggcagttcccctgcacaccctctcttgcctaccgccacgtaagatgtgtctttgctcctccttcacgttccgccatgattggtctgcacccaaatctcatcttgaattatagctcccataatccccatgtgttgtggagggacctggtgggaggtaattgaatcatgggggcaggtttttcccatgttgttctcatgatagtgaataagtctcataagatctgagggttttataaaaggcagttcccttgcacacgctttcttgcctaccatcatgtaagatatgcctttttctttataaattacccaatcttgggtatttcttcatagcagtatgaaaatggactaatacacatgggctaaactcatgagcaggcaactttatagaaaaacacaaatagctcataaacacaaaatatagattgagttcattattaattagggaaatgccttttatggatagaataaaatacctttttacactccctggattggcacaaagttttgtctgacagtctaaatgttgacagggatgttgattaactggaattcacacactgcttgaggaagcttgcattcactctgtcattcagcacttctgaaaagtgcacaccaataatcagcccaggaaggttcacaaaaacatcttcagcaaagatgtcctttcaccctagcaaagaaccagaaacaactcaactcaagtgtcctttgacaagagaatgggtaaaaagcaagtggtatgtcatgcaaaagagttctagacagaagggaaaattaaagaacatcaaagttagtaagtcttcgagatgatgttgagtaggaaaaaaactacagaaaatatattgtatgatactttcatcataaggttcaaaagcaagccaaggtaaacatgttgtgcatggacacctagggacccatgtcattttaggatcaaacatagatggatagatgatagatagagagatggattttttttttaactcaaaatagtgggagctaggattggagaagagacacttagctttgaaagtattgtcaaatgtagtgacctttctagttcattcaggtgctgggttcaggggtattcattttattatgcaccataatttatgtgagatgttttattatatcaaatatgttctaatcttacagtaagcagttgttaaacagatttataactacatataatgtgtgggtcttatgaacaatgctgctgtgaatatcagcacccaactacacttgtctctcacggccttttgtaagcctgcttagttgaggagtttcttccttatactggagatgagaactcaggtaatgcctcgactgttctgatcactgttaggggattacacattctacccagctgacttttcactgaaagcccagtctccactcaataagtaaaagtgtaataatcgaaactcccaacttgagttccacttaaaatcttctctcttcccctgccaggcctctctgcaccacctaggggagcatagtcccctctgtccctgcctgacagggctctcctcctcagcagcacagggaggtgtctgacccatctgtgtgtgtgtgtctatgtggtagggatggggaccacagcttgtcgtcctgccctctcaccctggctgttcattgtctcaagctgactttcttgggtgttttcatggcttcttctgaggcctccctctggaatattggatcgtaactccagcaatgcaagcaataggttttcctccagcttctggtcctcctcctcctcctcctccccaaatctggtttctagcagtcactccactgagactcagagtcttcttggcccagtgccagcctcttgcatgtcccaaatcttgtcctcaggaaacacttatgcatcctatgctggggtgaactcttaagagtgcaggatcacctcatggccacagcctagctaggccctgaaacccacactcagggtttctcagatatgagtcagccccaggccactgaacctctcccattgcagggaatccctgtcaagtggtgaaagccagcatctagacctggagtcacaggcacctgtagcataactcctcctgaaatggaggtttttctttagagattggtccatatcccttatccctggacttctgagaagggtggacaacctgtggactggacttgagtctggaatttagagaagtgtccatagtagggctgccagatgaggcaaacacaaatacaggatgcccaattaaatttgaatttcagataaatgatttttaatgtttgattctgtcccatgctaaaaattgcccactctttgtgataaatttaaatttattggtccattctgtactttatcaaggacacacgcaggaggaacagtagcacaaaggcttcccggtgccttgtgggtgaaagaaaggcttccagggaaaggagagaaacacccagggatcccggtgacgtcgggaagcagcacccagaccatgtaaggacaccgcctgcagggggagactcaaggcctgggaagattttctaatctgtgccttgagtcgggtggggggattttctgggaaaagaaaaccagacacagtaggacaaatattgtatagtttcacttatatgaaattgtcagaataggcaagttcgtagagacagaatgtagaacagggctttccgggggctagaggagaagacaaggagttcttgcctagtgggtacagagtttctgtttgaaatgatgaaaaacttctggaaatagattgtggtgacagttacacaccattgtgaacataattagtaccactgaactgtacacgttaaaatggttgtatgttatatatattttaccataataaaaactagggggaaaaaaaggaaggggagattgtctcaggctggctgactttccctgcatgtacgcacatcttcacatgtgcacacatgtaaaacaaaaatgaagcaaaaaaagaaattctaatttaaccagtaagcaatatgtaaaaggaaatctgaatgtcattacaattttaccaggttcagaataaaataccatccttcaaactgaacaaattacttattagctgaagagagctgaaaggaagtttggaattaacagtctagattttctcactaatgggtccaagtcattgacctctaaaattatcttagaacatgccctccctccttcctactttctggaaaactatcagtctcctaccctatgcttgcccatagagttccatgtacaaatcacaaatctgcatttgccgttccctggaagcccttacattcccaagggctggcctcatccactgccaccgaaaacagccccagttcaggcaaagcaatcactccctctccctctctgtcattcctttcttttcctttcactatctctctaccctcctcttccccactctctgtttcctctctctctctgtctctctctctcccccaccatctcctctctctttctctccccctgccttctctgtctctctctctgacacacacacacacacacacacacacacacacacacacacacacacacaccgcagcaacattctacctcagccagagttccaggccaataggtgtggactttgacttccagattcctctggcaagtgtcaacatcagtcctggtgctccctggcagagaaggacttggagtcaccttggccaagaagctgtctggaggtcacagtggcaccgcccatcggccccttggccggagctaggtgagggcagattcagcacagcagcaactctccccataaaaaaatccctcagtgtcttctctaacccttcttatactccttgtgagttttagagccacctaaactcttcttttttcccaatacatggagacaaaatttacataacatacagttagccattttcaagtatacagttccctgccatttggtacattcacagtgttttgcaagcaacacccatatcaagccccaaaacattttcattacccaggaaggaaacctgtcgtcatggaaccgtcactcctcacatccccttcccctcaaaccctggcaaccccaaaccgttcttgacagcctcctttggcatttcctcattttggtgtcagatctcacagcagaatttcttacctattatatccagtgcctcagtgtgaagttccggtttaacttcttgttaccacgagcccactatcttgccccaataataccctcccccaattcacaaacacacaagcattccctcctacagctttgggcctcctatctgagtccttcaggaaagaagtgcttgtaactcccttggcagtgagtgtagacttggtccaaggaagatgagcaccagtcagggcagctggtccctcttctctccctggccatcagcaaatcagcactgcccatcgatgcccaggcaatgggagcgtcgaccagcagaggacacacctgaatttctctgcctcatgggagggtggctgctgtgttgcatttaggaagaagctgtgcaagattcatactgttttgctaatgttaatcttgtgggcataataaacgtcgtacccacacttatttttctagatcagacaagattgggtgtgttcagggtggtatggccatagaccccacacttttttttttttttgagacggagtttcgctctcgcccaggctggagtgcagaggtgcaatctcgactcacagcaacctccgcctcccggattcaagccattctcctgcctcagcctccagagtagctgggattacaggcatgcgccaccacgcctggctaattttgtatttttagtagagatggggtttctccatgttggtcaggccggtctcgagctccggacctcaggtgatccgcctgcctcggcctcacaaagtggtgggattacaggcgtgagccaccgcgcctggccgaccccacacttagttttaatacacttctaggcaaggctgctgcactggtctggggatcacactttgagtagcaaaggactaaattgcaataactgccggggggggagaaaaaaaacaggatgaactgcttcctcctggctcagcagctagggagtcactgaactaacaaggggatggggtggggataagaatgttccaggcaaaaggaacaacatgcccaaaggcctcaagcagaaagagagcttcaggtattgaagaagtagaagtagctctgtgtggctggggagtgggcataagatggagcagaagaatgccaagtcctattcctgggtctgaatttccaggagcagaagccctgtcccctctagaaggtggatatagtctggatatttgtcttcaaatctcatgttgaaatgtgacccccagtgttagaggttggacctagtaggaggtgtttagatcataagaactggatccctcctgagtggtttggtgccctccctatgattgattgctctattagttcatgtgagagctggttgtttaaaaaagcgtggcatctctctggttccctctcttgccatgtgacacaactgctcccccttcacccttccccatgagtaaaagcttcctgagaccctcaccagaggcacatgccagcagtgtgattcttgtacagcctgaaaaactgtgagtcaaataaacctctttacattataaattacccagtctcagatatttctttatagcaatgcaaaatagactatcacagaaagttggtacggaggtgtagggcgttgctataaagatatccgaaaatgtggaagtggctttggaactgggtatgaggcagcggttggaaagagtctggagggcccagaagacagatgagaaaaggtttggaccttcttagagatttgtaatgtcgttgtaaccaaaatgtggacagaaatatgtacagtaaaggccagcctgatgaggtctcagatggaaatgaggaagttatggggaactggagcaaagatcacccttgttatgccctagcaaagaacttggctgcattgtatctatgtcctagggctttgtggaaggctgaacttaagagtaatgacttagggtatctggtagaagaaatgtctaagcagcaaagcattcaagaaatggcctggctgcttctaatagcctatgatcagatattggagcaaaggactgacttaaatttggaactattaaatcaaactaaaatctggcctgagaaagcctcctcactcccatacttgagtccttaaggatgaaccataacctaactttgtaggtaggacgactgaaaacctaacttagaaggatgcttctgtagcaataactgagcctcagccaatctcagcagccatacttcagtactcacaggcagcctattgttcaaaccatgttcaaataaggcaaacaccatgctgtaaccaatttggctatttctgtacctcactttggttttctgcatgtcactttcctttttctattcataaggttttttccgaccacatagcatctctggtgttgctctgaatctgctgtgattctagggactgcctgatttgtgaattgtctttttttttttctcctcaattaaattctgttaaatttagtttgtctaaggtttttcttttaccagatctggtgttggaagtgcagtttgaagtaaaactctggtgacctacaggaacaccaggtgaccaggccagttgtgctcactgatcttttgattgtaactggaggttgtagatgggttttctcttggattcagagttccactaatttgtgttctaattggagctctccaagtttatttgagcaatatttaagtggactgggttcaggatcaaatttgaatcaattaactggattgggttctgttagagcctcagacctctactaggcactttttttttttttttttttttgataatgggtttgtctcaatccaaggattctaggaatctaccttctgggactccatctaattttgtatataaaattttaggcccagaatgtgtgcatctttagaaaaacagattaaccttactagagaaaagatggccacaatggggaagttttaatttgtataaaattgtttatttgcaaggcatattagaaaaagggggatcaaaaccccacaaaaacattgggatatagtctttcattggtacatggaagcctctaaaagactaaacgaatgaaaaattccctccttaaaagatactttgcaaaagcaaatgaacagtttaagcaacaggctaaggacatgatgaaagagaactgtactctcactgaactaaccccaactgattcttctctttatccatctctacctacgtactctgagtctactaacctttttgctagatcaacctttcactctgaagatgatgaaaaaggaggaattagatgtacgcctcacaaaggaagaccttctgattatccaggcctgcctgctataaccactttcactccatggtctaaaactgagcttagagctattttagaagacttccctgatctaagagaaaatcctcaaaacttgactaaggagtttagactcctcataggagactatgatccaggactccctgacaaaggttcatatgacaatttattcatatgatatggaggcctggtgaagctcaaaaatggatggcagcagcagaatggaagaaacctgagaaagatattaaaacccccccccccgcaccccccccccgcaccccccccccccacacacacacacacaaagcctcctcatgagaaggcccaacaggagctagaaatattgctgaaaatcccagcctgggcaacatggcaaaagcctgtctatacgaaaaatacagatagttagctgggcatgatggcacatgcctgtagacccagctacttatgaggctgaagtgggaggatcacctgagcctgggagcttgaggctactgtgagccatgactgtgccactgcactccagcctggatagcagagcaagaccctgtctcaaaaaaaaaaaaaaaaaaaggaaaaaaaattgctgaaaatcttttaaattcaaatactaaattttttccacaaaagttgattggtacatcatacaatcttgtaaaccaaaaaaaaaaaaaaagaatgaaccagtgtcagattacagaacttgcttagaaataatgtgaaacattctgggcttaaagtactaacaaggagtatttcctgcagggactgaaatggtattaactactctatttataaatggacttcatcctgaacttagaaatttaattagaaaacgtaaactcggatgggaagttacagccatgactgaattggtggctctagctgaacattttgagaggactgtagagcaagaaaaaacccaaaaggctaacaagcttatgactcttcaattacaacagttacaggggccaagaccaaagggatcttctcattctcattttaaatcacaaccaagaggtattagaccaagaaattcttcaccctaagatgtctgccttattgcaaacaaccaggacagtggaatagggattgtgcccttttatatcagcccaccaatgaccctccctttaggccagcctgtttcaccactagagggagcacaagagaccttagctctcctgaagataatcagcattgaagcggctctgagggatttcacagtatattgctcctcataatacccttaaaagaatatggagaaacagaggttacagtaactggggactcatgtatagtcttggtggataccagagctaccttatctgccactaccataaaacacactttcataagccaacagatccctcagagttaaaaaggctatttctgtggttggggtttcaaatcaaattcaagagtttcccatatctgaacccgtccaattgactttggggcatttttcaaaaaatcacactgttgtactatgtgatagtgctccagtaaatttgctagagggagatttactttcaaagctgaaagggcatagaagactacctattgggtacattgtaaactgcttgggaaatgggcacactaaaatctcagaaatcagcagtaaagaacttgtccatgtaaccaaaaaccacctgtactcccaaaactattgaaattttttaaaataaaaaatttttaaaagtggggagggtgtaaaattttccttggaataagaatcagtttcctgattctcctgaaccagaattgttatgttctctataggcagatattaataggatcgaaactcaggcctgtaatgcctgatcttccaaaaatacctaaatatttatgggcctcttcctcaactgatacaggaagaatttaaagtgtgtcacctattaacctccaaatagaccactctaaacttttgcctaaagtacccaatatccactaaaactagaagcaattcaagggctcttgccaattgtaaaagatttcattagacagggacttataattctatgcaccagcccttataaccctccaatcctactggttaaaataaaccaaatggatgaggttgaagatttgttcaagatttatgggcagttaacaaaattgtagtagcaaggtttcctatagttccaaatcctaatacttcattatccaagatacatgctgattcttaagtggttcacaatattagttctctgctcagcctttttttacaatccagtttataaggagagtctatgcttatttgccttcacttggaaaagtcaacagtacccccagattgtgatgctgcaagggtttaccgaagcctctttgtgttttcccaggcattgcatcaggacttaatgacattatagtttcctcaaaattctactctcattcagtacgtggatgacttactgtatgctctcccactaaagagtactctgaaatggactcagtttaccttttacacaactcacacatcaaggtcacaaagcttcagtagaaaaacttcagttttcaaggggaaaaagtccattatttgggacattatttgatggtgtttccctctcacctaaaaagttaaaaactcttcaaaattttccttggcctgcaaccaaaagataattaagaggttttcttggacttgcaggatattttcttggacttgcaggatattttcttggacttgcaggatattttcttggacttgcaggatatattcttacacttgcaggatataaaattcctgggttctgaatttttccttaatagcctcaccgttgtatgagctcactaaaaaatgctgtaccagagcctttaccttgggatgatagtcatgagcaagcttttagctaaataaaattggccttataacagcctccagctttaggacttccaaattgcactaaactgtttacctaattggcttatgaaagtaataatcaggcatttaggagttcttacccaggaacatggggagaaacataggcccattgcatattatagcctgcaattagacccagtagctaaggtatatcctaactgtttaaaagcagtagcagcagcaaccaggctgaaagaagcttcagctgatgtggttttaggaaatgaacttaatatggaagtcccacatgctaaattccacccaaacccagcattttttagtaagtagactaacatcttatgaaatgctctgtctccctctaatctctatctaaaacactgcaatttacttaaccatgcccctgtattactgctgcctgatgatggtgaagaccacaattgcataagtgtagcatcagaaatagtggcccctcatgttgatttacaagttagtccattggacaatcctgagctaatacttttgttgatgggtcctatgccaaaagctcagaaggaaaatatcagctaggatatgctgttaccaaaatgagttaatagagaagggaatccttcctctattagtcagctctattagtctaagctctataagtcagcttaaccctcgaagatttttgccctcacccgagcttgtcatatagctaaagacaaaccggtaagtgtttagacagatagtagatacgctttaggagtagtacatgattttggcatgatagggaaactctaagggtttctcacttctagtgggatcaccatcaaaaaaggactccaattagataaaccactagattgctgttattgatcgaagctcatacctgcagaactgaacctgaatatcaagggaatgcttagcagatatttatgctaaatcagctagtactgaaactgttcagatatgcaacatgaacgaacttcgtaagattaatccaagccaactaccttacaatgacctatttaataaagaatgcaatgcacctcatttggaaaaacaaaactggtatctaaaaggatataaattcaatgttaagtgcaaacttgcagagggcctagatggctgcctggtctttcccgagtctttgaagcttccattgtcaaaagctttgaactccacaactcatcatggaacagaaaaattcatccaagttgtgaaaaaatacttgtgggatgactgttccaaaattgctagaatatttttttttttttgagatggggtctcactctgtcacccagggtggagtgcagtcgtgcgatcgtggctcactgcaccctccaccttcctggttcaagtgattctcctgcctcagtctcccgagtagctgggactacaggtatatgccaccacgcctggctaattttttttatttttagtagatatggggtttcaccatgttagccaggatggtctcgatctcctgacctcatgatccacctgcctcggcctcccaaagtgctgggattacaggtatgagccactgcacccagcctgctagaatattttataaccaatgttggctagtcaaacccatagtcctggaaaaacgaaagattcaggtggtgtattgtattttcaacacctaatggaccatttgaacaattatacatggacttcattcagttgccaccttcaatgtgggtctcagtatgttcttgtaatagtttccatgttttctggttggttagaggccttcctgtgtaggaaagctgatgctgtgacagtagctaagaaaatattagaaaatgtgttttctttatggagcatccctggaaaaatccccagcaatgggggaattcattttattaggcaagttataaagcagttaaacaaggttttatggacacaatggtactaccattatccctagcacactcagtcttctgaaaaggttgaaaaaacaaatgacatattaaaactgaaattggcaaagttaactgaatcagttgggttgccttggccaaaggtactatcattggatttaatggcagtcagatccactcactaaaaaacagaagtggaccccttatgaaatagtcactagaaggcctatgccccagtagtaaaacctcttgcatctttctctctctgaaattctgatataagtaagtactgcaaggccattataccaagtgtgctttcaccagataaaggaagcttttcaagatccaccaattgaggataatcaaaccttccacagtctagaacctggagattctgtcttctggaaatgacatcagaggaaggctgctcttgacaactctttggaagggactgtgccgagttcttctcaacacctacactgcagtgaagcttcagggccttgaaacttgggtccacacctcacaactcaaaaggacctcttcagactcctggaactgtacatctgctagagactttaaggtaaagctgtctaaggaaaaatctccagagcagatgacattctagttgtggacagctttcccaagatcacagatgaagacttctgtatcatcatgaaagccttacatttttttcttttctcctcatttcctgttgccctaattctttctttttccctacagaaaaatccatgggaccataaccagtggatgcctttagcttgagcatctgctgtagcacagaaccagagtttgtggattattgggtttgttgtttattgccaaaaaaatataaggaaacaattccactaatgccagtgcctctccatgttcccaatgagagtcaccccaaaattccaagggaaggatggaaagctatccttgatattctgaacaccactgctacttgctttcctgcactcactaaaaagaacactttaacttttcctttttttttttttttctttttttgagacagtctcactctgttgccaaggttggagtgcagtggcctgatcttggctcactgcagcctccacctcccaggttcaagggattctcctgcctcagccacccaagtagctgggactacaggcgtgcaccaccgcatccagctgatttttgaattttttgtagagacagggttttgctatgctggccaggcttgtcttgaacctcctggcctcaagtgatctgccagccttggtttcccaaagttctgggattacaggcgtgagccaccacaccctgccttaactgtttcaattaataacctgatcattaccaaagtatagaaaaccaatccaagtgatgactgcaaaagatatattgtgctttcaggcatcatgtactcaagatttgggatttacctatgtgagtacaagtaattgcttgtataatgtcaccagattaaattcaacagggtctctttttaccaaatgttttatatacccttacaacatgctataagaggggcacaaaaaggtcaatttctcaatggattttgttcagcagctatgcagatttattaatgaacaaatctaactgaaccctgctcaaatgcaactaggtagccctgtttttcaactcccaaggacctattttgggtctgtggataatctgcatatttcattctgcctcttcgttggctcagatcttgctatttggtctggctcactcctgcctttcaaatagtttcacctaaaaattctcctaacagctcttatgattggaggccaaaatagtcaataactgaaattagcactagctttgaaatagacaaagataagctagtttccactgaggaaagattccagtggggttcctgggggctcactcttggtggtagtgggatactagttgtatggaatccaaagctaatccatatgtggggtcttggattttatagccaatcaaacatcccagtgtttcagacaggtagacactactctccaaaaggtagatgtggcctggtgcaatggctcatgcctgtaatcccagcactttgggaggccaaggcaggtggatcgcctgaggtcaggagttcgagaccagcctgcccaccatggtgaaaccctgtctctactaagtacaaaaaattagccaggcatggtggcacgtgcctgtagtcccagctacttgggaggctgaggcaggagaattgcttgaacccaggagtcacaggttgcagtgagctgagatcaccccactgcactccagcctgggtgacagagcaagaccctgtctcaagaaaaaaaaaaaaaaaaaaaaacaagaggtagatgttacagttaatggaacatcatacagcttcatatctcctttttactcaagctggggcctgtgtttggtgttgaacaaaatagaatgctgcagttatctctctcctgattttgttactacagaaagcttaatgtaaaggtggctgatactgctgtttccttagacactgacaccaaatacaataaagaagtctctcaagaggaaaacatggtgtgtttacaggagcaactaacagttggtttgtaggcatcctaaatggtggatggcaagcttgggtttcccaaaggtttctagtctttatatgtcttctagtaggtgtccaggttagtatggcttgtgttaccagagcaacaatgaaaatggctacatctttaaatcaggccactttacagtgaactatgatccttaattgctaccacatttcaaacaagaactatgaccaattaaactttactgttgttgaactgcctatgttgcctgaactttgactggtttaagttggtttcatttatttaataagaacacttgttaaggagtgcatcttggtattttgatattattctcttgatagtcataataatagttccctagtgtgctgcatcctctcaagtcttaaatgttttgtatgcagccatacattgagaattgaatggtctcactttgactaggtcagcaagaacatagagaatcattcacctagtgtgaagctgtgacttgtgaacttcatagtgacaccagtaaagacttgtgaactggctgggcatggttgctcacgcctgtaatcccacacttttagaggccaaggcgggcggatcatgaggtcaggagatcaagaccatcctggctaacatggtgaaaccccgtctctactaaaaatatacagaaaaattagctgagcgtggtgatgggcacctgtagtcccagctacttgggaggctgaggcaggagaatggcgtgaacccaggaggcggagctacagtaagccaagatcatgccactgcactccagcctggtgacaaagcgagactttgactcagaaaaaaaaaaaaaaaaaagacttgtgaactccatactgagactaaaaagacttgtgaattccatactgagccaaattacaatagtgagagggtggcatcaatgcctaaagtttcggtctatctctctaaattgagagtctgaccaaaaggggggagttgataaatgaaactcaaatctggcctgaggacgccttcacattcccatatttgagttcttaagggcgaaccgtaacgtagttagtaggtaggcagactgaaaacttaacttaggaggatgcttctttagcaatagctgagtctcagccaatcccactggccatatttcaaccactcacaggcagccaactattcgaatcatgctcagataaggtaaacaccaagctgtaaacaatctggctgtttctgtacctcacttccattttctgtatgtcactttcctttttctgtccacaaatttgctttgacagcacagcatccctggtgtctgtctgtatctgctgtgattctgagggctgcccgatttgcaaattattcatttttcttgctcagttaaactctgtgaaatgtaatttgtctaatggatttcttttaacagcacttataattaaaatggaaagagggtgtaaatatttagaaaacttgcagcctggccatgtgatagacaaggaggaatccaagcagcctgctgagcaactatttgctagagacattagcatatctaaaagggagccaggtgctaatattcaagacagtgggaaaaggcctcagaggcatttcagaaggcctctcctatcacaggcccacaggcctaggaggactgaatggtttcagagaccaggcccagggcaccattgctgtatgtcacctcaggatgctacttcccacatcctagcttctccagctccagctttggctcaaagggcccccggtgcagctcagaccacttctttggagggtacaagccctaagccttgggagcttccatatggttgtttttttttctttttttgagacaatcttgccatgtcacccagtctggaatgtaacggtgcgatcttgactcactgcaacctctgactccagggttcaagcgattctgctgcctcagtatcccaagtatctgggattgcaggcatgcaccatcatgcccagctaattttgtatttttagtagagatggggtttcacagtgttggtcaggctggtctgcaactcctgacctcaggtgatccacgtgtgtcagcctcccagagtactggaattacatgtgtgagccaccatgcccagccttccatatggttttaggtctgcaggcttgcagaacgcaagagtgaaggaggcttgggagcttccacctagatttcagaggatgtatcaaaaagtctgggtgccctgacagaagcctgccacaggggcagaaacccacagagaacctctacaaggacagagccatgggaaaacatagggttggaaccccatacagagtccccactgggacattgcctagtggagctgtgggaatggggccaatgccctccagacagcagaaaggtagagtcaccagcagcttgcaacctcagcgtggaagacccacaggcattcaatcccaacctgtgagagcagccatgtgggctgcacccagcaaagccatagagacagggctgctcaaggccttgaaaggccactccactcaccagtatggagttacggaagatgattttggaagtttcagatttagtgtttgccctgctgggtttcagacatgtgtggggcctcttgcctctttcttttggccaacttctcccttttggaatgggaatgcctacccaatacctgtaacacaattatatcttggaagtatgtaacttgtttttgatataacagcctcataggtggaaggaatttgccttgaggctcagatgaaactatggattctggacttctgagttgatgctggaacaagctgagacattggagaactatcaggaagggatggtttcattttgcaatgtgagatgacatgagatttgggggtaaggggcagaccaatatagtttagatgtttgttctctccaaatcatatgttgaaatatgacccccaatgttagaggtggggcatagtgggaggtgtttcgctcataggggtggattcctcatgaatgggttggtgctttgtccatgataatgaatgagttctggctctattagttcacatgtaagctggttgtttgaaagcacctagcatctctcttgtgtgctgtctcgccatgtgacacatctgctcccccttcagcttccaacatgagtaaaagattcctgaggccctcaccagaagcagatgcctgcactgtgctttttgtacagcctgcaaaaccatgaaccaaatacacctcttttccttataaattacccagtctcaggtattctgttatgatcccctggaagagttatactacttccaggtctagcccatggtaaatcccacactcccctctgaggccaccttcttccctctgcagagaccctggagttagagtggattggttgtgtctctggccccaattcttctcccgaggctgcattccacccttttgctatgtgactttgcagcccttctcactgcagagacaggttcctctccttgagtttggggaagatgcaacacatgcaggggtttgaaaacacatttgtttgtttctgcttttgtttttgctcctctgcaatttccttgaaaccatgcctgaggtagcctgtgggaaaacgaaacagcaccattgacctagtctttcagccaatcccactgcatttgtacaatgcagtctgacaccagaggaactgcccagccaagctcaggcaaaattgctgacttcagactcatgaactaaataaatatttgtttcatttggtggttgttcttatgcaacattatgctggcactagaaaaatgaaacagatgggaatcaaaaagactgaatgaagcagaaacccagaagctaccccacaagcaatcacatgggactcagatgtgaatggacaataagcttttattgcattgaaaggtcattgcagtgaaaggttggggattgcttgctgctacagctgaatggattctattctgaccaatacacacagaaagagatcacagactccctaccttagaagaagggaggtggtagatgaaatgaactgtatgaagagccactagcctggcccacacacagaagaaggactggcccgtcttcttgaagcccatgctctggtagagggccatagcagagagctggatggtgccggtgtccaggataacttcactgtagccctggtcccgggcaaactggaggacagtcctgaccagggcttttgctatcccctgacgacggtgctcactgtccacaaagagatgaaacagctgcaaccgcttctccctcaaggtgggatcatcaacaggcagagctcctaccatgcccaccaccttctcttcagactcagccacccagaagcaggagccacgctcactcaggtaggatttggtaatgtcagacatgtctgtgcacaatgtcatgtccacatactccgtccagggttttttggcaaggaaccacagggcagggaagaggctgatgctgaacacgagggctagaagccaggatccagagaccaggagtagggcgaggggccccccaagtaagagtatgagggttcgaggcagcttcagcaattgccggaaggtggctggggcatgctcggccatcccccgggagagcaagcccacaacccactggcggtcgctctcctggtatttgcggatgtgacaaggagccatggacagacttctgtgtctgaaatctctaagtccaggagacagagctaggcctccctgcacgcctttacgtcagactggggaagagagagaaaatcatgtgagtgcctgcatggctcccagccttgcaggaacagggagacctgctcaagggtgtgtctttccgcgtttgcccatgaggaagcaggcctctgccactgggagaaggtagggtcagaaagacctgcattggaatctggttccatccctttctagctgtgtgaccttaggcatgtcacttaacctctctgagcctccatttcttcaactatagaacaggggagaagaaaacctgccttctagagttgttttgaagattgaataagatgccttctataaagccctggtagatgaactacccaccgctcttcctttttctgtttcctttttctggctctctcagcttctccgtgcattgagggggcacctacctcaggcaggccacacgtgcaacccttttgtccttctccagggaccatacatcgcaaaaattctctgtccaacacaagtttcaggaaaggctccctccagctttattgtaagtttttaggttgctgaagtggcataagagccaagcataaaaaacagcccagcctccagatcccaaggaaggtgcatgctcagagcactgtccttccacccagtcccctcacctgtcaccacaagagccttgagtgtccagggagcttcagctctcagccgctcactggcatccaggagataccgtctggggcttgctcacccctggaaagtagactgcctgccttattgcttggctcctgaatgtttggtaatcatcaaccctagggtcaaagagctgggctgtaacacagcagggagcccagtgtgcattcctctttgtggtcaaagggaacgacctggagggggagactcagggtctggaaccgcctgggttccttgcccttctggaaggcctgccacctctcttgcctgttcaggccccgtgcatctctctgccacctggcacacttagtactctcggtccctgtccacatatacgatccaccctggtccttgtacacatatacgacgagcctgagaactgtaccattcaccctgcaatcagtcaccattggccagaagccgagtccccatgcttggcaacaaggcccttcccagctaggcttctcctcgctgaggtttccctggtccacgaagcatctgttgttggcagttccttgcactcacctcaccaacccccatctccacccattcattgattcagtcaacagttgtgtgtcatgcacttcctgtgtcctggggtcatttcagccatgagcaaggttgacattcggcttgacaggcatggctgtgtgtggcttgtgtgtgttctggctgttggtcagaggctgtgccacacccgttgttaaatattttgcactgcagatgttggggaggcccaaattcatgctcttgtacccgacgtggagctgatcccaaacactgacacatgggtgcttggagatagagaaagttgtattcgatttggccaaagcaagaaggcaggagagcaagatctctcaacttcagttcaacaaagaagcagcagcagaatgtttttatgcagctagagagtgaggaagggggagttcagggggattgagaggaaaagtctgtgtttcctcagtctgagataacaccttgtgcaaccaggcttctcagtgtcagcagctggtcacagtgtccttcaaggcattcattcattctgcaaacttttcctgaccctgaaggaatgtcttcctgctgaacaaagaaacagtacatgagcagtttatcattatgttgtgggaacaaggaatattggaccaaaagcaagtggttaacatgtgcaagccagcaacggtctgatcagaatgttcattatttcagtcactaaaacatggtgtggtgctgaaatctcaaggggcccaattacaaggatgcaacatatacaagataggcaaaagcaaaacctaacagaaaaagcccttttccacaccaacttacatgctattgaagggaaacttcccttccaaagattagctaacctattgatgatcagccacgctgggcccgtcatctaggttaggtagtttggactctattccaagagcaaggggaggacattgatatgcacggaggggaggaggtgtgtgtgtgtgtgtgtgtgcgcacgcaagcatcactctggctgcaggtggacaatggatctgaggaggtaggaatggaagcagctacccctcaaggctcatgccacatccaggcaagagcggggcctccagtacagggatgcaggtgctcactgtacaagagcagctggcccagaggctctggtgggagctaaaactgggcctccctctggttgccaagccatgagccatggcacgaaactgagactggccaaaggaaaaggcaaccttttcttatttgctcaaaggcgttgcttgcctgctaaactctgcatccagtgggctgcatggcttgaggctgtgcctagctctgggttcacaaagaagccgcctgggttagcagaggcccatgagacagcagtggcttcaccagagggaggaagtcaagaaggtgagacatgagccgtatggaggtgcatttggggcagaaatggcaggagttggaatgattcacacagggaggaggatgaggaagggctcagagacgacagccaggcttctggtaggggcattcaggaaaacactggagagagcccagatctggaaaagtcaaggcttcccctaaaggtgctgccctgcagccttcttcagcagaaaccagaagctgggagtcagcagcaggaacccagggccagccagtggaggctgctgagattccagatctttggtacccaacaccacctccgaggaaaagtctaaggagcatcaagtctgggcaggaagtccatggctcatctttagagtgttctcgcaacatttcagtgtcaagcaggagttggcctaggaatcaaaagaggtgagcggtggctccaaagcaccactgagtacttctagtgtgagtctaaccaatattcagtcagaaacctgccggtcacgtggagggggagaggtttgtgctccgtccctgtgtgctgtctctccctccatgctcacccgtggctgtggcttcctcaaaacacagcgacctcacttgacattcaggcgtctcttgctcccaggatttactgtgacttctgcatttttgtagctttcctgataaaagcctgggctttccttgtcggacctgaaagattcatttattcattcaacaaacagtggccgaaggcttgttatgtgctgtgcctggacttggcatgaaggcaccagatgtcagaagcgaggctgctgcccaggaggacacacctgatggggctctgggaccaggacgatggtggagacatcacatccggaataactttgttaggaaggtgaacttgacagttaggacttagttctccttgaagacgtccttcacatcccttgtgagttggattcctaggtattttattctctttgaagcaattgtgaatgggagttcactcatgatttggctctctgtcttttattggtgtacttgtgatttttgcacattgattttgtatcctgagactttgctgaatttgcttgtcagcttaacgagatttttgggctgggatgatggagtttcttttgtgggcatttagtgctataaatttccctctacacactgctttaaatgtgtcccagagattctggtctttgttctcattggtttcaaagaacatctttatttctgccttcatttcattatgtacccagtagtcattcaggagcaggttcagtttccatgtagctgagtggttttgagtgagtttcttaatcctgagttctagtttgactgcaatgtggtctgagagacagtttgttgtaatttctgttcttttacatttgctgaggagtgctttacttccacctatgtggtcaattttggaataagtgcgatgtggtgctgagaagaatgtatattctgttgatttggggtggagacttctgtagatgtctattaggtctgcttggtgcagagctgagttcaattcctggatagccttgttaactttctgtctcgttgatctgtctaaagttgacagtggggtgttaaagtctcccattattattgtggtgggagtttaagtctctttgtaggtctctaaggacttgctttatgaatctgggtgctcctgtattgggtgcatatgtatttaggatagtgagctcttcttgttgaattgatccctttacctttatgtaatggccttctttgtctcttttgatctttgttggtttaaagtctgttttatcagagactaggattgcaacccctgcctttttttgttttccatttgcttggtagatcttcctccatccctttgttttgagcctgtgtgtgtctctgcacgtgtaatgggtctcctgatggtcacactgatgggtcttgactctttatccaatttgccagtctgtgtcttttaattggagcattcagcccatttacatttaaggttaatattgttatgtgtgaatttgatcctgtcattatgatgttagctggttattttgctcattagttgatgaagtttcttcctagcatcgacggtctttacaatttggcatgtttttgcagtggctggtaccggttgttcccttccaggtttagtgcttccttcaggagctcttgtagggcaggcatggtggtgacaaaatctctcagcatttgcttgtctgtaaagaattttatttctccttcacttacgaagcttagtttggctggatatgaaattctgggttgaaaattcttttctttaagaatgttgaatactggcccccactctcttctggattggagtttctgccaagagatcagctgttagtctgatgggcttccctttgtgggtaacccgacctttctctctggctgcccttaacattttgtccttcatttcaactttggtgaatctgacaattatgtgtcttggagttgctcttctcgaggagtatctttgtggcgttctctgtatttcctgaatttgaatgttggcctgccttgctaggttggggaagttctcctggataatatcctgcagagtattttccaacttggttccattctcctcatcactttcaggtacacaaattagatgtagatttggtcttttcacatagtctcatatttcttggaggctttgtttgtttctttttactcttttttctctaaacttctcttctcgcttcatttcattcatttgatcttcaatcactgataccctttcttccagttgatcgaattggctactgaggcttgtgcatttatcacatagttctcatgccatggttttcagctccatcaggtcatttaaggacttctctaccctggttattctagttagccattcgtctaatcttttttcaaactttttagcttctttgcgatgggtttgaactttctgctttagcttcgaaaagtttgattgtctgaagccttcttgtctcaactcgtcaaagtcattctctgtccagctttgttccgttgctggcaaggaggtgtgttcctttggagcgggagaggcgctctgattgttagaatttttagcttttctgctctggtttctccccatctttgtggttttatctacatttggtctttgatgatggtgatgtacagatggggttttggtgtggaggtcctttctgtttgttagttttccttctaacagtcaggaccctcagctgcaggtctgttggagtttgctggagctccactccagaccctgtttgtctgggtatcagcagcggaggctgcagaacagggaatattgctgaacagcaaatgttgctgcctgatcattcctctggaagctttgtctccgaggggtacccagccgtgcgaggtgtcagtctgcccctcctgggggatgcctcccagttagggtactcgggggtcagggacccacttgaggaggcagtctgtccattctcagatctcaaactccatgctgggagaaccactactctcttcaaagctgtcagacagggacatttaagtctgcagaggtttctgctgccttttgttcagctatgccctgcccccagtggtggagtctacagaggcaggcaggcctccttgagctgcggtaggctccacccagttcgtgcttcctggctgctttgtttacctactcaagcctcagcaatgatgggcgcccctctcccagcctcgctgccaccttgcagttcgatctcagactgctgtgctagcaaaaagcgaggcactgtgggcgtgggaccctccaagccaggcgcgggatataatctcctggtgtagtgtttgctaagaccattggaaaagcgcagtattggggtggaagtgatctgattttccaggtgctgtctgttacagcttcccttggctaggaaagggaattccctgaccccttgcacttcctgggtgaggcgatgccttgccctgcttcggctcatgctcagtgggctgcacccactgtcctgcacccactgtctgacaagtcccagtgagatgaacctggtacctcagttggaaatgcagaaatcacccgtcttctgcgtcgcttatgctgggaactgtagactggagctgttcctattcgctcatcttggaactgcccaacactccatccttatctataacccagcattcttctataccccatagaaagcaccccttacactcggacatgcacaggaaggaagctgctaattggagggaagactcactcacactgtccccaggctccactccaaaggtggccagtgcagctctttcacctccattacctccaagtggtgctgtcactcacagtaagcccagaagagcagtctgacctccacatagagccccctcatttccactcctcactgctggctgctggctccacggaccctctaaaaccacccttggtaagatcaccaggggtttcctaactgccaagtaccagtaatgctttatagtcctcaaacatgctgaacgctaagggagttcctgcattcataaagctgcttgcggtacctgggtgctatggtctgaatatgtcccccctaaactcctatgctgaaacctcatccccaaggtgatggtattcagaggtgggatcttgggaggtgattaggtcgtgaggatgaagtcttcacctatgggatcagtgcccttacagaaagacccaaaggagcttgtttgccccttccgccatatgaggatacacaaaaaccgccatttatgaggaacaggtcctcaccagacataaatctactggcactttgatcttggattcccagccttcagagctatgagaaataaatttctgtttacaagctaaatgtaaagtgtttaccagtttatgataattgtgttataacagaccgtatgaactaaggcactggagtcagtccaagagcttagatgtaagggaaataagatcagagcagagatgtgcaagagactggctcctagcagagcaactaagagtcaggcaaataatgtcaactgaggaggcttctgggtccagcagccacccagctgctttaacacatggctcttcatttcctatccccttctctcctggtctgtctttccctctttgatggttctttttcaagctttcattttgagtgttcctcaaatggaagttttcctcaaaacagaaaagttcatatttccttcctaataattaataggaaaaaaatgccaaatggatagaatggactccggataagaatggagaattttttaaagaagaagcaaatatggtttatgaatatattttaaaactgtgcaaaaaagaatatacaaaaaaactcactagaaatcaaataaataaaaatcaaattaatatccaggttttacctatcattgtaaagaattaaaagaatggtatgagctaatataggacaagcacaaagaaatgtgcagacacacacacttttgacggaatactgaaatgggagatccatgctggagaatcattacacagcatgtcaaaatcctttttttaaatcaaaacagagtctcgctccattgctcaggccagagcgcagtggcagagtcataactcactgtaacctcaagcttacaggttcaagtgagcctcggcccttgtcctcctctcaagtagctgggcctacaggcacccaccaccacatctggctaattaaaaaaaaagttttttttagagacggggtcctgctatattgcccaggctggtccctaatccttgggctcaagcagtcttcccacctcagcctcccaaagcactgagatttcaggagtgagccaccctgctcgacctcaaagtcctttgaaaagacttgatcgaaattcaacttctagggttagagctcaagaaagtaactggtcaaatgtgcaaagatgtttgcataaggatgtttatcactgtaaagtttattgtcatagaagcaaccgaaagtgcaacaagaggggactggctgaataaatcctgctctatttctgtagcacagtacctcacagccatgaccaatgctgatgagggtttactgacatgaaaaacattgcccaactatagcaccaagcaagaaaagattataaaacaatatgtttggatttatttttggaaaaatggtattgaaaaatgatatccacaaacaagcatgcagagagaaaagtctgagtggttccaccaaatgttggtagcagttacctctgaaaagaggaattgtgggggattctctgcataaaagataaactctgaaggtctttgcacattctaggcacttggccctgctttcagacagtgaaactgaatcttgggagtctaaagggactcagttatgattgtgagctcctaaaggtaagacccgtgttcggttttattttattatttcaattaatcctattaatgtgcacaattcagtggcatttagtacattcacaatattgtgcaacgaccacccctttcttgttccaaatcatttttatcaccgcaaaaggaaattccttattcattaagtggttcccccttatttccctctctccccagtccctggcaacctaatctgctttctgtctctatggatttatttattgtggatacttcatataaatggaattgtacaatatctgaccttttatgtgtggcttctttcagctgatgttttcaaaattcattcgtattgcatcatgcatcagtacttcattcctttttagggctcaataatattccatgtatgtttatactgcactttgtttatccattcattcattgatggacattaaggttgtttctacgttttggttattgtgaatggtgctgctgtgaacattcaggtacaagcagttatttgaatacctattatcaattctttcgatgtatacctaggagcaaaattgctggttcatattttaatcctgtttaacattttgaggaaatgccaaactgtcttccaaagtgtcagctgtagtttactttcccaccagcagtgaaccttattttactccactgcctgcctcagcactactgcctagaacataaagggaagaagcaatagctgtttttttaccaagaagttattagatgcccagcactgtgagaaagagggcttcccatgttgtgcaatttcatgttaacagccttggagctaaggttggtattattaacatccccatttggcaaatgggaaactgaggtccagaaacctgaggtttcctctccacggccccatatgggaagtgaaagctcaaacctaaggtagctctgactccaaagcctgcactgttttactttttcagtacagcatgctgaccctgcaggcagggatttgacaagtgtttagtgagtctgaaattcacataaagcctcacaagcccagagaagcagctctgtgtggaggcgcataagattcaccagtaaggccccatcacccccatcctgttgccagtggtcaggacacccttgaccaaattcccatatgtgactaaggctctgttccttcttcaagatccagtttagtgctacctgctccgtccacttctgaccacccaaccagatgaatctggtcccagtccctccctccttggaagcctttgcatttctcattaagcacttgtattgctgccatttgttgcttacttaataattaacttatcatagttaattagactagaaattaacagttcaactaccctgataaactattttgtctttctattgcctctatgaacctgattataataaatcattgtattgttggagtcactgttattcccagagctgtcaggtttcagagcagcgtgtcagtgccaccccatgcctgcatccgggaagatcactcctccactggaccagcccagcctggaaaggggccagtgaggcggaaggtggacaatccagggccaatacattctgggagtctcagaggaagagaaaaatcgattcagactcatttaacataggctcctggaaaggatatcatctcccccaatctctcatcaataacccaacatcagatcagtgccctggacttatcatatgaagcagaatgaaagtagacagataggtcatccttggttagatgtccagaaatccgaggatggtggtcctagctcttcagagaggacacgttctttaacctaaccttttaacaaattctctacagaagatatcaggtcccaacatttcaagtagcttcgtagatataggtaatcaaaggactaattatgaaagtgtagatgggtagaggggagcaacaaggatggcacaggaacccatagccctgaggaggagggagagttgttggaacctgaaaggacctgggcagagcaggctgcccttagagcaggcctggggccaaggcacaatcaataccaagcctgcctcacgctctcttccctccactcctcaacttgctgctggctttcagaaaagttaagaacttgccatttctacccctttgccattgttgttgtcttaccataaggcggggagcaatatagtatccagttttctagatatggaagtggaaatcaggtgtatatacccagtatattagtacctattagtattcttattggtattactttctagatgttatgtaactgtagctttggattttctatttaattcaaaagacatataagagttttttttttgctaattccatgttggatttattgtcactacttattaagttataattttaatgtattgtgattagagattgtactgtgttctatttgtgttttaaggatacatagagattttctttgtggctaaatataagtaatccatgggtgttttaaagaaggtagagtttccaaatagagtatgacattccagtcactgtcagattccacacaacatttcagtcagccacagaacatataaatgatagcagtcccatgagattaaaatagtatatttttcctacaccttttctttatgtagatattttagatacacaaatacttaccactgtgttaaaattacctacaatagtcagtagagtaacatgctgtacaggtttgtagcctaggagcaataggctataccttatagcctagatgtgtagtaggctgtaccatctaggtttgagtaagcatacgctgtgatgttcacacaacaatgaaatcacctaacaatgcatttcttaatatacatctattaaatcattttattaattaaattatattcttcctgaatattgattttcttaatctgtcgagaacttcaaagagtatattttaagttaattgaatatagcttttgttaattgaatggttttatatatatatatatatatatattttttttttttttttttttttttttttttgagaaggaatctgcctctgtcacccaggttggagggcagtggcgtgatctcgacttactgcaacctctgcctcctggattcaaacgattctcctgccacagcctcctgagtatcttggactataggtgtgcaccaccacacccggctaagttttgtatttttagtagagatggggtttcaccatgttggccaggatggtctcaaactcctgacctcaagcgttccacccacctcggcttcccaaagtgctgagattacaggcatgagccacagcacccggcctggttatatatatttttattttcggcacataaatattaatgacatgaatattaatgacacttttttcactgtgaatcataccataaatcatataaagtaacttttattttacattttgaatatgttttgccttttattctacttcatccaatacagttttattaaatgtggatttgtaccattgctctttgtccatccttttacttttggcttttctgagtcacttcactcaagtggaagtagcacctacttgaatttaacttttttcccccacccaatttgaaggtctttttttgaagtgagttcaacttaagttcattgttgtaacaaatatgtttggtttaaattttatcatttccctttttgtttttttttccttttctggtaaacttattaagttaatgtttgttaaactaaattccatttcaaacggtgggaaggagtaaaaaacacctaacataaagtttaccatcttaagcattcttaagtgtacaggtcagtaatgttaaatatattcacattgttgtgagacagatctccagaacatttgaatattgcaaacctgaaactctatacccattgaataacaacttcccttgtcctcctcccccagcttctggtaaccaccattctactttctgtctctgtgaatttgtcgataccaagtttggaagataagaaatatcttttaattcctgattttgtgtgataagagaaataaaatatctaccctttagtgactgactcatttcacttagcataagatccttaagattcatccatgttgtagcatgtgacaggatttcctccctttttaagtcaggataattttccgtgggttgtgtgtgccacatttatgtatccattcctcagtggatgggcatctgtgttgcttccacatttgcctattgtgagttgtgctgctgtgagcatgggtatgctgttagctttttaagtccctgcttactgtgcttttggagacatactcagaagagggattactgaatcaaatggtagttctaagtttcacctttggaggaatctcaatactgatttccagagcagttgtgccattttacattgccaccaacagcacacatgagacccattttatctacatcctcaccaatacttgttattttcttttttttttataatgaccgtcctagtgggtgtgaggtgatatttcattgttgttttgatttgcattgctctgatgattggcgatatagagcatcttttcacatgcttgttggccatttgtatatcaactttggagaaatgtctattcaattcctttgcccatttttggtcaggttattgattttgttgtcattgttgaattctgagagttatttgtgtattctgtatatcagaatcatgagttgcaaatattttctcccaatccgtaggttgtcttttactctgttgaatgcatcccctgaggcacaacttaagcctggtggtcccatttgtctatttttgcttttgttgcctgtgcttttggtgtcatattcaagaaagcactgccatattcaatgtcacaaagcctttcctctatgttttcttccaagactttgatagttttaggccttacacataggtctttaatccattttgagttaatttttttacataagactagagtccaacttcattcttttgcatgtgaatgtccaggtttccaagcatcatttgtgaaaaatctgttcttttcccattgagtggtcttggcacccttgtcaaaaatcattttggccagggacagtggctcacacctgtaatcccagcaatttgggaggccgaggtgggtggatcacctgaggtaaggagttcaagaccagcctggccaacatggtgaaaccctgtctctactaaaaatacaaaaaattagctgggtgtggtggcgcacatctgtagtcctagctactcaggaggctgagacaggagaatcacttgaacccaggaggcagaggttgcagagagccaagatcgtgccactgcactccagcctgggcgacagagcaagaccttgcctcaaaaaaaaaaaaaaaatcatttgaccatatgtgagagtttatttgtaggcactttatcatatttcgttggtctattcgtctgtctttatgctactatcacactattttgattattgcacctttggtaatatgtttgggttttcggttgttgttgattattttggtttgtttgttttttgtagagatggggtctcactgtgtttcccagtctgatatcaaatttctgggctcaagtgatcccccagtcttggtctccagaagtcctgagattacaggtctgagacgccgtgcccagccagtaatatgttttaaaatcaggaaatgtgagtcctccaacattgttgttctctttcaaaattgttttggctgttcaggatgtcttatgatttttactgatttcttgccatttccaatcttttttgttcattactatgtaatatatgcttcttattttattttatttttggagattgagtctcgctctgttacccaggctggagtacaggggcatgatcttggctcactgcaacctccacctcccgggttcaagctattctcttgcctcagcctcctgagtagctgggattacaggcatgtgccaccatgcccggctaatttttgtatttttagtagagatggggtttcaccatggtggtcaggctggtcttgaactcctgacctcgtgatctgcctgcctcagcctcccaaagtgctgggattacaggcgtgagccaccatgcccggccataatatatgtttctttgagctgtttttctgataacttagcggccatgtaatctaattaactaatatccttcaaccactttctcaatctatcactctcagacacgaaacagtatctgttgactctatcatatgaaaaattaagaaagtgactgcacttctccccacctgtgcccccacatacagacattcccccaactaaccaataatggttatattttgtggattttgtgtcagctcattgtagtagtattactacattgactagctttacttattcgtggattcttgtaggtttttaagattttatactatgcagccataaaaaatgatgagttcatgtcctttttagggacatggatgaaattggaaatcatcattctcagtaaactatcgcaagaacaagaaaccaaacaccgcatattctcactcataggtgggaattgaacaatgagatcacatggacacaggaaggggaatatcacactctggggactgttgtggggtggggggaggggggaggggtagcattgggagatatacctaatgctagatgacgagttagtgggtgcagcgcaccagcatggcacatgtatacatatgtaactaacctgcacaatgtgcacatgtaccctaaaacttaaagtataattaaaaaaaaattgaacaaaataaaaaaaaaagattttaagtctagttacttttctagggtatgtctcaatgtaggtcaagctctgtttttttattaattgagataaaattcacattccatacaattcttttttagtatataattcagcatgtttttgtatattcagaaagttatacatccttcacccctaattcctgaacatttttgtctccccaaaaagaaaccgtgtgcccattagcagttacctcccattacccttgttcttgcacccccgcctcgccctggcaaccactaatctactttctgtctctatgggtttgcctatatgagacattccacataaatggaattgtaaaatatgtggctttttgtatctagcttctttgacttaacatgatatttttaaggttgtagcgtatatcagtacttcattttttggtttgcagaataaaatccgttacgtggatttacaacattttatttatatattcatgaatggatggatacttgggttgtttgtacttttttactttcacaaataatgctgttataaattgttatagattgtgtacacgtttttgtgtggacataattttttcagttctcttggatatatacgtaggagtagaacagctgggtcatatggtaactctctgtttaacctttgagtaactccccaactgttttccaaagtggctgtgccgcgttatatttccaccaggatcatattagggtttcagtatttccacgtcctcgtcaataattgttattttctgatttttgattataaccatcctaatgagtgtgaagtgtcatctcattgtggttttgatttgcatttctctaatgactaataatgagaagcacctttttaagtgcctattagccatttgaatatcttctttgcagaaatttctattcagatcctatgctcatgtttaaattggcttatttttgttcttttattattgggttcgacgtgttctccatatattctgaacagcagtcccttattcactatgtaatttgaaaacatgtctcccattctttgggatgcttttcactttcgatggtatcatttgcagtacaaaatttttagttttgacaaagtccaatttatctacttttgtcatttgtgtttttcatgttgtgtttaaaactattgcctaacccaacatcacacacaaaaaaagtttacttctttgcttttttctaagagtattataattttagctcttaaattagggtctatgatcattttttagctaatttttgtatgtaatgtgaggcaggggatacagtttcattttttgcatatggatatccagttgaccccgcaccatttgttgcagagactattctttcttccattaaattgtctggggtcctttgccaaaaattaattgaccataaatgtaagagtttatttctagactcttaattctatttcattgatccatatgtctctgcaaatgccaccaccacaccgtcttgttgtggtgttcttgtggtaaattttgatgtgtgagtcctccaactttgtttttcttttataaaattgctttagctatttatgggttctgtgaactttaagatcagcttgccagtctctgaaaaaaaataaaaggcagcatgaatcttggtagggatgtcattgaatctatagttggaattggggagtactgacatcttaaaaatattaagacttctaatctatgaacatgggatctattcccatttagttaagtcttctttcatttctttcaacaatgtttatagtttttacttctttgcagttcggcacatctgtcattaaatgtatttctatatattttcttctttctaatgccattgtaaatgaatttgttttcctaatttcatttctaggttgtttactgctagtgtatagaaatacaactgatttttcttacattgatcatagattgtacaagcttgctgaacttgtttatcagctctaataatttgtgtgtgtatatgtgtgccttaggattttctatatagaataccatgtcatcttcaaatagagatggtttttctttctaatcttatgctattttttctttttctttgctgattgcctttgtttgaacctccagaactatgttgaatagaagtggcaagaatggatgtccttgtcttgttcctaatcttaaaagggaaagcttgacagtcattaagcataatatgagggatgagttatttgtagatgccctttaacaggttgaggaagttcccttccattcctagtttgttgagtgtttttaccatgaaagggttttttattttgccagaagccttttctatttctattgagctgattatatgttttccctttattctattaatatggtgcattatattgatttattcatatgataaaccaaccttgcattcctgagataaatcccacttggttttgatgtgtaatcccgtttctgtcttcttggattcagtgagctagtatttttgtagtattttacatctgtagtcacaagggatattggtctgcagttctgtgatatcttcgtctgcctttgatatcaggataatactggcctacatagaaggaatcaagaagtgatccttctgattatgttttttgtatgagattgtgaataattggtgttacctcttctttaaatatttggcagaacttatcagtgaagcttggtccaagctttacttttggagaagttttctgattattaactcagtcttttctcattataagcctactgagattttttatttcttcttgaatcgattttgatcatttctttatctagaaatttgtctacttaatctaggttgtctaatatcttggtatatgattgttcatagtatcccctcatccattcttaattttagtgatttgagtcttcggtttttccttggtgaatctagctaaagatttcagtttttgttgatgttttcaaagaccaaacttggtttctttaattttctcttgcattttcctaatctctgttttatttatttccattctaacatttagcatttccctccttctacttatttttgttttagttacatagaactttagttacgtagagctcatctttttctaattcctttttaaaataatttcaatttttattatagattaaaggacacatgtgcacatttattacatgggtatattgcttgacactgaggtttggggtataaataatcccatcacccaggtggtgagcatagtacccaatcggtggtttttcagcccattccctcctcctttcctcccatctagaagtcctcactttgtattattctcatctttatgtatattcagtgttcagctcccgcttgtaagtgagaacatgcagtatttggttttctgttcttgcattaattggcttaggataatggcctccagctccatccatgctgctgcaaagggcatgattttattttatttttatgtctgcacggtatttcgtggtgtatatacactacattttctttatccatccactgttggtggccacctaggttgatttcatgtctttgctattatgaatagtgcgggaaagagcatgtgagtacatgagattttttgacagaatgaattcttttccttcgaatatatacccagtagtgggatcgctgggttgaacagtggttctttttcagttctttgagaaatctcccaactgctttatacaatggctgaaccagtttgcattcctaccaacagtgtataagcattcccttttctctgcagccttgactgcatctgttattttttgactttttaataatcagaattctgactggtgtgagatgttatctcattgtggttttgatttgtatttctctgatgaatggtgatgttgagcattttttcatgtttgtcagctgcttgtacatctgcttttgagaagtgtcttcctgttctttgctccccctacttttttttttttttttttttttttgagaaagggtctcactctgtcacctaggcggtagtgcagtggcacgatcttggctcactgcaacctccgcctcccaggttcaagcaattcttgtgcctcagcctcctgagtagctgggattacagctaatttttaaatttttagtagagatgggatttcgccatattggccagctaattttttaaatttttagtagagatggggtttcgccatattggccaggctggtcttgaactcctggcctcaattgatcttcccaccttggcctcccaaagtgctgagattacaggtgtgagccaccgtgcccagcccctttgcccatttttacaaatggaattatttgatttttgctttttaaatttttacatttccttacagattctggacattagagatttgttggatgcataatttcctaatattttctcccattctgtgagttgtctcttgattgatcatttattttactgtacagaagctctttggtttaattaggtaccaatcatcaatttttttattttgttacaattgctattcaggacttagccataaactattttccaaagccaatattgagaagggtatttcctaggttttctgctaggatttttgtagtttgaggtcctccatttaaatctttaatgtaccttgatttttttaaatatagtgagaggtaagggtccagtttcttctgcatatggctagccagttatcccagcaccatttattgaatacagagttctttcttcattgcttatttttgccaattttttgaaggtcagatggttgtaggtgtgcaggtttatttctgagttctctactctgttccattggtctatttgtctgtttttgtaccggtaccatgctgttttggttactgtagccttacagtggagttttaagttgggtaatgtgatgcctttggctttggtgtttttgcttgggattgctttgactatttgggttcttgtttggttccaaatgaattttagaatacatttttctaattctgtgaaaaatgatatgctattttgatagggatagcattgaatctgtaaattgctttgggaagtgtggccattttaacaataatattgattcttctcatccataagcatggaatttttttccatttatttgtcatctctgatttcattcagcaatgttttatagttctccttgtagagatctttcacctccttgattaggtatttcattttttgtggttattgtaaatgggattgtgttcttcatttggttctcagctagaacgttactgatgtagagaaatgctactgattttgtatcctgaaactttaataatttgttcatcaggtccaggaggcttttggtggagtctttagggtattctatgtaactcatatcatcagcaaagagagatagtttgacatcttgttttcctatttggatgcctttcacttctttctcttccataattgctctggctcagactttcagtactagaagtggtgagaatgggcatccttgtcttgttccagttctcaaggggaatacttccaacttctgcctattcagtatgatgttggctctatgttagtcatagaaagctcttattattttgaggtgtgttccttcaatgccttgtctgttaagggtttttatcatgaaggggtgttggatttcatggaagatgttttgtgcatctaccaggctgctctggggtccaaggcctatggaggtcccttgtggacttgagtgttgcctccttatacactctgggcaggcagctccctgtatcagtctatatgcctgggggtcaggaggaatctcccatcccaggattgcacaggtccccgcaggaagtgtagatcccccaggggctctcactcactatggaaaccatttagttgcccatagtaatgtggttgaattctctttaactgccttatacatgtttataagttgctgtttgaatccccttcccaggacatgactggtaattgtgtgtggtttgtattaattttgttccttggagcttggagaagccagagagggagtaggtgattctaggactctgtgcaacctcctgaagtgacatttttctctgcgattgttttaaaactcacaaactgatggctgctttagcaattgcagggtaagggaaatacctagttctatgggacgtgcccttgaggttttgcttttgtacttctttaaggtggctgttggctgctttggtggcttcctagtgtacagtaatattgctgggcctgattttttttctagcaaatcttcataatgcagctaagctattcagatctaagagataattttctagacatttttctgatttctttctggctagaaggagtatatgattaatcttcattcaagctggggcagaaatgctgttttcttttttaaacagggttgcatgtctctagttgagaagtttatctcttaccttagtcaccttttctgtcaaaagtgggctatccgtagggtattttgaatgtaatgactcacctttcaccctctgtatgtatgatcctttgtggagaaagaactgtgtattagctctaaatttgtgatatgagttgagccccttctctggattctgaagattcttttcatgttttgtttgtttgtatatttcagtcatggaagaggagattcattatacctgtctatttcctaaaatccatcacctgttttctcagtgtttctctctctcctatttactcagcttctctgtatctataggctgcttcctcttcactggaaccatgctttagtcattcctcacatggactggggaggacaagtgtgacaggatggcttctgttgatgtttctactgccttacatctttctctttattaccaaatatgtaatcttttattaagtataaagataaaaggatagttaaatataaagagttcaacaacatagaaaataaaaaggccttgtaattcatccgtaattctccccatcctgaaatagtcctgattaacattttatatataatcttccagttttatattttatgattcacataccttgttttattttaaaaaatggaattataccaggtgatttgaaagtcttattttcaacttttgtttgttttgaaactgttcatttaatatatcataatagacatctttctgtgtgaaatacagatttatgtagtcttttttattgctttatgagttccattgtatacttatactgtttaattagttccctaataacagatatttaaatcatttaaaatatttcttttttattattattatactttaagttctagggtacatgtgtacaacgtgcaggtttgttacatatgtatacatgtgccatgttggtgtgctgcatccattaactggtcatttacattaggtatatctccgaatgctatccttcccccctccccccaccccacaacaggccccactgtgtgatgttccccatcctgtgtccaagtgttctcattgttcaattcccacctatgagtgacataggtgtttgtttttctatccttgcgatagtttgctcagaatgatggtttccagcttcatccatgtctctacaaaggacgtgaactcatccttttttatcactgcatagtattccatggtctatatgtgccacattttcttaatccagtctgtcattgatggacatttgggttggttccaggtctttgctattgtgagtagtgccacaataaatatacgtgtgcatgtgtcttgatagtagcatgatttataatcctttgggtatatacccagtaatgggatggctgggtcaaatggtatttctagttctagatccttgaggaatcaccacactgtcttccacaatggttgaactaatttacagtcccaacaacagtgtaaaagtgttcctatttctccatatcctctccagcacctgttgtttcctgactttttaatgatcaccattctaactgttgcgagatggtatctcattgtggttttgatttgcatttctctgatgggcagtgatgatgagcattttttcatgtgtctgttggctgcataaatgtcttcttttgggaagtgtctgttcatatccattgcctagttttgatggggttgtttgatttatttcttgtaaatttgtttacgttcgttgtagattctggatattagccctttgtcagatgggtagattgtaaaaattttctcccattctgtaggttgcctgttcactctgatggtggtttcttttgctctgcagaagctctttggtttaattagatcccatttgtctattttgtcttttgttgttattgcttttggtgttttagtcatgaagtccttgcccatgcccatgtcctgaatggtattgcctaggttttcttctagagtttttatggtggtaagtctgacatttaagtctttaatccatcttgaattaatttttgtataagctgtaaggaagggatccagtttcagctttctatatatgtctagccagttttcccagtaccatttattaaatagggaatcctttccccatttcttgtttttatcaggtttgtcaaaggtcagatggttgtagatgtgtggcattatttctgagggctctgttctgttccattggtctatatctctgttttggttaccagtaccatgctattttggttactgtagccttgtagtatagtttgaagtcaggtagcgtgatgcctccagctttgttcttttggcttaggattgtcttggcaatgcgggctcttttttgattccatatgaactttaaagtaggtttttccaattctacgaagaaagtcgttggtagcttgatggggatggtattgaatctataaattaccttgggcagtatggccattttcacaatattgattcttcctatccatgagcatggaatgttcttccatttgtttgtgtcctcttctatttcattgagcagtggtttgtagttccccttgaagaggtccttcacatcccttgtaagttggattcctaggtattttattctctttgaagcaattgtgaatgggagttcgctcatgatttggctctctgtttgtctgttattggtgtataggaacgcttgtgatttttgcacattgattttgtatcctgagactttgctgaagttgcttatcagcttaaggagatttggggctgaggcaatggagttttctaaatttacaatcatgtcatctgcaaacagggacaatttgacttcctcttttcctaattgaataccctttatttctttctcctgcctgattgccctggccagaacttccaacactgtgttgaataggagtggtgagagggcatctctgtcttgtgccagttttcaaagggaatgcttccagtttttgcccattcagtatgatattggctgtgtgtttgccataaacagctcttattattttgagatatgtcccatcaatacctagtttattgggagtttttaacatgaagggctgttgaattttgttgaaggccttttctgcatatattgagataatcatgtggtttttgtctttggttctgtgtatatgatggattacatttattgatttgcatatgttgaaccagccttgcatcccagggatgaagccaatgtgatcatggtggataagctttttgatgtgctgctggattcagttttccagtattttattgaggatttttgcatcaatgttcatcagggatattggtctaaaattctctttttttgttgtgtctctgccaggcctaggtatcaggatgatgctggccttgtaaaatgagttagggaggattccctctttttctgttgattggaatagtttcagaaggaatggtaccagctcctctttgtacctctgatagaattcaactgtgaatctgtctggtcctggactttttttggttgataggctattaattattgcctcattttcagagcctgttattggtctattcaaggattcaacttcttcctggtttagtcttgggagggtgtatgtgtccaggaatttatccatctcttctagattttctagtttatttacatagaggtgtttatagtattctccgatggtagtttgtatttctgtgggatcggtggtgatatgccctttatcattttttattgcatctatttgattcttctctcttttcttctttattagtcttgctagcggtctatcaattttgctgatcttttcaaaaaaccagctcctggattcattgatttttggaagggttttttgtgtctctatctccttcagttctgctctgatcttagttatttcttgccttctgctagcttttgaatgtatttgctgttgcttctctagtttttttaactgtgacattaggatgtcaattttagatctttcctgctttctcttgtgggcatttagtgctataaatttccctctacacactactttaaatgtgtcccagagattctggtatgttgtgtctttgttctcattggtttcaaagaacatctttatttctgccttcatttcgttatgtacccagtagtctttcagaagcaggttgttcagtttccatgtagttgagcggttttgagtgagcttcttaatcctgagttctaattgggttgcactgtggtctgagagacagtttgttatcatttctgttcttttacatttgctaaggagtgctttacttccacctatgtggtcaattttggaataagtgcgatgtggtgctgagaagactgtatattctgttgatttggggtggagagttctatagatgtctattaggtctgcttggtgcagagctgagttcaattcctggatatccttgttaactttctgtctcgttgatctgtctaatgttgacagtggggtgttaaattctccaattattattgtgtggaagtctaagtctctttgtaggtctctaaggacttgctttatgaatctgggtgctcctgtattgggtgcatatgtatttaggatagttagctcttcttgtttaattgattcttttaccatcatgtaatggccttctttgtctcttttgatctttgttggtttaaagtttgttttatcagagactaggattgcaacccctgcctttttttgttttccatttgcttggtagctcttcctccatgcctttattttgagcctatatgtgtctgtgcacatgagatgggtctcctgaatacagcatactgatgggtcttgactctttatccaatttgccagtctgtgtctttcaattggagcatttaggccatttacatttaaggctaatattgttatgtgtgaatttgatcctgtcattatgatattagctggttattttgctcgctagttgatggagtttcttcctagcattgatgttctttacaatttggcatacgtttgcagtggctggtaccggttgttcctttccatatttagtgcttccttcaggagctcttgtgagacaggcctggtggtgagaaaatctctcagcatttgtttgtctgtaaaggattttatttctccttcacttatgaagcttagtttggctggatatgaaattctaggttgaaaattcttttctttaagaatgttgaatattggcccccactcctttctggcttgtagagtttctgctgagagatcagttgttagtctgatgggcttccctttgtgggtaacctgacctttctgtctggctgcccttaagattttttccttcatttcagctttggtgaatctgacaattatgtgtcttggagttgcttttctcgaggagtatctttgtggcattctctgtatttcctgaatttgaatgttggcctgccttgctaggctggggaagttctcctgggtaatatcctgcagagtgttttccaacttggttccattctcctcatcactttcaggtacaccaatcagacatagatttggtcttttcacatagtcccatatttcttggaggctttgttcatttctttttactcttttttctctaaacttctcacttcatttcattcatttgatcttcaatcactgattctctttcttccacttgattgaatcagctactgaagcttgggcatctctcacatagttcacgtgccatggtttttagctccatcaggtcatttaaggtcttctctatgctgtttattctagttagccattcatctaatcttttttcaaggtttttagcttctttgcaatgggttcgaacatcctccttttgctcagagaagtttgttattaccaatcatctgatgcttacttctgtcaactcgtcaaagtcattctccatccagctttgttccattgctggaaagcagctttgttgctttggaggggaagaggtgctctgatttttagaattttcagctgttctgctctggtttctccccatgtttgtggttttatctacctttggtctttgacgatggtgatgtacagatggggttttggtgcggaggtcctttctgtttgttagttttccttctaacagtcaggaccctcagctgcaggtctgttggagtttgctggagctccactcctgaccctgttcacctgcgtatcaccagcggaggctgcagtacagcaaatattgcagtacagcaaatgttgcagcctgatccttcctctggaagctttgtctcagaggggcacccagctgtatgaggtgtcaagttgtcccctaatgggagatgtctcccagttagggtactcgggtgtcagggacccacttgaagaggcagtctgtccgttctcagatggcaaactccatgctgggagaaccactactcccttcaaagctgtcagacagggatgtttaagactgcagaagtttctgctgccttttgttcagctatgccctgccctagaggtggagtctacagaggcaggcaggccttgttgagctgtggtgggctccacccaattcatgctttccggctgctttgtttacctactcaagcctcagcactggcggacacccctcccccagcctcgctgccaccttgcagtttgatctcagactactgtgctagcagtgagtgaggctctgtgggcatgggaccctccaagccaggcgtggggtataatctcctggtgtgccgtttgcttagactgttggaaaggtacagtgttagggtatgagtgtcctgattttccaagtaccatctgtcacggcttcccttggctagtaaagggaattcccctaccacttgcacttcccggatgaggtgatgccccaccctgcttcggctcacactccatgggctgcacccactgtccaacaagccccattgagatgaacctcgtacctcggttggaaatgcagaaatgacctatcttctgcatccctcatgctgtgagctgtagactggagctattcctattcagctatcttggaaccctccggtggttttgaagttcatcactttttttagctatctccaggcctccttatctcacatgaaaattatatgcaagtgtcacttcagaatttccttctgaagatgcatcagactaatgtttggctagcattctcttcagagtttttccaggcccagtgaaaatagccaggctttttttgtcttaccaccttacaacaaacagcttaagtcctatccaactactacattctcccaacacagatacaaggcagcatgttcacattatctcaagcttcctccaaatacatctcaacagttagaattggattagaaagaggacttgttggcttgcttatcctgtgggtaactgtgacattgactgcaggtaataataacaagatctcttttatttttctaggtggcaaaccatgtgatttcttctgactctatttcctcttctgccagtagtttcctgagctcgaactctactttttgcaacaagcagaatgtacacatgttaaacaagggcacacaagcaggtaattacttgaatctgaactttttcattgaaatacattgaaatggctcttaaacatataagatactcaacctcaatcatagtaaaggacatgaagttaaaactatatccaactgccaattttcatcttatcagattggcgtaaattcaagtgtgatggcaagtttgtgggcaagagtgtagggaagtgtgttcccttgtatattccagtgggagtagaagttggtttactgtgaaagacaatttgacaggtcctgtcaaaattactttcactcagcatttttacctttgaaatgtctgctatagatatactatgaatatgtggattttaacctatagatatacttgcatatgtgcaaaatagtgtacaaaattaaccactgaaacattgttttttatagcagaagattaaaatgaatttaggtattcctcaatagctgaatgattacataaattattgtatatctggataatgaatgaaattctacatagaatgaggaagctctctctttactaacgtggaaagatctccaaggcatataactgattttaagaaaaggtacagaacagtgtatggcatactacaatatgagtttttttaaataaaagaaataaaggagggaagagaattgcttaaatgtgcaaaatgaattctctggaaccatatggggttgtgaacagagtaaatggagaccagaagtgggaaggaggatttttagctattgcctttctatacttttggtttcataaaataaaaataaataaactttcttgatttgtttcttcccctcaaatcctgtctttacttctgtcaacctcttccatacctgggtgactaccaaaaggactctaagtatttattgtctttctgccttgggatccaggtggaaattctttaccctttgaacccatattcattcttctttccctctcatacctaaaatgacttacaaataaaaccctttcttttttcttctccctctttttccctcctactctcccctgtccttccttcttttcctctctcttcccatcactccatcccacacaaagggattgtatttttgaaacattaaaaaggttcacatactcacttgaataaacctgtcaactcagcctcaaggttactcccagagacacctgtgatccttcccctcaggtaacttggagattgtgaacggtgccgaaaaacacactcgagccaactccaagttccagcgaggctaaattggaagaggacagtgatgtgacttcttggtcagaagaaaaacatgaagagaaaatgctctttaccagttatcctgaggacagaaagttaaaaaagaacaagaagaattcccatgaaggtcagtttctcattccagatcttgtagtagagaaactagtgaatttcaagtcccctgcaatgctgactctgtgtttccaagtgattctatgtgacccccacctacccccagtcaccaccttgtcagattttcgaagtccagcatggcagttacacagtcatccctgcagtgaaaccaaactcaagggcaccctgtggccgctgacagctgagacccctgagagcctgtattatatgcatctccttgatgaaagccaggtggggctgtaaaaagtgaaaattctgttttacttctaaaaactatttcctgtaggagtttcctggtttgttcctgtggaaaatgtggagtctagataaaaagaaggaaaacatgctcaagactcatgaccctggcatctcccggttggaaccagtaaccaagaccaagccgtggagggagccactgtgggagcggaactggcaggggcagcacctggacagtcggggctacctggcaggcccaggcagagaggatggcagaaacccactgaagctgtttgtgagagcaaccctgcaggtgcagtgacgtttacttaactttagccctacatgtagggagaagaagggtaaggcgcagagaagctggctctgtgacttggcgagctgaggtgtaggcctgagacgctcttttccagcacctccgcagttcacctgttttcacacgtgaggactgagtttctcatcatctgtcaaaggctgccttactcttagccatagatggtgcatcactgtcaggatctggagacaagggcacaggagggaggatttgtggctctgctcattgggtttgtgggtccctgtgtgtggagggcccagaggcctgatgcttgtgcagcctcatgcatgttcctccatgtccgcagctcctgtttctgctccatgtcggatttaaaggaaaccaagtctcttctgatgtgtccctctggggataaaggaaaagttattaatgatgtatttgtttgaatagatctttaaaatagcagcatgggcagagctgtattgcgttgatgtttcagagagttgtaatcgccatagtcttagttcctggcctatgctgaaggcatgtctttgatccctgtgggttccaagctgcagctctccgccatagagaggtgtcagaggtttcttcaactctaggtattttctcggcatttgaatcaggcttccccagacctcttgggtacgtggtgtgtttccagccatatggagagtagattgcatcattaatctgaggctgggtattttctctctctctctcttttttttttttttttttaaggaatcgcttcagtttcacagacctgacttcatctcccacattggggagcggataaagcgtctgaagttaatagtccaggagagaaagctgcagagcatgttaaagagcgagcgggatgcgctattcgacattgacagggaacggcagggccaccagaatcgcatgcgcccactacccaagagaggtacgccctgcccgttcactttcctgtgagtggaatagagaaggcaaggtctgctgctgtgctgcagagccctgctaaaggccagcccaggccagttacctgggcagacagcaaccttttgcttgccaagactcaaaccttcttagctaacagaacctccccaccttagccgtcagcacattcatgatccaagtaagcccagttactctagccctgtcactgcatcttaaaccagctctgtctgtgagcaagttagttccaaaagtatattgatttttcctgaaaagatggggctgattccaggcttgcctacctcttaggatggtaataatgataataataaacacatagtccttacatcttgcccaggcactgttcacattagcgcttacagcactctcagtaggtactattaatatatctactttatagtgagaaaactgaatcaaagggagattaactacctctcccaaggtgacatactcaggtgtgctgctctggagtctgcattcttaacaattatggtaagcagatcttgataaaataatgaaagctgtgaaaactacaaagtactatctaattctttcatcaagccttacctggaaactggccagttttttaattcaaagcctagtctttgctacagagtgttaggtccttagggccttaggctgctgtcctgttaccctagtctaaccctggcttgccttggtcattgctccccactcagtcttgccagctcagggtaggggcaccaagtcctagcagctccctctcccagctcttgcaccacaccctctgggttaagtcactgtccactgaaaaccctttcttctcttccttagtcttcctggctgtccagaagaacaagcctatcagcaagaaggaaatgattcagaggtccaaacggtaagaccaagaaaacaagagtttgtgtacaagtgttaaccaggccaccaagtggtcaggagctctggcttgcacccagaataaaggcattatgctcaaatttaaacattatgagaaagttgtgagagtcatttctcacttatggcactgaaaaaaaaagtagcacatcatggtatgaatgactcatatgcaaaagatgaaaaacttctcattaatttgcatatcttaggctcctctttattaacctaaatgtaaataaagaatattttaggccaggtgcggtggctcacgcctgtaatcccagcactttgggaggtcaaggtggataaatcacttgaggtcaggagttcgagaccagcctggccaacatggtgaaaccccgtctctactaaaaatacaaaaagtagccgggcgtggtggtgcacgcctgtaatcccaaccactcagaaagctgagccgggaggatcctttgaacctgggaggcagaggttgcagtgagcttagatggcactactacactccagtctgggtgacagagcgagactccatctcaaaaaaaaaagattattttaaaataaaattaaatatgtatttgggttattataccttttatcttagcaatcccatttcttggaattgatcccatagggtgagaaagaacagtgttctccagaacatattaagttaaacttttttaaaaaattaaattttcgaagtatgtcaacactgtttccatttgtggtctttaaaagtaagtatttgtgcctatgtagacgtgtatacttttatatgcatagaatatttctggaagacagaaaaacctgataatactgattgcctttggaaaggagaactgatgtaggatgtagtagattttttgactttttgaatgttgtgtcatgaggaacgaacattagttactcataaattaattcatttgagtgttggatgtggtggtacatttctgtaatcctacctactcaggagtcttaggtgagagaatctcttaagcccaggaatttgagactgcagtgagctatgatcacaccattacgctctggcctgggtaacagactgagactctgtatctaaaaaataataaataatattttaaaaaattaattcatttcagatattgatatattgatcatgggaatgccagagaataagttaaggttgacttttttaggtgttcattagagacttgctttaatgagaatagtttcttagttggcataatgcttctgaagtaaaggtactttaaaacagtttatcttacggactttaagcattcctcttgaaatcttgtttttactctttctagtcagtctttataggcaatccaaagggatgctttggatgctttagtccagtggttctcagagagtggtctgtggagtcctggaagtcgctgagaccctttcaggcaatttgcaaggtcaaaactaatttcagaatgacaccaggatgttctgtgccttttttgctgtgttggctatttgcactgatgatgcaagagtactgggagaggagtaaaaccattggtgtcttaccactattcaagacggtggcaccaaaccgtactagtctaataggcattgtatcctgcacagcctcccgccaggagtgtgatggaagaacaaggaagcagtgtaaatggaggtaaacgggaagcacttcctttgcaccgtgaaagatgatgactgtttcaaggaaaagcacttatgccatggtttgtgtcgtgagctgaaccagctgcctttgtcatggaacatgacttttaattgaaagagtaactgacagaaaaccaattattctgacttgtgtttagcagacattttcttgaaaatgaagaagtgactctctgacttcaggaaaatgtctgacagtatctgttgccaaagaaaatgtgagttttcaagccaacaatagaattctagaaaacttgtattcaccaccatgggcttgatgacatcttagtacttagacctttctgacgagatagcagtgacattaacaaatgtgacgtttttcatgttatctaaatacatttgtcaacatttggaagatctgcacaactccctggaccaggattttccaaatgattgttgctttttgttacaaaatcagggaatagatagaagattcaaatagtacaagatagactgatggatttttatgtaacagaataggaaaagtttgctgacatgctttcagattccaccttgcaactaatttttaagaaactaccacttgtcagccaggcacagtggctcacgcctcataaaatgagttggaatgaagtttttctttcttattctctagaagaatgtaatgtaatttattgcttaaataattaggagaattcaccagtgaaacccatctgggcctggattttcttttgtgggattcatttttttcaataactattcagatttcagtttttcttttatgccaaggtttagtaagttgtgttttttacgtaatttgttcatttcctagaaatttacaaattgatgatccaatgttataatatccttttgttatctttttttttttttttttttgagacggagtctcgctctgtcgccctcaggctggagtgcagtggcacgacctcggatcactgaaagctccacctcccaggttcacaccatcctcctgcctcagcctcccgagtaggtgggactacagacatccgccaccacgtctgactaattttttgtatgtttagtggagacagggtttcactgtgttagtcaggatgttctcgatctcctgacctcgtgatcagccctcttcggcctcccaaagtgctgggtttacaggcatgagcccccgcacctggccctttgttatctttttaatgtctggacgttctaggatctgtagtgatgttccgtttttattcctgatattatttttgcctcctgtcatttttacctgatcactctttccaaggggtatataaatttcactagtattgtcaaataacccactttgggttttattgatttttctctatcgtatattggtcttatattgattttttgttgtttattttggggccgtagcaggagccacagccagctccacatagttcagagggcctggcaagtatggaggaagacccttgctttcccctggaggtgctgagagtttaattcccgaggaagtaaggggattatttccctgtattggcaaaggttctagtttttgagtagaactagaggacctcacttctcttgaaggagaagtagagaggctcctcctcattcccttccctacaagtccccacgtgactgattcttcctctccaggggcttctagaattagcaacattgttccctttgatcctttggctgattacatatcctgcatttcttctgtagggctgggtgttgatagagggccataagacattatacataaggtattttatcccatttaccttgtttcttacaaaataagtcccagttggagaattgtagtattaatgtatgtaagtaactcatgttcagaccatctctcttgattccgtagggaacactggggccatatggtattacagtaatatatcatttgctttttggtcatggatatgggtatccaaatagtttccagtgctggaggaggcactgtaacgggctctcctccagtattgagacctggttccccatattctagggtctttgcccagaaggctgattccaccctccaagaagcttccactggggattagtgatgacacaaatcctgtaactcttagcctgtaggatatttctggtcctttcagcttgctgctacacgcaggccactatggagtggaggtgctgggaagctaggagaggacaataggggttgcctctccctggatcatgttcagtctgggtagcttccctggaaatctcatggagattccacagtagttccggatccagcttgagtggtgggaggtatgtaaatcaatcaagtaagcaaggtgttctttctggagagggtgcacactctgctcatgtaaagtctacatcactgtattttggttccaggaattcccacccaaactgccccattctaaattaggcctcttcaaatcaataacagttcttctgtgactgctggaatatggaagcccaatattttagggattaccacagactaaggagaagggaatccagacttcacatgcattcaggccacaaagaaacacaagtattggcaaaagtgacaagcattcagcctacagatataccagacagggacaaagtcttaaagatgagagtcctcgtctttatattagggcttcccctgttaaaatggatggagacatctctcaactcctagacctgaaggggagtcctgcccattcccaacagtcctggatcccttgagctaggtcctaaacctgacaacccagagtcctaataatcaaaaccaaaaccactcttttagtcagttcacagaaccaccaccacttactgcctcaaaattgcaccaaatgaaaggtccaagcagccacttctgctttcctggtgggctggggggatggggttggcatgcctgaggcccttcaccaatctccctcttggcaacttaacccactcatgcttcctcagactgctgcttcgcctgtaagcagcctataagggaggtcgagtcagggctcagtccatagtcccatctggatcaaccagatttgtttcccaggccctcccaccccactgcctccaaaaaaaaagcggggtttgtttgcctggtgagtaacaaaagtcgctgagaacacagactttgatcaataggagttttattacttgtcacaagtaggaagagctctgggaatattcgtcaaagcagtgtctcctaagagaaaatgacaggaaggctttatagagtgatcgagaggggagaaggtgcatcatcacatgaagaggaggggtcccagtgacacagatggagggagtcatcgtgccaccacataggacgcatgctatggtaatgaaactatagctctcccagggtgcaggatttagcattgtaatgaggaaagttcactgtaagtgagttcatctataagttgctggggtctgtcaggagcaggttccaactgactaggtgaccacatttcacacagggtttgagacaaaaccgtctgcaaggcagaaggctgtaaaacgggctaattgctcaaattcattaaattccttcactccctgggggcccgccctgtcttgtctatatagccagtccttaggaagtgaggccaagctgaactgttgacacagtctatgtctttacgagtctgggagctgggtgattttacctctgatgctagacttatttttactggttttgaagccttgcttacagccctttcccagtttctcacgaaatactcatgaaaggggatggtggggagtgggcatggggaaaagggaatgatattaaaattaaagctaacatcattggcctctagggggcactcatggctcatctccatagcccttggctcttgcagctgcccaagatgagagtcctgatctaaggaaactccaccaccaccaaggctggggtggtggcaatgggggcattattttccaaacctgactaggctacattttaaggggatcatagtcacagagaacatagatgtggactattaattttctttgcctgagcagccgtagccctatgtgctcctctccagcctttcagggagctatgatgggccaaaaaatgctcagtagcccaagccatctcctccatagacactggagtaaaatctctgcctatgggtgtgtgtgtcttgtcttactctatcctggatttgtttcctccaatgctggtggctttactttagatagtctgattgttgcagagatgagacccaaggctccactctggagaaaatagagccctcttgagaggtcagcccaacttgctagacttctgcatggggcttggggttcaatgttccagcctgtccccaaaaagtcatggccaaattcacatattcctttgatccctgatggtgtctcatcacacagttcgtctggcctcaccattgctccactaagtccaggaacctcaaggaactatgagacaggtagggctagacttaacaaaatggctatcacagtcttcatttcatgagacagaggtgaactgagaaaaggtggaaggtgcttcagagacttctctgtattcatccctaacttaggcagagagccttggccacacaggaaactggggaagacccctctcttactcaggagtcccccacactggtgccatcttatgacaggcaggcatcagcttcagagccagtcaaacagttaggaatagcaaacattgctccagaatttgttggggttttttttcccctgaatttgatagtaagctccacataggcaagaatgacaaccatcttgttctccttgtttctctggagtctaacacaacatgtagcacgtggttggcccaataagaatttgtcgaatgggggccagatgtggtggctcatgcctgtaatcccagcactttgggaggctgaggtgggagagtcgtttgaggcctggagttcaagatcaacctgttcaacatagtgagacccctatcttcttttttttttttttttttttaaagtggagtttgttgaatggcacacagaaaataaacaatccaaatgacagatcagaggtgctggaaaagaccagggagatctaatcgttaaatcacgtcatctaaatattaagaggagacatttggacatctgatagaatttggcattgaataagtgataaatacacagaaaactgaacaacggcaaaaagaaaagtatcagttctaagggaaaagatacaaatcaaaagccttcaggaaaggataagtcatcatggcttactgtatagcttagcttaaaataacacagaatagtgaagtagcccaaagggccatctaaccacactgggaagctgagcaggggaggagtgcgtatatgcatggaggagacttgtcttcaccctcctcagcagctgacagcaggcaggctagacctggacaatggagcaggagtcacacagcctgtgaaagaagtgctggggaatgtaggggctgctgtttcacatgacagagccattgaaccctggctgtttataatagttacatacataattttaatagaaataaaaaccaaaaagaaaaaagacttaaacatgaaaggcaaaaccattacaatttttagaggaaaatataagagagtaagaacagaaaaatgtcttaaggcacaaagtgctaaccataaagaaaaagattgataaattcatcttgataaattttttgtttatcaaaaggcaccataaagacagtgaaaagacaaggcacaaaatgggagaagataattgcagcacatctgaccagcaaagaatttgtatccagaatatataaagaacttctatgagtcggttttttaaaaacacaatttatatttaagtctctttaaaacaaatagacaattcatagaaatatggtgatatggtttggctgtgtccccacccaaatctcatcttgaattgtagcttccataatttccacatgttgtgggagggacccagtgggaggtaattgaatcatgggggcagatttttcccatgttcttgtgttagtgaataagtctcatgagagctgatggttttataaagagcagtgcccctgcacaccctctcttgcctaccaccacataagatgtgcctttgctcctccttcacgttctgctgtgattggtccgcatccaaatgtcatcttgaattgtagctcccataatcctcatgtgttgtgggagggacctggtgggaggtaattgaatcatgggggcagatttttcccattttcttgtgatagtgaataagtctcatgagagctgagggttttataaaaggcagttcccttgcacaagctttcttgcctaccatcatgtaagatatgcctttttcattataagttacccagtcttggatatttcttcacagtggtatgaaaatggactaatacacatgggctaaactcatgagcaggcaactttatagaaaaacacaaatagcccataaacataaaatgtagattgagttcattattaattagggtaatgcatgttatggatagaataaaatacctttttacattcactggattggcacaaagttttgtctttgtgccactctaaatgttgacagggatgttgatcaactggaattcacacactgcttgaggaagcttgcattcactctataattcagcattctgaaaagtgctcaccaataatcagcccaggaaggttcacaaggacatcttcaccaaatatgtcctttcaccctagcaaagaactagaaacaactcaagtgtcttttgacaagagaatgagtaaatggcaagtggtatgtcacgcaaaagaattctagacagaagagaaaattaaggaacatcaaacttagtgaatctttgagacgatgttgagtaggaaaaaaaattcagaagaatatattgtatgatacttttatcataaggtttaaaagcaagccaaggtaaacatgtgcagggacacctagggacccatgtcattttaggatcaaacagatagatggacagatgatagatagacagacagatggatttttttaactcaaaatagtggttacatccttagggagctgggattggagaagagacacttagctttgaaagtattgtcaaatatagtgacctttctagttcatttgggtgctgggttcaggggtattcattttattatgcaccataatttatgtgagacattttattatatcaaatattttctaatcttacagtaagcagtttggtaaacagattcataactgcatataatgtgtaggtcttgtgaacaatgctgctgtgaatatcaggacccaactgcacttgtctctcactgccttttgtaagcctgctcaattggggagttcttccccttatactggagatgagaactcaggtaatgcctcgactgttcggatcactgtcaggggattacatgttctaccgtgctgacttttcactgaaagcccagtctccactcaataagtaaaagtgtaataatcgaaactcccaatttgagttccacttaaaatcttctctcttcccctgccagacctctctgcaccatctaggggagcatagtcccctctgtccctgcctgacagggctctcctcctcagcagcacagggaggtgtctgacccatctgtgtgtgtgtgtctatgtggtggggatggggaccatagcttggcatcctgccctctcaccctggcagttcattgtctcaagctgactttcttgggtgttttcatggcttcttctgaggcctccctctggaatattggatcgtaactccagcaatgcaagcaataggttttcctccagcttctggtcctcctcctcctcctcctcctccccaaacctggtttctagcagtcactccactgagactcagagtcttcttggcccagtgccagcctcttgcatgtcccaaaccttgtcctcaggaaacacttatgcatcctatgctggggtgaactcttaagagtgcaggatcacctcatggccacagcctagctaggccctgaaacccacactctgggtttgtcagacgtgagtcagccccaggccactgaacctctcccactgcagggaatccctgtcaagtggtgaaagccagcatctagacctggagtcacaggcacctgtagcataactcctcctgaaatggaggtttttctttagagattggtccatatcccttatccctggacttctgagaagggtggacaacctgtggactggacttgagtctggaatttagagaagtgtccatagtagggctgccagatgaggcaaacacaaatacaggatgcccaattaaatttgaatttcagataaatgatttttaatgtttgattctgtcccatgctaaa

SEQ ID NO: 2 - DNA, 12598 bp, Homo sapiens
aggcgggcggcactgcgcctaagctgggccacaaccgccagtcagggctctccccttcccctccctccccccctcctcctcctcctctgccgcccagagcgagacaccaacatggagcccgaggatctgccatggccgggcgagctggaggaggaggaggaggaggaggaggaggaggaggaggaggaagaggaggaggctgcagcggcggcggcggcgaacgtggacgacgtagtggtcgtggaggaggtggaggaagaggcggggcgggagttggactccgactctcactacgggccccagcatctggaaagtatagacgacgaggaggacgaggaggccaaggcctggctgcaggcgcaccccggcaggattttgcctccgctgtcgcccccgcagcaccgctactcggagggcgagcggacctccctggagaagaaaacagaatcttggcattgtcttcctcaagaaatggactcttcccaaaccttggatacatcccagactaggtttaatgtgagaacggaagatactgaagtgacagacttcccctctctggaggagggcatattgacgcaatcagaaaatcaagtaaaggaacccaacagagatctcttctgttctccactgctagtcatacaagatagctttgcttctcctgatttgcctttgctgacctgtttgacacaagaccaagaatttgcgcctgattctttatttcatcaaagtgaactaagttttgcacctctgaggggaattcctgataagtctgaagatactgaatggtcttctcgaccatcggaagttagtgaagctttattccaggctactgcagaagtagcttcagacttagcaagcagtcgctttagtgtatctcagcacccgcttataggcagcacagctgttgggtctcagtgcccttttttaccttctgaacaagggaataatgaagagactatttcgtctgttgatgaactgaaaattcccaaagactgtgatcgttatgatgatctttgttcatatatgtcatggaagacacgaaaagatacacagtggcctgaaaacaatttagctgataaagatcaagtttcagttgcaacttcatttgacataactgatgaaaacatagctactaaaagaagtgaccattttgatgctgctcgttcatatgggcagtattggacacaggaagattcatctaagcaggcagaaacatatttaaccaagggcctgcaggggaaggttgagtctgacgtcattactctggatggcctaaatgaaaatgctgttgtatgcagtgaaagagttgctgaactacaaagaaagccaacaagagagtcggaatatcactcttcagatctcagaatgttgaggatgtctcctgacactgtgccaaaggctcctaaacatttaaaagcaggagacacttctaaaggaggcatagctaaagttactcaatccaacttgaagtcaggcatcactaccactcctgttgattcagacattggatctcatttatccttgtcccttgaggacctgtctcagttggctgtaagttctcctctagaaactactactggtcaacacactgatactctcaaccaaaagacattagcagatactcatctaactgaagagactctgaaagtcacagctattcctgaaccagctgaccagaagactgcaacaccaacagtactctctagttcccactcacatagggggaagcccagcattttctaccagcagggcttgccagacagtcatctaactgaagaggctttgaaagtttcagctgctcctggactagctgaccagacaactggcatgtcaactctaacctctacttcctactcacatagagagaagcctggtactttttaccaacaagagttaccagagagtaacttaaccgaagagcctttggaagtttcagctgctcctggcccagtggagcagaagacgggaatacctacagtatcctctacatcccactcacatggagaggacctcctctttttctatcgacagaccttgccagatggtcatctaactgatcaggctctgaaagtctcagctgtgtctggaccagctgaccagaagactgggacagcaacagtactctctactccccactcacatagagagaagcctggtattttttaccaacaagagtttgcagacagtcatcaaactgaagagactcttactaaagtttcagccactcctggaccagctgaccagaagactgagataccagcagtacagtctagttcttactcacaaagagaaaagcctagtattttgtacccacaggacttagcagacagtcatctacctgaagagggtctgaaagtttcagctgttgctggaccagctgaccagaagactggcctaccaacagtaccctctagtgcatactcacacagagagaagctccttgttttctaccaacaggccttgctggacagccatctacccgaagaggctctgaaagtttcagctgtttctggaccagctgacggaaagactgggacaccagctgtaacctctacttcctctgcgtcctcttcacttggagaaaagcccagtgctttctatcagcagaccttacccaatagtcatctaactgaagaggctctgaaagtatcaattgttcctggaccaggtgatcagaagactgggataccctcagcaccatctagtttctactcacacagagagaagcccattattttttcccagcagaccctgccagactttcttttccctgaagaagctctgaaggtttcagctgtttctgtattggctgcccagaagactgggacaccaacagtgtcctctaattctcactcacatagcgagaaatctagtgttttctaccagcaagagttgccagacagtgatctacctagagaatctctgaaaatgtctgctattcctggactgactgaccagaagactgtcccaacaccaacagtaccttcaggttccttctcacatagagagaagcccagtattttctatcaacaggagtggccagatagttatgcaactgaaaaggctctgaaagtttcaactggccctggaccagctgaccagaagactgagataccagcagtacagtctagttcttacccacagagggagaagcctagtgttttgtacccacaggtgttatcagacagtcatctacctgaagagagtctgaaagtttcagccttccctggaccagctgaccagatgactgacacaccagcagtaccgtctactttctactcacaaagagagaagcctggtattttctaccaacagaccttgccagagagtcatctgcctaaagaggctctgaaaatttcagtagctcctggactagcagaccagaagactggcacaccaactgtaacctcaacttcctactcacaacatagagaaaagcccagcattttccaccagcaggccttgccaggtactcatatacctgaagaggctcagaaagtttcagctgttactggaccaggtaaccagaagacttggataccaagagtactttctaccttctactcacaaagagagaaacctggtattttctatcaacagaccttgccaggtagtcacatacctgaagaggcacagaaagtttcacctgttcttggaccagctgaccagaagactgggacaccaactccaacctctgcttcttactcacacacagagaagcctggtattttctaccaacaggtcttgccagataatcatccaactgaagaggctctgaaaatttcagttgcctctgaaccagttgaccagacaactggcacaccagctgtaacctctacttcctactcacaatatagagagaagcccagcattttctaccaacagtcgttgccaagtagtcatctaactgaagaggctaagaatgtttcagcggttcctggaccagctgaccagaagactgtgataccaattttaccctctactttctactcacacacagagaagcctggtgttttctaccaacaggtcttgccacatagtcatccaactgaagaggctctgaaaatttcagttgcctctgaaccagttgaccagacaactggcacaccaactgtaacctctacttcttactcacaacatacagagaagccgagtattttctaccaacagtcgttgccaggtagtcatctaactgaagaggctaagaacgtttcagcggttcctggaccaggtgaccggaagactgggataccaactttaccctctactttctactcacacacagagaagcctggtagtttctaccaacaggtcttgccacatagtcatctacctgaagaggctttggaagtttcagttgctcctggaccagttgaccagacgattggcacaccaactgtaacctccccttccagctcatttggagagaagcccattgttatctacaaacaggcctttccagagggtcatctacctgaagagtctctgaaagtttcagttgctcctggaccagttggccagacaactggcgcaccaactataacctctccttcctactcacaacatagagcaaagtctggcagtttctaccaactggcattgctaggtagtcaaatacctgaagaggctctcagagtttcttctgctcctggaccagctgaccagacaactggcataccaaccataacctctacttcctactcatttggagagaagccgattgttaactacaaacaggcctttccagatggtcatctacctgaagaggctctgaaagtttccattgtttctggacctactgaaaaaaagactgacataccagcaggacctttaggttccagtgcacttggagagaagcccattactttctaccggcaggctctgctagacagtcctctaaataaagaggttgtgaaagtttcagctgctcctggaccagctgaccagaagactgagacattaccagtacattctactagctactcaaatagggggaagcctgtcattttctaccagcagaccctatcagacagtcatttacctgaagaagctctgaaagttccacctgttcctggaccagatgcccagaagactgagacaccatcagtatcctctagtttatactcatatagagagaagcccattgtcttctaccaacaggccctgccagacagtgagctaactcaagaagctctgaaagtttcagctgttcctcaaccagctgaccagaagactgggttatctactgtaacttcctctttctattcacatacagagaagcctaatatttcttaccagcaagagttgccagatagtcatctaactgaagaggctctgaaagtttcaaatgttcctggaccagctgaccagaagactggggtatcaacagtaacctctacttcctactcacacagagagaagcccattgtttcctaccagcgagagttgccgcattttactgaagcaggtttgaaaattttaagagttcctggaccagctgaccagaagactggaataaacatcctgccctctaattcctacccacagagagagcactctgtcatttcttatgagcaggagttgccagatcttactgaagtaactttgaaagcaataggggttcctgggcctgctgaccagaagactgggatacaaatagcatcctctagttcctactcaaatagagagaaggccagtatttttcatcagcaggagttgccagatgttactgaagaagctttaaatgtttttgttgttcctggacaaggtgaccggaagactgagataccaacagtacctttaagttactactcacgtagagagaagcccagtgttatctctcaacaggagttgccagacagtcatctcacagaagaggctctgaaagtttcacctgtttctataccagcagagcagaagactgggataccaataggactgtctagttcctactcacattcacataaagagaaactcaagatttcaactgtgcatataccagatgaccagaaaactgagtttccagcagctacccttagttcctactcacaaatagagaagcccaagatttcaactgtgattggaccaaatgaccagaagactccatcccagacagcttttcatagttcctattctcaaacagtaaagcccaatattttatttcaacagcagttgccagatagagatcaaagtaaaggtattctaaagatttcagctgtccctgaactaactgatgtgaatactggaaaaccagtatctctctctagttcttattttcacagagagaaatcgaatattttcagtccacaggaattgccaggtagacatgtaactgaagatgtgctgaaggtttcaacaattcctggaccagctggccagaaaacagtattaccaacagctcttcctagttccttttcacatcgagagaaaccagatattttctatcaaaaggatttgccagatagacatctaactgaagatgctctaaagatctcaagtgctcttgggcaagctgatcaaattaccggattacaaacagttccctctggtacttactcacatggtgagaatcacaagcttgtttcagaacatgtccaaaggctaatagataatttgaattcttctgactccagtgttagctcaaataatgtgcttttaaattctcaggctgatgacagagttgtaataaataaaccagaatctgcaggttttagagatgttggctctgaagaaatccaggatgcagaaaatagtgctaaaactcttaaggaaattcggacacttttgatggaggcagaaaatatggcactgaaacgatgcaattttcctgctccccttgcccgtttcagagatattagtgatatttcatttatacaatctaagaaggtggtttgcttcaaagaaccctcttccacgggtgtatctaatggtgatttgcttcacagacagccattcacagaggaaagcccaagcagcaggtgcatacagaaggatattggcacacagacgaatttgaaatgccggagaggcattgaaaattgggagtttattagttcaactacagttagaagtcctctacaggaagcagagagcaaagtcagtatggcattagaagaaactcttaggcaatatcaagcagccaaatctgtaatgaggtctgaacctgaagggtgtagtggaaccattgggaataaaattattatccctatgatgactgtcataaaaagtgattcaagtagtgatgccagtgatggaaatggttcctgctcgtgggacagtaatttaccagagtctttggaatcagtttctgatgttcttctaaacttctttccatatgtttcacccaagacaagtataacagatagcagggaggaagagggtgtgtcagagagtgaggatggtggtggtagcagtgtagattcactggctgcacatgtgaaaaaccttctgcaatgtgaatcctcactgaatcatgctaaagaaatactcagaaatgcagaggaagaggaaagccgggtacgagcacatgcctggaatatgaagttcaatttagcacatgattgtggatactccatttcagaattaaatgaagatgacaggaggaaagtagaagagatcaaggcagagttatttggtcatggaagaacaactgacttgtccaagggtttacagagtccacggggaatgggatgcaagccagaagctgtatgtagtcacattattattgagagccatgaaaagggatgtttccggactctaacttctgaacatccacaactagatagacacccttgtgctttcagatctgctggaccctcagaaatgaccagaggacggcagaacccatcatcatgcagagccaagcatgtcaacctttctgcatccttagaccagaacaactcccatttcaaagtttggaattccttgcagttaaaaagtcattccccatttcagaactttatacctgatgaattcaaaatcagcaaaggtcttcgaatgccattcgatgaaaagatggacccttggctgtcagaattagtagaacctgcttttgtgccacctaaagaagtggattttcattcttcatcacaaatgccgtccccagaacccatgaaaaagtttactacctccatcactttttcatctcaccgacattctaaatgcatttccaattcctctgttgttaaggttggtgttactgaaggtagccagtgtactggagcatctgtgggggtatttaattctcatttcactgaagaacaaaatcctcccagagatcttaaacagaaaacctcttccccttcatcatttaaaatgcatagtaattcacaagataaagaagtgactattttagcagaaggtagaaggcaaagccaaaaattacctgttgattttgagcgttcttttcaagaagaaaaacccttagaaagatcagattttacaggcagtcattctgagcccagtaccagggcaaattgtagcaatttcaaggaaattcagatttctgataaccatacccttattagcatgggcagaccaagttccaccctaggagtaaacagatcgagttccagactaggagtaaaagagaagaatgtaactataactccagatcttccttcttgcatttttcttgaacaacgagagctctttgaacaaagcaaagccccacgtgcagatgaccatgtgaggaaacaccattctccctctcctcaacatcaggattatgtagctccagaccttccttcttgcatttttcttgaacaacgagaactctttgaacagtgcaaagccccatatgtagatcatcaaatgagagaaaaccattctccccttcctcaaggtcaggattctatagcttcagaccttccgtctcccatttctcttgaacaatgccaaagcaaagcgccaggtgtagatgaccaaatgaataaacaccattttccccttcctcaaggtcaggattgtgtagtggaaaagaataatcaacataagcctaaatcacacatttctaatataaatgttgaagccaagttcaatactgtggtctcccagtcagccccaaatcactgtacattagcagcatctgcatctactcctccttcaaatagaaaagcactttcttgtgttcatataactctttgtcccaagacttcttccaagttggatagtggaactttagatgaaagattccattcattggatgctgcttctaaagcgaggatgaatagtgagtttaactttgacttacatactgtatcttcgagatcactggaaccaacctccaaattattgaccagtaaacctgtagcacaggatcaagaatctttaggttttctaggacctaaatcttcactggatttccaagtcgtacagccttctcttccagacagtaacactattactcaggacttgaaaaccataccttctcagaatagccagatagtaacctccaggcaaatacaagtgaacatttcagatttcgaaggacattccaatccagaggggaccccagtatttgcagatcgattaccagagaagatgaagaccccactttctgctttctctgaaaaattgtcatctgatgcagtcactcagataacaacagaaagtccagaaaagaccctattttcatctgagatttttattaatgctgaagatcgtggacatgaaattatagagcctggtaaccagaagctacgcaaagctcctgtcaagtttgcctcatcatcttcagtccaacaggttactttttctcgcggcacagatggccagcctttattattgccatataagccttctggtagtaccaagatgtattatgttccacaattaagacaaattcctccatctccggattccaaatcagataccaccgttgaaagctcccattcaggatccaatgatgccattgctccagacttcccagctcaggtgctaggcacaagagatgatgacctctcagccactgttaacattaaacataaagaaggaatctacagtaagagggtagtgactaaggcatccttgccagtgggagaaaaacccttgcagaatgaaaatgcagatgcctcagttcaagtgctaatcactggggatgagaacctctcagacaaaaaacagcaagagattcacagtacaagggcagtgactgaggctgcccaggctaaagaaaaagaatctttgcagaaagatactgcagattccagtgctgctgctgctgcagagcactcagctcaagtaggagacccagaaatgaagaacttgccagacactaaagccattacacagaaagaggagatccataggaagaagacagttcccgaggaagcctggccaaacaataaagaatccctacagatcaatattgaagagtccgaatgtcattcagaatttgaaaatactacccgttctgtcttcaggtcagcaaagttttacattcatcatcccgtacacctaccaagtgatcaagatatttgccatgaatctttgggaaagagtgttttcatgagacattcttggaaagatttctttcagcatcatccagacaaacatagagaacacatgtgtcttcctcttccttatcaaaacatggacaagactaagacagattataccagaataaagagcctcagcatcaatgtgaatttgggaaacaaagaagtgatggatactactaaaagtcaagttagagattatccaaaacataatggacaaattagtgatccacaaagggatcagaaggtcaccccagagcaaacaactcagcacactgtgagtttgaatgaactgtggaacaagtatcgggagcgacagaggcaacagagacagcctgagttgggtgacaggaaagaactgtccttggtggaccgacttgatcggttggctaaaattcttcagaatccaatcacacattctctccaggtctcagaaagtacacatgatgatagcagaggggaacgaagtgtgaaggaatggagtggtagacaacagcagagaaataagcttcagaaaaagaagcggtttaaaagcctagagaaaagccataaaaatacaggcgagcttaaaaaaagcaaggtgctttctcatcatcgagctgggaggtctaatcaaattaaaattgaacagattaaatttgataaatatattctgagtaaacagccaggttttaattatataagcaacacttcttcggattgtcggccctcagaggagagtgagctgctcacagatactaccaccaacatcctttccggcaccacttctactgtcgaatcagatatattgacccaaacagatagagaggtggctctgcacgaaaggagtagctctgtttccactattgacactgcccggctgattcaagcttttggccatgaaagagtatgcttgtcacccagacgaattaaattatatagcagcatcaccaaccaacagaggagataccttgagaagcggagcaaacacagcaagaaagtgctgaatacaggtcatcccctagtgacttctgagcacaccagaaggagacacatccaggtagcaaaccatgtgatttcttctgactctatttcctcttctgccagtagtttcctgagctcaaactctactttttgcaacaagcagaatgtacacatgttaaacaagggcatacaagcaggtaacttggagattgtgaacggtgccaaaaaacacactcgagatgttgggataactttcccaactccaagttccagcgaggctaaattggaagagaacagtgatgtgacttcttggtcagaagaaaaacgtgaagagaaaatgctctttaccggttatcctgaggacagaaagttaaaaaagaacaagaagaattcccatgaaggagtttcctggtttgttcctgtggaaaatgtggagtctagatcaaagaaggaaaacgtgcctaacacttgtggccctggcatctcctggtttgaaccaataaccaagaccagaccctggagggagccactgcgggagcagaactgtcaggggcagcacctggacggtcggggctacctggcaggcccaggcagagaggctggcagagacctactgaagccatttgtgagagcaacccttcaggaatcgcttcagtttcacagacctgacttcatctcccgctctggggagcggataaagcgcctgaagttaatagtccaggagaggaagctgcagagcatgttacagaccgagcgggatgcactattcaacattgacagggaacggcagggccaccagaatcgcatgtgcccgctgcccaagagagtcttcctggctatccagaagaacaagcctatcagcaagaaggaaatgattcagaggtccaaacggatttatgagcagcttccagaagtacagaaaaagagagaagaagagaagagaaaatcagaatataagtcataccggctgcgagcccagctatataaaaagagagtgaccaatcaacttctggggagaaaagttccctgggactgacacaagtttattttcctcagagccttggaattctattttatgaacctagagaagcagaatccttacttttgtgagtctggttgaataaagcttattctttgtc

SEQ ID NO: 3 - protein, 4127 aa, Homo sapiens
MEPEDLPWPGELEEEEEEEEEEEEEEEEEAAAAAAANVDDVVVVEEVEEEAGRELDSDSHYGPQHLESIDDEEDEEAKAWLQAHPGRILPPLSPPQHRYSEGERTSLEKKTESWHCLPQEMDSSQTLDTSQTRFNVRTEDTEVTDFPSLEEGILTQSENQVKEPNRDLFCSPLLVIQDSFASPDLPLLTCLTQDQEFAPDSLFHQSELSFAPLRGIPDKSEDTEWSSRPSEVSEALFQATAEVASDLASSRFSVSQHPLIGSTAVGSQCPFLPSEQGNNEETISSVDELKIPKDCDRYDDLCSYMSWKTRKDTQWPENNLADKDQVSVATSFDITDENIATKRSDHFDAARSYGQYWTQEDSSKQAETYLTKGLQGKVESDVITLDGLNENAVVCSERVAELQRKPTRESEYHSSDLRMLRMSPDTVPKAPKHLKAGDTSKGGIAKVTQSNLKSGITTTPVDSDIGSHLSLSLEDLSQLAVSSPLETTTGQHTDTLNQKTLADTHLTEETLKVTAIPEPADQKTATPTVLSSSHSHRGKPSIFYQQGLPDSHLTEEALKVSAAPGLADQTTGMSTLTSTSYSHREKPGTFYQQELPESNLTEEPLEVSAAPGPVEQKTGIPTVSSTSHSHGEDLLFFYRQTLPDGHLTDQALKVSAVSGPADQKTGTATVLSTPHSHREKPGIFYQQEFADSHQTEETLTKVSATPGPADQKTEIPAVQSSSYSQREKPSILYPQDLADSHLPEEGLKVSAVAGPADQKTGLPTVPSSAYSHREKLLVFYQQALLDSHLPEEALKVSAVSGPADGKTGTPAVTSTSSASSSLGEKPSAFYQQTLPNSHLTEEALKVSIVPGPGDQKTGIPSAPSSFYSHREKPIIFSQQTLPDFLFPEEALKVSAVSVLAAQKTGTPTVSSNSHSHSEKSSVFYQQELPDSDLPRESLKMSAIPGLTDQKTVPTPTVPSGSFSHREKPSIFYQQEWPDSYATEKALKVSTGPGPADQKTEIPAVQSSSYPQREKPSVLYPQVLSDSHLPEESLKVSAFPGPADQMTDTPAVPSTFYSQREKPGIFYQQTLPESHLPKEALKISVAPGLADQKTGTPTVTSTSYSQHREKPSIFHQQALPGTHIPEEAQKVSAVTGPGNQKTWIPRVLSTFYSQREKPGIFYQQTLPGSHIPEEAQKVSPVLGPADQKTGTPTPTSASYSHTEKPGIFYQQVLPDNHPTEEALKISVASEPVDQTTGTPAVTSTSYSQYREKPSIFYQQSLPSSHLTEEAKNVSAVPGPADQKTVIPILPSTFYSHTEKPGVFYQQVLPHSHPTEEALKISVASEPVDQTTGTPTVTSTSYSQHTEKPSIFYQQSLPGSHLTEEAKNVSAVPGPGDRKTGIPTLPSTFYSHTEKPGSFYQQVLPHSHLPEEALEVSVAPGPVDQTIGTPTVTSPSSSFGEKPIVIYKQAFPEGHLPEESLKVSVAPGPVGQTTGAPTITSPSYSQHRAKSGSFYQLALLGSQIPEEALRVSSAPGPADQTTGIPTITSTSYSFGEKPIVNYKQAFPDGHLPEEALKVSIVSGPTEKKTDIPAGPLGSSALGEKPITFYRQALLDSPLNKEVVKVSAAPGPADQKTETLPVHSTSYSNRGKPVIFYQQTLSDSHLPEEALKVPPVPGPDAQKTETPSVSSSLYSYREKPIVFYQQALPDSELTQEALKVSAVPQPADQKTGLSTVTSSFYSHTEKPNISYQQELPDSHLTEEALKVSNVPGPADQKTGVSTVTSTSYSHREKPIVSYQRELPHFTEAGLKILRVPGPADQKTGINILPSNSYPQREHSVISYEQELPDLTEVTLKAIGVPGPADQKTGIQIASSSSYSNREKASIFHQQELPDVTEEALNVFVVPGQGDRKTEIPTVPLSYYSRREKPSVISQQELPDSHLTEEALKVSPVSIPAEQKTGIPIGLSSSYSHSHKEKLKISTVHIPDDQKTEFPAATLSSYSQIEKPKISTVIGPNDQKTPSQTAFHSSYSQTVKPNILFQQQLPDRDQSKGILKISAVPELTDVNTGKPVSLSSSYFHREKSNIFSPQELPGRHVTEDVLKVSTIPGPAGQKTVLPTALPSSFSHREKPDIFYQKDLPDRHLTEDALKISSALGQADQITGLQTVPSGTYSHGENHKLVSEHVQRLIDNLNSSDSSVSSNNVLLNSQADDRVVINKPESAGFRDVGSEEIQDAENSAKTLKEIRTLLMEAENMALKRCNFPAPLARFRDISDISFIQSKKVVCFKEPSSTGVSNGDLLHRQPFTEESPSSRCIQKDIGTQTNLKCRRGIENWEFISSTTVRSPLQEAESKVSMALEETLRQYQAAKSVMRSEPEGCSGTIGNKIIIPMMTVIKSDSSSDASDGNGSCSWDSNLPESLESVSDVLLNFFPYVSPKTSITDSREEEGVSESEDGGGSSVDSLAAHVKNLLQCESSLNHAKEILRNAEEEESRVRAHAWNMKFNLAHDCGYSISELNEDDRRKVEEIKAELFGHGRTTDLSKGLQSPRGMGCKPEAVCSHIIIESHEKGCFRTLTSEHPQLDRHPCAFRSAGPSEMTRGRQNPSSCRAKHVNLSASLDQNNSHFKVWNSLQLKSHSPFQNFIPDEFKISKGLRMPFDEKMDPWLSELVEPAFVPPKEVDFHSSSQMPSPEPMKKFTTSITFSSHRHSKCISNSSVVKVGVTEGSQCTGASVGVFNSHFTEEQNPPRDLKQKTSSPSSFKMHSNSQDKEVTILAEGRRQSQKLPVDFERSFQEEKPLERSDFTGSHSEPSTRANCSNFKEIQISDNHTLISMGRPSSTLGVNRSSSRLGVKEKNVTITPDLPSCIFLEQRELFEQSKAPRADDHVRKHHSPSPQHQDYVAPDLPSCIFLEQRELFEQCKAPYVDHQMRENHSPLPQGQDSIASDLPSPISLEQCQSKAPGVDDQMNKHHFPLPQGQDCVVEKNNQHKPKSHISNINVEAKFNTVVSQSAPNHCTLAASASTPPSNRKALSCVHITLCPKTSSKLDSGTLDERFHSLDAASKARMNSEFNFDLHTVSSRSLEPTSKLLTSKPVAQDQESLGFLGPKSSLDFQVVQPSLPDSNTITQDLKTIPSQNSQIVTSRQIQVNISDFEGHSNPEGTPVFADRLPEKMKTPLSAFSEKLSSDAVTQITTESPEKTLFSSEIFINAEDRGHEIIEPGNQKLRKAPVKFASSSSVQQVTFSRGTDGQPLLLPYKPSGSTKMYYVPQLRQIPPSPDSKSDTTVESSHSGSNDAIAPDFPAQVLGTRDDDLSATVNIKHKEGIYSKRVVTKASLPVGEKPLQNENADASVQVLITGDENLSDKKQQEIHSTRAVTEAAQAKEKESLQKDTADSSAAAAAEHSAQVGDPEMKNLPDTKAITQKEEIHRKKTVPEEAWPNNKESLQINIEESECHSEFENTTRSVFRSAKFYIHHPVHLPSDQDICHESLGKSVFMRHSWKDFFQHHPDKHREHMCLPLPYQNMDKTKTDYTRIKSLSINVNLGNKEVMDTTKSQVRDYPKHNGQISDPQRDQKVTPEQTTQHTVSLNELWNKYRERQRQQRQPELGDRKELSLVDRLDRLAKILQNPITHSLQVSESTHDDSRGERSVKEWSGRQQQRNKLQKKKRFKSLEKSHKNTGELKKSKVLSHHRAGRSNQIKIEQIKFDKYILSKQPGFNYISNTSSDCRPSEESELLTDTTTNILSGTTSTVESDILTQTDREVALHERSSSVSTIDTARLIQAFGHERVCLSPRRIKLYSSITNQQRRYLEKRSKHSKKVLNTGHPLVTSEHTRRRHIQVANHVISSDSISSSASSFLSSNSTFCNKQNVHMLNKGIQAGNLEIVNGAKKHTRDVGITFPTPSSSEAKLEENSDVTSWSEEKREEKMLFTGYPEDRKLKKNKKNSHEGVSWFVPVENVESRSKKENVPNTCGPGISWFEPITKTRPWREPLREQNCQGQHLDGRGYLAGPGREAGRDLLKPFVRATLQESLQFHRPDFISRSGERIKRLKLIVQERKLQSMLQTERDALFNIDRERQGHQNRMCPLPKRVFLAIQKNKPISKKEMIQRSKRIYEQLPEVQKKREEEKRKSEYKSYRLRAQLYKKRVTNQLLGRKVPWD

SEQ ID NO: 4 - DNA, 24 bp, Artificial Sequence
  Primer
cctgtagcaaacctccgccctaag

SEQ ID NO: 5 - DNA, 19 bp, Artificial Sequence
  Primer
gcgcgttttctccgtcagg

SEQ ID NO: 6 - DNA, 24 bp, Artificial Sequence
  Primer
gatgtgtagtaatgggaagaggtc

SEQ ID NO: 7 - DNA, 24 bp, Artificial Sequence
  Primer
aacgggaagtgataaaatgagagg

SEQ ID NO: 8 - DNA, 22 bp, Artificial Sequence
  Primer
gctgctactgctgttgctttta

SEQ ID NO: 9 - DNA, 24 bp, Artificial Sequence
  Primer
actttgttaccttcagggcgtgtc

SEQ ID NO: 10 - DNA, 24 bp, Artificial Sequence
  Primer
aggagagctgtgtttttcaataca

SEQ ID NO: 11 - DNA, 22 bp, Artificial Sequence
  Primer
gtttctgggtggtgcaaagttc

SEQ ID NO: 12 - DNA, 24 bp, Artificial Sequence
  Primer
ctgatataggccaaggtgaaagtc

SEQ ID NO: 13 - DNA, 26 bp, Artificial Sequence
  Primer
acccaggaaacagtaaaaagtaaaat

SEQ ID NO: 14 - DNA, 24 bp, Artificial Sequence
  Primer
tcatttcttcgtaggtgggtcatt

SEQ ID NO: 15 - DNA, 24 bp, Artificial Sequence
  Primer
ttttcacaaggtatccgtaagtag

SEQ ID NO: 16 - DNA, 24 bp, Artificial Sequence
  Primer
tctcctttgatggctgtttcctta

SEQ ID NO: 17 - DNA, 23 bp, Artificial Sequence
  Primer
gtattcccgtcttctgctccact

SEQ ID NO: 18 - DNA, 18 bp, Artificial Sequence
  Primer
caactggcatgtcaactc

SEQ ID NO: 19 - DNA, 19 bp, Artificial Sequence
  Primer
cttcgggtagatggctgtc

SEQ ID NO: 20 - DNA, 19 bp, Artificial Sequence
  Primer
gtacccacaggacttagca

SEQ ID NO: 21 - DNA, 24 bp, Artificial Sequence
  Primer
actcctgttgatagaaaatactgg

SEQ ID NO: 22 - DNA, 24 bp, Artificial Sequence
  Primer
ctgaccagaagactgtcccaacac

SEQ ID NO: 23 - DNA, 21 bp, Artificial Sequence
  Primer
caaggcctgctggtggaaaat

SEQ ID NO: 24 - DNA, 21 bp, Artificial Sequence
  Primer
cacaccagcagtaccgtctac

SEQ ID NO: 25 - DNA, 26 bp, Artificial Sequence
  Primer
tcagagcctcttcagttggatgatta

SEQ ID NO: 26 - DNA, 20 bp, Artificial Sequence
  Primer
gaaagtttcacctgttcttg

SEQ ID NO: 27 - DNA, 19 bp, Artificial Sequence
  Primer
tggtccaggagcagaagaa

SEQ ID NO: 28 - DNA, 23 bp, Artificial Sequence
  Primer
tggcgcaccaactataacctctc

SEQ ID NO: 29 - DNA, 24 bp, Artificial Sequence
  Primer
gctggtagaaaatgacaggcttcc

SEQ ID NO: 30 - DNA, 22 bp, Artificial Sequence
  Primer
ctaaataaagaggttgtgaaag

SEQ ID NO: 31 - DNA, 23 bp, Artificial Sequence
  Primer
atgtgaatagaaagaggaagtta

SEQ ID NO: 32 - DNA, 21 bp, Artificial Sequence
  Primer
caggccctgcagaacagtgag

SEQ ID NO: 33 - DNA, 22 bp, Artificial Sequence
  Primer
ctggcaactcctgctgatgaaa

SEQ ID NO: 34 - DNA, 19 bp, Artificial Sequence
  Primer
ggttcctgggcctgctgac

SEQ ID NO: 35 - DNA, 24 bp, Artificial Sequence
  Primer
ttgggctttactgtttgagaatag

SEQ ID NO: 36 - DNA, 23 bp, Artificial Sequence
  Primer
tcacaaatagagaagcccaagat

SEQ ID NO: 37 - DNA, 24 bp, Artificial Sequence
  Primer
atgtgaaaaggaactaggaagagc

SEQ ID NO: 38 - DNA, 23 bp, Artificial Sequence
  Primer
atgtaactgaagatgtgctgaag

SEQ ID NO: 39 - DNA, 22 bp, Artificial Sequence
  Primer
ttctgcctccatcaaaagtgtc

SEQ ID NO: 40 - DNA, 21 bp, Artificial Sequence
  Primer
aaggatttgccagatagacat

SEQ ID NO: 41 - DNA, 22 bp, Artificial Sequence
  Primer
tcctctgtgaatggctgtctgt

SEQ ID NO: 42 - DNA, 22 bp, Artificial Sequence
  Primer
actcttaaggaaattcggacac

SEQ ID NO: 43 - DNA, 22 bp, Artificial Sequence
  Primer
ttacagatttggctgcttgata

SEQ ID NO: 44 - DNA, 22 bp, Artificial Sequence
  Primer
cctcttccacgggtgtatctaa

SEQ ID NO: 45 - DNA, 22 bp, Artificial Sequence
  Primer
accctcttcctccctgctatct

SEQ ID NO: 46 - DNA, 22 bp, Artificial Sequence
  Primer
gcaatatcaagcagccaaatct

SEQ ID NO: 47 - DNA, 23 bp, Artificial Sequence
  Primer
gatcttgcttgtatttttcattg

SEQ ID NO: 48 - DNA, 21 bp, Artificial Sequence
  Primer
ctaagcattgcagtgggtatt

SEQ ID NO: 49 - DNA, 26 bp, Artificial Sequence
  Primer
ccattctttcatatttactttcttca

SEQ ID NO: 50 - DNA, 23 bp, Artificial Sequence
  Primer
ttttgcttgctgtgctgtttgtc

SEQ ID NO: 51 - DNA, 23 bp, Artificial Sequence
  Primer
agttctgaaatggggaatgactt

SEQ ID NO: 52 - DNA, 24 bp, Artificial Sequence
  Primer
ttggctgtcagaattagtagaacc

SEQ ID NO: 53 - DNA, 24 bp, Artificial Sequence
  Primer
gaaaagaacgctcaaaatcaacag

SEQ ID NO: 54 - DNA, 24 bp, Artificial Sequence
  Primer
ttttaaatgatgaaggggaagagg

SEQ ID NO: 55 - DNA, 22 bp, Artificial Sequence
  Primer
aatgaccagaggacggcagaac

SEQ ID NO: 56 - DNA, 24 bp, Artificial Sequence
  Primer
aggaaggtctggagctacataatc

SEQ ID NO: 57 - DNA, 24 bp, Artificial Sequence
  Primer
gggggtatttaattctcatttcac

SEQ ID NO: 58 - DNA, 24 bp, Artificial Sequence
  Primer
cactattcatcctcgctttagaag

SEQ ID NO: 59 - DNA, 19 bp, Artificial Sequence
  Primer
cccacgtgcagatgaccat

SEQ ID NO: 60 - DNA, 24 bp, Artificial Sequence
  Primer
ttagccagacacaacctccactca

SEQ ID NO: 61 - DNA, 24 bp, Artificial Sequence
  Primer
gcatctgcatctactcctccttca

SEQ ID NO: 62 - DNA, 26 bp, Artificial Sequence
  Primer
acagctatgcaaataaaagacagata

SEQ ID NO: 63 - DNA, 26 bp, Artificial Sequence
  Primer
atattccttgaaaccacttttgtaag

SEQ ID NO: 64 - DNA, 23 bp, Artificial Sequence
  Primer
gaacactgcctgaaacatagaag

SEQ ID NO: 65 - DNA, 24 bp, Artificial Sequence
  Primer
tcactgaaaaattaaagagcactg

SEQ ID NO: 66 - DNA, 22 bp, Artificial Sequence
  Primer
atcgtgcctggcctgtagtgct

SEQ ID NO: 67 - DNA, 26 bp, Artificial Sequence
  Primer
attggatagtaatctcatttaggatg

SEQ ID NO: 68 - DNA, 20 bp, Artificial Sequence
  Primer
aattgtgggggaggattact

SEQ ID NO: 69 - DNA, 24 bp, Artificial Sequence
  Primer
ctgtttctgttcttaagggaatag

SEQ ID NO: 70 - DNA, 24 bp, Artificial Sequence
  Primer
accgctacctctttttctgactgt

SEQ ID NO: 71 - DNA, 24 bp, Artificial Sequence
  Primer
cagaatgaaatggaaacactaaca

SEQ ID NO: 72 - DNA, 22 bp, Artificial Sequence
  Primer
gttctttcctgtcacccaactc

SEQ ID NO: 73 - DNA, 22 bp, Artificial Sequence
  Primer
gcaggcagtgaattttctgata

SEQ ID NO: 74 - DNA, 24 bp, Artificial Sequence
  Primer
tgggaaacaaagaagtgatggata

SEQ ID NO: 75 - DNA, 24 bp, Artificial Sequence
  Primer
ctctaggcttttaaaccgcttctt

SEQ ID NO: 76 - DNA, 22 bp, Artificial Sequence
  Primer
agaagtggtgccggaaaggatg

SEQ ID NO: 77 - DNA, 23 bp, Artificial Sequence
  Primer
aaggtcaccccagagcaaacaac

SEQ ID NO: 78 - DNA, 24 bp, Artificial Sequence
  Primer
tctcactcttttccagttggtaag

SEQ ID NO: 79 - DNA, 24 bp, Artificial Sequence
  Primer
atcgagctgggaggtctaatcaaa

SEQ ID NO: 80 - DNA, 20 bp, Artificial Sequence
  Primer
aacccgcttccctacactct

SEQ ID NO: 81 - DNA, 21 bp, Artificial Sequence
  Primer
cctccaaatgcatctcaacag

SEQ ID NO: 82 - DNA, 23 bp, Artificial Sequence
  Primer
cttggaaacacagagtcagcatc

SEQ ID NO: 83 - DNA, 22 bp, Artificial Sequence
  Primer
acttgggatcagagttgaaatc

SEQ ID NO: 84 - DNA, 21 bp, Artificial Sequence
  Primer
acccaaccctcgtgcgtgaaa

SEQ ID NO: 85 - DNA, 22 bp, Artificial Sequence
  Primer
agctgagacccctgagaacctg

SEQ ID NO: 86 - DNA, 19 bp, Artificial Sequence
  Primer
tggcaagcaaaaggtcact

SEQ ID NO: 87 - DNA, 20 bp, Artificial Sequence
  Primer
gctgcagctctcccccatag

SEQ ID NO: 88 - DNA, 18 bp, Artificial Sequence
  Primer
gccagagctcccgaccac

SEQ ID NO: 89 - DNA, 24 bp, Artificial Sequence
  Primer
ctaagatttgaacccaggtgtgct

SEQ ID NO: 90 - DNA, 22 bp, Artificial Sequence
  Primer
gggaggtatagggagggatgat

SEQ ID NO: 91 - DNA, 20 bp, Artificial Sequence
  Primer
tggaaagggccttacaccat

SEQ ID NO: 92 - DNA, 24 bp, Artificial Sequence
  Primer
gatttgaataggaccacactgatt

SEQ ID NO: 93 - DNA, 24 bp, Artificial Sequence
  Primer
agaaagtattgctcccaaaccatt

SEQ ID NO: 94 - DNA, 10 bp, Homo sapiens
accaacatgg

SEQ ID NO: 95 - protein, 16 aa, Artificial Sequence
  Peptide sequence for polyclonal antibody production
CNKPISKKEMIQRSKR

SEQ ID NO: 96 - protein, 15 aa, Artificial Sequence
  Peptide sequence for polyclonal antibody production
CHREKPGTFYQQELK

SEQ ID NO: 97 - protein, 1602 aa, Homo sapiens
QKTLADTHLTEETLKVTAIPEPADQKTATPTVLSSSHSHRGKPSIFYQQGLPDSHLTEEALKVSAAPGLADQTTGMSTLTSTSYSHREKPGTFYQQELPESNLTEEPLEVSAAPGPVEQKTGIPTVSSTSHSHVEDLLFFYRQTLPDGHLTDQALKVSAVSGPADQKTGTATVLSTPHSHREKPGIFYQQEFADSHQTEETLTKVSATPGPADQKTEIPAVQSSSYSQREKPSILYPQDLADSHLPEEGLKVSAVAGPADQKTGLPTVPSSAYSHREKLLVFYQQALLDSHLPEEALKVSAVSGPADGKTGTPAVTSTSSASSSLGEKPSAFYQQTLPNSHLTEEALKVSIVPGPGDQKTGIPSAPSSFYSHREKPIIFSQQTLPDFLFPEEALKVSAVSVLAAQKTGTPTVSSNSHSHSEKSSVFYQQELPDSDLPRESLKMSAIPGLTDQKTVPTPTVPSGSFSHREKPSIFYQQEWPDSYATEKALKVSTGPGPADQKTEIPAVQSSSYPQREKPSVLYPQVLSDSHLPEESLKVSAFPGPADQMTDTPAVPSTFYSQREKPGIFYQQTLPESHLPKEALKISVAPGLADQKTGTPTVTSTSYSQHREKPSIFHQQALPGTHIPEEAQKVSAVTGPGNQKTWIPRVLSTFYSQREKPGIFYQQTLPGSHIPEEAQKVSPVLGPADQKTGTPTPTSASYSHTEKPGIFYQQVLPDNHPTEEALKISVASEPVDQTTGTPAVTSTSYSQYREKPSIFYQQSLPSSHLTEEAKNVSAVPGPADQKTVIPILPSTFYSHTEKPGVFYQQVLPHSHPTEEALKISVASEPVDQTTGTPTVTSTSYSQHTEKPSIFYQQSLPGSHLTEEAKNVSAVPGPGDRKTGIPTLPSTFYSHTEKPGSFYQQVLPHSHLPEEALEVSVAPGPVDQTIGTPTVTSPSSSFGEKPIVIYKQAFPEGHLPEESLKVSVAPGPVGQTTGAPTITSPSYSQHRAKSGSFYQLALLGSQIPEEALRVSSAPGPADQTTGIPTITSTSYSFGEKPIVNYKQAFPDGHLPEEALKVSIVSGPTEKKTDIPAGPLGSSALGEKPITFYRQALLDSPLNKEVVKVSAAPGPADQKTETLPVHSTSYSNRGKPVIFYQQTLSDSHLPEEALKVPPVPGPDAQKTETPSVSSSLYSYREKPIVFYQQALPDSELTQEALKVSAVPQPADQKTGLSTVTSSFYSHTEKPNISYQQELPDSHLTEEALKVSNVPGPADQKTGVSTVTSTSYSHREKPIVSYQRELPHFTEAGLKILRVPGPADQKTGINILPSNSYPQREHSVISYEQELPDLTEVTLKAIGVPGPADQKTGIQIASSSSYSNREKASIFHQQELPDVTEEALNVFVVPGQGDRKTEIPTVPLSYYSRREKPSVISQQELPDSHLTEEALKVSPVSIPAEQKTGIPIGLSSSYSHSHKEKLKISQQLPDRDQSKGILKISAVPELTDVNTGKPVSLSSSYFHREKSNIFSPQELPGSHVTEDVLKVSTIPGPAGQKTVLPTALPSSFSHREKPDIFYQKDLPDRHLTEDALKISSALGQADQITGLQTVPSGTYSHGENHKLVSE

SEQ ID NO: 98 - DNA, 1200 bp, Homo sapiens
cattgattatttcctttaattatcacacccatcctatgcgatggcctattatcatcgttttataaacacaaagtcacaccactttaagtggtgatgccaggattcaaactccgatctttctgatgccagtgcccagttcttaatccctctaacaacccgtgacacttttccacggttaggttcaagcgcccaccatacagcttgacactttaggagatactcaacctttaaaactttataatttgcatgtgtgattataacgggtacgtcctagagtttgatctcttctctgtgcaggggcgaaactcgaccccagccgagaactacactgtccttgcgcggggccctctgcgccccggaaggcgcccagtcccggtttattgtgagcttacagtacagttcgcaaggtcccggccggtgacggcggcgaggcggcaacgtcgcctgtagcaaacctccgccctaaggcgttcccgccgggcctcgcgcggcgtccctagcaacgcgcgcgcggtcttccggccccgcccaggcgggcggcactgcgcctaagctgggccacaaccgccagtcagggctctccccttcccctccctccccccctcctcctcctcctctgccgcccagagcgagacaccaacatggagcccgaggatctgccatggccgggcgagctggaggaggaggaggaggaggaggaggaggaggaggaggaggaagaggaggaggctgcagcggcggcggcggcgaacgtggacgacgtagtggtcgtggaggaggtggaggaagaggcggggcgggagttggactccgactctcactacgggccccagcatctggaaagtatagacgacgaggaggacgaggaggccaaggcctggctgcaggcgcaccccggcaggattttgcctccgctgtcgcccccgcagcaccgctactcggagggcgagcggacctccctggagaaggtgaggcgggccggggaggggtgtggagccgcggcgagttgggggtggaggctgggccccgagcgctccgcccgcccgcaggtcacgccgcctgacggagaaaacgcgcgcagctgaggctagtaggcggcccagactccagcccaggtgccggccggctgctccgcgtctcccaggctttgtggcaaagacttctcgtttcctccccctcggtgggtcctgggccttttga

SEQ ID NO: 99 - RNA, 10 nt, Artificial Sequence
  RBS
gccaccaugg

SEQ ID NO: 100 - DNA, 51 bp, Homo sapiens
gaggaggaggaggaggaggaggaggaggaggaggaggaggaagaggaggag